(19) United States
(12) Patent Application Publication
(10) Pub. No.: US 2026/0109978 A1
(43) Pub. Date: Apr. 23, 2026

(54) NUCLEIC ACID REGULATORY ELEMENTS FOR CONSTITUTIVE GENE EXPRESSION AND METHODS OF USE

(71) Applicant: MeiraGTx UK II Limited, London (GB)

(72) Inventors: Alexandria Forbes, New York, NY (US); Josefa Sullivan, New York, NY (US); Chenjin Jin, New York, NY (US); Enrico Mossotto, New York, NY (US); Matthew During, Rowayton, CT (US); Ce Feng Liu, New York, NY (US); Dustin Lee, New York, NY (US); Jessica Schmerler, New York, NY (US)

(21) Appl. No.: 19/117,712

(22) PCT Filed: Oct. 11, 2023
(86) PCT No.: PCT/IB2023/060252

§ 371 Date: Apr. 2, 2025

Related U.S. Application Data
(60) Provisional application No. 63/379,138, filed on Oct. 11, 2022.
(60) Provisional application No. 63/496,554, filed on Apr. 17, 2023.

(51) Int. Cl. C12N 15/113; A61K 31/7088; A61K 48/00; C12N 5/071; C12N 5/077; C12N 5/0793; C12N 15/864

(52) U.S. Cl. CPC C12N 15/113; A61K 31/7088; A61K 48/0058; C12N 5/0619; C12N 5/0652; C12N 5/067; C12N 15/8645; C12N 2750/14143; C12N 2830/008; C12N 2830/34; C12N 2830/50

(57) ABSTRACT

The application relates to nucleic acid regulatory elements that are able to enhance expression of genes constitutively in a variety of tissues, or in particular tissues including liver, muscle, and the CNS. The application further relates to methods employing these regulatory elements and uses of these elements. Expression cassettes and vectors containing these nucleic acid regulatory elements are also disclosed. These nucleic acid regulatory elements are particularly useful for applications using gene therapy.

### CROSS-REFERENCE TO RELATED APPLICATIONS

This application claims the benefit under 35 U.S.C. § 119(e) of the earlier filing date of U.S. Provisional Patent Application Ser. No. 63/379,138, filed on Oct. 11, 2022, and U.S. Provisional Patent Application Ser. No. 63/496,554, filed on Apr. 17, 2023, which are hereby incorporated by reference in their entirety.

### REFERENCE TO AN ELECTRONIC SEQUENCE LISTING

The contents of the electronic sequence listing (SeqList-162027-52976.xml; Size: 795,449 bytes; and Date of Creation: Oct. 10, 2023) is herein incorporated by reference in its entirety.

### FIELD

The application relates to nucleic acid regulatory elements that are able to enhance expression of genes in a variety of tissues, or in particular tissues including liver, muscle, and the CNS. The application further relates to methods employing these regulatory elements and uses of these elements. Expression cassettes and vectors containing these nucleic acid regulatory elements are also disclosed. These are particularly useful for applications using gene therapy.

### BACKGROUND

A promoter is a DNA region at which transcription of a gene is initiated. Since the promoter region controls when and where a gene of interest is expressed in an organism, promoters are crucial elements for regulating the level and specificity of transgene expression, particularly in the context of gene therapy.

The use of engineered nucleic acid regulatory elements (NAREs) (that may include various components, including, promoters, enhancers, etc.) that are particularly tailored to a given gene therapy provides a variety of benefits. To start, an optimized NARE allows for levels of gene expression that is desired for a specific therapeutic gene. Second, engineered NAREs with increased potency allow administration of smaller amounts of gene therapy vector, thus decreasing immune responses and associated safety risks. Third, for some gene therapies, it is desirable that gene expression is limited to a specific tissue or tissues. Accordingly, use of nucleic acid regulatory elements with tissue-specific expression can restrict unwanted transgene expression as well as facilitate persistent transgene expression in the tissue(s) or interest. Such tissue-specific NAREs can be used to eliminate the need for tissue-specific viral capsids used for gene delivery (or can be used in combination with tissue-specific viral capsids). Fourth, choosing an appropriate NARE also allows controlling the kinetics of gene expression, which in turn impact durability of gene therapy. Finally, it can be desirable to engineer NAREs with a reduced size (without sacrificing strength or specificity) to allow for efficient packaging of larger transgene cargo into viral vectors.

While efforts have been made to characterize and optimize NARE sequences through conventional low-throughput analyses (rational design) or newer high-throughput methodologies (e.g., MPRA), these approaches still require an expensive and time-consuming in vitro optimization. Accordingly, more efficient methods of engineering NAREs are needed as are nucleic acid regulatory elements with enhanced potency, reduced size, and/or tissue specificity.

### SUMMARY

Provided herein are nucleic acid regulatory elements (NAREs), including NAREs that are particularly suitable for the expression of an operably linked sequence (e.g., a protein or RNA coding sequence) in certain tissues, including liver, muscle, and the central nervous system. Also provided herein are methods for the expression of a transgene that is operably linked to one or more of the nucleic acid regulatory elements disclosed herein.

In embodiments, provided is a polynucleotide sequence that comprises a sequence that is at least 80% identical to a NARE sequence provided in any one of Tables 7-9. In embodiments, provided is a polynucleotide sequence that comprises a sequence that is at least 90% identical to a NARE sequence provided in any one of Tables 7-9. In embodiments, provided is a polynucleotide comprising a NARE sequence provided in any one of Tables 7-9.

In embodiments, provided is a polynucleotide sequence that comprises a sequence that is at least 80% identical to a NARE selected from the group consisting of C6, C7, C7b, C9-C12, C14, C14_L21, C16-C18, C21-C32, C34, C37-C91, C99-C102, C104-C108, C110-C127, C138-C194, and C202-C291 (NAREs are listed as in Table 9). In embodiments, provided is a polynucleotide sequence that comprises a sequence that is at least 90% identical to a NARE selected from the group consisting of C6, C7, C7b, C9-C12, C14, C14_L21, C16-C18, C21-C32, C34, C37-C91, C99-C102, C104-C108, C110-C127, C138-C194, and C202-C291. In embodiments, provided is a polynucleotide sequence that comprises a NARE selected from the group consisting of C6, C7, C7b, C9-C12, C14, C14_L21, C16-C18, C21-C32, C34, C37-C91, C99-C102, C104-C108, C110-C127, C138-C194, and C202-C291.

In embodiments, provided is a polynucleotide sequence that comprises a sequence that is at least 80% identical to a NARE selected from the group consisting of C21-C30 (NAREs are listed as in Table 9). In embodiments, provided is a polynucleotide sequence that comprises a sequence that is at least 90% identical to a NARE selected from the group consisting of C21-C30. In embodiments, provided is a polynucleotide sequence that comprises a NARE selected from the group consisting of C21-C30.

In embodiments, provided is a polynucleotide sequence that comprises a sequence that is at least 80% identical to a NARE selected from the group consisting of C118-C127 (NAREs are listed as in Table 9). In embodiments, provided is a polynucleotide sequence that comprises a sequence that is at least 90% identical to a NARE selected from the group consisting of C118-C127. In embodiments, provided is a polynucleotide sequence that comprises a NARE selected from the group consisting of C118-C127.

In embodiments, provided is a polynucleotide sequence that comprises a sequence that is at least 80% identical to a NARE selected from the group consisting of C139-C158 (NAREs are listed as in Table 9). In embodiments, provided is a polynucleotide sequence that comprises a sequence that is at least 90% identical to a NARE selected from the group consisting of C139-C158. In embodiments, provided is a polynucleotide sequence that comprises a NARE selected from the group consisting of C139-C158.

In embodiments, provided is a polynucleotide sequence that comprises a sequence that is at least 80% identical to a NARE selected from the group consisting of C183-C192 (NAREs are listed as in Table 9). In embodiments, provided is a polynucleotide sequence that comprises a sequence that is at least 90% identical to a NARE selected from the group consisting of C183-C192 In embodiments, provided is a polynucleotide sequence that comprises a NARE selected from the group consisting of C183-C192.

In embodiments, provided is a polynucleotide sequence that comprises a sequence that is at least 80% identical to a NARE selected from the group consisting of C53-C58, C63-C70, C83-C91, and C99-C102 (NAREs are listed as in Table 9). In embodiments, provided is a polynucleotide sequence that comprises a sequence that is at least 90% identical to a NARE selected from the group consisting of C53-C58, C63-C70, C83-C91, and C99-C102. In embodiments, provided is a polynucleotide sequence that comprises a NARE selected from the group consisting of C53-C58, C63-C70, C83-C91, and C99-C102.

In embodiments, provided is a polynucleotide sequence that comprises a sequence that is at least 80% identical to a NARE selected from the group consisting of C53-C58, C63-C70, C83-C91, C99-102, and C108 (NAREs are listed as in Table 9). In embodiments, provided is a polynucleotide sequence that comprises a sequence that is at least 90% identical to a NARE selected from the group consisting of C53-C58, C63-C70, C83-C91, C99-102, and C108. In embodiments, provided is a polynucleotide sequence that comprises a NARE selected from the group consisting of C53-C58, C63-C70, C83-C91, C99-102, and C108.

In embodiments, provided is a polynucleotide sequence that comprises a sequence that is at least 80% identical to a NARE selected from the group consisting of C85, C88, C90, C100-102, C108, C200 and C201 (NAREs are listed as in Table 9). In embodiments, provided is a polynucleotide sequence that comprises a sequence that is at least 90% identical to a NARE selected from the group consisting of C85, C88, C90, C100-102, C108, C200 and C201. In embodiments, provided is a polynucleotide sequence that comprises a NARE selected from the group consisting of C85, C88, C90, C100-102, C108, C200 and C201.

In embodiments, provided is a polynucleotide sequence that comprises a sequence that is at least 80% identical to a NARE selected from the group consisting of C173-C178, C187, C200, C210, and C218 (NAREs are listed as in Table 9). In embodiments, provided is a polynucleotide sequence that comprises a sequence that is at least 90% identical to a NARE selected from the group consisting of C173-C178, C187, C200, C210, and C218. In embodiments, provided is a polynucleotide sequence that comprises a NARE selected from the group consisting of C173-C177, C187, and C218.

Provided is a method for constitutive expression of a transgene that is operably linked to a constitutive NARE disclosed herein.

In embodiments, provided is a polynucleotide sequence that comprises a sequence that is at least 80% identical to a nucleic acid regulatory element selected from the group consisting of C9-C11, C21-C30, C53-C58, C63-C70, C83-C91, C99-C102, and C108 (NAREs are listed as in Table 9). In embodiments, provided is a polynucleotide sequence that comprises a sequence that is at least 90% identical to a nucleic acid regulatory element selected from the group consisting of C9-C11, C21-C30, C53-C58, C63-C70, C83-C91, C99-C102, and C108. In embodiments, provided is a polynucleotide sequence that comprises a nucleic acid regulatory element selected from the group consisting of C9-C11, C21-C30, C53-58, C63-C70, C83-C91, C99-C102, and C108.

Provided herein is a method for expression of a transgene in muscle cells, wherein the transgene is operably linked to a NARE disclosed herein particularly suitable to drive expression in the muscle.

In embodiments, provided is a polynucleotide sequence that comprises a sequence that is at least 80% identical to a nucleic acid regulatory element selected from the group consisting of C14, C16, C21, C23, C26, C36), C42, C46, and C47 (NAREs are listed as in Table 9). In embodiments, provided is a polynucleotide sequence that comprises a sequence that is at least 90% identical to a nucleic acid regulatory element selected from the group consisting of C14, C16, C21, C23, C26, C36), C42, C46, and C47. In embodiments, provided is a polynucleotide sequence that comprises a nucleic acid regulatory element selected from the group consisting of C14, C16, C21, C23, C26, C36, C42, C46, and C47.

In embodiments, provided is a polynucleotide sequence that comprises a sequence that is at least 80% identical to a nucleic acid regulatory element selected from the group consisting of C14, C36, C42, C249-C254, C259, and C260 (NAREs are listed as in Table 9). In embodiments, provided is a polynucleotide sequence that comprises a sequence that is at least 90% identical to a nucleic acid regulatory element selected from the group consisting of C14, C36, C42, C249-C254, C259, and C260. In embodiments, provided is a polynucleotide sequence that comprises a nucleic acid regulatory element selected from the group consisting of C14, C36, C42, C249-C254, C259, and C260.

Provided herein is a method for expression of a transgene in the CNS, wherein the transgene is operably linked to a NARE disclosed herein particularly suitable to drive expression in the CNS. Provided herein is a method for expression of a transgene in neuronal cells, wherein the transgene is operably linked to a NARE disclosed herein particularly suitable to drive expression in neuronal cells.

Provided herein is a polynucleotide that comprises a sequence that is at least 80% identical to a nucleic acid regulatory element selected from the group consisting of C9-C12, C31-C32, C35-C36, C39-C42, C49-C51, and C57-C58 (NAREs are listed as in Table 9). Provided herein is a polynucleotide that comprises a sequence that is at least 90% identical to a nucleic acid regulatory element selected from the group consisting of C9-C12, C31-C32, C35-C36, C39-C42, C49-C51, and C57-C58. Provided herein is a polynucleotide that comprises a nucleic acid regulatory element selected from the group consisting of C9-C12, C31-C32, C35-C36, C39-C42, C49-C51, and C57-C58.

Provided herein is a method for expression of a transgene in the hepatocytes (e.g., in the liver), wherein the transgene is operably linked to a NARE disclosed herein particularly suitable to drive expression in in hepatocytes (e.g., in the liver).

In embodiments, one or more nucleic acid regulatory elements disclosed herein is operably linked to a transgene.

In one aspect, provided is a nucleic acid regulatory element (NARE) comprising: (i) a sequence that is at least 90% identical to any one of SEQ ID NOs:344-349, 498, (ii) a sequence that is at least 90% identical to any one of SEQ ID NOs:337, 338, 355, 357, 358, 359, 407, 411, 429-431, 434, 501, 503, or 504; (iii) optionally, a sequence that is at least 90% identical to any one of SEQ ID NOs: 323, 420, or 488, and (iv) optionally, a sequence that is at least 90% identical to SEQ ID NO:402 or 444. In some embodiments, the NARE comprises: (i) a sequence that is at least 95% identical to any one of SEQ ID NOs:344-349, or 498, (ii) a sequence that is at least 95% identical to any one of SEQ ID NOs:337, 338, 355, 357, 358, 359, 407, 411, 429-431, 434, 501, 503, or 504; (iii) optionally, a sequence that is at least 95% identical to any one of SEQ ID NOs: 323, 420, 488, and (iv) optionally, a sequence that is at least 95% identical to SEQ ID NO:402 or 444. In some embodiments, the NARE comprises: (i) any one of SEQ ID NOs:344-349, or 498, (ii) any one of SEQ ID NOs:337, 338, 355, 357, 358, 359, 407, 411, 429-431, 434, 501, 503, or 504; (iii) optionally, any one of SEQ ID NOs: 323, 420, 488, and (iv) optionally, SEQ ID NO:402 or 444.

In one aspect, provided is a NARE comprising: (i) a sequence that is at least 90% identical to any one of SEQ ID NOs:381, 384, 392, 393, 394, 395, 396, 397, 400, or 401; (ii) a sequence that is at least 90% identical to SEQ ID NO: 334 or SEQ ID NO: 342; and (iii) a sequence that is at least 90% identical to SEQ ID NO:426. In some embodiments, the NARE comprises: (i) a sequence that is at least 95% identical to any one of SEQ ID NOs:381, 384, 392, 393, 394, 395, 396, 397, 400, or 401; (ii) a sequence that is at least 95% identical to SEQ ID NO: 334 or SEQ ID NO: 342; and (iii) a sequence that is at least 95% identical to SEQ ID NO:426. In some embodiments, the NARE comprises: (i) any one of SEQ ID NOs:381, 384, 392, 393, 394, 395, 396, 397, 400, or 401; and (ii) SEQ ID NO: 334 or SEQ ID NO: 342; and (iii) SEQ ID NO:426.

In one aspect, provided is A NARE comprising: (i) a sequence that is at least 90% identical to any one of SEQ ID NOs:344-349, or 498; (ii) a sequence that is at least 90% identical to SEQ ID NO:334 or SEQ ID NO: 342; (iii) a sequence that is at least 90% identical to any one of SEQ ID NOs:302-318, 320-322, 324-327, 331, 332, or 487, (iv) optionally, SEQ ID NO:333; and (v) optionally, a sequence that is at least 90% identical to SEQ ID NO:319. In some embodiments, the NARE comprises: (i) a sequence that is at least 95% identical to any one of SEQ ID NOs:344-349, or 498; (ii) a sequence that is at least 95% identical to SEQ ID NO:334 or SEQ ID NO: 342; (iii) a sequence that is at least 95% identical to any one of SEQ ID NOs:302-318, 320-322, 324-327, 331, 332, or 487, (iv) optionally, SEQ ID NO:333; and (v) optionally, a sequence that is at least 95% identical to SEQ ID NO:319. In some embodiments, the NARE comprises: (i) any one of SEQ ID NOs:344-349, or 498; (ii) SEQ ID NO:334 or SEQ ID NO: 342; (iii) any one of SEQ ID NOs:302-318, 320-322, 324-327, 331, 332, or 487, (iv) optionally, SEQ ID NO:333; and (v) optionally, SEQ ID NO:319.

In some embodiments, the NARE comprises: (i) a sequence that is at least 90% identical to any one of SEQ ID NOs:344-349, or 498; (ii) a sequence that is at least 90% identical to SEQ ID NO: 334 or SEQ ID NO: 342; and (iii) a sequence that is at least 90% identical to any one of SEQ ID NOs:302-317, 320-322, 324-327, 331, 332, or 487. In some embodiments, the NARE comprises: (i) a sequence that is at least 95% identical to any one of SEQ ID NOs:344-349, or 498; (ii) a sequence that is at least 95% identical to SEQ ID NO: 334 or SEQ ID NO: 342; and (iii) a sequence that is at least 95% identical to any one of SEQ ID NOs:302-317, 320-322, 324-327, 331, 332, or 487. In some embodiments, the NARE comprises: (i) any one of SEQ ID NOs:344-349, or 498; (ii) SEQ ID NO: 334 or SEQ ID NO: 342; and (iii) any one of SEQ ID NOs:302-317, 320-322, 324-327, 331, 332, or 487.

In some embodiments, the NARE comprises: (i) a sequence that is at least 90% identical to SEQ ID NOs:344-349, 498; (ii) a sequence that is at least 90% identical to SEQ ID NOs:334 or 342; and (iii) a sequence that is at least 90% identical to any one of SEQ ID NOs:302-317, 320-322, 324-327, 331, or 332. In some embodiments, the NARE comprises: (i) a sequence that is at least 95% identical to SEQ ID NOs:344-349, 498; (ii) a sequence that is at least 95% identical to SEQ ID NOs:334 or 342; and (iii) a sequence that is at least 95% identical to any one of SEQ ID NOs:302-317, 320-322, 324-327, 331, or 332. In some embodiments, the NARE comprises: (i) SEQ ID NOs:344-349, 498; (ii) SEQ ID NOs:334 or 342; and (iii) any one of SEQ ID NOs:302-317, 320-322, 324-327, 331, or 332.

In one aspect, provided is A NARE comprising: (i) a sequence that is at least 90% identical to any one of SEQ ID NOs:344-349, or 498; (ii) a sequence that is at least 90% identical to any one of SEQ ID NOs:351-356, 499, or 500; (iii) a sequence that is at least 90% identical to any one of SEQ ID NOs: 200, 370, 417-420, 502, 441, 482-485, 489, or 491-496; and (iv) optionally, a sequence that is at least 90% identical to any one of SEQ ID NOs:444, 452, or 488; (v) optionally, a sequence that is at least 90% identical to SEQ ID NO:402; and (vi) optionally, SEQ ID NO:433. In some embodiments, the NARE comprises: (i) a sequence that is at least 95% identical to any one of SEQ ID NOs:344-349, or 498; (ii) a sequence that is at least 95% identical to any one of SEQ ID NOs:351-356, 499, or 500; (iii) a sequence that is at least 95% identical to any one of SEQ ID NOs: 200, 370, 417-420, 502, 441, 482-485, 489, or 491-496; and (iv) optionally, a sequence that is at least 95% identical to any one of SEQ ID NOs:444, 452, or 488; (v) optionally, a sequence that is at least 95% identical to SEQ ID NO:402; and (vi) optionally, SEQ ID NO:433. In some embodiments, the NARE comprises: (i) any one of SEQ ID NOs:344-349, or 498; (ii) any one of SEQ ID NOs:351-356, 499, or 500; (iii) any one of SEQ ID NOs: 200, 370, 417-420, 502, 441, 482-485, 489, or 491-496; and (iv) optionally, any one of SEQ ID NOs:444, 452, or 488; (v) optionally, SEQ ID NO:402; and (vi) optionally, SEQ ID NO:433.

In some embodiments, the NARE comprises: (i) a sequence that is at least 90% identical to any one of SEQ ID NOs:344-349, or 498; (ii) a sequence that is at least 90% identical to any one of SEQ ID NOs:351-356, 499, or 500; (iii) a sequence that is at least 90% identical to any one of SEQ ID NOs: 200, 370, 417-420, 502, 482-485, 489, or 491-496; and (iv) optionally, a sequence that is at least 90% identical to SEQ ID NOs:444 or 452. In some embodiments, the NARE comprises: (i) a sequence that is at least 95% identical to any one of SEQ ID NOs:344-349, or 498; (ii) a sequence that is at least 95% identical to any one of SEQ ID NOs:351-356, 499, or 500; (iii) a sequence that is at least 95% identical to any one of SEQ ID NOs: 200, 370, 417-420, 502, 482-485, 489, or 491-496; and (iv) optionally, a sequence that is at least 95% identical to SEQ ID NOs:444 or 452. In some embodiments, the NARE comprises: (i) any one of SEQ ID NOs:344-349, 498; (ii) any one of SEQ ID NOs:351-356, 499, or 500; (iii) any one of SEQ ID NOs: 200, 370, 417-420, 502, 482-485, 489, or 491-496; and (iv) optionally, SEQ ID NOs:444 or 452.

In some embodiments, the NARE comprises: (i) a sequence that is at least 90% identical to any one of SEQ ID NOs:344-349, or 498; (ii) a sequence that is at least 90% identical to any one of SEQ ID NOs:351-356, 499, or 500; (iii) a sequence that is at least 90% identical to any one of SEQ ID NOs:200, 417-420, 502, or 485; and (iv) a sequence that is at least 90% identical to optionally, SEQ ID NOs:444 or 452. In some embodiments, the NARE comprises: (i) a sequence that is at least 95% identical to any one of SEQ ID NOs:344-349, or 498; (ii) a sequence that is at least 95% identical to any one of SEQ ID NOs:351-356, 499, or 500; (iii) a sequence that is at least 95% identical to any one of SEQ ID NOs:200, 417-420, 502, or 485; and (iv) a sequence that is at least 95% identical to optionally, SEQ ID NOs:444 or 452. In some embodiments, the NARE comprises: (i) any one of SEQ ID NOs:344-349, 498; (ii) any one of SEQ ID NOs:351-356, 499, or 500; (iii) any one of SEQ ID NOs:200, 417-420, 502, or 485; and (iv) optionally, SEQ ID NOs:444 or 452.

In some embodiments, the NARE comprises: (i) a sequence that is at least 90% identical to any one of SEQ ID NOs:344-349, or 498; (ii) a sequence that is at least 90% identical to any one of SEQ ID NOs:351-356, 499, or 500; and (iii) a sequence that is at least 90% identical to any one of SEQ ID NOs:370, 482-484, 489, or 491-496. In some embodiments, the NARE comprises: (i) a sequence that is at least 95% identical to any one of SEQ ID NOs:344-349, or 498; (ii) a sequence that is at least 95% identical to any one of SEQ ID NOs:351-356, 499, or 500; and (iii) a sequence that is at least 95% identical to any one of SEQ ID NOs:370, 482-484, 489, or 491-496. In some embodiments, the NARE comprises: (i) any one of SEQ ID NOs:344-349, 498; (ii) any one of SEQ ID NOs:351-356, 499, or 500; and (iii) any one of SEQ ID NOs:370, 482-484, 489, or 491-496.

In one aspect, provided is a NARE comprising: (i) a sequence that is at least 90% identical to any one of SEQ ID NOs:344-349, or 498; (ii) a sequence that is at least 90% identical to SEQ ID NO:335; and (iii) a sequence that is at least 90% identical to any one of SEQ ID NOs: 294, 295, 296, 299, 323, 328, 329, 330, 407, 501, 430, 431, 503, 434, or 504; and (iv) optionally, a sequence that is at least 90% identical to SEQ ID NO:444. In some embodiments, the NARE comprises: (i) a sequence that is at least 95% identical to any one of SEQ ID NOs:344-349, or 498; (ii) a sequence that is at least 95% identical to SEQ ID NO:335; and (iii) a sequence that is at least 95% identical to any one of SEQ ID NOs: 294, 295, 296, 299, 323, 328, 329, 330, 407, 501, 430, 431, 503, 434, or 504; and (iv) optionally, a sequence that is at least 95% identical to SEQ ID NO:444. In some embodiments, the NARE comprises: (i) any one of SEQ ID NOs:344-349, or 498; (ii) SEQ ID NO:335; and (iii) any one of SEQ ID NOs: 294, 295, 296, 299, 323, 328, 329, 330, 407, 501, 430, 431, 503, 434, or 504; and (iv) optionally, SEQ ID NO:444.

In some embodiments, the NARE comprises: (i) a sequence that is at least 90% identical to any one of SEQ ID NOs:344-349, or 498; (ii) a sequence that is at least 90% identical to SEQ ID NO:335; and (iii) a sequence that is at least 90% identical to any one of SEQ ID NOs: 294, 295, 296, 407, 501, 430, 431, 503, 434, or 504; and (iv) a sequence that is at least 90% identical to SEQ ID NO:444. In some embodiments, the NARE comprises: (i) a sequence that is at least 95% identical to any one of SEQ ID NOs:344-349, or 498; (ii) a sequence that is at least 95% identical to SEQ ID NO:335; and (iii) a sequence that is at least 95% identical to any one of SEQ ID NOs: 294, 295, 296, 407, 501, 430, 431, 503, 434, or 504; and (iv) a sequence that is at least 95% identical to SEQ ID NO:444. In some embodiments, the NARE comprises: (i) any one of SEQ ID NOs:344-349, or 498; (ii) SEQ ID NO:335; and (iii) any one of SEQ ID NOs: 294, 295, 296, 407, 501, 430, 431, 503, 434, or 504; and (iv) SEQ ID NO:444.

In one aspect, provided is a NARE comprising: (i) a sequence that is at least 90% identical to any one of SEQ ID NOs:339, 363-369, 378, 379, 382, 385, 387-389, 391, 398, or 399; and (ii) a sequence that is at least 90% identical to any one of SEQ ID NOs:430, 431, or 503. In some embodiments, the NARE comprises: (i) a sequence that is at least 95% identical to any one of SEQ ID NOs:339, 363-369, 378, 379, 382, 385, 387-389, 391, 398, or 399; and (ii) a sequence that is at least 95% identical to any one of SEQ ID NOs:430, 431, or 503. In some embodiments, the NARE comprises: (i) any one of SEQ ID NOs:339, 363-369, 378, 379, 382, 385, 387-389, 391, 398, or 399; and (ii) any one of SEQ ID NOs:430, 431, or 503.

In one aspect, provided is a NARE comprising a sequence that is at least 90% identical to any one of SEQ ID NOs:138-161. In some embodiments, the NARE comprises a sequence that is at least 95% identical to any one of SEQ ID NOs:138-161. In some embodiments, the NARE comprises any one of SEQ ID NOs:138-161.

In one aspect, provided is a NARE comprising: (i) a sequence that is at least 90% identical to SEQ ID NO:298; (ii) a sequence that is at least 90% identical to SEQ ID NO:444; and (iii) optionally, a sequence that is at least 90% identical to SEQ ID NO:475. In some embodiments, the NARE comprises: (i) a sequence that is at least 95% identical to SEQ ID NO:298; (ii) a sequence that is at least 95% identical to SEQ ID NO:444; and (iii) optionally, a sequence that is at least 95% identical to SEQ ID NO:475. In some embodiments, the NARE comprises: (i) SEQ ID NO:298; (ii) SEQ ID NO:444, and (iii) optionally, SEQ ID NO:475.

In one aspect, provided is a NARE comprising: (i) a sequence that is at least 90% identical to any one of SEQ ID NOs:339, 380, 383, 386, or 390; (ii) a sequence that is at least 90% identical to SEQ ID NOs:439 or 497; and (iii) optionally, a sequence that is at least 90% identical to SEQ ID NO:444. In some embodiments, the NARE comprises: (i) a sequence that is at least 95% identical to any one of SEQ ID NOs:339, 380, 383, 386, or 390; (ii) a sequence that is at least 95% identical to SEQ ID NOs:439 or 497; and (iii) optionally, a sequence that is at least 95% identical to SEQ ID NO:444. In some embodiments, the NARE comprises: (i) any one of SEQ ID NOs:339, 380, 383, 386, or 390; (ii) SEQ ID NOs:439 or 497; and (iii) optionally, SEQ ID NO:444.

In some embodiments, the NARE comprises: (i) a sequence that is at least 90% identical to any one of SEQ ID NOs:339, 380, 383, 386, or 390; and (ii) a sequence that is at least 90% identical to SEQ ID NOs:439 or 497. In some embodiments, the NARE comprises: (i) a sequence that is at least 95% identical to any one of SEQ ID NOs:339, 380, 383, 386, or 390; and (ii) a sequence that is at least 95% identical to SEQ ID NOs:439 or 497. In some embodiments, the NARE comprises: (i) any one of SEQ ID NOs:339, 380, 383, 386, or 390; and (ii) SEQ ID NOs:439 or 497.

In one aspect, provided is a NARE comprising: (i) a sequence that is at least 90% identical to any one of SEQ ID NOs:344-349, or 498; (ii) a sequence that is at least 90% identical to SEQ ID NO:336; and (iii) a sequence that is at least 90% identical to any one of SEQ ID NOs:201, 284, 285, 301, 362, 372, 373, 403-406, 408-410, 412, 427, 444, 453-456, 486, or 490. In some embodiments, the NARE comprises: (i) a sequence that is at least 95% identical to any one of SEQ ID NOs:344-349, or 498; (ii) a sequence that is at least 95% identical to SEQ ID NO:336; and (iii) a sequence that is at least 95% identical to any one of SEQ ID NOs:201, 284, 285, 301, 362, 372, 373, 403-406, 408-410, 412, 427, 444, 453-456, 486, or 490. In some embodiments, the NARE comprises: (i) any one of SEQ ID NOs:344-349, or 498; (ii) SEQ ID NO:336; and (iii) any one of SEQ ID NOs:201, 284, 285, 301, 362, 372, 373, 403-406, 408-410, 412, 427, 444, 453-456, 486, or 490.

In one aspect, provided is a NARE comprising: (i) a sequence that is at least 90% identical to any one of SEQ ID NOs:374-376; (ii) optionally, a sequence that is at least 90% identical to any one of SEQ ID NOs:344-349, 498 or 393; (iii) a sequence that is at least 90% identical to any one of SEQ ID NOs:93-98, 103, 109, 128-136, 195-198, 201, 284, 285, 301, 362, 371, 373, 403-406, 408-410, 412, 417-420, 502, 427, 453-456, 460-474, 486, or 490; and (iv) optionally, a sequence that is at least 90% identical to SEQ ID NOs:443 or 448. In some embodiments, the NARE comprises: (i) a sequence that is at least 95% identical to any one of SEQ ID NOs:374-376; (ii) optionally, a sequence that is at least 95% identical to any one of SEQ ID NOs:344-349, 498 or 393; (iii) a sequence that is at least 95% identical to any one of SEQ ID NOs:93-98, 103, 109, 128-136, 195-198, 201, 284, 285, 301, 362, 371, 373, 403-406, 408-410, 412, 417-420, 502, 427, 453-456, 460-474, 486, or 490; and (iv) optionally, a sequence that is at least 95% identical to SEQ ID NOs:443 or 448. In some embodiments, the NARE comprises: (i) any one of SEQ ID NOs:374-376; (ii) optionally, any one of SEQ ID NOs:344-349, 498 or 393; (iii) any one of SEQ ID NOs:93-98, 103, 109, 128-136, 195-198, 201, 284, 285, 301, 362, 371, 373, 403-406, 408-410, 412, 417-420, 502, 427, 453-456, 460-474, 486, or 490; and (iv) optionally, SEQ ID NOs:443 or 448.

In some embodiments, the NARE comprises: (i) a sequence that is at least 90% identical to any one of SEQ ID NOs:374-376; and (ii) a sequence that is at least 90% identical to any one of SEQ ID NOs:93-98, 103, 109, 128-136, 195-198, 201, 284, 285, 301, 362, 371, 373, 403-406, 408-410, 412, 427, 453-456, 460-474, 486, or 490. In some embodiments, the NARE comprises: (i) a sequence that is at least 95% identical to any one of SEQ ID NOs:374-376; and (ii) a sequence that is at least 95% identical to any one of SEQ ID NOs:93-98, 103, 109, 128-136, 195-198, 201, 284, 285, 301, 362, 371, 373, 403-406, 408-410, 412, 427, 453-456, 460-474, 486, or 490. In some embodiments, the NARE comprises: (i) any one of SEQ ID NOs:374-376; and (ii) any one of SEQ ID NOs:93-98, 103, 109, 128-136, 195-198, 201, 284, 285, 301, 362, 371, 373, 403-406, 408-410, 412, 427, 453-456, 460-474, 486, or 490.

In some embodiments, the NARE comprises: (i) a sequence that is at least 90% identical to any one of SEQ ID NOs:374-376; and (ii) a sequence that is at least 90% identical to any one of SEQ ID NOs:201, 284, 285, 301, 362, 371, 373, 403-406, 408-410, 412, 427, 453-456, 486, or 490. In some embodiments, the NARE comprises: (i) a sequence that is at least 95% identical to any one of SEQ ID NOs:374-376; and (ii) a sequence that is at least 95% identical to any one of SEQ ID NOs:201, 284, 285, 301, 362, 371, 373, 403-406, 408-410, 412, 427, 453-456, 486, or 490. In some embodiments, the NARE comprises: (i) any one of SEQ ID NOs:374-376; and (ii) any one of SEQ ID NOs:201, 284, 285, 301, 362, 371, 373, 403-406, 408-410, 412, 427, 453-456, 486, or 490.

In some embodiments, the NARE comprises: (i) a sequence that is at least 90% identical to any one of SEQ ID NOs:374-376; and (ii) a sequence that is at least 90% identical to any one of SEQ ID NOs:93-98, 103, 109, 128-136, 195-198, or 460-474. In some embodiments, the NARE comprises: (i) a sequence that is at least 95% identical to any one of SEQ ID NOs:374-376; and (ii) a sequence that is at least 95% identical to any one of SEQ ID NOs:93-98, 103, 109, 128-136, 195-198, or 460-474. In some embodiments, the NARE comprises: (i) any one of SEQ ID NOs:374-376; and (ii) any one of SEQ ID NOs:93-98, 103, 109, 128-136, 195-198, or 460-474.

In some embodiments, the NARE comprises: (i) a sequence that is at least 90% identical to any one of SEQ ID NOs:344-349, 498, or 393; (ii) a sequence that is at least 90% identical to any one of SEQ ID NOs:374-376; (iii) a sequence that is at least 90% identical to any one of SEQ ID NOs:417-420, or 502; and (iv) optionally, a sequence that is at least 90% identical to SEQ ID NOs:443 or 448. In some embodiments, the NARE comprises: (i) a sequence that is at least 95% identical to any one of SEQ ID NOs:344-349, 498 or 393; (ii) a sequence that is at least 95% identical to any one of SEQ ID NOs:374-376; (iii) a sequence that is at least 95% identical to any one of SEQ ID NOs:417-420, or 502; and (iv) optionally, a sequence that is at least 95% identical to SEQ ID NOs:443 or 448. In some embodiments, the NARE comprises: (i) any one of SEQ ID NOs:344-349, 498 or 393; (ii) any one of SEQ ID NOs:374-376; (iii) any one of SEQ ID NOs:417-420, or 502; and (iv) optionally, SEQ ID NOs:443 or 448.

In one aspect, provided is a NARE comprising: (i) a sequence that is at least 90% identical to SEQ ID NOs:478 or 479; (ii) a sequence that is at least 90% identical to SEQ ID NO:350; (iii) a sequence that is at least 90% identical to any one of SEQ ID NOs:351-356, 499, or 500; and (iv) a sequence that is at least 90% identical to any one of SEQ ID NOs:361, 421, 422, 423, 428, 444, or 480. In some embodiments, the NARE comprises: (i) a sequence that is at least 95% identical to SEQ ID NOs:478 or 479; (ii) a sequence that is at least 95% identical to SEQ ID NO:350; (iii) a sequence that is at least 95% identical to any one of SEQ ID NOs:351-356, 499, or 500; and (iv) a sequence that is at least 95% identical to any one of SEQ ID NOs:361, 421, 422, 423, 428, 444, or 480. In some embodiments, the NARE comprises: (i) SEQ ID NOs:478 or 479; (ii) SEQ ID NO:350; (iii) any one of SEQ ID NOs:351-356, 499, or 500; and (iv) any one of SEQ ID NOs:361, 421, 422, 423, 428, 444, or 480.

In one aspect, provided is a NARE comprising: (i) a sequence that is at least 90% identical to SEQ ID NO:377; and (ii) a sequence that is at least 90% identical to any one of SEQ ID NOs:444, 457, 458, 469, or 470. In some embodiments, the NARE comprises: (i) a sequence that is at least 95% identical to SEQ ID NO:377; and (ii) a sequence that is at least 95% identical to any one of SEQ ID NOs:444, 457, 458, 469, or 470. In some embodiments, the NARE comprises: (i) SEQ ID NO:377; and (ii) any one of SEQ ID NOs:444, 457, 458, 469, or 470.

In one aspect, provided is a NARE comprising: (i) a sequence that is at least 90% identical to any one of SEQ ID NOs:199, 476, or 477; and (ii) a sequence that is at least 90% identical to SEQ ID NO:300. In some embodiments, the NARE comprises: (i) a sequence that is at least 95% identical to any one of SEQ ID NOs:199, 476, or 477; and (ii) a sequence that is at least 95% identical to SEQ ID NO:300. In some embodiments, the NARE comprises: (i) any one of SEQ ID NOs:199, 476, or 477; and (ii) SEQ ID NO:300.

In one aspect, provided is a NARE comprising: (i) a sequence that is at least 90% identical to SEQ ID NO:474.; (ii) a sequence that is at least 90% identical to SEQ ID NO:340 or 341; and (iii) optionally, a sequence that is at least 90% identical to SEQ ID NO:393. In some embodiments, the NARE comprises: (i) a sequence that is at least 95% identical to SEQ ID NO:474.; (ii) a sequence that is at least 95% identical to SEQ ID NO:340 or 341; and (iii) optionally, a sequence that is at least 95% identical to SEQ ID NO:393. In some embodiments, the NARE comprises: (i) NO:474.; (ii) SEQ ID NO:340 or 341; and (iii) optionally, SEQ ID NO:393.

In one aspect, provided is a NARE comprising a sequence that is at least 90% identical to any one of SEQ ID NOs: 6, 7, 9-12, 14, 16-18, 21-32, 34, 37-91, 99-102, 104-108, 110-127, 138-194, 202-283, or 286-293. In some embodiments, the NARE comprises a sequence that is at least 95% identical to any one of SEQ ID NOs: 6, 7, 9-12, 14, 16-18, 21-32, 34, 37-91, 99-102, 104-108, 110-127, 138-194, 202-283, or 286-293. In some embodiments, the NARE comprises any one of SEQ ID NOs: 6, 7, 9-12, 14, 16-18, 21-32, 34, 37-91, 99-102, 104-108, 110-127, 138-194, 202-283, or 286-293. In some embodiments, the NARE comprises a sequence that is at least 90% identical to any one of SEQ ID NOs: 6, 14, 16, 18, 34, 48, 104-107, 162-172, 179, 180, 183, 184, or 293. In some embodiments, the NARE comprises a sequence that is at least 95% identical to any one of SEQ ID NOs: 6, 14, 16, 18, 34, 48, 104-107, 162-172, 179, 180, 183, 184, or 293. In some embodiments, the NARE comprises any one of SEQ ID NOs: 6, 14, 16, 18, 34, 48, 104-107, 162-172, 179, 180, 183, 184, or 293.

In one aspect, provided is an expression construct comprising a NARE disclosed herein and an operatively linked transgene. In one embodiment, the expression construct further comprises a polyadenylation sequence.

In one aspect, provided is a vector comprising an expression construct disclosed herein. In one embodiment, the vector is a non-viral vector. In one embodiment, the vector is a viral vector. In one embodiment, the vector is an adeno-associated virus (AAV) vector. In one embodiment, the vector comprises a nucleic acid sequence comprising (i) the expression construct disclosed herein and (ii) one or more inverted terminal repeats (ITR). In one embodiment, the vector comprises a 5′ ITR and a 3′ ITR. In one embodiment, the 5′ ITR and a 3′ ITR are derived from AAV serotype AAV2.

In one aspect, provided is a cell comprising an expression construct or a vector disclosed herein. In some embodiments, the cell is a neuronal, muscle, or liver cell.

In one aspect, provided is a pharmaceutical composition comprising (i) an expression construct disclosed herein or a vector disclosed herein and (ii) a pharmaceutically acceptable excipient.

In one aspect, provided is a method for expressing a transgene in a cell comprising an expression construct disclosed herein or a vector disclosed herein. In one aspect, provided is method for regulating transgene expression in a cell comprising an expression construct disclosed herein or a vector disclosed herein. In some embodiments, the cell is a neuronal, muscle, or liver cell. In one aspect, provided is a method of treating a disease comprising administering to a subject in need an expression construct disclosed herein, a vector disclosed herein, or a pharmaceutical composition disclosed herein, wherein the expression construct, vector, or pharmaceutical composition comprises a transgene useful for treating the disease.

### BRIEF DESCRIPTION OF THE FIGURES

FIG. 1. Diagram of bioinformatics pipeline for the design and optimization of a given promoter sequence utilizing AI. The pipeline is divided in two parallel approaches: (i) Promoter Enhancement, whereby all known/annotated enhancer fragments were collected from the ENCODE database and inserted upstream the promoter sequence; and (ii) Saturation Mutagenesis, in which all possible point mutations (no indels) were generated in silico for each base pair in the original input promoter sequence. Best candidates from both approaches were then tested in vitro in the preferred cell line and activities recorded through fluorescence. Best in vitro performers from both strategies were then combined through rational design in a final in vitro screen.

FIGS. 2A and 2B. Dual reporter design and measurement. FIG. 2A illustrates the design of the dual reporter construct. Candidate nucleic acid regulatory elements were cloned upstream (5′) of the mClover3 coding sequence. Each construct contains a constant region that includes a tdTomato transgene that is used as a normalization control. FIG. 2B provides an example of flow cytometry data obtained with the dual reporter system, in which mClover3 expression in tdTomato+ cells reflects the level of NARE activity.

FIGS. 3A, 3B, and 3C. Generation of nucleic acid regulatory elements obtained by modification of the CMV enhancer region of the CAG promoter. FIG. 3A: Schematic of CAG enhancer modifications that were performed. FIG. 3B: Comparison of selected CAG-derived nucleic acid regulatory elements. Expression data was obtained via flow cytometry using the dual reporter system illustrated in FIG. 2B. Expression is provided relative to expression driven by the CAG (C19) promoter in various transfected cell lines. The indicated CAG-derived nucleic acid regulatory elements resulted in up to four-fold improvement in potency in muscle and neuronal cell lines as compared to the CAG promoter while reducing the nucleic acid regulatory element size by up to ˜900 bp. FIG. 3C: Tissue/cell selective expression of indicated nucleic acid regulatory elements in various cell lines. Cell line species is indicated as (H) for human or (M) for mouse.

FIGS. 4A and 4B. Generation of nucleic acid regulatory elements obtained by modification of the P actin promoter region and/or intron of the CAG promoter. FIG. 4A: Expression of indicated nucleic acid regulatory elements in HEK293T cells relative to the CAG promoter in the dual reporter system. FIG. 4B: Potency of the indicated nucleic acid regulatory elements relative to the CAG promoter in the dual reporter system. The indicated nucleic acid regulatory elements exhibit sequential modifications (from left to right) that improved expression of the reporter transgene.

FIGS. 5A, 5B, 5C, and 5D. In vitro validation of nucleic acid regulatory elements designed with methods described herein. FIG. 5A: Results from the first generation of nucleic acid regulatory elements showed that most of the Enhancer+ NARE constructs perform as well, or better, than the parent JeTmd (C6) promoter. In particular, nucleic acid regulatory elements C101 and C124 exhibited an improved potency of about 30% and about 40% respectively as compared to C6. Similarly, point mutations resulted in a boost in performance between about 10% and about 30%. Hyper-mutated nucleic acid regulatory elements (with either an iterative or greedy approach) disrupted NARE activity in HEK293T cells. Based on results from first generation constructs, a new set of nucleic acid regulatory elements was designed combining those elements that had provided the highest in vitro expression (marked with an asterisk). The second generation was tested in HEK293T cells. All nucleic acid regulatory elements exhibited higher expression as compared to the parent C6. In particular, nucleic acid regulatory element C187 provided about 2-fold higher expression compared to the parent C6. Expression levels were tested using the dual reporter system using flow cytometry. FIG. 5B: Potency of a third generation of nucleic acid regulatory elements as compared to the CMV promoter (C1) and the CAG promoter (C19) as determined in HEK293T cells using the dual reporter system. FIG. 5C: Potency of selected nucleic acid regulatory elements as compared to the CMV promoter (C1) and the CAG promoter (C19) as determined in HEK293T cells using the dual reporter system. FIG. 5D: Potency of selected nucleic acid regulatory elements as compared to the CMV promoter (C1), the CAG promoter (C19), and the JeTmd (C6) promoter as determined in HEK293T cells using the dual reporter system.

FIG. 6. In vivo expression of NARE in the muscle. The gastrocnemius muscle of C57BL/6 mice was transduced with AAV8-eGFP constructs driven by either reference promoter CAG or NARE C85 via IM injection at a dose of 5×10¹⁰ GC/muscle. The gastrocnemius muscle was sectioned five weeks post-injection and six serial slices were imaged for eGFP fluorescence/expression.

FIGS. 7A, 7B, 7C and 7D. Potency of selected NAREs in muscle cells. FIG. 7A: Potency of selected synthetic CAG-based dual NAres as compared to the CMV promoter (C1) and the CAG promoter (C19) in C2C12 cells. FIG. 7B: Primary human myotubes were transduced with AAV8-miRFP713-WPRE-SV40polyA containing the indicated NAREs. FIG. 7C: Potency of indicated NAREs as compared to the CAG promoter using AAV8-eGFP-WPRE3-bGHpolyA (C19) in C2C12 myoblast cells. FIG. 7D: Potency of the indicated nucleic acid regulatory elements in C2C12 cells relative to CAG using the dual reporter system in primary human myotubes. C8, C9, C10 and C11 provide an example of a series of sequential modifications resulting in small, ubiquitous NARE, C11, with 5-fold higher activity than CAG in vitro. C8 is the parent ubiquitous promoter (AdML), nucleic acid regulatory element C9 adds an enhancer, nucleic acid regulatory element C10 adds a start site modification to C9, and nucleic acid regulatory element C11 adds an intron to C10.

FIGS. 8A, 8B, 8C, 8D, 8E, 8F, 8G, 8H, and 8I. Potency of selected NAREs in neuronal cells. FIG. 8A: Potency of selected NAREs measured using the dual reporter assay in transfected N2a cells, a mouse neuroblastoma cell line. FIG. 8B: Potency of selected hSyn1-derived NAREs in transfected N2a cells as compared to the CMV promoter (C1), the CAG promoter (C19), and the hSyn1_v2 (B5) promoter. FIG. 8C: Tissue specificity for selected NAREs (relative expression in N2a cells v. relative expression in HEK293T cells). FIG. 8D: Potency of NSEmin (B2)-derived NAREs as compared to the CMV promoter (C1), CAG promoter (C19), and NSEmin promoter (B2) in transfected N2a cells. FIG. 8E: Tissue specificity for selected NAREs (relative expression in N2a cells v. relative expression in HEK293T cells). FIG. 8F: Relative expression of mIRFP713 from NAREs normalized to the synapsin promoter in transduced primary mouse cortical neurons. FIG. 8G: Potency of selected NAREs in transfected BE2M17 cells. FIG. 8H and FIG. 8I: Potency of selected NAREs in transfected N2a cells. FIG. 8J: Relative expression of NAREs in mouse N2A (left bars), human HEK293T (middle bars) and human HuH7 (right bars) cells using the dual reporter assay.

FIGS. 9A, 9B, and 9C. Expression and specificity of selected NAREs in primary neuronal culture. FIG. 9A: On day 4 in vitro, primary mouse cortical neurons were transduced at 50,000 multiplicity of infection (MOI) with AAV9-miRFP713 driven by candidate NAREs. After 7 days, neurons were imaged to assess miRFP713. FIG. 9B: Transduction efficiency was consistent across constructs. At 7 days post infection (DPI), all NAREs lead to higher expression of miRFP713 than synapsin (hSyn) and showed fewer miRFP713-positive astrocytes than CAG. FIG. 9C: Representative images of neuronal specificity by immunocytochemistry (miRFP713, NeuN, GFAP indicated). Arrows indicate miRFP713-negative astrocytes. Scale bar: 50 μm.

FIGS. 10A, 10B, and 10C. Selected NAREs are highly specific for neuronal cells in vivo. Male C57Bl/6J mice were injected intravenously with 5e12 GC/kg AAV9-miRFP713 driven by candidate NAREs. After 4 weeks, fluorescence was measured for each organ ex vivo and genomes harvested for qPCR to quantify transduction. FIG. 10A shows representative images of ex vivo fluorescence of whole brain showing higher expression driven by selected NAREs compared to CAG. FIGS. 10B and 10C: Peripheral tissue expression normalized to viral genomes shows low off-target expression in the mouse liver and heart driven by NAREs.

FIGS. 11A and 11B. Potency of selected nucleic acid regulatory elements in liver cells in vitro. FIG. 11A: Numerous of the indicated nucleic acid regulatory elements were ≥2-fold stronger than CAG in liver hepatocytes. Numerous of the indicated nucleic acid regulatory elements were ≥30% stronger than CAG in liver hepatocytes with C31 being 9-fold stronger than CAG. Expression levels were tested using the dual reporter system using flow cytometry and presented relative to CAG. FIG. 11B: Primary mouse hepatocytes were transduced with AAV8-miRFP713-WPRE-SV40polyA containing the indicated nucleic acid regulatory elements.

FIG. 12. Nucleic acid regulatory elements C174 and C177 drive sustained gene expression in the liver in vivo. Expression was analyzed 85 days after virus was injected i.v.

### DETAILED DESCRIPTION

Nucleic acid regulatory elements (NAREs) including promoters are essential components of a gene therapy that control the expression level and durability of a therapeutic gene. NAREs can drive cell-specific expression of transgenes independent of, for example, capsid choice. Incorporation of stronger NAREs can increase potency and efficacy at lower viral vector doses, thereby potentially decreasing safety risks, immune responses, and reducing cost of vector production. Moreover, reducing NARE size while maintaining strength and specificity allows efficient packaging of larger transgenes or expression cassettes into AAV. The present application provides libraries of nucleic acid regulatory elements to improve the safety, efficacy, and durability of therapeutic transgene expression.

While many NAREs and particularly promoter sequences have been characterized/optimized through conventional low-throughput analyses (rational design) or newer high-throughput methodologies (e.g., MPRA), these approaches still require an expensive and time-consuming in vitro optimization. Here, using advanced artificial intelligence models, a convolutional neural network (CNN) was repurposed and optimized to predict NARE potency. A library of nucleic acid regulatory elements was computationally constructed by cloning all known enhancer elements reported in the ENCODE database upstream of a potent, compact constitutive promoter. Subsequently, the correct spacing between the particularly well-performing enhancer elements and the promoter sequence was optimized. In parallel, in silico saturated mutability was performed, whereby all possible point mutations within the NARE sequence were introduced and those with the best impact on the NARE's potency were selected. Particularly well-performing performers were produced and tested in vitro. While most of the nucleic acid regulatory elements exhibited better or equal performance than the original promoter, the best enhancer elements and point mutation were selected and combined in a second optimization round. After synthesizing and testing this new set in vitro, enhanced nucleic acid regulatory elements that exhibit increased potency were obtained.

### NAREs

Provided herein are NAREs that may include as part of their sequence a promoter and/or an enhancer. Traditionally, promoters are defined as DNA regions where transcription is initiated. Promoters include specific DNA motifs that transcription factors (TFs) and their complexes can access. On the other hand, enhancers are defined as DNA regions that amplify transcription initiation by directly interplaying with their target promoters. Likewise, the enhancer sequences, distal from their target promoters, contain DNA motifs that act as binding sites for TFs and cofactors. At times, the term promoter may be used as a shorthand to refer to a nucleic acid sequence that comprises multiple regulatory elements. CAG, for example, contains a CMV enhancer, R actin promoter region and intron, but may be referred to as a promoter. Specifically, the CAG promoter consists of (1) the cytomegalovirus (CMV) early enhancer element, (2) the promoter, the first exon and the first intron of chicken beta-actin gene, and (3) the splice acceptor of the rabbit beta-globin gene. As used herein, the term “nucleic acid regulatory element” (NARE) can refer to a promoter defined in the traditional sense as well as a combination of elements that comprises a promoter and/or other nucleic acid regulatory elements that modulate the expression of sequence that encodes an RNA or protein that is operably linked to the element(s). A nucleic acid regulatory element may be, or include one or more of, e.g., promoters, enhancers, translation initiation signals, introns, and/or splicing enhancers.

As used herein, the term “NARE” can refer to a promoter defined in the traditional sense as well as a combination of nucleic acid regulatory elements that comprises a promoter and/or other nucleic acid regulatory elements that modulate the expression of a gene that is operably linked to the element(s). A nucleic acid regulatory element may be, or include one or more of, e.g., promoters, enhancers, translation initiation signals, introns, and/or splicing enhancers.

As used herein, “transgene” refers to a gene (in particular the coding sequence of the gene) that is transferred into one or more cells of an organism, for example using a vector described herein. A transgene can encode a protein or RNA that is normally expressed in cells of the target organism, or may encode a protein or RNA from a different organism. The transgene may be integrated into the genome of the target cell, or may exist as part of an extrachromosomal expression construct.

As used herein, “operatively linked” refers to a first molecule joined to a second molecule, wherein the molecules are so arranged that the first molecule affects the function of the second molecule. The two molecules may or may not be part of a single contiguous molecule and may or may not be adjacent. For example, a NARE is operatively linked to a transcribable polynucleotide molecule if the NARE modulates transcription of the transcribable polynucleotide molecule of interest in a cell. Additionally, two portions of a transcription regulatory element are operatively linked to one another if they are joined such that the transcription-activating functionality of one portion is not adversely affected by the presence of the other portion. Two transcription regulatory elements may be operatively linked to one another by way of a linker nucleic acid (e.g., an intervening non-coding nucleic acid) or may be operatively linked to one another with no intervening nucleotides present.

Provided herein are engineered regulatory elements that can drive transgene expression in a variety of different tissues, including the muscle, central nervous system, and liver. For example, enhanced CAG-derived nucleic acid regulatory elements were developed that are both stronger and smaller than the original CAG sequence. The NARE engineering platform described herein has yielded numerous nucleic acid regulatory elements that can be used to drive the expression of numerous therapeutic transgenes. This platform has also been applied to different tissues and cell types of interest, including the muscle, the central nervous system and liver, as disclosed herein.

### Constitutive NAREs

This disclosure provides nucleic acid regulatory elements for constitutive expression of an operably linked transgene, that is, expression of the transgene is maintained at a constant level. Constitutive NAREs may drive expression of an operably linked transgene in a variety of cell types and tissues. As used herein, NAREs starting with “C” are constitutive NAREs. Constitutive NAREs disclosed herein may be, for example, useful for the expression of genes in the liver, muscle, and/or central nervous system.

Examples of constitutive nucleic acid regulatory elements and their components are provided in Tables 7-9.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element provided in Tables 7 or 9. In embodiments, provided is a polynucleotide comprising one or more nucleic acid regulatory elements provided in Tables 7 or 9. As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree identity between nucleic acid or amino acid sequences is a function of the number of identical or matching nucleotides or amino acids at shared positions. Methods and computer programs for determining both sequence identity and similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C6, C7, C7b, C9-C12, C14, C14_L21, C16-C18, C21-C32, C34, C37-C91, C99-C102, C104-C108, C110-C127, C138-C194, and C202-C291. In embodiments, provided is a polynucleotide sequence that comprises a nucleic acid regulatory element selected from the group consisting of C6, C7, C7b, C9-C12, C14, C14_L21, C16-C18, C21-C32, C34, C37-C91, C99-C102, C104-C108, C110-C127, C138-C194, and C202-C291.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C21-C30. In embodiments, provided is a polynucleotide sequence that comprises a nucleic acid regulatory element selected from the group consisting of C21-C30.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C118-C127. In embodiments, provided is a polynucleotide sequence that comprises a nucleic acid regulatory element selected from the group consisting of C118-C127.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C139-C158. In embodiments, provided is a polynucleotide sequence that comprises a nucleic acid regulatory element selected from the group consisting of C139-C158.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C183-C192. In embodiments, provided is a polynucleotide sequence that comprises a nucleic acid regulatory element selected from the group consisting of C183-C192.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C53-C58, C63-C70, C83-C91, and C99-C102. In embodiments, provided is a polynucleotide sequence that comprises a nucleic acid regulatory element selected from the group consisting of C53-C58, C63-C70, C83-C91, and C99-C102.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C53-C58, C63-C70, C83-C91, C99-102, and C108. In embodiments, provided is a polynucleotide sequence that comprises a nucleic acid regulatory element selected from the group consisting of C53-C58, C63-C70, C83-C91, C99-102, and C108.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C85, C88, C90, C100-102, and C108. In embodiments, provided is a polynucleotide sequence that comprises a nucleic acid regulatory element selected from the group consisting of C85, C88, C90, C100-102, and C108.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C173-C178, C187, C200, C210, and C218. In embodiments, provided is a polynucleotide sequence that comprises a nucleic acid regulatory element selected from the group consisting of C173-C177, C187, and C218.

Provided herein is a method for constitutive expression of a transgene that is operably linked to a constitutive nucleic acid regulatory element disclosed herein.

Provided herein is a method for constitutive expression of a transgene that is operably linked to a sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element provided herein. Provided herein is a method for constitutive expression of a transgene that is operably linked to one or more nucleic acid regulatory elements provided herein.

Provided herein is a method for constitutive expression of a transgene that is operably linked to a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C6, C7, C7b, C9-C12, C14, C14_L21, C16-C18, C21-C32, C34, C37-C91, C99-C102, C104-C108, C110-C127, C138-C194, and C202-C291. Provided herein is a method for constitutive expression of a transgene that is operably linked to a sequence that selected from the group consisting of C6, C7, C7b, C9-C12, C14, C14_L21, C16-C18, C21-C32, C34, C37-C91, C99-C102, C104-C108, C110-C127, C138-C194, and C202-C291.

### Nucleic Acid Regulatory Elements for Driving Gene Expression in the Muscle

Provided herein are nucleic acid regulatory elements particularly suitable to drive expression in the muscle. Muscle NAREs can be muscle-specific, meaning that they show significantly increased, or preferential, expression of an operably linked transgene in the muscle as compared to in other tissues.

Tables 7 or 9 also provide nucleic acid regulatory elements suitable for driving expression in a variety of tissues, including in the muscle.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element selected from the group consisting of C9-C11, C21-C30, C53-58, C63-C70, C83-C91, C99-C102, and C108. In embodiments, provided is a polynucleotide sequence that comprises a nucleic acid regulatory element selected from the group consisting of C9-C11, C21-C30, C53-C58, C63-C70, C83-C91, C99-C102, and C108.

Provided herein is a method for expression of a transgene in muscle cells, wherein the transgene is operably linked to a nucleic acid regulatory element disclosed herein particularly suitable to drive expression in the muscle.

Provided herein is a method for expression of a transgene in muscle cells, wherein the transgene is operably linked to a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element provided herein. Provided herein is a method for expression of a transgene in muscle cells, wherein the transgene is operably linked to a nucleic acid regulatory element provided herein.

### NAREs for Driving Gene Expression in the Central Nervous System (CNS)

Provided herein are NAREs particularly suitable to drive expression in the CNS. CNS NAREs can be CNS-specific, meaning that they show significantly increased, or preferential, expression of an operably linked transgene in the CNS as compared to in other tissues. Tables 7 or 9 also provide nucleic acid regulatory elements suitable for driving expression in a variety of tissues, including in the CNS. In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a NARE selected from the group consisting of C14, C16, C21, C23, C26, C36), C42, C46, and C47. In embodiments, provided is a polynucleotide sequence that comprises a NARE selected from the group consisting of C14, C16, C21, C23, C26, C36, C42, C46, and C47.

In embodiments, provided is a polynucleotide sequence that comprises a sequence that is at least 80%, at least 85%, at least 90%, at least 91%, at least 92%, at least 93%, at least 94%, at least 95%, at least 96%, at least 97%, at least 98%, or at least 99%, identical to a NARE selected from the group consisting of C14, C36, C42, C249-C254, C259, and C260. In embodiments, provided is a polynucleotide sequence that comprises a NARE selected from the group consisting of C14, C36, C42, C249-C254, C259, and C260. Provided herein is a method for expression of a transgene in the CNS, wherein the transgene is operably linked to a NARE disclosed herein particularly suitable to drive expression in the CNS.

Provided herein is a method for expression of a transgene in neuronal cells, wherein the transgene is operably linked to a NARE disclosed herein particularly suitable to drive expression in neuronal cells. Provided herein is a method for expression of a transgene in the CNS, wherein the transgene is operably linked to a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element provided herein. Provided herein is a method for expression of a transgene in the CNS, wherein the transgene is operably linked to a nucleic acid regulatory element provided herein. Provided herein is a method for expression of a transgene in neuronal cells, wherein the transgene is operably linked to a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element provided herein. Provided herein is a method for expression of a transgene in neuronal cells, wherein the transgene is operably linked to a nucleic acid regulatory element provided herein.

In embodiments the transgene is UPF1 or BDNF.

### NAREs for Driving Gene Expression in the Liver

Provided herein are NAREs particularly suitable to drive expression in the liver. Liver NAREs can be liver-specific, meaning that they show significantly increased, or preferential, expression of an operably linked transgene in the liver as compared to in other tissues. Tables 7 or 9 also provide nucleic acid regulatory elements suitable for driving expression in a variety of tissues, including in the liver.

Provided herein is a polynucleotide that comprises a sequence that is at least 80%, at least 85%, at least 90%, at least 91%, at least 92%, at least 93%, at least 94%, at least 95%, at least 96%, at least 97%, at least 98%, or at least 99% identical to a nucleic acid regulatory element selected from the group consisting of C9-C12, C31-C32, C35-C36, C39-C42, C49-C51, and C57-C58. Provided herein is a polynucleotide that comprises a nucleic acid regulatory element selected from the group consisting of C9-C12, C31-C32, C35-C36, C39-C42, C49-C51, and C57-C58.

Provided herein is a method for expression of a transgene in the hepatocytes (e.g., in the liver), wherein the transgene is operably linked to a nucleic acid regulatory element disclosed herein particularly suitable to drive expression in in hepatocytes (e.g., in the liver).

Provided herein is a method for expression of a transgene in the liver, wherein the transgene is operably linked to a sequence that is at least 80%, at least 85%, at least 90%, at least 91%, at least 92%, at least 93%, at least 94%, at least 95%, at least 96%, at least 97%, at least 98%, or at least 99%, identical to a nucleic acid regulatory element provided herein. Provided herein is a method for expression of a transgene in the liver, wherein the transgene is operably linked to a nucleic acid regulatory element provided herein.

In embodiments, the transgene is ATP7B or an ATB7B minigene.

### NARE Modifications

A person skilled in the art will appreciate that certain modifications can be made to the nucleic acid regulatory elements disclosed herein without eliminating the ability of the nucleic acid regulatory element to drive gene expression in the desired tissue/cell type. Various in vitro and in vivo methods of confirming that a modified NARE is still capable of driving gene expression are known in the art, including, but not limited to, the methods used herein. For example, the strength of a NARE may be assessed by operatively linking the NARE to a transgene encoding a protein and measuring transgene expression, for example by detecting the mRNA encoding the protein, or by measuring presence or activity of the protein, e.g., by ELISA, Western Blot, fluorescence, enzymatic activity of the protein, etc.

Provided herein are NAREs comprising one or more components. Provided herein are NAREs comprising one or more sequence components (or sequence variants thereof) listed in any one of Tables 7-9. Provided herein are NAREs comprising one or more sequence components (or sequence variants thereof) listed in Tables 8 and 9. Provided herein is a NARE that comprise one or more components (or sequence variants thereof) of a full-length NARE provided in Table 9. Merely to illustrate in a non-limiting example, provided is a NARE that comprises one or more of the components (or sequence variants thereof) of NARE C22 (EH38E2531636-actb-CBh): (1) EH38E2531636; (2) CBA promoter variant 2, and (3) hybrid intron. In embodiments, provided is a NARE that comprises one or more components (or sequence variants thereof) of a full-length NARE provided in Table 9, whereby the components (or sequence variants thereof) are arranged from 5′ to 3′ as presented as in Table 9. In embodiments, provided is a NARE that comprises one or more components (or sequence variants thereof) of a full-length NARE provided in Table 9, whereby the components (or sequence variants thereof) are not arranged from 5′ to 3′ as presented as in Table 9, but in a different order.

In embodiments, in NARE of Tables 7 or 9, one or more of the components have been replaced with a different sequence, including a component (or sequence variants thereof) from a different NARE of Tables 7 or 9. Further, also contemplated are NAREs that comprise components (or sequence variants thereof) derived from two or more NAREs disclosed in Tables 7 or 9.

Moreover, contemplated are NARE variants that include sequence in addition (e.g., added at either end or inserted within the NARE sequence) to the elements of a NARE disclosed herein. Likewise, provided are NARE variants in which certain sequence has been removed from NAREs disclosed herein (e.g., as a terminal or internal deletion). In embodiments, provided are NAREs comprising one or more components without any additional nucleic acid sequence(s) joining the NARE's components. In embodiments, provided are NAREs comprising one or more components, whereby the individual components are connected by additional nucleic acid sequences. These additional nucleic acid sequences may or may not be relevant for expression of an operatively linked transgene. In embodiments, some of the components are directly linked, while other components are linked to other components via an intervening sequence. As such, contemplated are variants of the NAREs disclosed in Tables 7 or 9, wherein additional sequence has been added between the different components. Further contemplated are variants of the NAREs disclosed in Tables 7 or 9, which disclose the same components of a given NARE in Tables 7 or 9, but differ in the sequence(s) connecting the individual components.

In some embodiments, the NARE comprises a sequence that is at least 80% identical, at least 85% identical, at least 90% identical, at least 95% identical, at least 96% identical, at least 97% identical, at least 98% identical, or at least 99% identical to a sequence selected from the group consisting of SEQ ID NOs: 6, 7, 9-12, 14, 16-18, 21-32, 34, 37-91, 99-102, 104-108, 110-127, 138-194, 202-283, 286-293.

In some embodiments, the NARE comprises a sequence selected from the group consisting of SEQ ID NOs: 6, 7, 9-12, 14, 16-18, 21-32, 34, 37-91, 99-102, 104-108, 110-127, 138-194, 202-283, 286-293.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498, (ii) a sequence that is at least 80% identical, at least 85% identical, at least 90% identical, at least 95% identical, at least 96% identical, at least 97% identical, at least 98% identical, or at least 99% identical to any one of SEQ ID NOs:337, 338, 355, 357, 358, 359, 407, 501, 411, 429, 430, 431, 503, 434, 504; (iii) optionally a sequence that is at least 80% identical, at least 85% identical, at least 90% identical, at least 95% identical, at least 96% identical, at least 97% identical, at least 98% identical, or at least 99% identical to any one of SEQ ID NOs: 323, 420, 488, and (iv) optionally a sequence that is at least 80% identical, at least 85% identical, at least 90% identical, at least 95% identical, at least 96% identical, at least 97% identical, at least 98% identical, or at least 99% identical to SEQ ID NO:402 or 444.

In some embodiments, the NARE comprises (i) any one of SEQ ID NOs:344-349, 498, (ii) any one of SEQ ID NOs:337, 338, 355, 357, 358, 359, 407, 501, 411, 429, 430, 431, 503, 434, 504; (iii) optionally any one of SEQ ID NOs: 323, 420, 488, and (iv) optionally SEQ ID NO:402 or 444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:429; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498; (ii) SEQ ID NO:429; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to any one of SEQ ID NOs:429; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:349; (ii) SEQ ID NO:429; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:430, 431, 503; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498; (ii) any one of SEQ ID NOs:430, 431, 503; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:431; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NO:349; (ii) SEQ ID NO:431; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; and (ii) a sequence that is at least 80% identical, at least 85% identical, at least 90% identical, at least 95% identical, at least 96% identical, at least 97% identical, at least 98% identical, or at least 99% identical to SEQ ID NO:357.

In some embodiments, the NARE comprises (i) any one of SEQ ID NOs:344-349, 498; and (ii) SEQ ID NO:357.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; and (ii) a sequence that is at least 80% identical, at least 85% identical, at least 90% identical, at least 95% identical, at least 96% identical, at least 97% identical, at least 98% identical, or at least 99% identical to SEQ ID NO:357.

In some embodiments, the NARE comprises (i) SEQ ID NO:349; and (ii) SEQ ID NO:357.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; and (ii) a sequence that is at least 80% identical, at least 85% identical, at least 90% identical, at least 95% identical, at least 96% identical, at least 97% identical, at least 98% identical, or at least 99% identical to SEQ ID NO:358.

In some embodiments, the NARE comprises (i) any one of SEQ ID NOs:344-349, 498; and (ii) SEQ ID NO:358.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; and (ii) a sequence that is at least 80% identical, at least 85% identical, at least 90% identical, at least 95% identical, at least 96% identical, at least 97% identical, at least 98% identical, or at least 99% identical to SEQ ID NO:358.

In some embodiments, the NARE comprises (i) SEQ ID NO:349; and (ii) SEQ ID NO:358.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; and (ii) a sequence that is at least 80% identical, at least 85% identical, at least 90% identical, at least 95% identical, at least 96% identical, at least 97% identical, at least 98% identical, or at least 99% identical to SEQ ID NO:359.

In some embodiments, the NARE comprises (i) any one of SEQ ID NOs:344-349, 498; and (ii) SEQ ID NO:359.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; and (ii) a sequence that is at least 80% identical, at least 85% identical, at least 90% identical, at least 95% identical, at least 96% identical, at least 97% identical, at least 98% identical, or at least 99% identical to SEQ ID NO:359.

In some embodiments, the NARE comprises (i) SEQ ID NO:349; and (ii) SEQ ID NO:359.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:411; and (iii) a sequence that is at least 80% identical, at least 85% identical, at least 90% identical, at least 95% identical, at least 96% identical, at least 97% identical, at least 98% identical, or at least 99% identical to SEQ ID NO:420.

In some embodiments, the NARE comprises (i) any one of SEQ ID NOs:344-349, 498; (ii) SEQ ID NO:411; and (iii) SEQ ID NO:420.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:498; (ii) a sequence that is at least 80% identical, at least 85% identical, at least 90% identical, at least 95% identical, at least 96% identical, at least 97% identical, at least 98% identical, or at least 99% identical to SEQ ID NO:411; and (iii) a sequence that is at least 80% identical, at least 85% identical, at least 90% identical, at least 95% identical, at least 96% identical, at least 97% identical, at least 98% identical, or at least 99% identical to SEQ ID NO:420.

In some embodiments, the NARE comprises (i) SEQ ID NO:498; (ii) SEQ ID NO:411; and (iii) SEQ ID NO:420.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:407, 434, 501, 504; and (iii) a sequence that is at least 80% identical, at least 85% identical, at least 90% identical, at least 95% identical, at least 96% identical, at least 97% identical, at least 98% identical, or at least 99% identical to SEQ ID NO:323.

In some embodiments, the NARE comprises (i) any one of SEQ ID NOs:344-349, 498; (ii) SEQ ID NOs:407, 434, 501, 504; and (iii) SEQ ID NO:323.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to any one of SEQ ID NOs:407, 434, 501, 504; and (iii) a sequence that is at least 80% identical, at least 85% identical, at least 90% identical, at least 95% identical, at least 96% identical, at least 97% identical, at least 98% identical, or at least 99% identical to SEQ ID NO:323.

In some embodiments, the NARE comprises (i) SEQ ID NO:348; (ii) any one of SEQ ID NOs:407, 434, 501, 504; and (iii) SEQ ID NO:323.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:407 or 501; and (iii) a sequence that is at least 80% identical, at least 85% identical, at least 90% identical, at least 95% identical, at least 96% identical, at least 97% identical, at least 98% identical, or at least 99% identical to SEQ ID NO:323.

In some embodiments, the NARE comprises (i) any one of SEQ ID NOs:344-349, 498; (ii) SEQ ID NO:407 or 501; and (iii) SEQ ID NO:323.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:407; and (iii) a sequence that is at least 80% identical, at least 85% identical, at least 90% identical, at least 95% identical, at least 96% identical, at least 97% identical, at least 98% identical, or at least 99% identical to SEQ ID NO:323.

In some embodiments, the NARE comprises (i) SEQ ID NO:348; (ii) SEQ ID NO:407; and (iii) SEQ ID NO:323.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:434 or 504; and (iii) a sequence that is at least 80% identical, at least 85% identical, at least 90% identical, at least 95% identical, at least 96% identical, at least 97% identical, at least 98% identical, or at least 99% identical to SEQ ID NO:323.

In some embodiments, the NARE comprises (i) any one of SEQ ID NOs:344-349, 498; (ii) SEQ ID NOs:434 or 504; and (iii) SEQ ID NO:323.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:434; and (iii) a sequence that is at least 80% identical, at least 85% identical, at least 90% identical, at least 95% identical, at least 96% identical, at least 97% identical, at least 98% identical, or at least 99% identical to SEQ ID NO:323.

In some embodiments, the NARE comprises (i) SEQ ID NO:348; (ii) SEQ ID NO:434; and (iii) SEQ ID NO:323.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498, (ii) a sequence that is at least 80% identical, at least 85% identical, at least 90% identical, at least 95% identical, at least 96% identical, at least 97% identical, at least 98% identical, or at least 99% identical to SEQ ID NO:337; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498, (ii) SEQ ID NO:337; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:344, (ii) a sequence that is at least 80% identical, at least 85% identical, at least 90% identical, at least 95% identical, at least 96% identical, at least 97% identical, at least 98% identical, or at least 99% identical to SEQ ID NO:337; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:344, (ii) SEQ ID NO:337; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498, (ii) a sequence that is at least 80% identical, at least 85% identical, at least 90% identical, at least 95% identical, at least 96% identical, at least 97% identical, at least 98% identical, or at least 99% identical to SEQ ID NO:338; (iii) a sequence that is at least 80% identical, at least 85% identical, at least 90% identical, at least 95% identical, at least 96% identical, at least 97% identical, at least 98% identical, or at least 99% identical to SEQ ID NO:488, and (iv) a sequence that is at least 80% identical, at least 85% identical, at least 90% identical, at least 95% identical, at least 96% identical, at least 97% identical, at least 98% identical, or at least 99% identical to SEQ ID NO:402.

In some embodiments, the NARE comprises (i) any one of SEQ ID NOs:344-349, 498, (ii) SEQ ID NO:338; (iii) SEQ ID NO:488, and (iv) SEQ ID NO:402.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348, (ii) a sequence that is at least 80% identical, at least 85% identical, at least 90% identical, at least 95% identical, at least 96% identical, at least 97% identical, at least 98% identical, or at least 99% identical to SEQ ID NO:338; (iii) a sequence that is at least 80% identical, at least 85% identical, at least 90% identical, at least 95% identical, at least 96% identical, at least 97% identical, at least 98% identical, or at least 99% identical to SEQ ID NO:488, and (iv) a sequence that is at least 80% identical, at least 85% identical, at least 90% identical, at least 95% identical, at least 96% identical, at least 97% identical, at least 98% identical, or at least 99% identical to SEQ ID NO:402.

In some embodiments, the NARE comprises (i) SEQ ID NO:348, (ii) SEQ ID NO:338; (iii) SEQ ID NO:488, and (iv) SEQ ID NO:402.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498, (ii) a sequence that is at least 80% identical, at least 85% identical, at least 90% identical, at least 95% identical, at least 96% identical, at least 97% identical, at least 98% identical, or at least 99% identical to SEQ ID NO:338; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NOs:344-349, 498, (ii) SEQ ID NO:338; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348, (ii) a sequence that is at least 80% identical, at least 85% identical, at least 90% identical, at least 95% identical, at least 96% identical, at least 97% identical, at least 98% identical, or at least 99% identical to SEQ ID NO:338; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:348, (ii) SEQ ID NO:338; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:381, 384, 392, 393, 394, 395, 396, 397, 400, 401; (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) any one of SEQ ID NOs:381, 384, 392, 393, 394, 395, 396, 397, 400, 401; (ii) SEQ ID NOs: 334 or 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:381, 384, 392, 393, 394, 395, 396, 397, 400, 401; (ii) a sequence that is at least 80% identical, at least 85% identical, at least 90% identical, at least 95% identical, at least 96% identical, at least 97% identical, at least 98% identical, or at least 99% identical to SEQ ID NO: 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) any one of SEQ ID NOs:381, 384, 392, 393, 394, 395, 396, 397, 400, 401; (ii) SEQ ID NO: 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:396; and (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:396; and (ii) SEQ ID NOs: 334 or 342; and (iii) SEQ ID NO:426

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:396; and (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:396; and (ii) SEQ ID NO:342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:397; and (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:397; and (ii) SEQ ID NOs: 334 or 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:397; and (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:397; and (ii) SEQ ID NO:342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:393; and (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:393; and (ii) SEQ ID NOs: 334 or 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:393; and (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:393; and (ii) SEQ ID NO:342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:394; and (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:394; and (ii) SEQ ID NOs: 334 or 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:394; and (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:394; and (ii) SEQ ID NO:342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:395; and (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:395; and (ii) SEQ ID NOs: 334 or 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:395; and (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:395; and (ii) SEQ ID NO:342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:384; and (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:384; and (ii) SEQ ID NOs: 334 or 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:384; and (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:384; and (ii) SEQ ID NO:342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:401; and (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:401; and (ii) SEQ ID NOs: 334 or 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:401; and (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:401; and (ii) SEQ ID NO:342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:400; and (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:400; and (ii) SEQ ID NOs: 334 or 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:400; and (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:400; and (ii) SEQ ID NO:342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:381; and (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:381; and (ii) SEQ ID NOs: 334 or 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:381; and (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:381; and (ii) SEQ ID NO:342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:392; and (ii) a sequence that is at least 80% identical, at least 85% identical, at least 90% identical, at least 95% identical, at least 96% identical, at least 97% identical, at least 98% identical, or at least 99% identical to SEQ ID NOs: 334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:392; and (ii) SEQ ID NOs: 334 or 342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:392; and (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:426.

In some embodiments, the NARE comprises (i) SEQ ID NO:392; and (ii) SEQ ID NO:342; and (iii) SEQ ID NO:426.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; (iii) a sequence that is at least 80% identical, at least 85% identical, at least 90% identical, at least 95% identical, at least 96% identical, at least 97% identical, at least 98% identical, or at least 99% identical to any one of SEQ ID NOs:302-318, 320-322, 324-327, 331, 332, or 487, (iv) optionally a sequence that is at least 80% identical, at least 85% identical, at least 90% identical, at least 95% identical, at least 96% identical, at least 97% identical, at least 98% identical, or at least 99% identical to SEQ ID NO:333; and (v) optionally a sequence that is at least 80% identical, at least 85% identical, at least 90% identical, at least 95% identical, at least 96% identical, at least 97% identical, at least 98% identical, or at least 99% identical to SEQ ID NO:319.

In some embodiments, the NARE comprises (i) any one of SEQ ID NOs:344-349, 498; (ii) SEQ ID NOs:334 or 342; (iii) any one of SEQ ID NOs:302-318, 320-322, 324-327, 331, 332, or 487, (iv) optionally SEQ ID NO:333; and (v) optionally SEQ ID NO:319.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:345 or 348; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; (iii) a sequence that is at least 80% identical, at least 85% identical, at least 90% identical, at least 95% identical, at least 96% identical, at least 97% identical, at least 98% identical, or at least 99% identical to any one of SEQ ID NOs:302-318, 320-322, 324-327, 331, 332, or 487, (iv) optionally a sequence that is at least 80% identical, at least 85% identical, at least 90% identical, at least 95% identical, at least 96% identical, at least 97% identical, at least 98% identical, or at least 99% identical to SEQ ID NO:333; and (v) optionally a sequence that is at least 80% identical, at least 85% identical, at least 90% identical, at least 95% identical, at least 96% identical, at least 97% identical, at least 98% identical, or at least 99% identical to SEQ ID NO:319.

In some embodiments, the NARE comprises (i) to any one of SEQ ID NOs:345 or 348; (ii) SEQ ID NOs:334 or 342; (iii) any one of SEQ ID NOs:302-318, 320-322, 324-327, 331, 332, or 487, (iv) optionally SEQ ID NO:333; and (v) SEQ ID NO:319.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:302-317, 320-322, 324-327, 331, 332, or 487.

In some embodiments, the NARE comprises (i) any one of SEQ ID NOs:344-349, 498; (ii) SEQ ID NOs:334 or 342; and (iii) any one of SEQ ID NOs:302-317, 320-322, 324-327, 331, 332, or 487.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NOs:345 or 348; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 442; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:302-317, 320-322, 324-327, 331, 332, or 487.

In some embodiments, the NARE comprises (i) SEQ ID NOs:345 or 348; (ii) SEQ ID NOs:334 or 442; and (iii) any one of SEQ ID NOs:302-317, 320-322, 324-327, 331, 332, or 487.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:302-317, 320-322, 324-327, 331, 332.

In some embodiments, the NARE comprises (i) SEQ ID NOs:344-349, 498; (ii) SEQ ID NOs:334 or 342; and (iii) any one of SEQ ID NOs:302-317, 320-322, 324-327, 331, 332.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:302-317, 320-322, 324-327, 331, 332.

In some embodiments, the NARE comprises (i) SEQ ID NOs:348; (ii) SEQ ID NO:334; and (iii) any one of SEQ ID NOs:302-317, 320-322, 324-327, 331, 332.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; (iii) a sequence that is at least 80% identical, at least 85% identical, at least 90% identical, at least 95% identical, at least 96% identical, at least 97% identical, at least 98% identical, or at least 99% identical to SEQ ID NO:318; (iv) a sequence that is at least 80% identical, at least 85% identical, at least 90% identical, at least 95% identical, at least 96% identical, at least 97% identical, at least 98% identical, or at least 99% identical to SEQ ID NO:333; and (v) a sequence that is at least 80% identical, at least 85% identical, at least 90% identical, at least 95% identical, at least 96% identical, at least 97% identical, at least 98% identical, or at least 99% identical to SEQ ID NO:319.

In some embodiments, the NARE comprises (i) any one of SEQ ID NOs:344-349, 498; (ii) SEQ ID NOs:334 or 342; (iii) SEQ ID NO:318; (iv) SEQ ID NO:333; and (v) SEQ ID NO:319.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; (iii) a sequence that is at least 80% identical, at least 85% identical, at least 90% identical, at least 95% identical, at least 96% identical, at least 97% identical, at least 98% identical, or at least 99% identical to SEQ ID NO:318; (iv) a sequence that is at least 80% identical, at least 85% identical, at least 90% identical, at least 95% identical, at least 96% identical, at least 97% identical, at least 98% identical, or at least 99% identical to SEQ ID NO:333; and (v) a sequence that is at least 80% identical, at least 85% identical, at least 90% identical, at least 95% identical, at least 96% identical, at least 97% identical, at least 98% identical, or at least 99% identical to SEQ ID NO:319.

In some embodiments, the NARE comprises (i) SEQ ID NO:348; (ii) SEQ ID NO:334; (iii) SEQ ID NO:318; (iv) SEQ ID NO:333; and (v) SEQ ID NO:319.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:304.

In some embodiments, the NARE comprises (i) any one of SEQ ID NOs:344-349, 498; (ii) SEQ ID NOs:334 or 342; and (iii) SEQ ID NO:304.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:304.

In some embodiments, the NARE comprises (i) SEQ ID NO:348; (ii) SEQ ID NO:334; and (iii) SEQ ID NO:304.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:305.

In some embodiments, the NARE comprises (i) any one of SEQ ID NOs:344-349, 498; (ii) SEQ ID NOs:334 or 342; and (iii) SEQ ID NO:305.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:305.

In some embodiments, the NARE comprises (i) SEQ ID NO:348; (ii) SEQ ID NO:334; and (iii) SEQ ID NO:305.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:332.

In some embodiments, the NARE comprises (i) any one of SEQ ID NOs:344-349, 498; (ii) SEQ ID NOs:334 or 342; and (iii) SEQ ID NO:332.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:332.

In some embodiments, the NARE comprises (i) SEQ ID NO:348; (ii) SEQ ID NO:334; and (iii) SEQ ID NO:332.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:303.

In some embodiments, the NARE comprises (i) any one of SEQ ID NOs:344-349, 498; (ii) SEQ ID NOs:334 or 342; and (iii) SEQ ID NO: 303.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:303.

In some embodiments, the NARE comprises (i) SEQ ID NO:348; (ii) SEQ ID NO:334; and (iii) SEQ ID NO:303.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:302.

In some embodiments, the NARE comprises (i) any one of SEQ ID NOs:344-349, 498; (ii) SEQ ID NOs:334 or 342; and (iii) SEQ ID NO: 302.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:302.

In some embodiments, the NARE comprises (i) SEQ ID NO:348; (ii) SEQ ID NO:334; and (iii) SEQ ID NO:302.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:306.

In some embodiments, the NARE comprises (i) any one of SEQ ID NOs:344-349, 498; (ii) SEQ ID NOs:334 or 342; and (iii) SEQ ID NO:306.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:306.

In some embodiments, the NARE comprises (i) SEQ ID NO:348; (ii) SEQ ID NO:334; and (iii) SEQ ID NO:306.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:307.

In some embodiments, the NARE comprises (i) any one of SEQ ID NOs:344-349, 498; (ii) SEQ ID NOs:334 or 342; and (iii) SEQ ID NO:307.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:307.

In some embodiments, the NARE comprises (i) SEQ ID NO:348; (ii) SEQ ID NO:334; and (iii) SEQ ID NO:307.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:309.

In some embodiments, the NARE comprises (i) any one of SEQ ID NOs:344-349, 498; (ii) SEQ ID NOs:334 or 342; and (iii) SEQ ID NO:309.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:309.

In some embodiments, the NARE comprises (i) SEQ ID NO:348; (ii) SEQ ID NO:334; and (iii) SEQ ID NO:309.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:308.

In some embodiments, the NARE comprises (i) any one of SEQ ID NOs:344-349, 498; (ii) SEQ ID NOs:334 or 342; and (iii) SEQ ID NO:308.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:308.

In some embodiments, the NARE comprises (i) SEQ ID NO:348; (ii) SEQ ID NO:334; and (iii) SEQ ID NO:308.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:310.

In some embodiments, the NARE comprises (i) any one of SEQ ID NOs:344-349, 498; (ii) SEQ ID NOs:334 or 342; and (iii) SEQ ID NO:310.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:310.

In some embodiments, the NARE comprises (i) SEQ ID NO:348; (ii) SEQ ID NO:334; and (iii) SEQ ID NO:310.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:317.

In some embodiments, the NARE comprises (i) any one of SEQ ID NOs:344-349, 498; (ii) SEQ ID NOs:334 or 342; and (iii) SEQ ID NO:317.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:317.

In some embodiments, the NARE comprises (i) SEQ ID NO:348; (ii) SEQ ID NO:334; and (iii) SEQ ID NO:317.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:315.

In some embodiments, the NARE comprises (i) any one of SEQ ID NOs:344-349, 498; (ii) SEQ ID NOs:334 or 342; and (iii) SEQ ID NO:315.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:315.

In some embodiments, the NARE comprises (i) SEQ ID NO:348; (ii) SEQ ID NO:334; and (iii) SEQ ID NO:315.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:312.

In some embodiments, the NARE comprises (i) any one of SEQ ID NOs:344-349, 498; (ii) SEQ ID NOs:334 or 342; and (iii) SEQ ID NO:312.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:312.

In some embodiments, the NARE comprises (i) SEQ ID NO:348; (ii) SEQ ID NO:334; and (iii) SEQ ID NO:312.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:316.

In some embodiments, the NARE comprises (i) any one of SEQ ID NOs:344-349, 498; (ii) SEQ ID NOs:334 or 342; and (iii) SEQ ID NO:316.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:316.

In some embodiments, the NARE comprises (i) SEQ ID NO:348; (ii) SEQ ID NO:334; and (iii) SEQ ID NO:316.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:314.

In some embodiments, the NARE comprises (i) any one of SEQ ID NOs:344-349, 498; (ii) SEQ ID NOs:334 or 342; and (iii) SEQ ID NO:314.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:314.

In some embodiments, the NARE comprises (i) SEQ ID NO:348; (ii) SEQ ID NO:334; and (iii) SEQ ID NO:314.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:313.

In some embodiments, the NARE comprises (i) any one of SEQ ID NOs:344-349, 498; (ii) SEQ ID NOs:334 or 342; and (iii) SEQ ID NO:313.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:313.

In some embodiments, the NARE comprises (i) SEQ ID NO:348; (ii) SEQ ID NO:334; and (iii) SEQ ID NO:313.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:311.

In some embodiments, the NARE comprises (i) any one of SEQ ID NOs:344-349, 498; (ii) SEQ ID NOs:334 or 342; and (iii) SEQ ID NO:311.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:311.

In some embodiments, the NARE comprises (i) SEQ ID NO:348; (ii) SEQ ID NO:334; and (iii) SEQ ID NO:311.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:321.

In some embodiments, the NARE comprises (i) any one of SEQ ID NOs:344-349, 498; (ii) SEQ ID NOs:334 or 342; and (iii) SEQ ID NO:321.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:321.

In some embodiments, the NARE comprises (i) SEQ ID NO:348; (ii) SEQ ID NO:334; and (iii) SEQ ID NO:321.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:322.

In some embodiments, the NARE comprises (i) any one of SEQ ID NOs:344-349, 498; (ii) SEQ ID NOs:334 or 342; and (iii) SEQ ID NO:322.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:322.

In some embodiments, the NARE comprises (i) SEQ ID NO:348; (ii) SEQ ID NO:334; and (iii) SEQ ID NO:322.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:320.

In some embodiments, the NARE comprises (i) any one of SEQ ID NOs:344-349, 498; (ii) SEQ ID NOs:334 or 342; and (iii) SEQ ID NO:320.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:320.

In some embodiments, the NARE comprises (i) SEQ ID NO:348; (ii) SEQ ID NO:334; and (iii) SEQ ID NO:320.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:324.

In some embodiments, the NARE comprises (i) any one of SEQ ID NOs:344-349, 498; (ii) SEQ ID NOs:334 or 342; and (iii) SEQ ID NO:324.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:324.

In some embodiments, the NARE comprises (i) SEQ ID NO:348; (ii) SEQ ID NO:334; and (iii) SEQ ID NO:324.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:331.

In some embodiments, the NARE comprises (i) any one of SEQ ID NOs:344-349, 498; (ii) SEQ ID NOs:334 or 342; and (iii) SEQ ID NO:331.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:331.

In some embodiments, the NARE comprises (i) SEQ ID NO:348; (ii) SEQ ID NO:334; and (iii) SEQ ID NO:331.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:327.

In some embodiments, the NARE comprises (i) any one of SEQ ID NOs:344-349, 498; (ii) SEQ ID NOs:334 or 342; and (iii) SEQ ID NO:327.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:327.

In some embodiments, the NARE comprises (i) SEQ ID NO:348; (ii) SEQ ID NO:334; and (iii) SEQ ID NO:327.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:325.

In some embodiments, the NARE comprises (i) any one of SEQ ID NOs:344-349, 498; (ii) SEQ ID NOs:334 or 342; and (iii) SEQ ID NO:325.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:325.

In some embodiments, the NARE comprises (i) SEQ ID NO:348; (ii) SEQ ID NO:334; and (iii) SEQ ID NO:325.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:326.

In some embodiments, the NARE comprises (i) any one of SEQ ID NOs:344-349, 498; (ii) SEQ ID NOs:334 or 342; and (iii) SEQ ID NO:326.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:334; and (iii) a sequence that is at least 80% identical, at least 85% identical, at least 90% identical, at least 95% identical, at least 96% identical, at least 97% identical, at least 98% identical, or at least 99% identical to SEQ ID NO:326.

In some embodiments, the NARE comprises (i) SEQ ID NO:348; (ii) SEQ ID NO:334; and (iii) SEQ ID NO:326.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NOs:334 or 342; and (iii) a sequence that is at least 80% identical, at least 85% identical, at least 90% identical, at least 95% identical, at least 96% identical, at least 97% identical, at least 98% identical, or at least 99% identical to SEQ ID NO:487.

In some embodiments, the NARE comprises (i) any one of SEQ ID NOs:344-349, 498; (ii) SEQ ID NOs:334 or 342; and (iii) SEQ ID NO:487.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:345; (ii) a sequence that is at least 80% identical, at least 85% identical, at least 90% identical, at least 95% identical, at least 96% identical, at least 97% identical, at least 98% identical, or at least 99% identical to SEQ ID NO:342; and (iii) a sequence that is at least 80% identical, at least 85% identical, at least 90% identical, at least 95% identical, at least 96% identical, at least 97% identical, at least 98% identical, or at least 99% identical to SEQ ID NO:487.

In some embodiments, the NARE comprises (i) SEQ ID NO:345; (ii) SEQ ID NO:342; and (iii) SEQ ID NO:487.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; (iii) a sequence that is at least 80% identical, at least 85% identical, at least 90% identical, at least 95% identical, at least 96% identical, at least 97% identical, at least 98% identical, or at least 99% identical to any one of SEQ ID NOs: 200, 370, 417-420, 502, 441, 482-485, 489, 491-496; and (iv) optionally a sequence that is at least 80% identical, at least 85% identical, at least 90% identical, at least 95% identical, at least 96% identical, at least 97% identical, at least 98% identical, or at least 99% identical to any one of SEQ ID NOs:444, 452, 488; (v) optionally a sequence that is at least 80% identical, at least 85% identical, at least 90% identical, at least 95% identical, at least 96% identical, at least 97% identical, at least 98% identical, or at least 99% identical to SEQ ID NO:402; and (vi) optionally a sequence that is at least 80% identical, at least 85% identical, at least 90% identical, at least 95% identical, at least 96% identical, at least 97% identical, at least 98% identical, or at least 99% identical to SEQ ID NO:433.

In some embodiments, the NARE comprises (i) any one of SEQ ID NOs:344-349, 498; (ii) any one of SEQ ID NOs:351-356, 499, 500; (iii) any one of SEQ ID NOs: 200, 370, 417-420, 502, 441, 482-485, 489, 491-496; and (iv) optionally any one of SEQ ID NOs:444, 452, or 488; (v) optionally SEQ ID NO:402; and (vi) optionally SEQ ID NO:433.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; (iii) a sequence that is at least 80% identical, at least 85% identical, at least 90% identical, at least 95% identical, at least 96% identical, at least 97% identical, at least 98% identical, or at least 99% identical to any one of SEQ ID NOs: 200, 370, 417-420, 502, 482-485, 489, 491-496; and (iv) optionally a sequence that is at least 80% identical, at least 85% identical, at least 90% identical, at least 95% identical, at least 96% identical, at least 97% identical, at least 98% identical, or at least 99% identical to SEQ ID NOs:444 or 452.

In some embodiments, the NARE comprises (i) any one of SEQ ID NOs:344-349, 498; (ii) any one of SEQ ID NOs:351-356, 499, 500; (iii) any one of SEQ ID NOs: 200, 370, 417-420, 502, 482-485, 489, 491-496; and (iv) optionally SEQ ID NOs:444 or 452.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; (iii) a sequence that is at least 80% identical, at least 85% identical, at least 90% identical, at least 95% identical, at least 96% identical, at least 97% identical, at least 98% identical, or at least 99% identical to any one of SEQ ID NOs: 200, 370, 417, 482-485, 489, 491-496; and (iv) optionally a sequence that is at least 80% identical, at least 85% identical, at least 90% identical, at least 95% identical, at least 96% identical, at least 97% identical, at least 98% identical, or at least 99% identical to SEQ ID NOs:444 or 452.

In some embodiments, the NARE comprises (i) any one of SEQ ID NOs:344-349, 498; (ii) any one of SEQ ID NOs:351-356, 499, 500; (iii) any one of SEQ ID NOs: 200, 370, 417, 482-485, 489, 491-496; and (iv) optionally SEQ ID NOs:444 or 452.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; (iii) a sequence that is at least 80% identical, at least 85% identical, at least 90% identical, at least 95% identical, at least 96% identical, at least 97% identical, at least 98% identical, or at least 99% identical to any one of SEQ ID NOs:200, 417-420, 502, 485; and (iv) optionally a sequence that is at least 80% identical, at least 85% identical, at least 90% identical, at least 95% identical, at least 96% identical, at least 97% identical, at least 98% identical, or at least 99% identical to SEQ ID NOs:444 or 452.

In some embodiments, the NARE comprises (i) any one of SEQ ID NOs:344-349, 498; (ii) any one of SEQ ID NOs:351-356, 499, 500; (iii) any one of SEQ ID NOs:200, 417-420, 502, 485; and (iv) optionally SEQ ID NOs:444 or 452.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; (iii) a sequence that is at least 80% identical, at least 85% identical, at least 90% identical, at least 95% identical, at least 96% identical, at least 97% identical, at least 98% identical, or at least 99% identical to any one of SEQ ID NOs:200, 417, or 485; and (iv) optionally a sequence that is at least 80% identical, at least 85% identical, at least 90% identical, at least 95% identical, at least 96% identical, at least 97% identical, at least 98% identical, or at least 99% identical to SEQ ID NOs:444 or 452.

In some embodiments, the NARE comprises (i) any one of SEQ ID NOs:344-349, 498; (ii) any one of SEQ ID NOs:351-356, 499, 500; (iii) any one of SEQ ID NOs:200, 417, or 485; and (iv) optionally SEQ ID NOs:444 or 452.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:370, 482-484, 489, 491-496.

In some embodiments, the NARE comprises (i) any one of SEQ ID NOs:344-349, 498; (ii) any one of SEQ ID NOs:351-356, 499, 500; and (iii) any one of SEQ ID NOs:370, 482-484, 489, 491-496.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NOs:353; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:370, 482-484, 489, 491-496.

In some embodiments, the NARE comprises (i) SEQ ID NO:349; (ii) SEQ ID NOs:353; and (iii) any one of SEQ ID NOs:370, 482-484, 489, 491-496.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:355; and (iii) a sequence that is at least 80% identical, at least 85% identical, at least 90% identical, at least 95% identical, at least 96% identical, at least 97% identical, at least 98% identical, or at least 99% identical to SEQ ID NO:200.

In some embodiments, the NARE comprises (i) any one of SEQ ID NOs:344-349, 498; (ii) SEQ ID NO:355; and (iii) SEQ ID NO:200.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:355; and (iii) a sequence that is at least 80% identical, at least 85% identical, at least 90% identical, at least 95% identical, at least 96% identical, at least 97% identical, at least 98% identical, or at least 99% identical to SEQ ID NO:200.

In some embodiments, the NARE comprises (i) SEQ ID NO:349; (ii) SEQ ID NO:355; and (iii) SEQ ID NO:200.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; (iii) a sequence that is at least 80% identical, at least 85% identical, at least 90% identical, at least 95% identical, at least 96% identical, at least 97% identical, at least 98% identical, or at least 99% identical to any one of SEQ ID NOs:417-420, 502; and (iv) a sequence that is at least 80% identical, at least 85% identical, at least 90% identical, at least 95% identical, at least 96% identical, at least 97% identical, at least 98% identical, or at least 99% identical to SEQ ID NOs:452.

In some embodiments, the NARE comprises (i) any one of SEQ ID NOs:344-349, 498; (ii) any one of SEQ ID NOs:351-356, 499, 500; (iii) any one of SEQ ID NOs:417-420, 502; and (iv) SEQ ID NOs:452.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:346; (ii) a sequence that is at least 80% identical, at least 85% identical, at least 90% identical, at least 95% identical, at least 96% identical, at least 97% identical, at least 98% identical, or at least 99% identical to SEQ ID NO:500; (iii) a sequence that is at least 80% identical, at least 85% identical, at least 90% identical, at least 95% identical, at least 96% identical, at least 97% identical, at least 98% identical, or at least 99% identical to SEQ ID NO:417; and (iv) a sequence that is at least 80% identical, at least 85% identical, at least 90% identical, at least 95% identical, at least 96% identical, at least 97% identical, at least 98% identical, or at least 99% identical to SEQ ID NOs:452.

In some embodiments, the NARE comprises (i) SEQ ID NO:346; (ii) SEQ ID NO:500; (iii) SEQ ID NO:417; and (iv) SEQ ID NOs:452.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; (iii) a sequence that is at least 80% identical, at least 85% identical, at least 90% identical, at least 95% identical, at least 96% identical, at least 97% identical, at least 98% identical, or at least 99% identical to any one of SEQ ID NOs:417-420, 502; and (iv) a sequence that is at least 80% identical, at least 85% identical, at least 90% identical, at least 95% identical, at least 96% identical, at least 97% identical, at least 98% identical, or at least 99% identical to SEQ ID NOs:444.

In some embodiments, the NARE comprises (i) any one of SEQ ID NOs:344-349, 498; (ii) any one of SEQ ID NOs:351-356, 499, 500; (iii) any one of SEQ ID NOs:417-420, 502; and (iv) SEQ ID NOs:452.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:354; (iii) a sequence that is at least 80% identical, at least 85% identical, at least 90% identical, at least 95% identical, at least 96% identical, at least 97% identical, at least 98% identical, or at least 99% identical to SEQ ID NO:502; and (iv) a sequence that is at least 80% identical, at least 85% identical, at least 90% identical, at least 95% identical, at least 96% identical, at least 97% identical, at least 98% identical, or at least 99% identical to SEQ ID NOs:444.

In some embodiments, the NARE comprises (i) SEQ ID NO:349; (ii) SEQ ID NO:354; (iii) SEQ ID NO:502; and (iv) SEQ ID NOs:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; (iii) a sequence that is at least 80% identical, at least 85% identical, at least 90% identical, at least 95% identical, at least 96% identical, at least 97% identical, at least 98% identical, or at least 99% identical to SEQ ID NO:441; (iv) a sequence that is at least 80% identical, at least 85% identical, at least 90% identical, at least 95% identical, at least 96% identical, at least 97% identical, at least 98% identical, or at least 99% identical to SEQ ID NO:488; (v) a sequence that is at least 80% identical, at least 85% identical, at least 90% identical, at least 95% identical, at least 96% identical, at least 97% identical, at least 98% identical, or at least 99% identical to SEQ ID NO:402; and (vi) a sequence that is at least 80% identical, at least 85% identical, at least 90% identical, at least 95% identical, at least 96% identical, at least 97% identical, at least 98% identical, or at least 99% identical to SEQ ID NO:433.

In some embodiments, the NARE comprises (i) any one of SEQ ID NOs:344-349, 498; (ii) any one of SEQ ID NOs:351-356, 499, 500; (iii) SEQ ID NO:441; (iv) SEQ ID NO:488; (v) SEQ ID NO:402; and (vi) SEQ ID NO:433.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:347; (ii) a sequence that is at least 80% identical, at least 85% identical, at least 90% identical, at least 95% identical, at least 96% identical, at least 97% identical, at least 98% identical, or at least 99% identical to SEQ ID NO:351; (iii) a sequence that is at least 80% identical, at least 85% identical, at least 90% identical, at least 95% identical, at least 96% identical, at least 97% identical, at least 98% identical, or at least 99% identical to SEQ ID NO:441; (iv) a sequence that is at least 80% identical, at least 85% identical, at least 90% identical, at least 95% identical, at least 96% identical, at least 97% identical, at least 98% identical, or at least 99% identical to SEQ ID NO:488; (v) a sequence that is at least 80% identical, at least 85% identical, at least 90% identical, at least 95% identical, at least 96% identical, at least 97% identical, at least 98% identical, or at least 99% identical to SEQ ID NO:402; and (vi) a sequence that is at least 80% identical, at least 85% identical, at least 90% identical, at least 95% identical, at least 96% identical, at least 97% identical, at least 98% identical, or at least 99% identical to SEQ ID NO:433.

In some embodiments, the NARE comprises (i) SEQ ID NO:347; (ii) SEQ ID NO:351; (iii) SEQ ID NO:441; (iv) SEQ ID NO:488; (v) SEQ ID NO:402; and (vi) SEQ ID NO:433.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:489.

In some embodiments, the NARE comprises (i) any one of SEQ ID NOs:344-349, 498; (ii) any one of SEQ ID NOs:351-356, 499, 500; and (iii) SEQ ID NO:489.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:353; and (iii) a sequence that is at least 80% identical, at least 85% identical, at least 90% identical, at least 95% identical, at least 96% identical, at least 97% identical, at least 98% identical, or at least 99% identical to SEQ ID NO:489.

In some embodiments, the NARE comprises (i) SEQ ID NO:349; (ii) SEQ ID NO:353; and (iii) SEQ ID NO:489.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:370.

In some embodiments, the NARE comprises (i) any one of SEQ ID NOs:344-349, 498; (ii) any one of SEQ ID NOs:351-356, 499, 500; and (iii) SEQ ID NO:370.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:353; and (iii) a sequence that is at least 80% identical, at least 85% identical, at least 90% identical, at least 95% identical, at least 96% identical, at least 97% identical, at least 98% identical, or at least 99% identical to SEQ ID NO:370.

In some embodiments, the NARE comprises (i) SEQ ID NO:349; (ii) SEQ ID NO:353; and (iii) SEQ ID NO:370.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:483.

In some embodiments, the NARE comprises (i) any one of SEQ ID NOs:344-349, 498; (ii) any one of SEQ ID NOs:351-356, 499, 500; and (iii) SEQ ID NO:483.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:353; and (iii) a sequence that is at least 80% identical, at least 85% identical, at least 90% identical, at least 95% identical, at least 96% identical, at least 97% identical, at least 98% identical, or at least 99% identical to SEQ ID NO:483.

In some embodiments, the NARE comprises (i) SEQ ID NO:349; (ii) SEQ ID NO:353; and (iii) SEQ ID NO:483.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:484.

In some embodiments, the NARE comprises (i) any one of SEQ ID NOs:344-349, 498; (ii) any one of SEQ ID NOs:351-356, 499, 500; and (iii) SEQ ID NO:484.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:353; and (iii) a sequence that is at least 80% identical, at least 85% identical, at least 90% identical, at least 95% identical, at least 96% identical, at least 97% identical, at least 98% identical, or at least 99% identical to SEQ ID NO:484.

In some embodiments, the NARE comprises (i) SEQ ID NO:349; (ii) SEQ ID NO:353; and (iii) SEQ ID NO:484.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:482.

In some embodiments, the NARE comprises (i) any one of SEQ ID NOs:344-349, 498; (ii) any one of SEQ ID NOs:351-356, 499, 500; and (iii) SEQ ID NO:482.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:353; and (iii) a sequence that is at least 80% identical, at least 85% identical, at least 90% identical, at least 95% identical, at least 96% identical, at least 97% identical, at least 98% identical, or at least 99% identical to SEQ ID NO:482.

In some embodiments, the NARE comprises (i) SEQ ID NO:349; (ii) SEQ ID NO:353; and (iii) SEQ ID NO:482.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:491.

In some embodiments, the NARE comprises (i) any one of SEQ ID NOs:344-349, 498; (ii) any one of SEQ ID NOs:351-356, 499, 500; and (iii) SEQ ID NO:491.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:353; and (iii) a sequence that is at least 80% identical, at least 85% identical, at least 90% identical, at least 95% identical, at least 96% identical, at least 97% identical, at least 98% identical, or at least 99% identical to SEQ ID NO:491.

In some embodiments, the NARE comprises (i) SEQ ID NO:349; (ii) SEQ ID NO:353; and (iii) SEQ ID NO:491.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:492.

In some embodiments, the NARE comprises (i) any one of SEQ ID NOs:344-349, 498; (ii) any one of SEQ ID NOs:351-356, 499, 500; and (iii) SEQ ID NO:492.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:353; and (iii) a sequence that is at least 80% identical, at least 85% identical, at least 90% identical, at least 95% identical, at least 96% identical, at least 97% identical, at least 98% identical, or at least 99% identical to SEQ ID NO:492.

In some embodiments, the NARE comprises (i) SEQ ID NO:349; (ii) SEQ ID NO:353; and (iii) SEQ ID NO:492.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:495.

In some embodiments, the NARE comprises (i) any one of SEQ ID NOs:344-349, 498; (ii) any one of SEQ ID NOs:351-356, 499, 500; and (iii) SEQ ID NO:495.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:353; and (iii) a sequence that is at least 80% identical, at least 85% identical, at least 90% identical, at least 95% identical, at least 96% identical, at least 97% identical, at least 98% identical, or at least 99% identical to SEQ ID NO:495.

In some embodiments, the NARE comprises (i) SEQ ID NO:349; (ii) SEQ ID NO:353; and (iii) SEQ ID NO:495.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:493.

In some embodiments, the NARE comprises (i) any one of SEQ ID NOs:344-349, 498; (ii) any one of SEQ ID NOs:351-356, 499, 500; and (iii) SEQ ID NO:493.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:499; and (iii) a sequence that is at least 80% identical, at least 85% identical, at least 90% identical, at least 95% identical, at least 96% identical, at least 97% identical, at least 98% identical, or at least 99% identical to SEQ ID NO:493.

In some embodiments, the NARE comprises (i) SEQ ID NO:349; (ii) SEQ ID NO:499; and (iii) SEQ ID NO:493.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:494.

In some embodiments, the NARE comprises (i) any one of SEQ ID NOs:344-349, 498; (ii) any one of SEQ ID NOs:351-356, 499, 500; and (iii) SEQ ID NO:494.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:353; and (iii) a sequence that is at least 80% identical, at least 85% identical, at least 90% identical, at least 95% identical, at least 96% identical, at least 97% identical, at least 98% identical, or at least 99% identical to SEQ ID NO:494.

In some embodiments, the NARE comprises (i) SEQ ID NO:349; (ii) SEQ ID NO:353; and (iii) SEQ ID NO:494.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:496.

In some embodiments, the NARE comprises (i) any one of SEQ ID NOs:344-349, 498; (ii) any one of SEQ ID NOs:351-356, 499, 500; and (iii) SEQ ID NO:496.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:353; and (iii) a sequence that is at least 80% identical, at least 85% identical, at least 90% identical, at least 95% identical, at least 96% identical, at least 97% identical, at least 98% identical, or at least 99% identical to SEQ ID NO:496.

In some embodiments, the NARE comprises (i) SEQ ID NO:349; (ii) SEQ ID NO:353; and (iii) SEQ ID NO:496.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iii) a sequence that is at least 80% identical, at least 85% identical, at least 90% identical, at least 95% identical, at least 96% identical, at least 97% identical, at least 98% identical, or at least 99% identical to SEQ ID NO:485.

In some embodiments, the NARE comprises (i) any one of SEQ ID NOs:344-349, 498; (ii) any one of SEQ ID NOs:351-356, 499, 500; and (iii) SEQ ID NO:485.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349; (ii) a sequence that is at least 80% identical, at least 85% identical, at least 90% identical, at least 95% identical, at least 96% identical, at least 97% identical, at least 98% identical, or at least 99% identical to SEQ ID NO:352; and (iii) a sequence that is at least 80% identical, at least 85% identical, at least 90% identical, at least 95% identical, at least 96% identical, at least 97% identical, at least 98% identical, or at least 99% identical to SEQ ID NO:485.

In some embodiments, the NARE comprises (i) SEQ ID NO:349; (ii) SEQ ID NO:352; and (iii) SEQ ID NO:485.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:294, 295, 296, 299, 323, 328, 329, 330, 407, 501, 430, 431, 503, 434, 504; and (iv) optionally a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498; (ii) SEQ ID NO:335; and (iii) any one of SEQ ID NOs: 294, 295, 296, 299, 323, 328, 329, 330, 407, 501, 430, 431, 503, 434, 504; and (iv) optionally SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:294, 296, 299, 323, 328, 329, 330, 407, 430, 434; and (iv) optionally a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:348; (ii) SEQ ID NO:335; and (iii) any one of SEQ ID NOs:294, 296, 299, 323, 328, 329, 330, 407, 430, 434; and (iv) optionally SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 294, 295, 296, 407, 501, 430, 431, 503, 434, 504; and (iv) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498; (ii) SEQ ID NO:335; and (iii) any one of SEQ ID NOs: 294, 295, 296, 407, 501, 430, 431, 503, 434, 504; and (iv) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to any one of SEQ ID NOs: 294, 296, 407, 430, 434; and (iv) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:348; (ii) SEQ ID NO:335; and (iii) any one of SEQ ID NOs: 294, 296, 407, 430, 434; and (iv) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to SEQ ID NO:323.

In some embodiments, the NARE comprises (i) any one of SEQ ID NOs:344-349, 498; (ii) SEQ ID NO:335; and (iii) SEQ ID NO:323.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to SEQ ID NO:323.

In some embodiments, the NARE comprises (i) SEQ ID NO:348; (ii) SEQ ID NO:335; and (iii) SEQ ID NO:323.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to SEQ ID NO:328.

In some embodiments, the NARE comprises (i) any one of SEQ ID NOs:344-349, 498; (ii) SEQ ID NO:335; and (iii) SEQ ID NO:328.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO: 348; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to SEQ ID NO:328.

In some embodiments, the NARE comprises (i) SEQ ID NO: 348; (ii) SEQ ID NO:335; and (iii) SEQ ID NO:328.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to SEQ ID NO:330.

In some embodiments, the NARE comprises (i) any one of SEQ ID NOs:344-349, 498; (ii) SEQ ID NO:335; and (iii) SEQ ID NO:330.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO: 348; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to SEQ ID NO:330.

In some embodiments, the NARE comprises (i) SEQ ID NO: 348; (ii) SEQ ID NO:335; and (iii) SEQ ID NO:330.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to SEQ ID NO:329.

In some embodiments, the NARE comprises (i) any one of SEQ ID NOs:344-349, 498; (ii) SEQ ID NO:335; and (iii) SEQ ID NO:329.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO: 348; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to SEQ ID NO:329.

In some embodiments, the NARE comprises (i) SEQ ID NO: 348; (ii) SEQ ID NO:335; and (iii) SEQ ID NO:329.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to SEQ ID NO:299.

In some embodiments, the NARE comprises (i) any one of SEQ ID NOs:344-349, 498; (ii) SEQ ID NO:335; and (iii) SEQ ID NO:299.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO: 348; (ii) a sequence that is at least 80% identical, at least 85% identical, at least 90% identical, at least 95% identical, at least 96% identical, at least 97% identical, at least 98% identical, or at least 99% identical to SEQ ID NO:335; and (iii) a sequence that is at least 80% identical, at least 85% identical, at least 90% identical, at least 95% identical, at least 96% identical, at least 97% identical, at least 98% identical, or at least 99% identical to SEQ ID NO:299.

In some embodiments, the NARE comprises (i) SEQ ID NO: 348; (ii) SEQ ID NO:335; and (iii) SEQ ID NO:299.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; (iii) a sequence that is at least 80% identical, at least 85% identical, at least 90% identical, at least 95% identical, at least 96% identical, at least 97% identical, at least 98% identical, or at least 99% identical to any one of SEQ ID NOs:430, 431, 503; and (vi) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498; (ii) SEQ ID NO:335; (iii) any one of SEQ ID NOs:430, 431, 503; and (vi) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:335; (iii) a sequence that is at least 80% identical, at least 85% identical, at least 90% identical, at least 95% identical, at least 96% identical, at least 97% identical, at least 98% identical, or at least 99% identical to SEQ ID NO:503; and (vi) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:348; (ii) SEQ ID NO:335; (iii) SEQ ID NO:503; and (vi) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; (iii) a sequence that is at least 80% identical, at least 85% identical, at least 90% identical, at least 95% identical, at least 96% identical, at least 97% identical, at least 98% identical, or at least 99% identical to SEQ ID NOs:294 or 295; and (vi) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498; (ii) SEQ ID NO:335; (iii) SEQ ID NOs:294 or 295; and (vi) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:335; (iii) a sequence that is at least 80% identical, at least 85% identical, at least 90% identical, at least 95% identical, at least 96% identical, at least 97% identical, at least 98% identical, or at least 99% identical to SEQ ID NO:294; and (vi) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:348; (ii) SEQ ID NO:335; (iii) SEQ ID NO:294; and (vi) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; (iii) a sequence that is at least 80% identical, at least 85% identical, at least 90% identical, at least 95% identical, at least 96% identical, at least 97% identical, at least 98% identical, or at least 99% identical to SEQ ID NO:407 or 501; and (vi) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498; (ii) SEQ ID NO:335; (iii) SEQ ID NO:407 or 501; and (vi) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:335; (iii) a sequence that is at least 80% identical, at least 85% identical, at least 90% identical, at least 95% identical, at least 96% identical, at least 97% identical, at least 98% identical, or at least 99% identical to SEQ ID NO:501; and (vi) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:348; (ii) SEQ ID NO:335; (iii) SEQ ID NO:501; and (vi) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; (iii) a sequence that is at least 80% identical, at least 85% identical, at least 90% identical, at least 95% identical, at least 96% identical, at least 97% identical, at least 98% identical, or at least 99% identical to SEQ ID NOs:434 or 504; and (vi) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498; (ii) SEQ ID NO:335; (iii) SEQ ID NOs:434 or 504; and (vi) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:335; (iii) a sequence that is at least 80% identical, at least 85% identical, at least 90% identical, at least 95% identical, at least 96% identical, at least 97% identical, at least 98% identical, or at least 99% identical to SEQ ID NO:504; and (vi) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:348; (ii) SEQ ID NO:335; (iii) SEQ ID NO:504; and (vi) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:335; (iii) a sequence that is at least 80% identical, at least 85% identical, at least 90% identical, at least 95% identical, at least 96% identical, at least 97% identical, at least 98% identical, or at least 99% identical to SEQ ID NO:296; and (vi) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498; (ii) SEQ ID NO:335; (iii) SEQ ID NO:296; and (vi) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:335; (iii) a sequence that is at least 80% identical, at least 85% identical, at least 90% identical, at least 95% identical, at least 96% identical, at least 97% identical, at least 98% identical, or at least 99% identical to SEQ ID NO:296; and (vi) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:348; (ii) SEQ ID NO:335; (iii) SEQ ID NO:296; and (vi) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:339, 363-369, 378, 379, 382, 385, 387-389, 391, 398, 399; and (ii) a sequence that is at least 80% identical, at least 85% identical, at least 90% identical, at least 95% identical, at least 96% identical, at least 97% identical, at least 98% identical, or at least 99% identical to any one of SEQ ID NOs:430, 431, 503.

In some embodiments, the NARE comprises (i) any one of SEQ ID NOs:339, 363-369, 378, 379, 382, 385, 387-389, 391, 398, 399; and (ii) any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:339, 363-369, 378, 379, 382, 385, 387-389, 391, 398, 399; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) any one of SEQ ID NOs:339, 363-369, 378, 379, 382, 385, 387-389, 391, 398, 399; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:369; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:369; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:369; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:369; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:364; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:364; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:364; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:364; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:365; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:365; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:365; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:365; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:366; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:366; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:366; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:366; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:367; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:367; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:367; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:367; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:368; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:368; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:368; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:368; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:363; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:363; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:363; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:363; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:378; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:378; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:378; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:378; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:379; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:379; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:379; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:379; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:382; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:382; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:382; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:382; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:385; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:385; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:385; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:385; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:387; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:387; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:387; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:387; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:388; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:388; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:388; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:388; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:389; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:389; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:389; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:389; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:391; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:391; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:391; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:391; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:398; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:398; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:398; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:398; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:399; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:399; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:399; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:399; and (ii) SEQ ID NO:430.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:339; and (ii) a sequence that is at least 80% identical, at least 85% identical, at least 90% identical, at least 95% identical, at least 96% identical, at least 97% identical, at least 98% identical, or at least 99% identical to Any one of SEQ ID NOs:430, 431, 503.

In some embodiments, the NARE comprises (i) SEQ ID NO:339; and (ii) Any one of SEQ ID NOs:430, 431, 503.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:339; and (ii) a sequence that is at least 80% identical, at least 85% identical, at least 90% identical, at least 95% identical, at least 96% identical, at least 97% identical, at least 98% identical, or at least 99% identical to SEQ ID NO:430.

In some embodiments, the NARE comprises (i) SEQ ID NO:339; and (ii) SEQ ID NO:430.

In some embodiments, the NARE comprises a sequence that is at least 80% identical, at least 85% identical, at least 90% identical, at least 95% identical, at least 96% identical, at least 97% identical, at least 98% identical, or at least 99% identical to any one of SEQ ID NOs:138-161.

In some embodiments, the NARE comprises any one of SEQ ID NOs:138-161.

In some embodiments, the NARE comprises (i) optionally a sequence that is at least 80% identical, at least 85% identical, at least 90% identical, at least 95% identical, at least 96% identical, at least 97% identical, at least 98% identical, or at least 99% identical to SEQ ID NO:475; (ii) a sequence that is at least 80% identical, at least 85% identical, at least 90% identical, at least 95% identical, at least 96% identical, at least 97% identical, at least 98% identical, or at least 99% identical to SEQ ID NO:298, and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) optionally SEQ ID NO:475; (ii) SEQ ID NO:298, and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:298, and (ii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:298, and (ii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:475; (ii) a sequence that is at least 80% identical, at least 85% identical, at least 90% identical, at least 95% identical, at least 96% identical, at least 97% identical, at least 98% identical, or at least 99% identical to SEQ ID NO:298, and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:475; (ii) SEQ ID NO:298, and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:339, 380, 383, 386, 390; (ii) a sequence that is at least 80% identical, at least 85% identical, at least 90% identical, at least 95% identical, at least 96% identical, at least 97% identical, at least 98% identical, or at least 99% identical to SEQ ID NOs:439 or 497; and (iii) optionally a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:339, 380, 383, 386, 390; (ii) SEQ ID NOs:439 or 497; and (iii) optionally SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:339, 380, 383, 386, 390; and (ii) a sequence that is at least 80% identical, at least 85% identical, at least 90% identical, at least 95% identical, at least 96% identical, at least 97% identical, at least 98% identical, or at least 99% identical to SEQ ID NOs:439 or 497.

In some embodiments, the NARE comprises (i) any one of SEQ ID NOs:339, 380, 383, 386, 390; and (ii) SEQ ID NOs:439 or 497.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:339; and (ii) a sequence that is at least 80% identical, at least 85% identical, at least 90% identical, at least 95% identical, at least 96% identical, at least 97% identical, at least 98% identical, or at least 99% identical to SEQ ID NOs:439 or 497.

In some embodiments, the NARE comprises (i) SEQ ID NO:339; and (ii) SEQ ID NOs:439 or 497.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:339; and (ii) a sequence that is at least 80% identical, at least 85% identical, at least 90% identical, at least 95% identical, at least 96% identical, at least 97% identical, at least 98% identical, or at least 99% identical to SEQ ID NO:439.

In some embodiments, the NARE comprises (i) SEQ ID NO:339; and (ii) SEQ ID NO:439.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:380; and (ii) a sequence that is at least 80% identical, at least 85% identical, at least 90% identical, at least 95% identical, at least 96% identical, at least 97% identical, at least 98% identical, or at least 99% identical to c.

In some embodiments, the NARE comprises (i) SEQ ID NO:380; and (ii) SEQ ID NOs:439 or 497.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:380; and (ii) a sequence that is at least 80% identical, at least 85% identical, at least 90% identical, at least 95% identical, at least 96% identical, at least 97% identical, at least 98% identical, or at least 99% identical to SEQ ID NO:439.

In some embodiments, the NARE comprises (i) SEQ ID NO:380; and (ii) SEQ ID NO:439.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:383; and (ii) a sequence that is at least 80% identical, at least 85% identical, at least 90% identical, at least 95% identical, at least 96% identical, at least 97% identical, at least 98% identical, or at least 99% identical to SEQ ID NOs:439 or 497.

In some embodiments, the NARE comprises (i) SEQ ID NO:383; and (ii) SEQ ID NOs:439 or 497.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:383; and (ii) a sequence that is at least 80% identical, at least 85% identical, at least 90% identical, at least 95% identical, at least 96% identical, at least 97% identical, at least 98% identical, or at least 99% identical to SEQ ID NO:439.

In some embodiments, the NARE comprises (i) SEQ ID NO:383; and (ii) SEQ ID NO:439.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:386; and (ii) a sequence that is at least 80% identical, at least 85% identical, at least 90% identical, at least 95% identical, at least 96% identical, at least 97% identical, at least 98% identical, or at least 99% identical to SEQ ID NOs:439 or 497.

In some embodiments, the NARE comprises (i) SEQ ID NO:386; and (ii) SEQ ID NOs:439 or 497.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:386; and (ii) a sequence that is at least 80% identical, at least 85% identical, at least 90% identical, at least 95% identical, at least 96% identical, at least 97% identical, at least 98% identical, or at least 99% identical to SEQ ID NO:439.

In some embodiments, the NARE comprises (i) SEQ ID NO:386; and (ii) SEQ ID NO:439.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:390; and (ii) a sequence that is at least 80% identical, at least 85% identical, at least 90% identical, at least 95% identical, at least 96% identical, at least 97% identical, at least 98% identical, or at least 99% identical to SEQ ID NOs:439 or 497.

In some embodiments, the NARE comprises (i) SEQ ID NO:390; and (ii) SEQ ID NOs:439 or 497.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:390; and (ii) a sequence that is at least 80% identical, at least 85% identical, at least 90% identical, at least 95% identical, at least 96% identical, at least 97% identical, at least 98% identical, or at least 99% identical to SEQ ID NO:497.

In some embodiments, the NARE comprises (i) SEQ ID NO:390; and (ii) SEQ ID NO:497.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:390; and (ii) a sequence that is at least 80% identical, at least 85% identical, at least 90% identical, at least 95% identical, at least 96% identical, at least 97% identical, at least 98% identical, or at least 99% identical to SEQ ID NO:439.

In some embodiments, the NARE comprises (i) SEQ ID NO:390; and (ii) SEQ ID NO:439.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:390; (ii) a sequence that is at least 80% identical, at least 85% identical, at least 90% identical, at least 95% identical, at least 96% identical, at least 97% identical, at least 98% identical, or at least 99% identical to SEQ ID NOs:439 or 497; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:390; (ii) SEQ ID NO:439 or 497; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:390; (ii) a sequence that is at least 80% identical, at least 85% identical, at least 90% identical, at least 95% identical, at least 96% identical, at least 97% identical, at least 98% identical, or at least 99% identical to SEQ ID NO:439; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:390; (ii) SEQ ID NO:439; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to any one of SEQ ID NO:201, 284, 285, 301, 362, 372, 373, 403-406, 408-410, 412, 427, 444, 453-456, 486, 490.

In some embodiments, the NARE comprises (i) any one of SEQ ID NOs:344-349, 498; (ii) SEQ ID NO:336; and (iii) any one of SEQ ID NO:201, 284, 285, 301, 362, 372, 373, 403-406, 408-410, 412, 427, 444, 453-456, 486, 490.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to any one of SEQ ID NO:201, 284, 285, 301, 362, 372, 373, 403-406, 408-410, 412, 427, 444, 453-456, 486, 490.

In some embodiments, the NARE comprises (i) SEQ ID NO:348; (ii) SEQ ID NO:336; and (iii) any one of SEQ ID NO:201, 284, 285, 301, 362, 372, 373, 403-406, 408-410, 412, 427, 444, 453-456, 486, 490.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) any one of SEQ ID NOs:344-349, 498; (ii) SEQ ID NO:336; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:348; (ii) SEQ ID NO:336; and (iii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:372.

In some embodiments, the NARE comprises (i) any one of SEQ ID NOs:344-349, 498; (ii) SEQ ID NO:336; and (iii) SEQ ID NO:372.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:372.

In some embodiments, the NARE comprises (i) SEQ ID NO:348; (ii) SEQ ID NO:336; and (iii) SEQ ID NO:372.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:373.

In some embodiments, the NARE comprises (i) any one of SEQ ID NOs:344-349, 498; (ii) SEQ ID NO:336; and (iii) SEQ ID NO:373.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:373.

In some embodiments, the NARE comprises (i) SEQ ID NO:348; (ii) SEQ ID NO:336; and (iii) SEQ ID NO:373.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:486.

In some embodiments, the NARE comprises (i) any one of SEQ ID NOs:344-349, 498; (ii) SEQ ID NO:336; and (iii) SEQ ID NO:486.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:486.

In some embodiments, the NARE comprises (i) SEQ ID NO:348; (ii) SEQ ID NO:336; and (iii) SEQ ID NO:486.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:456.

In some embodiments, the NARE comprises (i) any one of SEQ ID NOs:344-349, 498; (ii) SEQ ID NO:336; and (iii) SEQ ID NO:456.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:456.

In some embodiments, the NARE comprises (i) SEQ ID NO:348; (ii) SEQ ID NO:336; and (iii) SEQ ID NO:456.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:455.

In some embodiments, the NARE comprises (i) any one of SEQ ID NOs:344-349, 498; (ii) SEQ ID NO:336; and (iii) SEQ ID NO:455.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:455.

In some embodiments, the NARE comprises (i) SEQ ID NO:348; (ii) SEQ ID NO:336; and (iii) SEQ ID NO:455.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:406.

In some embodiments, the NARE comprises (i) any one of SEQ ID NOs:344-349, 498; (ii) SEQ ID NO:336; and (iii) SEQ ID NO:406.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:406.

In some embodiments, the NARE comprises (i) SEQ ID NO:348; (ii) SEQ ID NO:336; and (iii) SEQ ID NO:406.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:490.

In some embodiments, the NARE comprises (i) any one of SEQ ID NOs:344-349, 498; (ii) SEQ ID NO:336; and (iii) SEQ ID NO:490.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:490.

In some embodiments, the NARE comprises (i) SEQ ID NO:348; (ii) SEQ ID NO:336; and (iii) SEQ ID NO:490.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:201.

In some embodiments, the NARE comprises (i) any one of SEQ ID NOs:344-349, 498; (ii) SEQ ID NO:336; and (iii) SEQ ID NO:201.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:201.

In some embodiments, the NARE comprises (i) SEQ ID NO:348; (ii) SEQ ID NO:336; and (iii) SEQ ID NO:201.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:301.

In some embodiments, the NARE comprises (i) any one of SEQ ID NOs:344-349, 498; (ii) SEQ ID NO:336; and (iii) SEQ ID NO:301.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:301.

In some embodiments, the NARE comprises (i) SEQ ID NO:348; (ii) SEQ ID NO:336; and (iii) SEQ ID NO:301.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:403.

In some embodiments, the NARE comprises (i) any one of SEQ ID NOs:344-349, 498; (ii) SEQ ID NO:336; and (iii) SEQ ID NO:403.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:403.

In some embodiments, the NARE comprises (i) SEQ ID NO:348; (ii) SEQ ID NO:336; and (iii) SEQ ID NO:403.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:362.

In some embodiments, the NARE comprises (i) any one of SEQ ID NOs:344-349, 498; (ii) SEQ ID NO:336; and (iii) SEQ ID NO:362.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:362.

In some embodiments, the NARE comprises (i) SEQ ID NO:348; (ii) SEQ ID NO:336; and (iii) SEQ ID NO:362.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:405.

In some embodiments, the NARE comprises (i) any one of SEQ ID NOs:344-349, 498; (ii) SEQ ID NO:336; and (iii) SEQ ID NO:405.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:405.

In some embodiments, the NARE comprises (i) SEQ ID NO:348; (ii) SEQ ID NO:336; and (iii) SEQ ID NO:405.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:453.

In some embodiments, the NARE comprises (i) any one of SEQ ID NOs:344-349, 498; (ii) SEQ ID NO:336; and (iii) SEQ ID NO:453.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:453.

In some embodiments, the NARE comprises (i) SEQ ID NO:348; (ii) SEQ ID NO:336; and (iii) SEQ ID NO:453.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:284.

In some embodiments, the NARE comprises (i) any one of SEQ ID NOs:344-349, 498; (ii) SEQ ID NO:336; and (iii) SEQ ID NO:284.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:284.

In some embodiments, the NARE comprises (i) SEQ ID NO:348; (ii) SEQ ID NO:336; and (iii) SEQ ID NO:284.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:285.

In some embodiments, the NARE comprises (i) any one of SEQ ID NOs:344-349, 498; (ii) SEQ ID NO:336; and (iii) SEQ ID NO:285.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:285.

In some embodiments, the NARE comprises (i) SEQ ID NO:348; (ii) SEQ ID NO:336; and (iii) SEQ ID NO:285.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:412.

In some embodiments, the NARE comprises (i) any one of SEQ ID NOs:344-349, 498; (ii) SEQ ID NO:336; and (iii) SEQ ID NO:412.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:412.

In some embodiments, the NARE comprises (i) SEQ ID NO:348; (ii) SEQ ID NO:336; and (iii) SEQ ID NO:412.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:404.

In some embodiments, the NARE comprises (i) any one of SEQ ID NOs:344-349, 498; (ii) SEQ ID NO:336; and (iii) SEQ ID NO:404.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:404.

In some embodiments, the NARE comprises (i) SEQ ID NO:348; (ii) SEQ ID NO:336; and (iii) SEQ ID NO:404.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:408.

In some embodiments, the NARE comprises (i) any one of SEQ ID NOs:344-349, 498; (ii) SEQ ID NO:336; and (iii) SEQ ID NO:408.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:408.

In some embodiments, the NARE comprises (i) SEQ ID NO:348; (ii) SEQ ID NO:336; and (iii) SEQ ID NO:408.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:409.

In some embodiments, the NARE comprises (i) any one of SEQ ID NOs:344-349, 498; (ii) SEQ ID NO:336; and (iii) SEQ ID NO:409.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:409.

In some embodiments, the NARE comprises (i) SEQ ID NO:348; (ii) SEQ ID NO:336; and (iii) SEQ ID NO:409.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:410.

In some embodiments, the NARE comprises (i) any one of SEQ ID NOs:344-349, 498; (ii) SEQ ID NO:336; and (iii) SEQ ID NO:410.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:410.

In some embodiments, the NARE comprises (i) SEQ ID NO:348; (ii) SEQ ID NO:336; and (iii) SEQ ID NO:410.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:427.

In some embodiments, the NARE comprises (i) any one of SEQ ID NOs:344-349, 498; (ii) SEQ ID NO:336; and (iii) SEQ ID NO:427.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:427.

In some embodiments, the NARE comprises (i) SEQ ID NO:348; (ii) SEQ ID NO:336; and (iii) SEQ ID NO:427.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:454.

In some embodiments, the NARE comprises (i) any one of SEQ ID NOs:344-349, 498; (ii) SEQ ID NO:336; and (iii) SEQ ID NO:454.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:348; (ii) a sequence that is at least 80% identical, at least 85% identical, at least 90% identical, at least 95% identical, at least 96% identical, at least 97% identical, at least 98% identical, or at least 99% identical to SEQ ID NO:336; and (iii) a sequence that is at least 80% identical, at least 85% identical, at least 90% identical, at least 95% identical, at least 96% identical, at least 97% identical, at least 98% identical, or at least 99% identical to SEQ ID NO:454.

In some embodiments, the NARE comprises (i) SEQ ID NO:348; (ii) SEQ ID NO:336; and (iii) SEQ ID NO:454.

In some embodiments, the NARE comprises (i) optionally a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498 or 393; (ii) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:93-98, 103, 109, 128-136, 195-198, 201, 284, 285, 301, 362, 371, 373, 403-406, 408-410, 412, 417-420, 502, 427, 453-456, 460-474, 486, 490; and (iv) optionally a sequence that is at least 80% identical, at least 85% identical, at least 90% identical, at least 95% identical, at least 96% identical, at least 97% identical, at least 98% identical, or at least 99% identical to SEQ ID NOs:443 or 448.

In some embodiments, the NARE comprises (i) optionally any one of SEQ ID NOs:344-349, 498 or 393; (ii) any one of SEQ ID NOs:374-376; (iii) any one of SEQ ID NOs:93-98, 103, 109, 128-136, 195-198, 201, 284, 285, 301, 362, 371, 373, 403-406, 408-410, 412, 417-420, 502, 427, 453-456, 460-474, 486, 490; and (iv) optionally SEQ ID NOs:443 or 448.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to any one of SEQ ID NOs:93-98, 103, 109, 128-136, 195-198, 201, 284, 285, 301, 362, 371, 373, 403-406, 408-410, 412, 427, 453-456, 460-474, 486, 490.

In some embodiments, the NARE comprises (i) any one of SEQ ID NOs:374-376; and (ii) any one of SEQ ID NOs:93-98, 103, 109, 128-136, 195-198, 201, 284, 285, 301, 362, 371, 373, 403-406, 408-410, 412, 427, 453-456, 460-474, 486, 490.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to any one of SEQ ID NOs:201, 284, 285, 301, 362, 371, 373, 403-406, 408-410, 412, 427, 453-456, 486, 490.

In some embodiments, the NARE comprises (i) any one of SEQ ID NOs:374-376; and (ii) any one of SEQ ID NOs:201, 284, 285, 301, 362, 371, 373, 403-406, 408-410, 412, 427, 453-456, 486, 490.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to any one of SEQ ID NOs:201, 284, 285, 301, 362, 371, 373, 403-406, 408-410, 412, 427, 453-456, 486, 490.

In some embodiments, the NARE comprises (i) SEQ ID NO:376; and (ii) any one of SEQ ID NOs:201, 284, 285, 301, 362, 371, 373, 403-406, 408-410, 412, 427, 453-456, 486, 490.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to any one of SEQ ID NOs:93-98, 103, 109, 128-136, 195-198, 460-474.

In some embodiments, the NARE comprises (i) any one of SEQ ID NOs:374-376; and (ii) any one of SEQ ID NOs:93-98, 103, 109, 128-136, 195-198, 460-474.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to any one of SEQ ID NOs:93-98, 103, 109, 128-136, 195-198, 460-469, 471-474.

In some embodiments, the NARE comprises (i) SEQ ID NO:375; and (ii) any one of SEQ ID NOs:93-98, 103, 109, 128-136, 195-198, 460-469, 471-474.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498 or 393; (ii) a sequence that is at least 80% identical, at least 85% identical, at least 90% identical, at least 95% identical, at least 96% identical, at least 97% identical, at least 98% identical, or at least 99% identical to any one of SEQ ID NOs:374-376; (iii) a sequence that is at least 80% identical, at least 85% identical, at least 90% identical, at least 95% identical, at least 96% identical, at least 97% identical, at least 98% identical, or at least 99% identical to any one of SEQ ID NOs:417-420, 502; and (iv) optionally a sequence that is at least 80% identical, at least 85% identical, at least 90% identical, at least 95% identical, at least 96% identical, at least 97% identical, at least 98% identical, or at least 99% identical to SEQ ID NOs:443 or 448.

In some embodiments, the NARE comprises (i) any one of SEQ ID NOs:344-349, 498 or 393; (ii) any one of SEQ ID NOs:374-376; (iii) any one of SEQ ID NOs:417-420, 502; and (iv) optionally SEQ ID NOs:443 or 448.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:349 or 393; (ii) a sequence that is at least 80% identical, at least 85% identical, at least 90% identical, at least 95% identical, at least 96% identical, at least 97% identical, at least 98% identical, or at least 99% identical to SEQ ID NOs:374; (iii) a sequence that is at least 80% identical, at least 85% identical, at least 90% identical, at least 95% identical, at least 96% identical, at least 97% identical, at least 98% identical, or at least 99% identical to SEQ ID NO:420; and (iv) optionally a sequence that is at least 80% identical, at least 85% identical, at least 90% identical, at least 95% identical, at least 96% identical, at least 97% identical, at least 98% identical, or at least 99% identical to SEQ ID NOs:443 or 448.

In some embodiments, the NARE comprises (i) a SEQ ID NO:349 or 393; (ii) SEQ ID NOs:374; (iii) SEQ ID NO:420; and (iv) optionally SEQ ID NOs:443 or 448.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:417-420, 502.

In some embodiments, the NARE comprises (i) any one of SEQ ID NOs:344-349, 498; (ii any one of SEQ ID NOs:374-376; and (iii) any one of SEQ ID NOs:417-420, 502.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:498; (ii) a sequence that is at least 80% identical, at least 85% identical, at least 90% identical, at least 95% identical, at least 96% identical, at least 97% identical, at least 98% identical, or at least 99% identical to SEQ ID NO:374; and (iii) a sequence that is at least 80% identical, at least 85% identical, at least 90% identical, at least 95% identical, at least 96% identical, at least 97% identical, at least 98% identical, or at least 99% identical to SEQ ID NO:420.

In some embodiments, the NARE comprises (i) SEQ ID NO:498; (ii) SEQ ID NO:374; and (iii) SEQ ID NO:420.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:344-349, 498; (ii) a sequence that is at least 80% identical, at least 85% identical, at least 90% identical, at least 95% identical, at least 96% identical, at least 97% identical, at least 98% identical, or at least 99% identical to any one of SEQ ID NOs:374-376; (iii) a sequence that is at least 80% identical, at least 85% identical, at least 90% identical, at least 95% identical, at least 96% identical, at least 97% identical, at least 98% identical, or at least 99% identical to any one of SEQ ID NOs:417-420, 502; and (vi) a sequence that is at least 80% identical, at least 85% identical, at least 90% identical, at least 95% identical, at least 96% identical, at least 97% identical, at least 98% identical, or at least 99% identical to SEQ ID NO:443.

In some embodiments, the NARE comprises (i) any one of SEQ ID NOs:344-349, 498; (ii) any one of SEQ ID NOs:374-376; (iii) any one of SEQ ID NOs:417-420, 502; and (vi) SEQ ID NO:443.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:498; (ii) a sequence that is at least 80% identical, at least 85% identical, at least 90% identical, at least 95% identical, at least 96% identical, at least 97% identical, at least 98% identical, or at least 99% identical to SEQ ID NO:374; (iii) a sequence that is at least 80% identical, at least 85% identical, at least 90% identical, at least 95% identical, at least 96% identical, at least 97% identical, at least 98% identical, or at least 99% identical to SEQ ID NO:420; and (vi) a sequence that is at least 80% identical, at least 85% identical, at least 90% identical, at least 95% identical, at least 96% identical, at least 97% identical, at least 98% identical, or at least 99% identical to SEQ ID NO:443.

In some embodiments, the NARE comprises (i) SEQ ID NO:498; (ii) SEQ ID NO:374; (iii) SEQ ID NO:420; and (vi) SEQ ID NO:443.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:393; (ii) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (iii) a sequence that is at least 80% identical, at least 85% identical, at least 90% identical, at least 95% identical, at least 96% identical, at least 97% identical, at least 98% identical, or at least 99% identical to any one of SEQ ID NOs:417-420, 502.

In some embodiments, the NARE comprises (i) any one of SEQ ID NO:393; (ii) any one of SEQ ID NOs:374-376; and (iii) any one of SEQ ID NOs:417-420, 502.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:393; (ii) a sequence that is at least 80% identical, at least 85% identical, at least 90% identical, at least 95% identical, at least 96% identical, at least 97% identical, at least 98% identical, or at least 99% identical to SEQ ID NO:374; and (iii) a sequence that is at least 80% identical, at least 85% identical, at least 90% identical, at least 95% identical, at least 96% identical, at least 97% identical, at least 98% identical, or at least 99% identical to SEQ ID NO:420.

In some embodiments, the NARE comprises (i) SEQ ID NO:393; (ii) SEQ ID NO:374; and (iii) SEQ ID NO:420.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:393; (ii) a sequence that is at least 80% identical, at least 85% identical, at least 90% identical, at least 95% identical, at least 96% identical, at least 97% identical, at least 98% identical, or at least 99% identical to any one of SEQ ID NOs:374-376; (iii) a sequence that is at least 80% identical, at least 85% identical, at least 90% identical, at least 95% identical, at least 96% identical, at least 97% identical, at least 98% identical, or at least 99% identical to any one of SEQ ID NOs:417-420, 502; and (iv) a sequence that is at least 80% identical, at least 85% identical, at least 90% identical, at least 95% identical, at least 96% identical, at least 97% identical, at least 98% identical, or at least 99% identical to SEQ ID NO:448.

In some embodiments, the NARE comprises (i) any one of SEQ ID NO: 393; (ii) any one of SEQ ID NOs:374-376; (iii) any one of SEQ ID NOs:417-420, 502; and (iv) SEQ ID NO:448.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:393; (ii) a sequence that is at least 80% identical, at least 85% identical, at least 90% identical, at least 95% identical, at least 96% identical, at least 97% identical, at least 98% identical, or at least 99% identical to SEQ ID NO:374; (iii) a sequence that is at least 80% identical, at least 85% identical, at least 90% identical, at least 95% identical, at least 96% identical, at least 97% identical, at least 98% identical, or at least 99% identical to SEQ ID NO:420; and (iv) a sequence that is at least 80% identical, at least 85% identical, at least 90% identical, at least 95% identical, at least 96% identical, at least 97% identical, at least 98% identical, or at least 99% identical to SEQ ID NO:448.

In some embodiments, the NARE comprises (i) SEQ ID NO:393; (ii) SEQ ID NO:374; (iii) SEQ ID NO:420; and (iv) SEQ ID NO:448.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:371.

In some embodiments, the NARE comprises (i) any one of SEQ ID NOs:374-376; and (ii) SEQ ID NO:371.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:371.

In some embodiments, the NARE comprises (i) SEQ ID NO:376; and (ii) SEQ ID NO:371.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:373.

In some embodiments, the NARE comprises (i) any one of SEQ ID NOs:374-376; and (ii) SEQ ID NO:373.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:373.

In some embodiments, the NARE comprises (i) SEQ ID NO:376; and (ii) SEQ ID NO:373.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:486.

In some embodiments, the NARE comprises (i) any one of SEQ ID NOs:374-376; and (ii) SEQ ID NO:486.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:486.

In some embodiments, the NARE comprises (i) SEQ ID NO:376; and (ii) SEQ ID NO:486.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:456.

In some embodiments, the NARE comprises (i) any one of SEQ ID NOs:374-376; and (ii) SEQ ID NO:456.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:456.

In some embodiments, the NARE comprises (i) SEQ ID NO:376; and (ii) SEQ ID NO:456.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:455.

In some embodiments, the NARE comprises (i) any one of SEQ ID NOs:374-376; and (ii) SEQ ID NO:455.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:455.

In some embodiments, the NARE comprises (i) SEQ ID NO:376; and (ii) SEQ ID NO:455.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:406.

In some embodiments, the NARE comprises (i) any one of SEQ ID NOs:374-376; and (ii) SEQ ID NO:406.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:406.

In some embodiments, the NARE comprises (i) SEQ ID NO:376; and (ii) SEQ ID NO:406.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:490.

In some embodiments, the NARE comprises (i) any one of SEQ ID NOs:374-376; and (ii) SEQ ID NO:490.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:490.

In some embodiments, the NARE comprises (i) SEQ ID NO:376; and (ii) SEQ ID NO:490.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:201.

In some embodiments, the NARE comprises (i) any one of SEQ ID NOs:374-376; and (ii) SEQ ID NO:201.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:201.

In some embodiments, the NARE comprises (i) SEQ ID NO:376; and (ii) SEQ ID NO:201.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:301.

In some embodiments, the NARE comprises (i) any one of SEQ ID NOs:374-376; and (ii) SEQ ID NO:301.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:301.

In some embodiments, the NARE comprises (i) SEQ ID NO:376; and (ii) SEQ ID NO:301.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:403.

In some embodiments, the NARE comprises (i) any one of SEQ ID NOs:374-376; and (ii) SEQ ID NO:403.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:403.

In some embodiments, the NARE comprises (i) SEQ ID NO:376; and (ii) SEQ ID NO:403.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:362.

In some embodiments, the NARE comprises (i) any one of SEQ ID NOs:374-376; and (ii) SEQ ID NO:362.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:362.

In some embodiments, the NARE comprises (i) SEQ ID NO:376; and (ii) SEQ ID NO:362.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:405.

In some embodiments, the NARE comprises (i) any one of SEQ ID NOs:374-376; and (ii) SEQ ID NO:405.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:405.

In some embodiments, the NARE comprises (i) SEQ ID NO:376; and (ii) SEQ ID NO:405.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:453.

In some embodiments, the NARE comprises (i) any one of SEQ ID NOs:374-376; and (ii) SEQ ID NO:453.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:453.

In some embodiments, the NARE comprises (i) SEQ ID NO:376; and (ii) SEQ ID NO:453.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:284.

In some embodiments, the NARE comprises (i) any one of SEQ ID NOs:374-376; and (ii) SEQ ID NO:284.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:284.

In some embodiments, the NARE comprises (i) SEQ ID NO:376; and (ii) SEQ ID NO:284.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:285.

In some embodiments, the NARE comprises (i) any one of SEQ ID NOs:374-376; and (ii) to SEQ ID NO:285.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:285.

In some embodiments, the NARE comprises (i) SEQ ID NO:376; and (ii) SEQ ID NO:285.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:412.

In some embodiments, the NARE comprises (i) any one of SEQ ID NOs:374-376; and (ii) to SEQ ID NO:412.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:412.

In some embodiments, the NARE comprises (i) SEQ ID NO:376; and (ii) SEQ ID NO:412.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:404.

In some embodiments, the NARE comprises (i) any one of SEQ ID NOs:374-376; and (ii) to SEQ ID NO:404.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:404.

In some embodiments, the NARE comprises (i) SEQ ID NO:376; and (ii) SEQ ID NO:404.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:408.

In some embodiments, the NARE comprises (i) any one of SEQ ID NOs:374-376; and (ii) to SEQ ID NO:408.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:408.

In some embodiments, the NARE comprises (i) SEQ ID NO:376; and (ii) SEQ ID NO:408.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:409.

In some embodiments, the NARE comprises (i) any one of SEQ ID NOs:374-376; and (ii) to SEQ ID NO:409.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:409.

In some embodiments, the NARE comprises (i) SEQ ID NO:376; and (ii) SEQ ID NO:409.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:410.

In some embodiments, the NARE comprises (i) any one of SEQ ID NOs:374-376; and (ii) to SEQ ID NO:410.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:410.

In some embodiments, the NARE comprises (i) SEQ ID NO:376; and (ii) SEQ ID NO:410.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:427.

In some embodiments, the NARE comprises (i) any one of SEQ ID NOs:374-376; and (ii) to SEQ ID NO:427.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:427.

In some embodiments, the NARE comprises (i) SEQ ID NO:376; and (ii) SEQ ID NO:427.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:454.

In some embodiments, the NARE comprises (i) any one of SEQ ID NOs:374-376; and (ii) to SEQ ID NO:454.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:376; and (ii) a sequence that is at least 80% identical, at least 85% identical, at least 90% identical, at least 95% identical, at least 96% identical, at least 97% identical, at least 98% identical, or at least 99% identical to SEQ ID NO:454.

In some embodiments, the NARE comprises (i) SEQ ID NO:376; and (ii) SEQ ID NO:454.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:472.

In some embodiments, the NARE comprises (i) any one of SEQ ID NOs:374-376; and (ii) to SEQ ID NO:472.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:472.

In some embodiments, the NARE comprises (i) SEQ ID NO:375; and (ii) SEQ ID NO:472.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NOs:469 or 470.

In some embodiments, the NARE comprises (i) any one of SEQ ID NOs:374-376; and (ii) to SEQ ID NOs:469 or 470.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:469.

In some embodiments, the NARE comprises (i) SEQ ID NO:375; and (ii) SEQ ID NO:469.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:471.

In some embodiments, the NARE comprises (i) any one of SEQ ID NOs:374-376; and (ii) to SEQ ID NO:471.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:471.

In some embodiments, the NARE comprises (i) SEQ ID NO:375; and (ii) SEQ ID NO:471.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:462.

In some embodiments, the NARE comprises (i) any one of SEQ ID NOs:374-376; and (ii) to SEQ ID NO:462.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:462.

In some embodiments, the NARE comprises (i) SEQ ID NO:375; and (ii) SEQ ID NO:462.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:464.

In some embodiments, the NARE comprises (i) any one of SEQ ID NOs:374-376; and (ii) to SEQ ID NO:464.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:464.

In some embodiments, the NARE comprises (i) SEQ ID NO:375; and (ii) SEQ ID NO:464.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:467.

In some embodiments, the NARE comprises (i) any one of SEQ ID NOs:374-376; and (ii) to SEQ ID NO:467.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:467.

In some embodiments, the NARE comprises (i) SEQ ID NO:375; and (ii) SEQ ID NO:467.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:468.

In some embodiments, the NARE comprises (i) any one of SEQ ID NOs:374-376; and (ii) to SEQ ID NO:468.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:468.

In some embodiments, the NARE comprises (i) SEQ ID NO:375; and (ii) SEQ ID NO:468.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:461.

In some embodiments, the NARE comprises (i) any one of SEQ ID NOs:374-376; and (ii) to SEQ ID NO:461.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:461.

In some embodiments, the NARE comprises (i) SEQ ID NO:375; and (ii) SEQ ID NO:461.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:460.

In some embodiments, the NARE comprises (i) any one of SEQ ID NOs:374-376; and (ii) to SEQ ID NO:460.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:460.

In some embodiments, the NARE comprises (i) SEQ ID NO:375; and (ii) SEQ ID NO:460.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:465.

In some embodiments, the NARE comprises (i) any one of SEQ ID NOs:374-376; and (ii) to SEQ ID NO:465.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:465.

In some embodiments, the NARE comprises (i) SEQ ID NO:375; and (ii) SEQ ID NO:465.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:466.

In some embodiments, the NARE comprises (i) any one of SEQ ID NOs:374-376; and (ii) to SEQ ID NO:466.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:466.

In some embodiments, the NARE comprises (i) SEQ ID NO:375; and (ii) SEQ ID NO:466.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:98.

In some embodiments, the NARE comprises (i) any one of SEQ ID NOs:374-376; and (ii) to SEQ ID NO:98.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:98.

In some embodiments, the NARE comprises (i) SEQ ID NO:375; and (ii) SEQ ID NO:98.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:103.

In some embodiments, the NARE comprises (i) any one of SEQ ID NOs:374-376; and (ii) to SEQ ID NO:103.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 103.

In some embodiments, the NARE comprises (i) SEQ ID NO:375; and (ii) SEQ ID NO:103.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:197.

In some embodiments, the NARE comprises (i) any one of SEQ ID NOs:374-376; and (ii) to SEQ ID NO:197.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 197.

In some embodiments, the NARE comprises (i) SEQ ID NO:375; and (ii) SEQ ID NO:197.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:196.

In some embodiments, the NARE comprises (i) any one of SEQ ID NOs:374-376; and (ii) to SEQ ID NO:196.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 196.

In some embodiments, the NARE comprises (i) SEQ ID NO:375; and (ii) SEQ ID NO:196.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:195.

In some embodiments, the NARE comprises (i) any one of SEQ ID NOs:374-376; and (ii) to SEQ ID NO:195.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 195.

In some embodiments, the NARE comprises (i) SEQ ID NO:375; and (ii) SEQ ID NO:195.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:129.

In some embodiments, the NARE comprises (i) any one of SEQ ID NOs:374-376; and (ii) to SEQ ID NO:129.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 129.

In some embodiments, the NARE comprises (i) SEQ ID NO:375; and (ii) SEQ ID NO:129.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:198.

In some embodiments, the NARE comprises (i) any one of SEQ ID NOs:374-376; and (ii) to SEQ ID NO:198.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 198.

In some embodiments, the NARE comprises (i) SEQ ID NO:375; and (ii) SEQ ID NO:198.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:93.

In some embodiments, the NARE comprises (i) any one of SEQ ID NOs:374-376; and (ii) to SEQ ID NO:93.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:93.

In some embodiments, the NARE comprises (i) SEQ ID NO:375; and (ii) SEQ ID NO:93.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:96.

In some embodiments, the NARE comprises (i) any one of SEQ ID NOs:374-376; and (ii) to SEQ ID NO:96.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:96.

In some embodiments, the NARE comprises (i) SEQ ID NO:375; and (ii) SEQ ID NO:96.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:109.

In some embodiments, the NARE comprises (i) any one of SEQ ID NOs:374-376; and (ii) to SEQ ID NO:109.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 109.

In some embodiments, the NARE comprises (i) SEQ ID NO:375; and (ii) SEQ ID NO:109.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:97.

In some embodiments, the NARE comprises (i) any one of SEQ ID NOs:374-376; and (ii) to SEQ ID NO:97.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:97.

In some embodiments, the NARE comprises (i) SEQ ID NO:375; and (ii) SEQ ID NO:97.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:128.

In some embodiments, the NARE comprises (i) any one of SEQ ID NOs:374-376; and (ii) to SEQ ID NO:128.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 128.

In some embodiments, the NARE comprises (i) SEQ ID NO:375; and (ii) SEQ ID NO:128.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:135.

In some embodiments, the NARE comprises (i) any one of SEQ ID NOs:374-376; and (ii) to SEQ ID NO:135.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 135.

In some embodiments, the NARE comprises (i) SEQ ID NO:375; and (ii) SEQ ID NO:135.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:94.

In some embodiments, the NARE comprises (i) any one of SEQ ID NOs:374-376; and (ii) to SEQ ID NO:94.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:94.

In some embodiments, the NARE comprises (i) SEQ ID NO:375; and (ii) SEQ ID NO:94.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:95.

In some embodiments, the NARE comprises (i) any one of SEQ ID NOs:374-376; and (ii) to SEQ ID NO:95.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:95.

In some embodiments, the NARE comprises (i) SEQ ID NO:375; and (ii) SEQ ID NO:95.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:133.

In some embodiments, the NARE comprises (i) any one of SEQ ID NOs:374-376; and (ii) to SEQ ID NO:133.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 133.

In some embodiments, the NARE comprises (i) SEQ ID NO:375; and (ii) SEQ ID NO:133.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:130.

In some embodiments, the NARE comprises (i) any one of SEQ ID NOs:374-376; and (ii) to SEQ ID NO:130.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 130.

In some embodiments, the NARE comprises (i) SEQ ID NO:375; and (ii) SEQ ID NO:130.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:131.

In some embodiments, the NARE comprises (i) any one of SEQ ID NOs:374-376; and (ii) to SEQ ID NO:131.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:131.

In some embodiments, the NARE comprises (i) SEQ ID NO:375; and (ii) SEQ ID NO:131.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:132.

In some embodiments, the NARE comprises (i) any one of SEQ ID NOs:374-376; and (ii) to SEQ ID NO:132.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 132.

In some embodiments, the NARE comprises (i) SEQ ID NO:375; and (ii) SEQ ID NO:132.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:134.

In some embodiments, the NARE comprises (i) any one of SEQ ID NOs:374-376; and (ii) to SEQ ID NO:134.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 134.

In some embodiments, the NARE comprises (i) SEQ ID NO:375; and (ii) SEQ ID NO:134.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:136.

In some embodiments, the NARE comprises (i) any one of SEQ ID NOs:374-376; and (ii) to SEQ ID NO:136.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO: 136.

In some embodiments, the NARE comprises (i) SEQ ID NO:375; and (ii) SEQ ID NO:136.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:463.

In some embodiments, the NARE comprises (i) any one of SEQ ID NOs:374-376; and (ii) to SEQ ID NO:463.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:463.

In some embodiments, the NARE comprises (i) SEQ ID NO:375; and (ii) SEQ ID NO:463.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:473.

In some embodiments, the NARE comprises (i) any one of SEQ ID NOs:374-376; and (ii) to SEQ ID NO:473.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:473.

In some embodiments, the NARE comprises (i) SEQ ID NO:375; and (ii) c.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:374-376; and (ii) a sequence that is at least 80% identical, at least 85% identical, at least 90% identical, at least 95% identical, at least 96% identical, at least 97% identical, at least 98% identical, or at least 99% identical to SEQ ID NO:474.

In some embodiments, the NARE comprises (i) any one of SEQ ID NOs:374-376; and (ii) to SEQ ID NO:474.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:375; and (ii) a sequence that is at least 80% identical, at least 85% identical, at least 90% identical, at least 95% identical, at least 96% identical, at least 97% identical, at least 98% identical, or at least 99% identical to SEQ ID NO:474.

In some embodiments, the NARE comprises (i) SEQ ID NO:375; and (ii) SEQ ID NO:474.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NOs:478 or 479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iv) a sequence that is at least 80% identical, at least 85% identical, at least 90% identical, at least 95% identical, at least 96% identical, at least 97% identical, at least 98% identical, or at least 99% identical to any one of SEQ ID NOs:361, 421, 422, 423, 428, 444, 480.

In some embodiments, the NARE comprises (i) SEQ ID NOs:478 or 479; (ii) SEQ ID NO:350; (iii) any one of SEQ ID NOs:351-356, 499, 500; and (iv) any one of SEQ ID NOs:361, 421, 422, 423, 428, 444, 480.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to SEQ ID NOs:354 or 356; and (iv) a sequence that is at least 80% identical, at least 85% identical, at least 90% identical, at least 95% identical, at least 96% identical, at least 97% identical, at least 98% identical, or at least 99% identical to any one of SEQ ID NOs:361, 421, 422, 423, 428, 444, 480.

In some embodiments, the NARE comprises (i) SEQ ID NO:479; (ii) SEQ ID NO:350; (iii) SEQ ID NOs:354 or 356; and (iv) any one of SEQ ID NOs:361, 421, 422, 423, 428, 444, 480.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NOs:478 or 479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iv) a sequence that is at least 80% identical, at least 85% identical, at least 90% identical, at least 95% identical, at least 96% identical, at least 97% identical, at least 98% identical, or at least 99% identical to SEQ ID NO:361.

In some embodiments, the NARE comprises (i) SEQ ID NOs:478 or 479; (ii) SEQ ID NO:350; (iii) any one of SEQ ID NOs:351-356, 499, 500; and (iv) SEQ ID NO:361.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to SEQ ID NO:354; and (iv) a sequence that is at least 80% identical, at least 85% identical, at least 90% identical, at least 95% identical, at least 96% identical, at least 97% identical, at least 98% identical, or at least 99% identical to SEQ ID NO:361.

In some embodiments, the NARE comprises (i) SEQ ID NO:479; (ii) SEQ ID NO:350; (iii) SEQ ID NO:354; and (iv) SEQ ID NO:361.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NOs:478 or 479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iv) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NOs:478 or 479; (ii) SEQ ID NO:350; (iii) any one of SEQ ID NOs:351-356, 499, 500; and (iv) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to SEQ ID NO:356; and (iv) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:479; (ii) SEQ ID NO:350; (iii) SEQ ID NO:356; and (iv) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NOs:478 or 479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iv) a sequence that is at least 80% identical, at least 85% identical, at least 90% identical, at least 95% identical, at least 96% identical, at least 97% identical, at least 98% identical, or at least 99% identical to SEQ ID NO:428.

In some embodiments, the NARE comprises (i) SEQ ID NOs:478 or 479; (ii) SEQ ID NO:350; (iii) any one of SEQ ID NOs:351-356, 499, 500; and (iv) SEQ ID NO:428.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to SEQ ID NO:356; and (iv) a sequence that is at least 80% identical, at least 85% identical, at least 90% identical, at least 95% identical, at least 96% identical, at least 97% identical, at least 98% identical, or at least 99% identical to SEQ ID NO:428.

In some embodiments, the NARE comprises (i) SEQ ID NO:479; (ii) SEQ ID NO:350; (iii) SEQ ID NO:356; and (iv) SEQ ID NO:428.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NOs:478 or 479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iv) a sequence that is at least 80% identical, at least 85% identical, at least 90% identical, at least 95% identical, at least 96% identical, at least 97% identical, at least 98% identical, or at least 99% identical to SEQ ID NO:480.

In some embodiments, the NARE comprises (i) SEQ ID NOs:478 or 479; (ii) SEQ ID NO:350; (iii) any one of SEQ ID NOs:351-356, 499, 500; and (iv) SEQ ID NO:480.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to SEQ ID NO:356; and (iv) a sequence that is at least 80% identical, at least 85% identical, at least 90% identical, at least 95% identical, at least 96% identical, at least 97% identical, at least 98% identical, or at least 99% identical to SEQ ID NO:480.

In some embodiments, the NARE comprises (i) SEQ ID NO:479; (ii) SEQ ID NO:350; (iii) SEQ ID NO:356; and (iv) SEQ ID NO:480.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NOs:478 or 479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iv) a sequence that is at least 80% identical, at least 85% identical, at least 90% identical, at least 95% identical, at least 96% identical, at least 97% identical, at least 98% identical, or at least 99% identical to SEQ ID NO:421.

In some embodiments, the NARE comprises (i) SEQ ID NOs:478 or 479; (ii) SEQ ID NO:350; (iii) any one of SEQ ID NOs:351-356, 499, 500; and (iv) SEQ ID NO:421.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to SEQ ID NO:356; and (iv) a sequence that is at least 80% identical, at least 85% identical, at least 90% identical, at least 95% identical, at least 96% identical, at least 97% identical, at least 98% identical, or at least 99% identical to SEQ ID NO:421.

In some embodiments, the NARE comprises (i) SEQ ID NO:479; (ii) SEQ ID NO:350; (iii) SEQ ID NO:356; and (iv) SEQ ID NO:421.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NOs:478 or 479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iv) a sequence that is at least 80% identical, at least 85% identical, at least 90% identical, at least 95% identical, at least 96% identical, at least 97% identical, at least 98% identical, or at least 99% identical to SEQ ID NO:422.

In some embodiments, the NARE comprises (i) SEQ ID NOs:478 or 479; (ii) SEQ ID NO:350; (iii) any one of SEQ ID NOs:351-356, 499, 500; and (iv) SEQ ID NO:422.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to SEQ ID NO:356; and (iv) a sequence that is at least 80% identical, at least 85% identical, at least 90% identical, at least 95% identical, at least 96% identical, at least 97% identical, at least 98% identical, or at least 99% identical to SEQ ID NO:422.

In some embodiments, the NARE comprises (i) SEQ ID NO:479; (ii) SEQ ID NO:350; (iii) SEQ ID NO:356; and (iv) SEQ ID NO:422.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NOs:478 or 479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to any one of SEQ ID NOs:351-356, 499, 500; and (iv) a sequence that is at least 80% identical, at least 85% identical, at least 90% identical, at least 95% identical, at least 96% identical, at least 97% identical, at least 98% identical, or at least 99% identical to SEQ ID NO:423.

In some embodiments, the NARE comprises (i) SEQ ID NOs:478 or 479; (ii) SEQ ID NO:350; (iii) any one of SEQ ID NOs:351-356, 499, 500; and (iv) SEQ ID NO:423.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:479; (ii) a sequence that is at least 80% identical, at least 85% identical, at least 90% identical, at least 95% identical, at least 96% identical, at least 97% identical, at least 98% identical, or at least 99% identical to SEQ ID NO:350; (iii) a sequence that is at least 80% identical, at least 85% identical, at least 90% identical, at least 95% identical, at least 96% identical, at least 97% identical, at least 98% identical, or at least 99% identical to SEQ ID NO:356; and (iv) a sequence that is at least 80% identical, at least 85% identical, at least 90% identical, at least 95% identical, at least 96% identical, at least 97% identical, at least 98% identical, or at least 99% identical to SEQ ID NO:423.

In some embodiments, the NARE comprises (i) SEQ ID NO:479; (ii) SEQ ID NO:350; (iii) SEQ ID NO:356; and (iv) SEQ ID NO:423.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:377; (ii) a sequence that is at least 80% identical, at least 85% identical, at least 90% identical, at least 95% identical, at least 96% identical, at least 97% identical, at least 98% identical, or at least 99% identical to any one of SEQ ID NOs:444, 457, 458, 469, 470.

In some embodiments, the NARE comprises (i) SEQ ID NO:377; (ii) any one of SEQ ID NOs:444, 457, 458, 469, 470.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:377; (ii) a sequence that is at least 80% identical, at least 85% identical, at least 90% identical, at least 95% identical, at least 96% identical, at least 97% identical, at least 98% identical, or at least 99% identical to any one of SEQ ID NOs:444, 457, 458, 470.

In some embodiments, the NARE comprises (i) SEQ ID NO:377; (ii) any one of SEQ ID NOs:444, 457, 458, 470.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:377; (ii) a sequence that is at least 80% identical, at least 85% identical, at least 90% identical, at least 95% identical, at least 96% identical, at least 97% identical, at least 98% identical, or at least 99% identical to SEQ ID NO:444.

In some embodiments, the NARE comprises (i) SEQ ID NO:377; (ii) SEQ ID NO:444.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:377; (ii) a sequence that is at least 80% identical, at least 85% identical, at least 90% identical, at least 95% identical, at least 96% identical, at least 97% identical, at least 98% identical, or at least 99% identical to SEQ ID NO:457.

In some embodiments, the NARE comprises (i) SEQ ID NO:377; (ii) SEQ ID NO:457.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:377; (ii) a sequence that is at least 80% identical, at least 85% identical, at least 90% identical, at least 95% identical, at least 96% identical, at least 97% identical, at least 98% identical, or at least 99% identical to SEQ ID NOs:469 or 470.

In some embodiments, the NARE comprises (i) SEQ ID NO:377; (ii) SEQ ID NOs:469 or 470.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:377; (ii) a sequence that is at least 80% identical, at least 85% identical, at least 90% identical, at least 95% identical, at least 96% identical, at least 97% identical, at least 98% identical, or at least 99% identical to SEQ ID NO:470.

In some embodiments, the NARE comprises (i) SEQ ID NO:377; (ii) SEQ ID NO:470.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:377; and (ii) a sequence that is at least 80% identical, at least 85% identical, at least 90% identical, at least 95% identical, at least 96% identical, at least 97% identical, at least 98% identical, or at least 99% identical to SEQ ID NO:458.

In some embodiments, the NARE comprises (i) SEQ ID NO:377; and (ii) SEQ ID NO:458.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to any one of SEQ ID NOs:199, 476, 477; and (ii) a sequence that is at least 80% identical, at least 85% identical, at least 90% identical, at least 95% identical, at least 96% identical, at least 97% identical, at least 98% identical, or at least 99% identical to SEQ ID NO:300.

In some embodiments, the NARE comprises (i) any one of SEQ ID NOs:199, 476, 477; and (ii) SEQ ID NO:300.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:199; and (ii) a sequence that is at least 80% identical, at least 85% identical, at least 90% identical, at least 95% identical, at least 96% identical, at least 97% identical, at least 98% identical, or at least 99% identical to SEQ ID NO:300.

In some embodiments, the NARE comprises (i) SEQ ID NO:199; and (ii) SEQ ID NO:300.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:476; and (ii) a sequence that is at least 80% identical, at least 85% identical, at least 90% identical, at least 95% identical, at least 96% identical, at least 97% identical, at least 98% identical, or at least 99% identical to SEQ ID NO:300.

In some embodiments, the NARE comprises (i) SEQ ID NO:476; and (ii) SEQ ID NO:300.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:477; and (ii) a sequence that is at least 80% identical, at least 85% identical, at least 90% identical, at least 95% identical, at least 96% identical, at least 97% identical, at least 98% identical, or at least 99% identical to SEQ ID NO:300.

In some embodiments, the NARE comprises (i) SEQ ID NO:477; and (ii) SEQ ID NO:300.

In some embodiments, the NARE comprises (i) optionally a sequence that is at least 80% identical, at least 85% identical, at least 90% identical, at least 95% identical, at least 96% identical, at least 97% identical, at least 98% identical, or at least 99% identical to SEQ ID NO:393; (ii) a sequence that is at least 80% identical, at least 85% identical, at least 90% identical, at least 95% identical, at least 96% identical, at least 97% identical, at least 98% identical, or at least 99% identical to SEQ ID NO:340 or 341; and (iii) a sequence that is at least 80% identical, at least 85% identical, at least 90% identical, at least 95% identical, at least 96% identical, at least 97% identical, at least 98% identical, or at least 99% identical to SEQ ID NO:474.

In some embodiments, the NARE comprises (i) optionally SEQ ID NO:393; (ii) SEQ ID NO:340 or 341; and (iii) SEQ ID NO:474.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:340; and (ii) a sequence that is at least 80% identical, at least 85% identical, at least 90% identical, at least 95% identical, at least 96% identical, at least 97% identical, at least 98% identical, or at least 99% identical to SEQ ID NO:474.

In some embodiments, the NARE comprises (i) SEQ ID NO:340; and (ii) SEQ ID NO:474.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:393; (ii) a sequence that is at least 80% identical, at least 85% identical, at least 90% identical, at least 95% identical, at least 96% identical, at least 97% identical, at least 98% identical, or at least 99% identical to SEQ ID NO:340; and (iii) a sequence that is at least 80% identical, at least 85% identical, at least 90% identical, at least 95% identical, at least 96% identical, at least 97% identical, at least 98% identical, or at least 99% identical to SEQ ID NO:474.

In some embodiments, the NARE comprises (i) SEQ ID NO:393; (ii) SEQ ID NO:340; and (iii) SEQ ID NO:474.

In some embodiments, the NARE comprises (i) a sequence that is at least 80% identical, at least 85% identical, at least 90% identical, at least 95% identical, at least 96% identical, at least 97% identical, at least 98% identical, or at least 99% identical to SEQ ID NO:341; and (ii) a sequence that is at least 80% identical, at least 85% identical, at least 90% identical, at least 95% identical, at least 96% identical, at least 97% identical, at least 98% identical, or at least 99% identical to SEQ ID NO:474.

In some embodiments, the NARE comprises (i) SEQ ID NO:341; and (ii) SEQ ID NO:474.

In some embodiments, the NARE comprises a sequence that is at least 80% identical, at least 85% identical, at least 90% identical, at least 95% identical, at least 96% identical, at least 97% identical, at least 98% identical, or at least 99% identical to any one of SEQ ID NOs: 6, 14, 16, 18, 34, 48, 104-107, 162-172, 179, 180, 183, 184, 293.

In some embodiments, the NARE comprises any one of SEQ ID NOs: 6, 14, 16, 18, 34, 48, 104-107, 162-172, 179, 180, 183, 184, 293.

In embodiments, the nucleic acid regulatory element comprises an intron or a portion of an intron. The intron or portion of an intron may be selected from: ACTA1 intron, ACTA1e intron, ACTC1 intron, ACTC1.3 intron, ACTC1e intron, ACTC1e-ACTC1p intron, ALDOA intron, APOC intron, APOC1 intron, ATF5 intron, CAMK2A intron, CBA intron, chimeric intron, CMV-rabbit beta globlin intron, CRYAB intron, DES.4 intron, EEF1A1 intron, EEF1B2 intron, EF1a intron, FHL1 intron, FLOT1 intron, FXYD1 intron, GFAP intron, HBB intron, hCPE intron, hEfla2-intron, HPD intron, IFI27L2 intron, Murine IgG chimeric intron, MVM intron, MVMi-AATp intron, MVMi-SynE-mTTRp intron, Rabit beta globin intron, RBP4 intron, RPL26 intron, RPL27 intron, S100A6 intron, sEEF1A1 intron, SV40 intron, TMSB10 intron, and UCHL1 intron.

In embodiments, the nucleic acid regulatory element comprises an enhancer or a portion of an enhancer. The enhancer or a portion of an enhancer may be selected from: ACTA1 enhancer, ACTC1 enhancer, CKM enhancer, CMV enhancer, CMV enhancer, MCK enhancer, mDES enhancer, mDES.1 enhancer, minCKM enhancer, minCKM2 enhancer, minDes enhancer, NRGN enhancer 1.1, NRGN enhancer 1.2, NRGN enhancer 2.1, NRGN enhancer 2.2, SV40 enhancer, and SV40 enhancer (SV40e).

In embodiments, the nucleic acid regulatory element comprises a UTR or a portion of a UTR. The UTR or a portion of a UTR may be selected from: CTNNB1 UTR, hEfla2-utr1, hEfla2-utr2, hNSE utr1, hNSE utr2, hSyn1_utr1, hSyn1_utr2, HTLV 5′ UTR, HTLV 5′UTR, L21 UTR, TMSB10_utr1, and TMSB10_utr2.

### Nucleic Acid Constructs and Vectors

In one aspect, provided are nucleic acid constructs and vectors and their use for the introduction of a transgene or an expression construct into a cell. Nucleic acid constructs include expression constructs including plasmids. The term “expression construct” refers to a recombinant polynucleotide construct that includes a nucleic acid coding for an RNA capable of being transcribed in a cell. Methods for constructing expression constructs and plasmids through standard recombinant techniques are known in the art.

In some embodiments, the vectors comprise recombinant DNA constructs that include additional DNA elements, including DNA segments that provide for the replication of the DNA in a host cell and expression of the target gene in target cells at appropriate levels. “Vector,” as used herein, means a vehicle that comprises a polynucleotide to be delivered into a host cell, either in vitro or in vivo. Non-limiting examples of vectors include a recombinant plasmid, yeast artificial chromosome (YAC), mini chromosome, DNA mini-circle, or a virus (including virus derived sequences). A vector may also refer to a virion comprising a nucleic acid to be delivered into a host cell, either in vitro or in vivo. In some embodiments, a vector refers to a virion comprising a recombinant viral genome, wherein the viral genome comprises one or more ITRs and a transgene.

In one embodiment, the vector is a viral vector or a combination of multiple viral vectors. In one aspect, provided is a vector comprising any of the nucleic acid constructs disclosed herein.

Provided herein are nucleic acid constructs and vectors comprising a nucleic acid regulatory element disclosed herein operatively linked to a transgene. In embodiments, the nucleic acid constructs or vectors disclosed herein comprise additional regulatory elements, including, but not limited to, promoters, enhancers, translation initiation signals, introns, and/or splicing enhancers. In embodiments, the nucleic acid constructs or vectors disclosed herein comprise a polyadenylation sequence. In embodiments, the nucleic acid constructs or vectors disclosed herein comprise an internal ribosome entry site (IRES). An IRES sequence may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. In one embodiment, the IRES is located 3′ of the transgene.

### Viral Vectors

Viral vectors for the expression of a target gene in a target cell, tissue, or organism are known in the art and include, for example, an AAV vector, adenovirus vector, lentivirus vector, retrovirus vector, poxvirus vector, baculovirus vector, herpes simplex virus vector, vaccinia virus vector, or a synthetic virus vector (e.g., a chimeric virus, mosaic virus, or pseudotyped virus, and/or a virus that contains a foreign protein, synthetic polymer, nanoparticle, or small molecule).

### AAV Vectors

Adeno-associated viruses (AAV) are small, single-stranded DNA viruses which require helper virus to facilitate efficient replication. The 4.7 kb genome of AAV is characterized by two inverted terminal repeats (ITR) and two open reading frames which encode the Rep proteins and Cap proteins, respectively. The Rep reading frame encodes four proteins of molecular weight 78 kD, 68 kD, 52 kD, and 40 kD. These proteins function mainly in regulating AAV replication and rescue and integration of the AAV into a host cell's chromosomes. The Cap reading frame encodes three structural proteins of molecular weight 85 kD (VP 1), 72 kD (VP2), and 61 kD (VP3), which form the virion capsid. More than 80% of total proteins in AAV virion comprise VP3. Flanking the rep and cap open reading frames at the 5′ and 3′ ends are about 145 bp long inverted terminal repeats (ITRs). The two ITRs are the only cis elements essential for AAV replication, rescue, packaging, and integration of the AAV genome. The entire rep and cap domains can be excised and replaced with a therapeutic or reporter transgene.

Recombinant adeno-associated virus “rAAV” vectors include any vector derived from any adeno-associated virus serotype. rAAV vectors can have one or more of the AAV wild-type genes deleted in whole or in part, preferably the Rep and/or Cap genes, but retain functional flanking ITR sequences.

In some embodiments, the viral vector is an rAAV virion, which comprises an rAAV genome and one or more capsid proteins. In some embodiments, the rAAV genome comprises a nucleic acid construct disclosed herein.

In some embodiments, the viral vector disclosed herein comprises a nucleic acid comprising an AAV 5′ ITR and 3′ ITR located 5′ and 3′ of the sequence encoding a transgene, respectively. In embodiments, the transgene is UPF1, BDNF, or ATP7B. However, in certain embodiments, it may be desirable for the nucleic acid to contain the 5′ ITR and 3′ ITR sequences arranged in tandem, e.g., 5′ to 3′ or a head-to-tail, or in another alternative configuration. In still other embodiments, it may be desirable for the nucleic acid to contain multiple copies of the ITRs or to have 5′ ITRs (or conversely, 3′ ITRs) located both 5′ and 3′ to transgene. The ITRs sequences may be located immediately upstream and/or downstream of the heterologous molecule, or there may be intervening sequences. The ITRs need not be the wild-type nucleotide sequences, and may be altered (e.g., by the insertion, deletion, or substitution of nucleotides) so long as the sequences provide for functional rescue, replication, and packaging. The ITRs may be selected from AAV2, or from among the other AAV serotypes, as described herein.

In some embodiments, provided is a vector comprising a nucleic acid sequence comprising (i) a nucleic acid construct disclosed herein and (ii) one or more inverted terminal repeats (ITR). In one embodiment, the nucleic acid sequence comprises a 5′ ITR and a 3′ ITR. In one embodiment, the 5′ ITR and a 3′ ITR are derived from adeno-associated virus (AAV) serotype AAV2.

In some embodiments, the viral vector is an AAV vector, such as an AAV1 (i.e., an AAV containing AAV1 ITRs and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments, the viral vector is a pseudotyped AAV vector, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/10 (i.e., an AAV containing AAV2 ITRs and AAV10 capsid proteins).

In some embodiments, the pseudotyped AAV is AAV2/7m8 (i.e., an AAV containing AAV2 ITRs and AAV7m8 capsid proteins).

In some embodiments, the AAV vector contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In embodiments, the capsid is a variant AAV capsid such as the AAV2 variant rAAV2-retro (SEQ ID NO:44 from WO 2017/218842, incorporated herein by reference).

In some embodiments, the AAV vector contains two or more capsid proteins selected from different serotypes. In some embodiments, the AAV vector contains an rAAV2-retro and an AAVrh.10 capsid protein. In some embodiments, the AAV vector contains rAAV2-retro and AAVrh.10 capsid proteins respectively, in a ratio of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50. In some embodiments, the AAV vector contains AAVrh.10 and rAAV2-retro capsid proteins, respectively, in a ratio of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50.

In some embodiments, a mixture of (1) AAV vectors comprising rAAV2-retro and (2) AAV vectors comprising AAVrh.10 is used. In some embodiments, a ratio of the (1) AAV vectors comprising rAAV2-retro and (2) AAV vectors comprising AAVrh.10, respectively, of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50 is used. In some embodiments, a ratio of the (1) AAV vectors comprising AAVrh.10 and (2) AAV vectors comprising rAAV2-retro, respectively, of 1:1, 1:2, 1:3, 1:4, 1:5, 1:6, 1:7, 1:8, 1:9, 1:10, 1:15, 1:20, 1:25, 1:30, 1:35, 1:40, 1:45, or 1:50 is used.

### Other Viral Vectors

Other viral vectors include adenoviral (AV) vectors, for example, those based on human adenovirus type 2 and human adenovirus type 5 that have been made replication defective through deletions in the E1 and E3 regions. The transcriptional cassette can be inserted into the E1 region, yielding a recombinant E1/E3-deleted AV vector. Adenoviral vectors also include helper-dependent high-capacity adenoviral vectors (also known as high-capacity, “gutless” or “gutted” vectors), which do not contain viral coding sequences. These vectors contain the cis-acting elements needed for viral DNA replication and packaging, mainly the inverted terminal repeat sequences (ITR) and the packaging signal (CY). These helper-dependent AV vector genomes have the potential to carry from a few hundred base pairs up to approximately 36 kb of foreign DNA.

Alternatively, other systems such as lentiviral vectors can be used. Lentiviral-based systems can transduce nondividing as well as dividing cells making them useful for applications targeting, for example, the nondividing cells of the CNS. Lentiviral vectors are derived from the human immunodeficiency virus and, like that virus, integrate into the host genome providing the potential for very long-term gene expression.

Polynucleotides, including plasmids, YACs, minichromosomes and minicircles, carrying the target gene containing the expression cassette can also be introduced into a cell or organism by nonviral vector systems using, for example, cationic lipids, polymers, or both as carriers. Conjugated poly-L-lysine (PLL) polymer and polyethylenimine (PEI) polymer systems can also be used to deliver the vector to cells. Other methods for delivering the vector to cells include hydrodynamic injection and electroporation and use of ultrasound, both for cell culture and for organisms. For a review of viral and non-viral delivery systems for gene delivery see Nayerossadat, N. et al. (Adv Biomed Res. 2012; 1:27) incorporated herein by reference.

rAAV Virion Production

The rAAV virions disclosed herein may be constructed and produced using the materials and methods described herein, as well as those known to those of skill in the art. Such engineering methods used to construct any embodiment of this disclosure are known to those with skill in nucleic acid manipulation and include genetic engineering, recombinant engineering, and synthetic techniques. See, e.g., Sambrook et al, “Molecular Cloning. A Laboratory Manual”, 2d ed., Cold Spring Harbor Laboratory, New York (1989), and Ausubel et al., Current Protocols in Molecular Biology, John Wiley & Sons, New York, 1989); and International Patent Publication No. WO 95/13598. Further, methods suitable for producing a rAAV cassette in an adenoviral capsid have been described in U.S. Pat. Nos. 5,856,152 and 5,871,982.

Briefly, in order to package the rAAV genome into a rAAV virion, a host cell is used that contains sequences necessary to express AAV rep and AAV cap or functional fragments thereof as well as helper genes essential for AAV production. The AAV rep and cap sequences are obtained from an AAV source as identified herein. The AAV rep and cap sequences may be introduced into the host cell in any manner known to one in the art, including, without limitation, transfection, electroporation, liposome delivery, membrane fusion techniques, high velocity DNA-coated pellets, viral infection, and protoplast fusion. In one embodiment, the rep and cap sequences may be transfected into the host cell by one or more nucleic acid molecules and exist stably in the cell as an episome. In another embodiment, the rep and cap sequences are stably integrated into the genome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

The rep and cap sequences, along with their expression control sequences, may be supplied on a single vector, or each sequence may be supplied on its own vector. Preferably, the rep and cap sequences are supplied on the same vector. Alternatively, the rep and cap sequences may be supplied on a vector that contains other DNA sequences that are to be introduced into the host cells. Preferably, the promoter used in this construct may be any suitable constitutive, inducible or native promoters known to one of skill in the art. The molecule providing the rep and cap proteins may be in any form which transfers these components to the host cell. Desirably, this molecule is in the form of a plasmid, which may contain other non-viral sequences, such as those for marker genes. This molecule does not contain the AAV ITRs and generally does not contain the AAV packaging sequences. To avoid the occurrence of homologous recombination, other virus sequences, particularly those of adenovirus, are avoided in this plasmid. This plasmid is desirably constructed so that it may be stably transfected into a cell.

Although the molecule providing rep and cap may be transiently transfected into the host cell, it is preferred that the host cell be stably transformed with sequences necessary to express functional rep/cap proteins in the host cell, e.g., as an episome or by integration into the chromosome of the host cell. Depending upon the promoter controlling expression of such stably transfected host cell, the rep/cap proteins may be transiently expressed (e.g., through use of an inducible promoter).

The methods employed for constructing embodiments of this disclosure are conventional genetic engineering or recombinant engineering techniques such as those described in the references above. For example, the rAAV may be produced utilizing a triple transfection method using either the calcium phosphate method (Clontech) or Effectene reagent (Qiagen, Valencia, Calif.), according to manufacturer's instructions. See, also, Herzog et al, 1999, Nature Medic., 5(1):56-63, for the method used in the following examples, employing the plasmid with the transgene, a helper plasmid containing AAV rep and cap, and a plasmid supplying adenovirus helper functions of E2A, E4Orf6 and VA. While this specification provides illustrative examples of specific constructs, using the information provided herein, one of skill in the art may select and design other suitable constructs, using a choice of spacers, promoters, and other elements, including at least one translational start and stop signal, and the optional addition of polyadenylation sites.

The rAAV virions can be produced by culturing a host cell containing a rAAV virus as described herein which contains a rAAV genome to be packaged into a rAAV virion, an AAV rep sequence and an AAV cap sequence under the control of regulatory sequences directing expression thereof. Suitable viral helper genes, e.g., adenovirus E2A, E4Orf6 and VA, among other possible helper genes, may be provided to the culture in a variety of ways known to the art, preferably on a separate plasmid. Thereafter, the recombinant AAV virion which directs expression of the transgene is isolated from the cell or cell culture in the absence of contaminating helper virus or wildtype AAV.

Expression of a transgene may be measured in ways known in the art. For example, a target cell may be infected in vitro, and the number of copies of the transgene in the cell monitored by Southern blotting or quantitative polymerase chain reaction (PCR). The level of RNA expression may be monitored by Northern blotting or quantitative reverse transcriptase (RT)-PCR; and the level of protein expression may be monitored by Western blotting, immunohistochemistry, enzyme-linked immunosorbent assay (ELISA), radioimmunoassay (RIA) or by the specific methods detailed below in the Examples.

### Cells

In embodiments, the nucleic acid constructs and vectors disclosed herein are used to deliver a nucleic acid regulatory element operatively linked to a transgene to a cell. Provided herein are cells comprising a nucleic acid regulatory element, a nucleic acid construct, or a vector disclosed herein. In embodiments, the cell is a muscle cell. In embodiments, the cell is a liver cell. In embodiments, the cell is a neuronal cell. The cell may be a mammalian cell. The cell may be a human cell. The cell may be isolated.

### Pharmaceutical Compositions

Provided herein are pharmaceutical compositions comprising a nucleic acid construct or vector disclosed herein and a pharmaceutically acceptable excipient.

The nucleic acid construct or vector disclosed herein is preferably assessed for contamination by conventional methods and then formulated into a pharmaceutical composition suitable for storage and/or administration to a patient.

Formulations of the nucleic acid constructs or vectors disclosed herein disclosed herein involve the use of a pharmaceutically and/or physiologically acceptable vehicle or carrier, particularly one suitable for subretinal injection, such as buffered saline or other buffers, e.g., HEPES, to maintain pH at appropriate physiological levels. The nucleic acid constructs or vectors disclosed herein can be formulated into pharmaceutical compositions. These compositions may comprise, in addition to the vector, a pharmaceutically and/or physiologically acceptable excipient, carrier, buffer, stabilizer, antioxidants, preservative, or other additives well known to those skilled in the art. Such materials should be non-toxic and should not interfere with the efficacy of the active ingredient. The precise nature of the carrier or other material may be determined by the skilled person according to the route of administration. The pharmaceutical composition is typically in liquid form. Liquid pharmaceutical compositions generally include a liquid carrier such as water, petroleum, animal or vegetable oils, mineral oil or synthetic oil. Additional carriers are provided in International Patent Publication No. WO 00/15822, incorporated herein by reference. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used. In some cases, Ringer's Injection, Lactated Ringer's Injection, or Hartmann's solution is used. Preservatives, stabilizers, buffers, antioxidants and/or other additives may be included, as required.

Pharmaceutical compositions comprising a nucleic acid construct or vector disclosed herein may formulated with one or more pharmaceutically-acceptable excipients, which can be a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent is a compound which adds mass to a pharmaceutical formulation and contributes to the physical structure of the formulation in lyophilized form. Suitable bulking agents according to the present invention include mannitol, glycine, polyethylene glycol and sorbitol.

The use of a surfactant can reduce aggregation of the reconstituted protein and/or reduce the formation of particulates in the reconstituted formulation. The amount of surfactant added is such that it reduces aggregation of the reconstituted protein and minimizes the formation of particulates after reconstitution. Suitable surfactants according to the present invention include polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g. Pluronics, PF68, etc.).

Preservatives may be used in formulations of invention. Suitable preservatives for use in the formulation of the invention include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995).

For delayed release, nucleic acid construct or vector disclosed herein may be included in a pharmaceutical composition which is formulated for slow release, such as in microcapsules formed from biocompatible polymers or in liposomal carrier systems according to methods known in the art.

If a vector is to be stored long-term, it may be frozen in the presence of glycerol.

### Methods

Provided herein are methods for inducing expression of a transgene in a cell and/or a tissue. Provided herein is a method of inducing the expression of transgene, the method comprising providing a cell comprising a nucleic acid construct comprising a nucleic acid regulatory element disclosed herein operatively linked to a transgene and cultivating the cells under conditions allowing for the expression of the transgene. In embodiments, the cell is a muscle cell. In embodiments, the cell is a liver cell. In embodiments, the cell is a neuronal cell. Provided herein is a method for inducing expression of a transgene in vivo. Provided herein is a method for inducing expression of a transgene in vitro. Provided herein is a method for inducing expression of a transgene ex vivo.

Provided herein are methods of treating a disease in a subject in need thereof using nucleic acid constructs, vectors, and pharmaceutical compositions disclosed herein.

In some embodiments, the subject is a mammal. The term “mammal” as used herein is intended to include, but is not limited to, humans, laboratory animals, domestic pets, and farm animals. Mammals, include, but are not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of one or more symptoms of the condition, disorder or disease state; and remission (whether partial or total), or enhancement or improvement of the condition, disorder or disease. Treatment includes eliciting a clinically significant response without excessive levels of side effects. Treatment also includes prolonging survival as compared to expected survival if not receiving treatment. The terms “prevent”, “prevention”, and the like refer to acting prior to overt disease or disorder onset, to prevent the disease or disorder from developing or to minimize the extent of the disease or disorder or slow its course of development.

In some embodiments, treatment refers to increased survival (e.g., survival time). For example, treatment can result in an increased life expectancy of a patient. In some embodiments, treatment results in an increased life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to the average life expectancy of one or more control individuals with ALS (amyotrophic lateral sclerosis) without treatment. In some embodiments, treatment results in an increased life expectancy of a patient by more than about 6 months, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals with ALS without treatment. In some embodiments, treatment results in long term survival of a patient. As used herein, the term “long term survival” refers to a survival time or life expectancy longer than about 40 years, 45 years, 50 years, 55 years, 60 years, or longer.

In embodiments, the subject has the potential to develop ALS. In some instances, a subject to be treated is genetically predisposed to developing ALS. For example, a subject to be treated has a mutation in a SOD1 gene, ALS2 gene, VAPB gene, SETX gene, TDP-43 gene, FUS/TLS gene, C9orf72 gene, and/or OPTN gene.

Methods of administration include, but are not limited to, intracisternal magna, intracerebroventricular, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. The mode of administration is left to the discretion of the practitioner.

In some instances, the nucleic acid construct or vector described herein is administered locally. This can be achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as silastic membranes, or fibers. In some situations, the nucleic acid construct or vector described herein is introduced into the central nervous system, circulatory system or gastrointestinal tract by any suitable route, including intraventricular injection, intrathecal injection, paraspinal injection, epidural injection, enema, and by injection adjacent to a peripheral nerve.

The compositions described herein can be administered as single administrations or as multiple administrations. Such compositions can be administered at regular intervals, depending on the nature, severity and extent of the subject's condition. In some embodiments, a therapeutically effective amount of the nucleic acid construct or vector is administered intrathecally periodically at regular intervals (e.g., once every year, once every six months, once every five months, once every three months, bimonthly (once every two months), monthly (once every month), biweekly (once every two weeks), or weekly).

The amount of the nucleic acid construct or vector described herein that is effective for treating disease can be determined using standard clinical techniques known to those with skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner.

An effective amount of an rAAV carrying a nucleic acid sequence encoding a transgene (including, but not limited to UPF1, ATP7B, and BDNF) under the control of the nucleic acid regulatory element may, for example, range between about 1×10⁹ to about 1×10¹⁴ rAAV genome particles (vg)/kg body weight. A “genome particle” is defined herein as an AAV capsid that contains a single stranded DNA molecule that can be quantified with a sequence specific method (such as qPCR or ddPCR). In some embodiments, the rAAV is administered at about 1×10¹² to about 1×10¹³ rAAV vg/kg body weight. In some embodiments, the rAAV is administered at about 5×10¹¹ to about 5×10¹² to vg/mL of cerebrospinal fluid (CSF) volume. In some embodiments, the rAAV is administered at about 7.5×10¹³ to 7.5×10¹⁴ vg total per patient.

In some embodiments, the rAAV is administered to an animal at about 1×10¹¹ to about 1×10¹⁴ rAAV genome particles (vg)/kg body weight.

In some embodiments, the rAAV genome particles are provided in a volume of between about 20 uL to about 50 mL. In some embodiments, the rAAV genome particles are provided in a volume of between about 30 uL to about 30 mL. In some embodiments, the rAAV genome particles are provided in a volume of about 30, about 40, about 50, about 60, about 70, about 80, about 90, about 100, about 200, about 300, about 400, about 500, about 600, about 700, about 800, about 900, about 1000 uL. In some embodiments, the rAAV genome particles are provided in a volume of about 1, about 2, about 3, about 4, about 5, about 6, about 7, about 8, about 9, about 10, about 15, about 20, about 25, about 30, about 35, about 40, about 45, about or 50 mL.

Still other dosages in these ranges may be selected by the attending physician. It is to be understood that for any particular subject, specific dosage regimens can be adjusted over time according to the individual need and the professional judgment of the person administering or supervising the administration of the nucleic acid construct or vector and that dosage ranges set forth herein are exemplary only and are not intended to limit the scope or practice of the claimed invention.

The nucleic acid construct or vectors disclosed herein can also be advantageously provided to a cell ex vivo, followed by administration of the living cell to the subject. Methods for treating disease by implanting a cell that has been modified to express a recombinant protein are also well known. See, for example, U.S. Pat. No. 5,399,346, disclosing methods for introducing a nucleic acid into a primary human cell for introduction into a human. Although use of human cells for ex vivo therapy is preferred in some embodiments, other cells such as bacterial cells may be implanted in a subject's vasculature, continuously releasing a therapeutic agent. See, for example, U.S. Pat. Nos. 4,309,776 and 5,704,910.

This application is related to U.S. Provisional Application No. 63/379,109 entitled “UPF1 EXPRESSION CONSTRUCTS”, U.S. Provisional Application No. 63/379,113, entitled “ATP7B GENE THERAPY” and U.S. Provisional Application No. 63/379,114, entitled “BDNF GENE THERAPY” all filed on Oct. 11, 2022 by the same Applicant, and each of which is incorporated herein by reference in its entirety.

It is to be understood that this invention is not limited to the particular molecules, compositions, methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present invention, the following Examples of specific embodiments are given. The following examples should not be read to limit or define the entire scope of the invention.

### EXAMPLES

### Example 1: Nucleic Acid Regulatory Element Design

### Materials and Methods

### AI-Driven Nucleic Acid Regulatory Element Design

Core prediction model: The prediction of construct potency was based on the predictions from the Xpresso model (xpresso.gs.washington.edu). The original Xpresso model was developed by considering the 10.5 kb region around the transcription start site (TSS, 7.5 kb upstream, 3 kb downstream the TSS). Because of size constraints (fragments longer than ˜1.5 kb were not be used), the algorithm was modified to include a “padding” step before the calculation of the predicted activity. This padding step took a sequence and added ambiguous (“N”) nucleotides upstream and downstream of the shorter input fragment according to the expected/annotated TSS. The final model took a sequence, scanned for an upper-case base pair (convention used to label the TSS), padded the sequence to length, converted it into a one-hot-encoded data frame and then passed it through the convolutional neural network (CNN) with weights and structure from Xpresso.

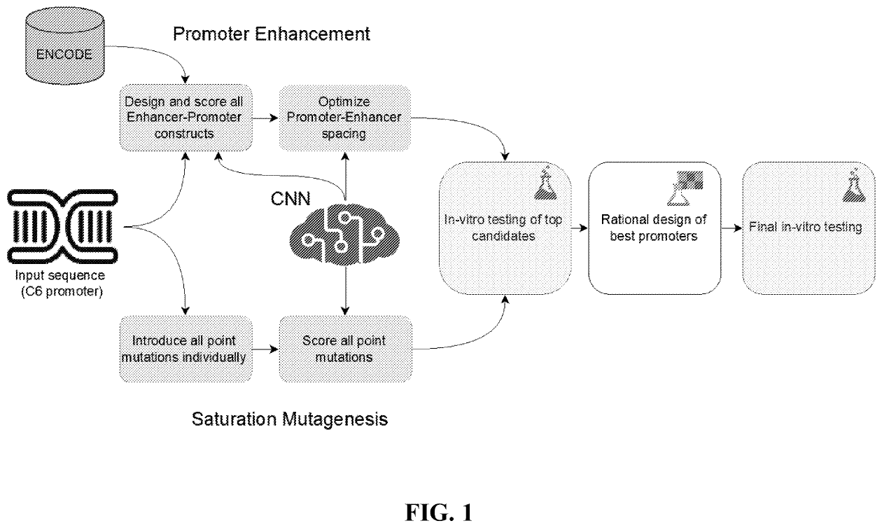

Custom software was written to create and automate the scoring for the following analyses (FIG. 1):

ENCODE+JeT(C6): ENCODE fragment coordinates were downloaded and subsequently used to parse such elements from the reference human genome (GRCh38). All the ENCODE fragments were individually positioned upstream a JeT promoter and then tested through the modified prediction model.

ENCODE+JeTspacing optimization: a custom script was used to insert 300 bp, 400 bp, 500 bp or 600 bp spacing elements between the top ˜2,000 scoring ENCODE elements and the JeT promoter. New “spaced” constructs were again tested with the modified prediction model.

Point-mutation Saturation mutagenesis: a custom script was used to generate all possible point-mutated versions of an input sequence. Resulting sequences were then scored with the modified prediction model. Custom scripts were also developed to iterate this process in through a greedy search (introduce the best result each round).

Through the CNN model, the effect of 708,157 enhancer fragments was predicted in-silico cloned up-stream of the C6 promoter sequence. The majority of enhancers were predicted to have a positive effect on C6 potency with ˜2,000 constructs producing a ˜2×improvement. This subset was further analyzed to identify the optimal spacing between the enhancer element and the promoter sequence. Introducing a spacing sequence improved the potency of the construct with the largest improvement observed with a spacing of 400 bp. Selected constructs were synthesized and tested in vitro (C118 to C127).

### NARE Syntax and Rational Design of Nucleic Acid Regulatory Elements

Potential nucleic acid regulatory element candidates were also identified from genes that are highly expressed in humans in the target tissues (muscle, brain, and liver). Promoter regions were then defined or cis-regulatory elements identified based on chromatin marks, accessibility, conservation, and other genome-wide datasets.

Motif ten element (MTE)/downstream promoter element (DPE) modifications were incorporated to increase transcription, as were various cellular, viral, or synthetic 5′ UTR motifs to increase transcription/translation. In CAG-based gene regulation elements (but also in others described herein), intron choice was changed to include, for example, short cellular introns that naturally have a unique standalone transcriptional start site, which indicates the presence of a promoter within this particular intron. In addition, introns were inserted at different positions of the sequence, and/or introns were truncated while maintaining splicing elements. In addition, individual or multiple point mutations were introduced to increase expression or prevent inactivation. Also, different transcriptional enhancers were introduced upstream of the core promoter. Transcriptional motif insertions, replacements, and/or shuffling was employed in certain instances as was removal of CpG sequences to lower immunogenicity while maintaining NARE activity.

Gene regulation elements were also optimized by the following methods (i) highly expressed transcription factor binding sites (TFBS) were identified, shuffled and cloned upstream of core promoters; (ii) iterative mutation and scoring of promoters using artificial intelligence (AI) algorithms.

### Example 2: Dual-Reporter Protein Expression Assay

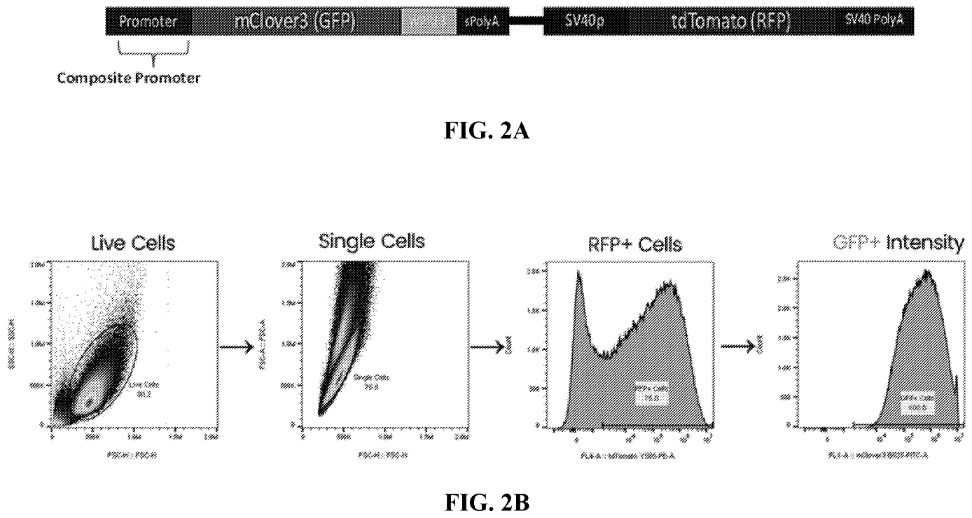

To test the ability of nucleic acid regulatory element candidates to drive gene expression, a dual reporter assay using flow cytometry was utilized. A diagram of the dual reporter expression construct and assay is provided in FIG. 2A. Different synthetic candidate NARE sequences were cloned upstream of a transgene encoding mClover3 (a green/yellow fluorescent protein), which in turn was located upstream of a posttranscriptional regulatory element of woodchuck hepatitis virus (WPRE250) and a synthetic polyadenylation (polyA) signal. mClover3 fluorescence was used as a measure of NARE strength. The plasmid also contained a separate expression cassette (from 5′ to 3′: SV40 promoter—tdTomato (a red fluorescent protein)—SV40 polyA), which was the same in all nucleic acid regulatory element test constructs (FIG. 2A). tdTomato fluorescence was used as an internal normalization control, allowing to account for variations in transfection efficiency.

Various cell lines were transiently transfected with dual reporter plasmids using TurboFect® transfection reagent. After 48 hours, cells were trypsinized and resuspended for flow cytometry. Raw data was processed to select only live, single cells for analysis. A sample comprising untransfected cells was used to set the gate for tdTomato signal. This gate was then applied to all live single cells to identify the tdTomato-positive (and thus transfected) cells (FIG. 2B). Then, mClover3 fluorescence was determined for the tdTomato-positive population. Because transfection varied slightly between different experiments, the relative NARE potency was calculated by the ratio of mClover3 fluorescence to tdTomato fluorescence. Candidate NAREs were tested along with known constitutive and tissue-specific reference promoters such as CAG, CMV, or AAT.

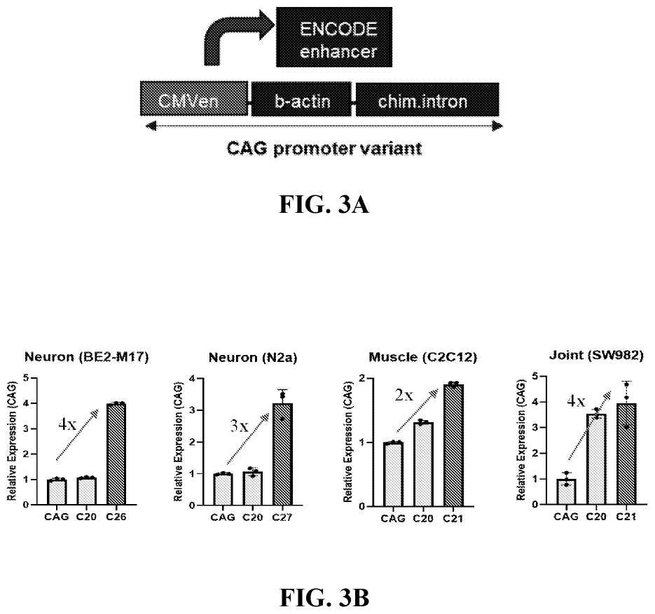

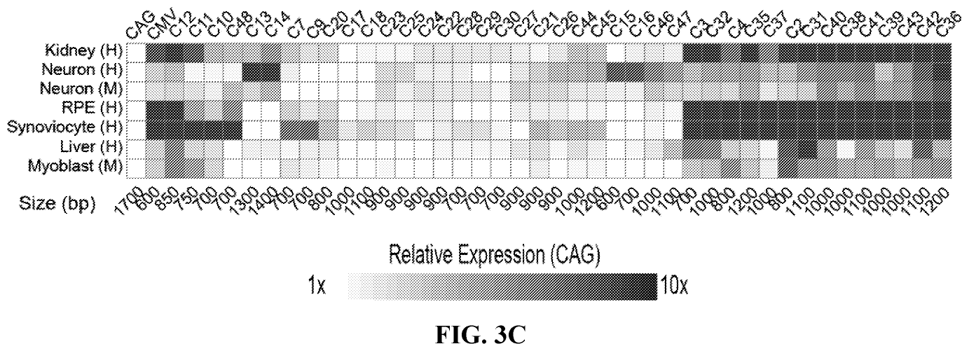

Relative NARE strength of selected, constitutive nucleic acid regulatory elements as compared to a CAG (C19) in different cell types is shown in Table 1 and FIG. 3B. In these nucleic acid regulatory elements, the CMV enhancer region of CAG had been modified as shown in FIG. 3A. This set of CAG variants resulted in up to four-fold improvement in potency in muscle and neurons while reducing size by up to ˜900 bp.

The tissue/cell specificity of selected nucleic acid regulatory elements is shown in FIG. 3C.

TABLE 1
Expression of selected, constitutive nucleic acid regulatory elements as compared to a CAG
(C19) in different cell types. Expression data was obtained via flow cytometry using the
dual reporter system illustrated in FIG. 2. Expression is provided relative to CAG.

| | | | Neuron | | Muscle | Kidney | RPE | Joint |
|---|---|---|---|---|---|---|---|---|
| Name | NARE | Size (bp) | BE2-M17 | N2a | C2C12 | HEK293 | ARPE19 | SW982 |
| C19 | CAG | 1761 | 1 | 1 | 1 | 1 | 1 | 1 |
| C20 | CBh | 818 | 1.1 | 1.1 | 1.3 | 2.6 | 2.7 | 3.7 |
| C21 | ENCODE1-CBh | 883 | 3.1 | 2.7 | 1.9 | 1.5 | 1.9 | 4.1 |
| C22 | ENCODE2-CBh | 882 | 1.9 | 1.9 | 1.0 | 2.2 | 1.7 | 1.9 |
| C23 | ENCODE3-CBh | 878 | 2.8 | 2.6 | 1.3 | 1.6 | 1.8 | 2.9 |
| C24 | ENCODE4-CBh | 883 | 1.9 | 2.3 | 1.1 | 2.3 | 1.7 | 1.6 |
| C25 | ENCODE5-CBh | 883 | 2.6 | 1.7 | 1.2 | 1.6 | 1.5 | 2.4 |
| C26 | ENCODE6-CBh | 882 | 4 | 2.7 | 1.1 | 2.1 | 1.9 | 3.4 |
| C27 | ENCODE7-CBh | 889 | 2.2 | 3.2 | 1.3 | 1.9 | 1.7 | 1.1 |
| C28 | ENCODE8-CBh | 696 | 1.7 | 2.1 | 1.2 | 2.1 | 1.3 | 2.4 |
| C29 | ENCODE9-CBh | 716 | 1.2 | 2 | 1.1 | 2.1 | 1.2 | 2.4 |
| C30 | ENCODE10-CBh | 705 | 1.2 | 1.5 | 1.2 | 2.2 | 0.8 | 1.4 |

RPE = retinal pigment epithelium.

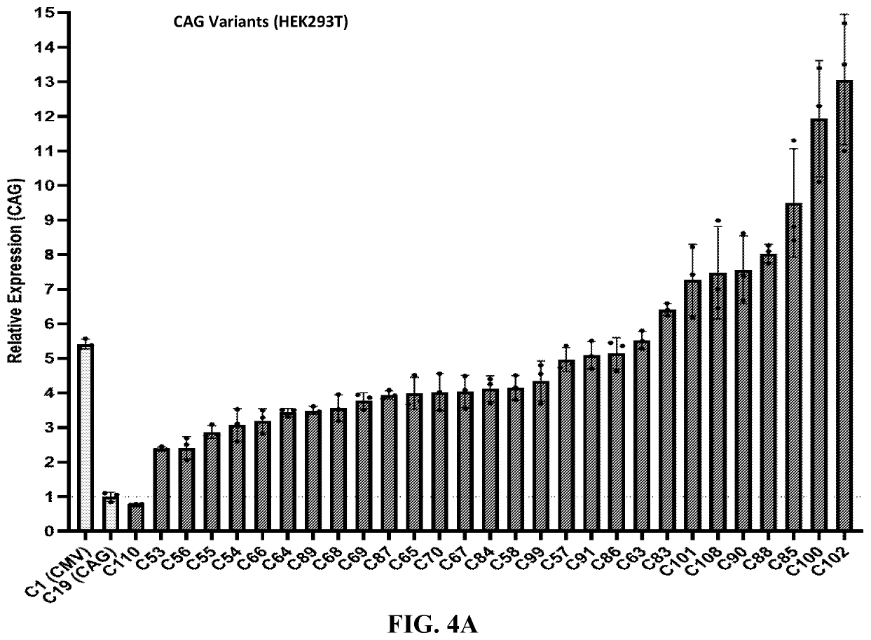

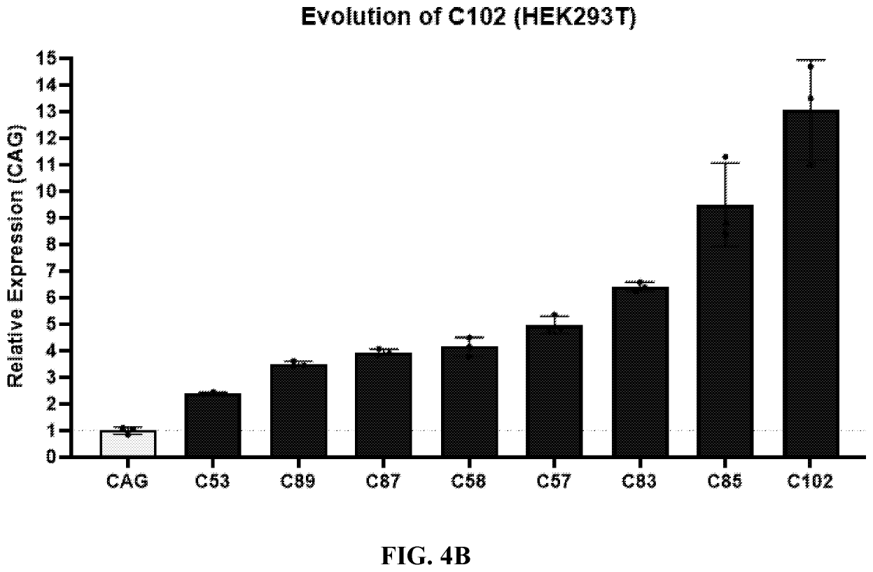

Relative NARE strength of selected, constitutive nucleic acid regulatory elements as compared to a CAG (C19) and/or CMV (C1) promoter is shown in FIGS. 4A and 4B (HEK293T (human embryonic kidney) cells) and in Table 2. In these nucleic acid regulatory elements, the beta actin region and/or the chimeric intron of the CAG promoter had been modified. The HEK293T genome contains the SV40 large T antigen, which enables the production of recombinant proteins within plasmid vectors containing an SV40 promoter. C2C12 cells are murine skeletal muscle myoblasts derived from satellite cells of the thigh muscle of a two-month-old female C3H mouse.

### NARE Modifications Included:

ATGlessCMVmd (Mod 15): C83 vs C57 (potency increased by 1.3×)
ATGlessCBAmd (Mod 18): C86 vs C84 (potency increased by 1.3×)
Additional BA promoter to CAG promoter (dual beta actin promoters): C84 vs C53 (Mod 16, CBAmd, potency increased by 1.7×); C87 vs C53 (Mod 19, HBAmd, potency increased by 1.7×); C89 vs C53 (Mod 21, MBAmd, potency increased by 1.5×)
Additional HBAmd and ATGlessCMVmd on C84 (quad NARE): C91 vs C83 (Mods 16, 19, potency increased by 1.2×)
Additional ATGlessCBAmd on C91: C99 vs C91 (Mod 18, potency decreased to 0.85×
Replacing CBAmd in C85 with another BA promoter: C88 vs C85 (Mod 17 vs 20, HBAmd, potency decreased to 0.85×); C90 vs C85 (Mod 17 vs 22, MBAmd, potency decreased to 0.80×)

TABLE 2
Sequential nucleic acid regulatory element modifications and
expression in HEK293T (see Table 9 for the specific modifications
and sequences). Dual reporter plasmids with different
NARE candidates were transiently transfected into C2C12 cells.
GFP expression level in transfected (RFP+) cells was
measured by flow cytometry. Mod1: Replaced chimeric
intron in CAG with native CBA intron. Mod5: Insertion of
CMVp into intron. Mod6: MTE/DPE insertion into CMVp. Mod15:
Modification of CMVmd promoter into ATGless form. Mod17:
MTE/DPE insertion into original CBAp of CAG. Mod19: Insertion of HBAmd
promoter into intron. Mod21: Insert of MBAmd promoter into intron.
Mod23: Insertion of beta catenin 5′-UTR (CTBBN1).
Mod24: Insertion of tubulin 5′-UTR (TUUB). Mod25:
Modification of MBAmd promoter into ATGless form.

| | | Potency |
|---|---|---|
| NARE | Size (bp) | relative to CAG |
| C1 (CMV) | 700 | 5.3 |
| C19 (CAG) | 1761 | 1.0 |
| C53 (CAG + Mod 1) | 1672 | 2.4 |
| C54 (CAG + Mod 1, 2) | 2055 | 3 |
| C55 (CAG + Mod 1, 3) | 1862 | 2.9 |
| C56 (CAG + Mod 1, 4) | 1746 | 2.4 |
| C57 (CAG + Mod 1, 5, 6) | 1930 | 5.0 |
| C58 (CAG + Mod 1, 5) | 1929 | 4.2 |
| C63 (CAG + Mod 1, 7) | 1959 | 5.5 |
| C64 (CAG + Mod 1, 7, 8) | 1714 | 3.4 |
| C65 (CAG + Mod 1, 7, 8, 9) | 1509 | 4.0 |
| C66 (CAG + Mod 1, 7, 8, 9, 10) | 1139 | 3.2 |
| C67 (CAG + Mod 1, 5, 6, 11) | 1680 | 4.0 |
| C68 (CAG + Mod 1, 5, 6, 11, 12) | 1439 | 3.6 |
| C69 (CAG + Mod 1, 5, 6, 11, 12, 13) | 1180 | 3.8 |
| C70 (CAG + Mod 1, 5, 6, 11, 12, 13, 14) | 1070 | 4.0 |
| C83 (CAG + Mod 1, 5, 6, 15) | 1930 | 6.4 |
| C84 (CAG + Mod 1, 16) | 1990 | 4.1 |
| C85 (CAG + Mod 1, 17, 5, 6) | 1979 | 9.5 |
| C86 (CAG + Mod 1, 16, 18) | 1990 | 5.2 |
| C87 (CAG + Mod 1, 19) | 1959 | 4.0 |
| C88 (CAG + Mod 1, 20, 5, 6) | 1948 | 8.0 |
| C89 (CAG + Mod 1, 21) | 1980 | 3.5 |
| C90 (CAG + Mod 1, 22, 5, 6) | 1969 | 7.6 |
| C91 (CAG + Mod 1, 19, 5, 6, 15, 16) | 2223 | 5.1 |
| C99 (CAG + Mod 1, 19, 5, 6, 15, 16, 18) | 2223 | 4.4 |
| C100 (CAG + Mod 1, 19, 16, 18, 5, 6, 15) | 1994 | 11.9 |
| C101 (CAG + Mod 1, 17, 19, 23, 16, 18, 24, 5, 6, 15) | 1964 | 7.3 |
| C102 (CAG + Mod 1, 17, 19, 23, 21, 25, 24, 5, 6, 15) | 1954 | 13.1 |
| C108 (CAG + Mod 1, 17, 19, 23, 21, 25, 24) | 1689 | 7.5 |

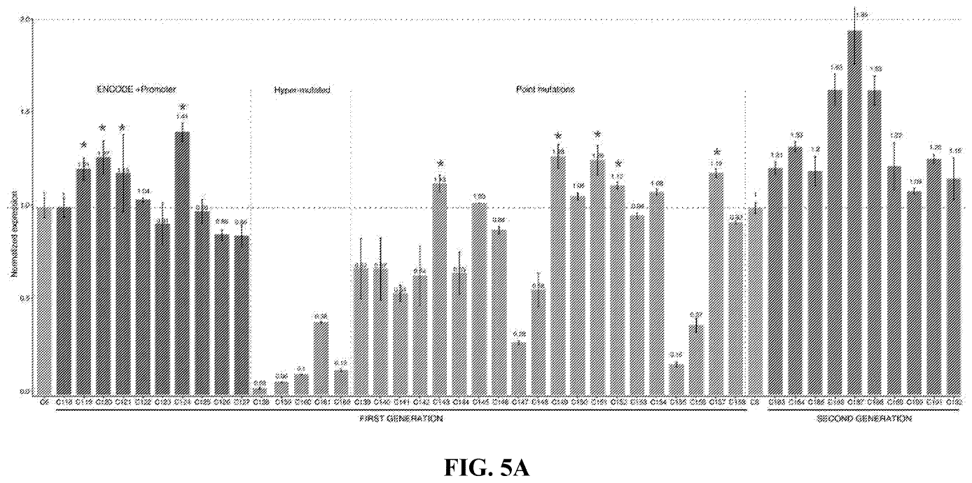

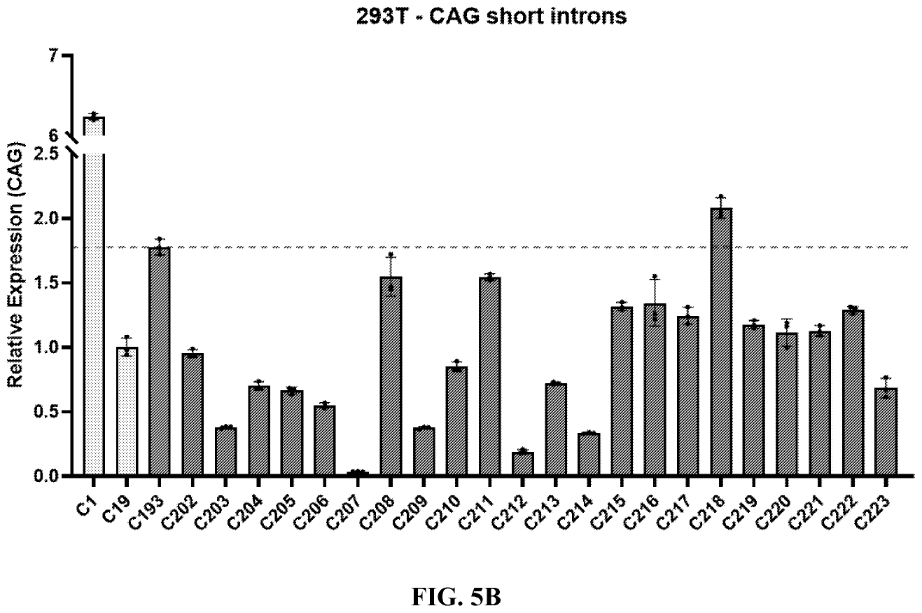

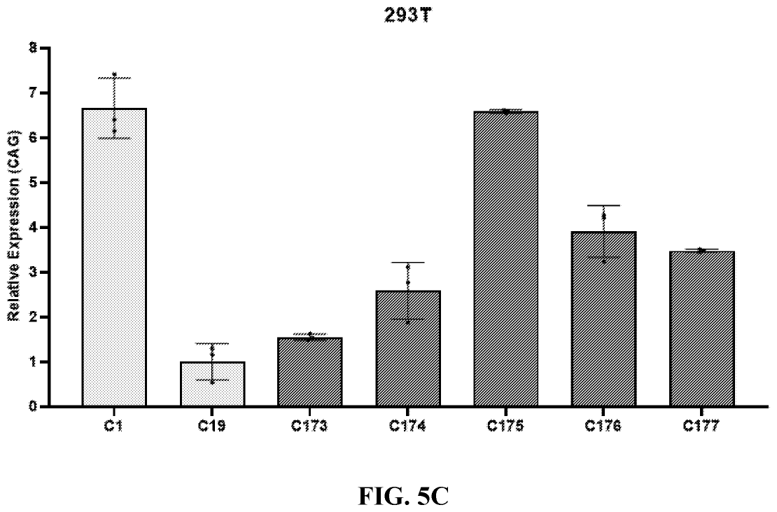

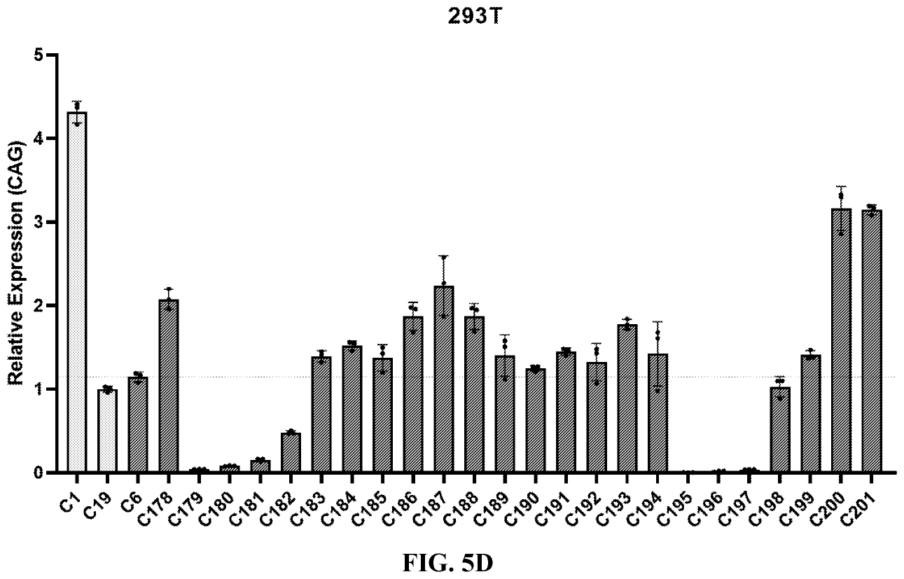

Relative NARE strength of selected, constitutive nucleic acid regulatory elements as compared to the JeTmd promoter (C6) is shown in FIG. 5A. These NAREs were developed using the AI methods described herein.

Different CAG-derived nucleic acid regulatory elements were created by replacing the intron of a CAG variant (C193) with compact, promoter-containing introns. NARE potency was assessed in BTEK293T cells (FIGS. 5B and 5C). C218 is twice as strong as CAG (C19), but is shorter (C218 is 1092 bp).

Potency of additional nucleic acid regulatory elements in BTEK293T cells is shown in FIG. 5D.

### Example 3: Strength of Different NAREs in Muscle Cells (In Vitro)

### Materials and Methods

### Myotube Models

The strength of selected muscle specific nucleic acid regulatory elements was tested using two cellular models of myotubes. One model is the C2C12 immortalized cell line. C2C12 cells are murine skeletal muscle myoblasts derived from satellite cells of the thigh muscle of a two-month-old female C3H mouse. C2C12 cells were plated on Collagen I-coated vessels and grown in expansion media: DMEM (high glucose, no Sodium Pyruvate)+10% Fetal Bovine Serum+2 mM L-Glutamine+100 units/mL Penicillin and 100 μg/mL Streptomycin. After reaching confluence, the C2C12 cells underwent a media change with differentiation media: DMEM (high glucose, no Sodium Pyruvate)+2% Horse Serum+2 mM L-Glutamine+1 μM Insulin+100 units/mL Penicillin and 100 μg/mL Streptomycin. The C2C12 cells were cultured in differentiation media for about 7 days to form long, striated myotubes, which was the result of myoblastic fusion during myogenesis.

The second model used primary human skeletal myoblasts (Thermo Fisher Scientific), derived from biopsies of quadriceps of a human donor lot. The primary human skeletal myoblasts were plated on collagen I-coated vessels and thawed in differentiation media: DMEM (high glucose, no Sodium Pyruvate)+2% Horse Serum+2 mM L-Glutamine+1 μM Insulin+100 units/mL Penicillin and 100 μg/mL Streptomycin. The primary human skeletal myoblasts were cultured in differentiation media for 2-3 days to undergo rapid differentiation.

After successful differentiation of myoblasts into myotubes, the cells were AAV-transduced with candidate NAREs driving expression of fluorescent proteins such as mClover3 and mIRFP713. Candidate promoters were tested along with known constitutive and tissue-specific reference promoters, such as CAG, CMV, tMCK, and creatine kinase 8 promoter (CK8e). With consistent Multiplicity of Infection in triplicate samples, the AAV candidates were diluted in differentiation media to replace the media containing the plated adherent myotubes. After 4 hours of incubation at 37° C. with 5% C02, differentiation media supplemented with 200 nM doxorubicin was added at the same volume as the AAV-containing media to have a final concentration of 100 nM doxorubicin, as an agent to improve transduction efficiency. After 1-2 days of infection, the AAV- and doxorubicin-containing differentiation media was changed for regular differentiation media to remove the AAV and doxorubicin. Themyotubes' fluorescent protein expression was detected via Agilent's/BioTek's Cytation 5 Cell Imaging Multimode Reader. The myotubes' intensity of fluorescence is measured as an indicator of NARE strength, with candidate NAREs to be compared among the known reference promoters.

### Results

A library of nucleic acid regulatory elements suitable for the expression of genes in the muscle was developed.

Some nucleic acid regulatory elements were derived from genes highly expressed in human skeletal muscle and were modified with various elements to increase NARE strength. The sizes of these NAREs ranged from 450 bp to 1.6 kb. Dual-Reporter plasmids with different NARE candidates were transiently transfected into C2C12 cells.

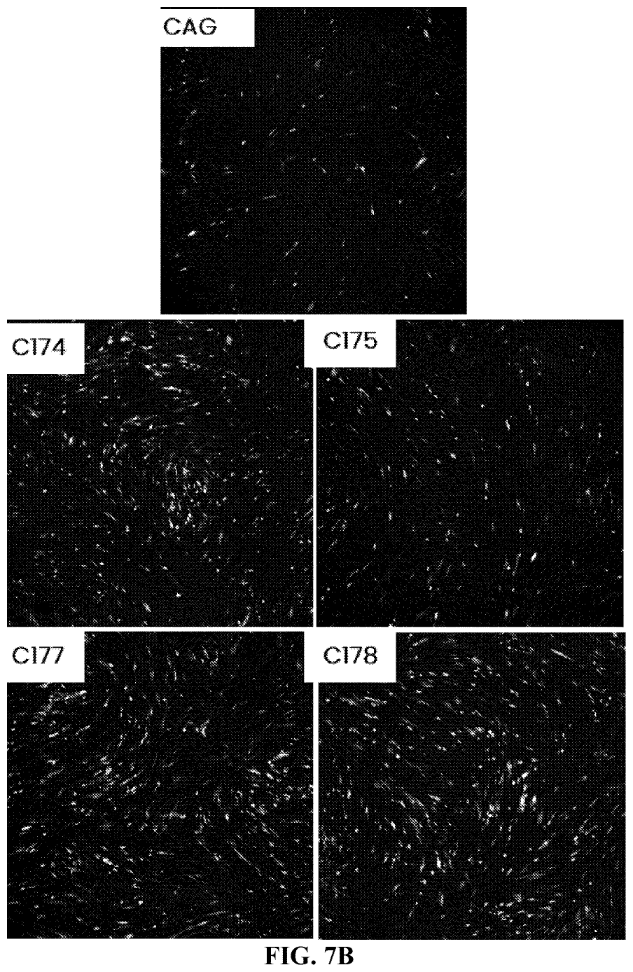

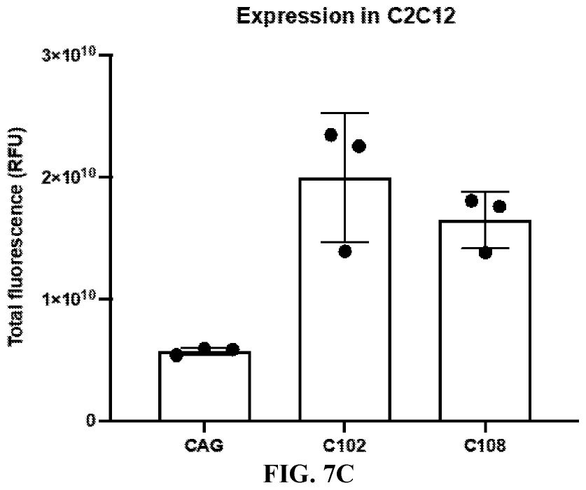

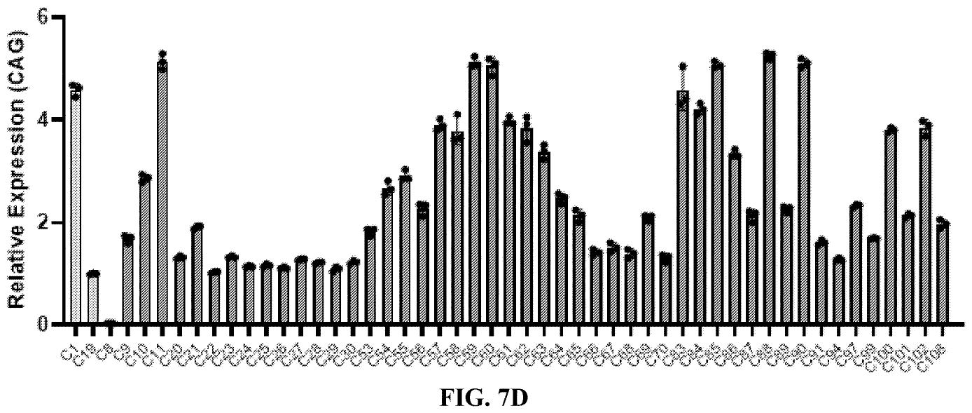

The potency of selected CAG-derived nucleic acid regulatory elements is shown in Tables 3-5 (C2C12 cells), FIG. 7C (C2C12 myoblasts), and FIG. 7D (AAV8-miRFP713 transduction, primary human myotubes). The potency of selected additional NAREs in C2C12 cells is shown in FIGS. 7C and 7D. C8, C9, C10 and C11 provide an example of a series of sequential modifications resulting in small, ubiquitous NARE, C11, with 5-fold higher activity than CAG in vitro. C8 is the parent ubiquitous promoter (AdML), nucleic acid regulatory element C9 adds an enhancer, nucleic acid regulatory element C10 adds a start site modification to C9, and nucleic acid regulatory element C11 adds an intron to C10 (FIG. 7D).

TABLE 3
Sequential NARE modifications and expression in C2C12
cells (see Table 9 for the specific modifications and sequences).
Dual reporter plasmids with different nucleic acid regulatory
element candidates were transiently transfected into C2C12 cells.
GFP expression level in transfected (RFP+) cells was measured
by flow cytometry. Mod1: Replaced chimeric intron in CAG with
native CBA intron. Mod5: Insertion of CMVp into intron. Mod6:
MTE/DPE insertion into CMVp. Mod15: Modification of CMVmd
promoter into ATGless form. Mod17: MTE/DPE insertion
into original CBAp of CAG. Mod19: Insertion of HBAmd
promoter into intron. Mod21: Insert of MBAmd promoter into
intron. Mod23: Insertion of beta catenin 5′-UTR
(CTBBN1). Mod24: Insertion of tubulin 5′-UTR (TUUB).
Mod25: Modification of MBAmd promoter into ATGless form.

| | Size | Potency |
|---|---|---|
| NARE | (bp) | (C2C12) |
| C1 (CMV) | 700 | 5.2 |
| C19 (CAG) | 1761 | 1.0 |
| C53 (CAG + Mod 1) | 1672 | 1.8 |
| C54 (CAG + Mod 1, 2) | 2055 | 2.7 |
| C55 (CAG + Mod 1, 3) | 1862 | 2.9 |
| C56 (CAG + Mod 1, 4) | 1746 | 1.4 |
| C57 (CAG + Mod 1, 5, 6) | 1930 | 3.9 |
| C58 (CAG + Mod 1, 5) | 1929 | 3.8 |
| C63 (CAG + Mod 1, 7) | 1959 | 1.4 |
| C64 (CAG + Mod 1, 7, 8) | 1714 | 2.5 |
| C65 (CAG + Mod 1, 7, 8, 9) | 1509 | 2.1 |
| C66 (CAG + Mod 1, 7, 8, 9, 10) | 1139 | 1.4 |
| C67 (CAG + Mod 1, 5, 6, 11) | 1680 | 1.5 |
| C68 (CAG + Mod 1, 5, 6, 11, 12) | 1439 | 1.4 |
| C69 (CAG + Mod 1, 5, 6, 11, 12, 13) | 1180 | 1.1 |
| C70 (CAG + Mod 1, 5, 6, 11, 12, 13, 14) | 1070 | 1.3 |
| C83 (CAG + Mod 1, 5, 6, 15) | 1930 | 3.9 |
| C84 (CAG + Mod 1, 16) | 1990 | 3.4 |
| C85 (CAG + Mod 1, 17, 5, 6) | 1979 | 5.1 |
| C86 (CAG + Mod 1, 16, 18) | 1990 | 3.3 |
| C87 (CAG + Mod 1, 19) | 1959 | 2.1 |
| C88 (CAG + Mod 1, 20, 5, 6) | 1948 | 5.3 |
| C89 (CAG + Mod 1, 21) | 1980 | 2.3 |
| C90 (CAG + Mod 1, 22, 5, 6) | 1969 | 5.1 |
| C91 (CAG + Mod 1, 19, 5, 6, 15, 16) | 2223 | 1.6 |
| C99 (CAG + Mod 1, 19, 5, 6, 15, 16, 18) | 2223 | 1.7 |
| C100 (CAG + Mod 1, 19, 16, 18, 5, 6, 15) | 1994 | 3.8 |
| C101 (CAG + Mod 1, 17, 19, 23, 16, 18, 24, 5, 6, 15) | 1964 | 2.1 |
| C102 (CAG + Mod 1, 17, 19, 23, 21, 25, 24, 5, 6, 15) | 1954 | 3.9 |
| C108 (CAG + Mod 1, 17, 19, 23, 21, 25, 24) | 1689 | 2.0 |

TABLE 4
Potency of CAG-derived nucleic acid
regulatory elements in C2C12 myoblasts.
Plasmid transfections.

| | | Potency relative to |
|---|---|---|
| NARE | Size (bp) | CAG in C2C12 myoblasts |
| C19 (CAG) | 1761 | 1 |
| C174 | 1021 | 1.9 |
| C175 | 1089 | 2.8 |
| C177 | 1139 | 1.5 |
| C178 | 1149 | 1.8 |

TABLE 5
Generation of selected CAG-derived promoters.
Indicated nucleic acid regulatory elements were generated
by replacing the CAG intron of CAG variant C85 (CAGmd-CMVmd
promoter) with the indicated introns.

| NARE | CAG intron replacement | Size (bp) |
|---|---|---|
| C173 | AdMLmd-MVMi | 1089 |
| C174 | JeTmd-MVMi | 1021 |
| C175 | CMVmd-MVMi | 1089 |
| C176 | HB Amd-MVMi | 1118 |
| C177 | MBAmd-MVMi | 1139 |
| C178 | CBAmd-MVMi | 1149 |

### Example 4: Strength of a Selected NARE in Muscle Cells (In Vivo)

### Materials and Methods

### Intramuscular Injections

Male C57BL6 mice, aged 6-8 weeks were kept in standard cages with ad libitum water, and were changed from the standard food chow to a purified chow containing no alfalfa to minimize background autofluorescence. Mice were anesthetized with 2-3% isoflurane with 1.5 LPM of oxygen. One leg for each mouse was shaved to remove all fur and cleaned with an alcohol prep pad, and the gastrocnemius was targeted using a 27G needle, being careful to avoid any blood vessels. Mice were allowed to recover in a clean warm cage and returned to their home cage after they were awake and mobile.

### Intravenous Injections

Male C57BL6 mice, aged 6-8 weeks were kept in standard cages with ad libitum water, and were changed from the standard food chow to a purified chow containing no alfalfa to minimize background autofluorescence. Mice were restrained and placed under a warming light to increase vasodilation and either tail vein was targeted using a 27G needle. Mice are allowed to recover in a clean warm cage and returned to their home cage after making sure they were not bleeding.

### Bioimaging Protocol

Mice were anesthetized using 2-3% isoflurane with 1.5 LPM oxygen and imaged using a LICOR Pearl System on the white channel and the 700 nm channel. Animals were imaged weekly for 5 weeks and then every two months for up to 12 months. Mice were allowed to recover in a clean warm cage and returned to their home cage after they were awake and mobile. Data analysis is performed using the LICOR Image Studio Software: a region of interest (ROI) was selected for background normalization on a part of the animal that had no fluorescence. An ROI encompassing the torso or both legs was drawn and normalized against the background ROI. The same area was used across animals on each timepoint to minimize artifacts in data collection. Absolute fluorescence was recorded for every imaging session.

eGFP mClover3 Imaging of IM Injections

Four weeks after injection, animals were euthanized, and the gastrocnemius was dissected and drop-fixed overnight in 4% Paraformaldehyde in PBS at 4° C. The next morning, muscles were changed to 15% sucrose in PBS and incubated at 4° C. overnight. Muscles were changed to 30% sucrose in PBS and incubated at 4° C. overnight. Muscles were then covered in OCT and allowed to equilibrate for 1 hour at RT in freezing molds. Samples were frozen on dry ice and then stored at −80° C. until being serially sliced for histology using the cryostat. Serial sections 25 μm thick were collected at 600 um intervals to gain a full representation of expression across the muscle. After n=3 per group were sectioned, slides air-dried for 10 minutes to complete mounting process. Slides were then washed 3× with PBS and mounted with Prolong anti-fade with NucBlue and imaged on the BioTek imager to create tilescans of all slices

### Results

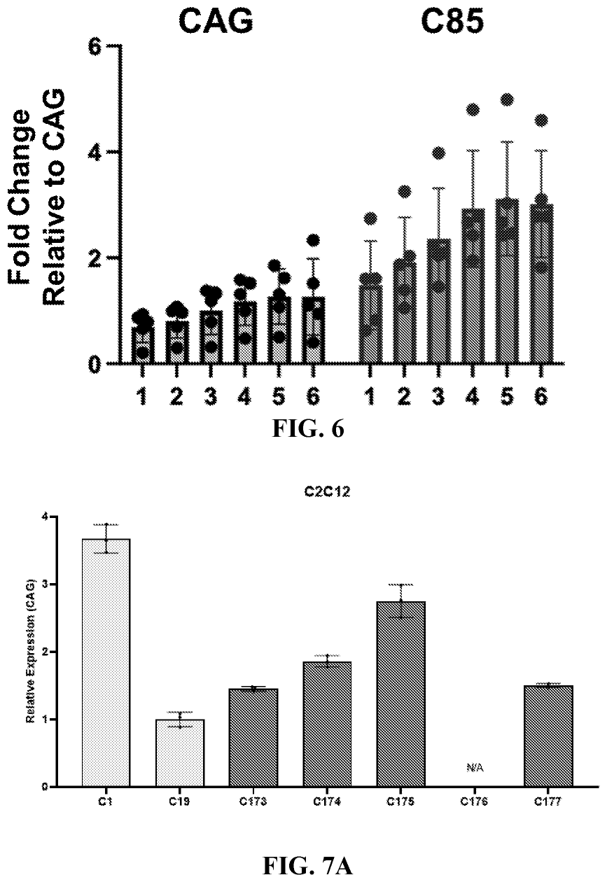

Gastrocnemius muscle of C57BL/6 mice was transduced with AAV8-eGFP constructs driven by either CAG or CAG-derived NARE C85 via IM injection at a dose of 5×10¹⁰ GC/muscle. The gastrocnemius muscle was sectioned five weeks post injection and imaged for eGFP fluorescence/expression. NARE C85 was up to 3 times stronger in skeletal muscle than the CAG promoter (FIG. 6).

### Example 5: Strength of Different Nucleic Acid Regulatory Elements in Neuronal Cells (In Vitro)

### Materials and Methods

### Cell Culture of Cell Lines

Neuro2A (N2A, mouse), BE2-M17 (human), SH-SY5Y (human), and Huh7 (human) immortalized cell lines were maintained at 37° C. in 5% CO₂. N2A is a murine neuroblastoma cell line. BE2-M17 is a human neuroblastoma cell line. SH-SY5Y is a thrice-subcloned cell line derived from the SK-N-SH neuroblastoma cell line. The Huh7 cell line is a hepatocyte-derived cellular carcinoma cell line (control). Cells were maintained in Dulbecco's Modified Eagle Medium (DMEM), 10% fetal bovine serum, 2 mM L-glutamine, and no sodium pyruvate. Cells were seeded at a density of 30,000 cells/cm². After 24 hours, cells were transiently transfected using TurboFect™ and with dual reporter plasmids that contain two expression cassettes (FIG. 2A). Two days post-transfection, cells were washed with Dulbecco's Phosphate Buffered Saline, trypsinized with 0.25% Trypsin-EDTA for 5 min at 37° C., neutralized with the growth medium, pelleted, and resuspended in FACS buffer. Cell suspensions are then analyzed on the flow cytometer. Live, single, tdTomato+ cells were selected for further analysis. Median Fluorescence Intensities of mClover3 and tdTomato was quantified and used to calculate the relative expression for each candidate NRE.

### Primary Neuronal Culture

Embryonic day 18 (E18) timed-pregnant female mice were anesthetized with CO₂ and sacrificed by cervical dislocation. In a dissection hood, 24-26 embryos per experiment were collected through an incision of the mother's abdomen, taken out of the amniotic sacs, and decapitated in ice-cold Hank's Balanced Salt Solution (HBSS). Using fine scissors and forceps, brains were rapidly dissected and the cortex cleared from meninges and isolated under a dissection microscope. Cortices were collected in ice-cold HBSS and kept on ice until all embryos had been dissected. In a tissue culture hood, HBSS was removed and the cortex tissue was digested by 0.25% Trypsin-EDTA for 12 min at 37° C., followed by DNaseI treatment for 10 min at 37° C. The tissue was dissociated by serial trituration with a 25-ml serological pipette, followed by trituration with 10 and 5 ml serological pipettes. The cell suspension was washed once with DMEM medium, supplemented with 10% FBS and 1% penicillin/streptomycin, and passed through a 40 μM cell strainer before being counted on a hemocytometer. Single cells were seeded on poly-D-lysine (0.1 mg ml⁻¹)-coated wells at a density of 400,000 cells per well on a 24-well plate. Cells were grown in neurobasal medium, complemented with B27 supplement, N2 supplement, and 0.5 mM L-glutamine and maintained at 37° C. in 5% CO₂. After four days, adeno-associated virus (AAV serotype 9) expressing mIRFP713e under the control of a candidate NARE was added to the cultures at a multiplicity of infection of 50,000. Cells were imaged using the Biotek Cytation after 7 days of infection. Cells were then fixed with 4% paraformaldehyde for 10 minutes. Coverslips were washed with PBS three times for 5 minutes, permeabilized with PBS+1.0% Triton for 10 minutes, followed by blocking with 0.1% Tritonx-100+5% Normal Donkey Serum+10×PBS in water. Slides were incubated with primary antibody (anti-NeuN, anti-GFAP) in 5% Normal Donkey Serum+10×PBS+0.1% Tritonx-100 in water overnight at 4° C. Slides were then washed and incubated with Alexa Fluor conjugated secondary antibodies in 5% Normal Donkey Serum+10×PBS+0.1% Tritonx-100 in water for one hour at room temperature. Slides were washed and cover-slipped using Prolong-gold anti-fade with DAPI (Invitrogen). Total miRFP713 fluorescence per image was normalized to the untransduced control and quantified using the Biotek Cytation software. ImageJ software and Cell Counter plugin was used to count cell numbers.

### Results

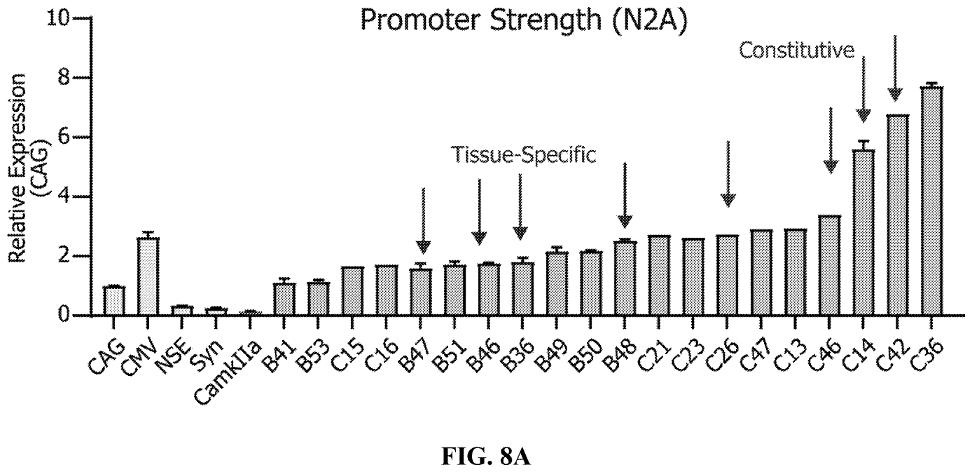

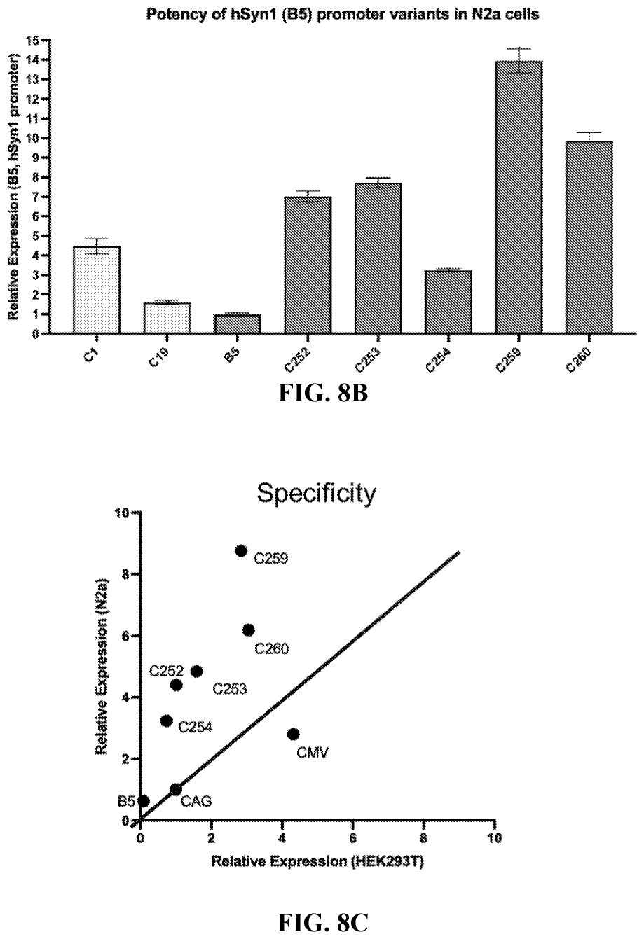

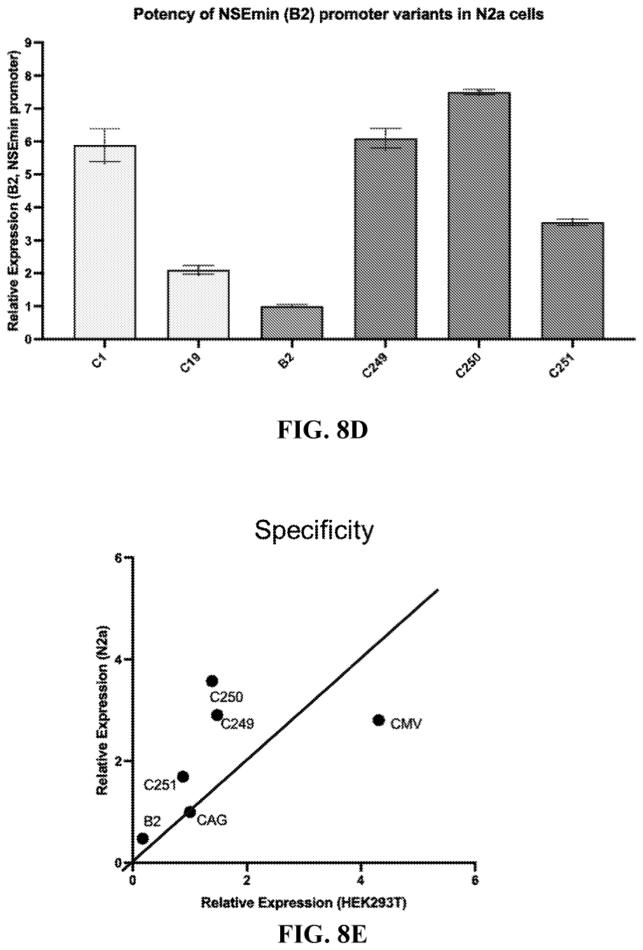

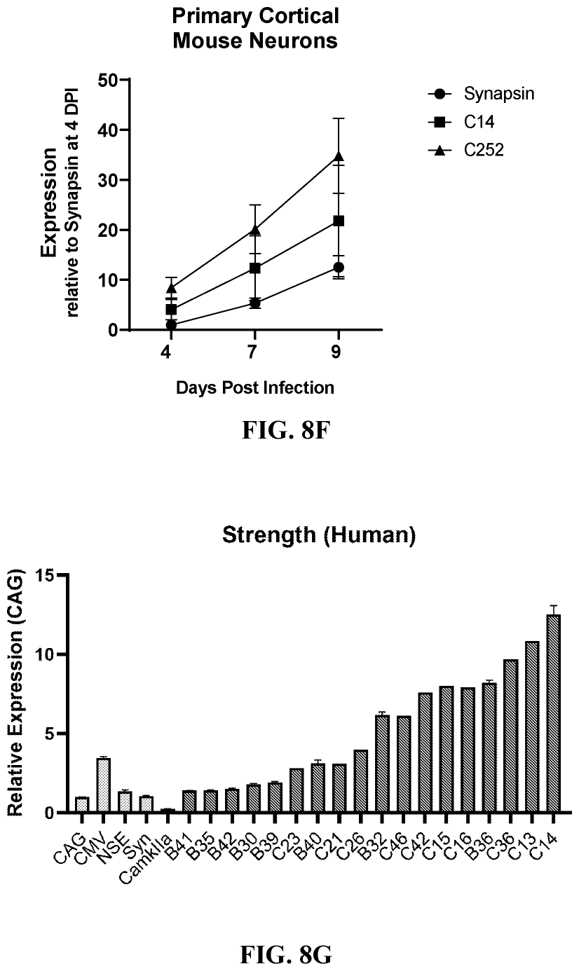

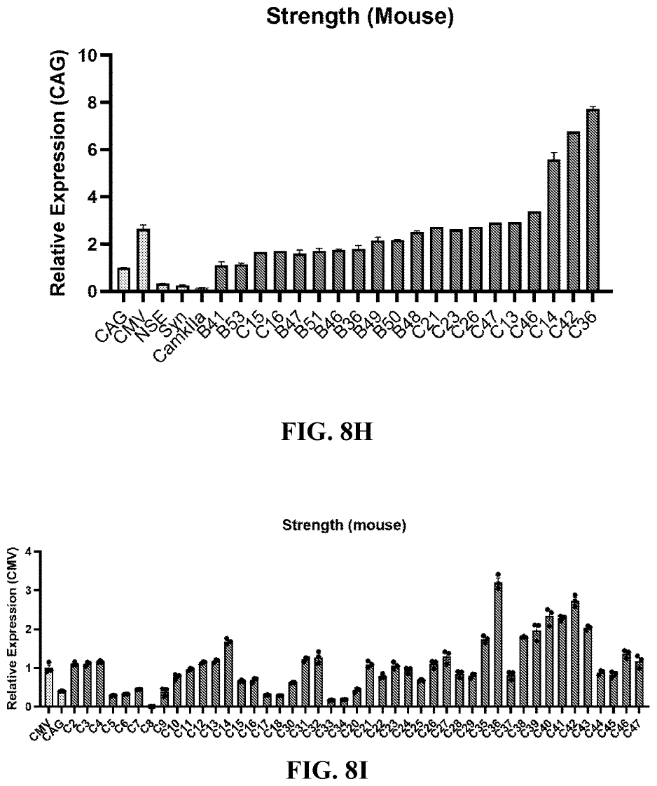

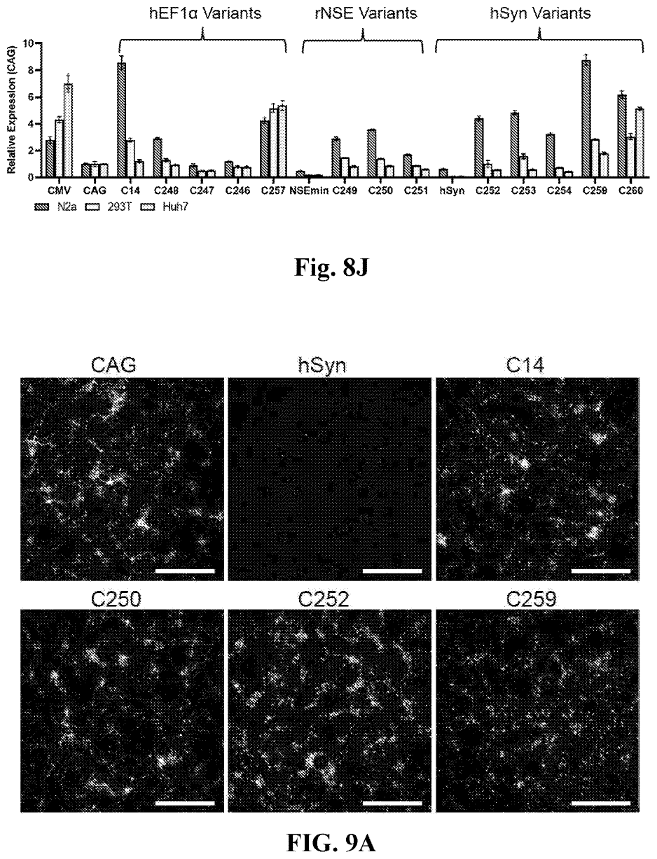

Several candidates NARE for tested for their ability to drive expression in neuronal cells (FIG. 8A). These NAREs showed strong expression in mouse N2A cells compared to commonly used reference promoters (NSE, Syn, CAMKIIalpha). Specifically, these identified NAREs were equal to or greater in NARE strength than CMV, and some were even stronger than CAG (FIG. 8A). Reference promoters: The neuron-specific enolase (NSE) is a neuron-specific promoter that contains a TATA-like sequence, no CAAT box and sequences for the AP-1 binding motif, AP-2 binding element, SP-1 binding sequence and cAMP response element. Syn (or human synapsin 1 promoter) and α-Calcium/calmodulin-dependent kinase II (CAMKIIalpha) are also neuron-specific promoters.

FIG. 8B shows the potency of hSyn1 (B5)-derived nucleic acid regulatory elements that exhibit greater potency and specificity than CMV (C1) and CAG (C19) promoters. C252 showed a 7-fold increase in potency relative to the original hSyn1 (B5) promoter, while maintaining 59% of hSyn1's neuronal specificity (C252 has a 4.5-fold improved neuronal specificity compared to CAG). C259 showed a 14-fold increase in potency relative to the original hSyn1 (B5) promoter, while maintaining 41% of hSyn1 neuronal specificity (C259 has a 3-fold improved neuronal specificity compared to CAG). Additional potency data are shown in FIG. 8J.

FIG. 8D shows the potency of NSEmin (B2)-derived nucleic acid regulatory elements that exhibit greater potency and specificity than the CMV promoter (C1), CAG promoter (C19), and NSEmin promoter (B2). C250 showed a 7-fold increase in potency relative to the original NSEmin (B2) promoter with almost no decrease in neuronal specificity. Additional potency data are shown in FIG. 8J.

FIG. 8J shows the potency of human EF1alpha-derived NAREs in different cell lines.

Neuron-specificity of selected NAREs is shown in (FIGS. 8C and 8E).

Nucleic acid regulatory elements C252 and C14 driving mIRFP713 expression showed increased potency in primary mouse cortical neurons as compared to the neuronal synapsin promoter (hSyn1, B4) (FIG. 8F). Four days post transduction, C252 and C14 showed 8-fold and 4-fold higher expression, respectively, as compared to the human synapsin promoter (B4).

The potency of additional nucleic acid regulatory elements in human and murine neuronal cell lines is shown in FIGS. 8G-8I.

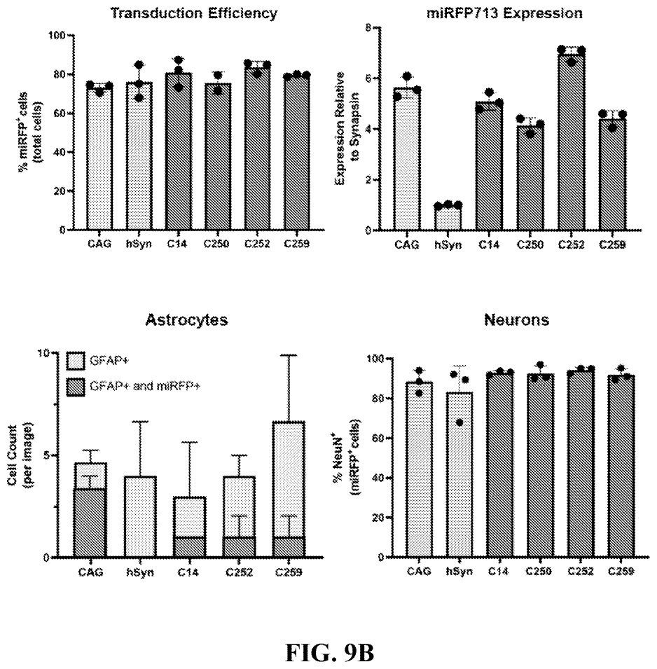

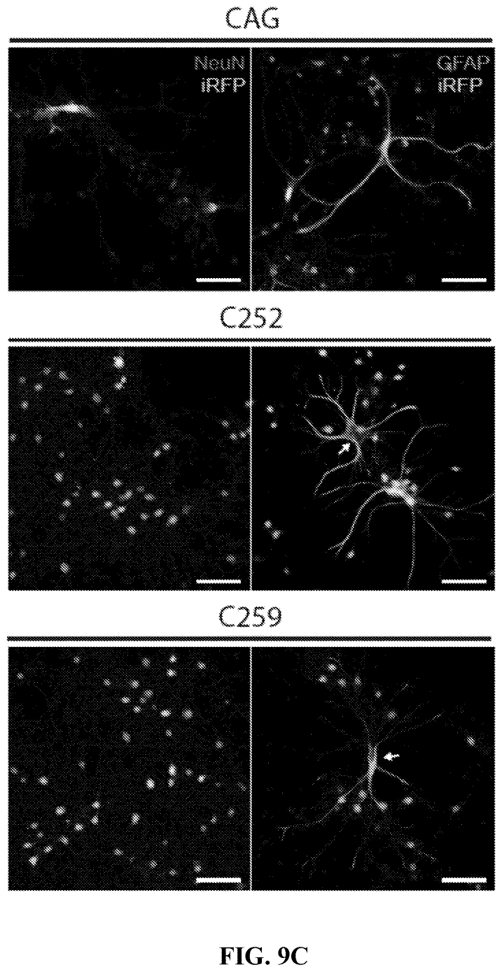

FIGS. 9A-9C show that selected NAREs drive high expression in primary cortical neurons. On day 4 in vitro, primary mouse cortical neurons were transduced at 50,000 multiplicity of infection (MOI) with AAV9-miRFP713 driven by candidate NAREs. After 7 days, neurons were imaged to assess miRFP713 (FIG. 9A). Transduction efficiency was consistent across constructs. At 7 days post infection (DPI), all NAREs lead to higher expression of miRFP713 than synapsin (hSyn) and showed fewer miRFP713-positive astrocytes than CAG (FIGS. 9B and 9C).

### Example 6: Strength of Different Nucleic Acid Regulatory Elements In Vivo

### Materials and Methods

Male C57Bl/6J mice (aged 8 weeks, n=5 per group) were injected intravenously with 5e12 GC/kg AAV9-miRFP713 driven by candidate NAREs. After 4 weeks, animals were perfused with PBS and organs rapidly dissected. Organs were immediately imaged using the Licor Pearl system to quantify miRFP713 fluorescence. Organs were then snap frozen in liquid nitrogen for molecular analysis. Liver samples (30 mg) are lysed using the NextAdvance Bead Lysis kit (#PINKE1-RNA). Whole hearts are lysed using the (NextAdvance: GREENE1-RNA). Samples are then homogenized for 3 minutes (speed 8 for liver, speed 10 for heart), incubated at 4° C. for 5 minutes, and homogenized a second time for 2 minutes (speed 10 for liver, speed 12 for heart). Samples are then treated with proteinase K for 30-90 minutes at room temperature and pelleted at 15,000×g for 10 minutes at room temperature. Nucleic acids were then isolated from sample lysates using the Zymo Quick-DNA/RNA Miniprep Plus kit according to manufacturer's instructions.

Viral genomes were quantified via qPCR. 300-600 ng of DNA were used to amplify the WPRE sequence. Total copy numbers were determined against a 10-fold serially diluted double stranded WPRE standard curve ranging from 1e9 to 1e3 copies/well. Reactions were run using a QuantStudio 6 Real Time PCR System (Applied Biosystems) and a Taqman primer probe against WPRE using the TaqPath ProAmp Master Mix (Applied Biosystems). Run parameters used were: 10 minutes at 95° C. for initial denaturation and enzyme activation, followed by 40 cycles of 15 seconds at 95° C. to denature and 60 seconds at 60° C. for annealing and extending.

### Results

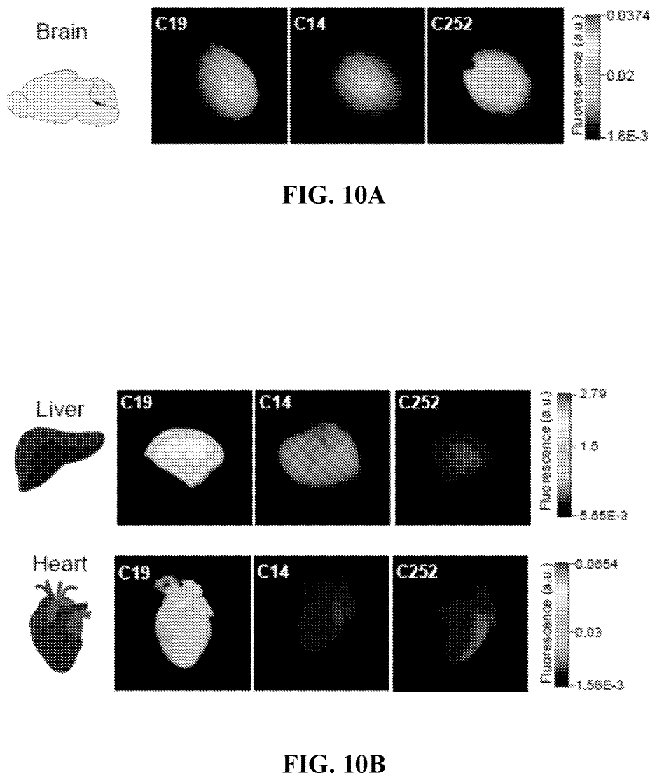

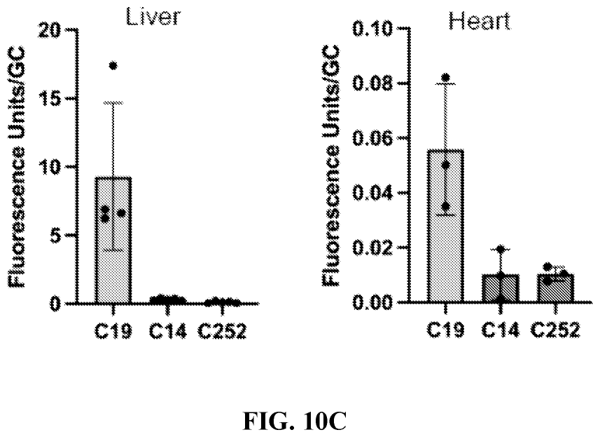

FIGS. 10A-10C show that selected NAREs are highly specific in vivo. Male C57Bl/6J mice were injected intravenously with 5e12 GC/kg AAV9-miRFP713 driven by candidate NAREs. After 4 weeks, fluorescence was measured for each organ ex vivo and genomes harvested for qPCR to quantify transduction. FIG. 10A shows representative images of ex vivo fluorescence of whole brain showing higher expression driven by selected NAREs compared to CAG. Peripheral tissue expression normalized to viral genomes shows low off-target expression in the mouse liver and heart driven by NAREs (FIGS. 10B and 10C).

### Example 7: Strength of Different Nucleic Acid Regulatory Elements in Liver Cells (In Vitro)

### Materials and Methods

### Cell Culture

Huh-7 immortalized cell lines were maintained 37° C. in 5% CO₂. The Huh7 cell line is a hepatocyte-derived cellular carcinoma cell line. HepG2 is a hepatoblastoma cell line. Cells were transiently transfected with plasmids using TurboFect™. After 48 hours, the cell lysates were collected for protein analysis. After 20 passages, a new aliquot of cells was thawed and passaged twice before using for subsequent experiments.

### Primary Hepatocyte Cultures

Mouse and human hepatocyte cell lines from Gibco were thawed and placed in suspended medium followed by centrifugation at 100×g for 10 minutes at 4° C. Supernatants were aspirated, and cells were resuspended in maintenance medium (+10% FBS) followed by plating at 400,000 cells per well on a 24-well plate. Four hours post plating, the plate was gently tapped to loosen debris, followed by aspiration of the medium, and replacement with non-FBS containing maintenance medium. Cells were maintained at 37° C. in 5% C02. Every day, full media changes were performed to feed the cultures.

### Transduction

On day 3 in vitro, AAV8 particles were added to the medium at a multiplicity of infection (MOI) of 50,000. Doxorubicin containing medium was added on to the cells to speed up transduction. Media was changed 72 hours later to non-doxorubicin containing medium according to standard protocol.

### Results

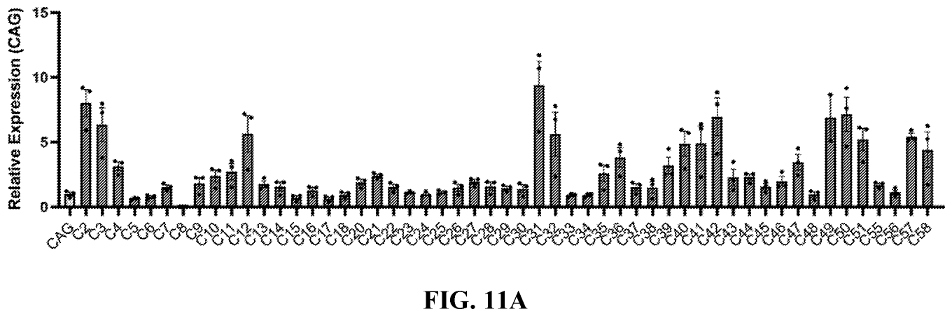

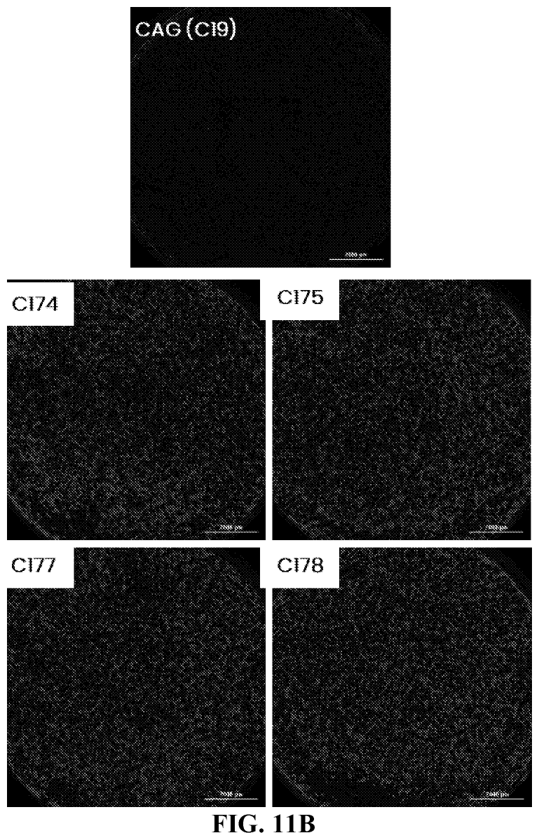

A library of nucleic acid regulatory elements suitable for the expression of genes in the liver was developed. Expression levels were tested using the dual reporter system using flow cytometry and presented relative to CAG (FIG. 11). The NAREs in the NARE library were of equal or greater strength than CAG while also being significantly smaller.

Potency of CAG-derived NAREs in transduced primary mouse hepatocytes is shown in FIG. 11B. Potency of CAG-derived NAREs in transfected Huh7 cells is shown in Table 6.

TABLE 6
Potency in Huh7 cells. Plasmid transfections.

| NARE | Size (bp) | Potency relative to CAG |
|---|---|---|
| C19 (CAG) | 1761 | 1 |
| C174 | 1021 | 2.2 |
| C175 | 1089 | 6 |
| C177 | 1139 | 1.2 |
| C178 | 1149 | 2.5 |

### Example 8: Strength of Different NAREs in Liver Cells (In Vivo)

Expression constructs containing an mIRFP713 transgene were under the control of C174 (1021 bp), C177 (1139 bp), or CAG (C19, 1761 bp), respectively, were packaged into AAV8 and administered to C57BL/6 mice by tail vein injection at a dose of 5×10¹² GC/kg. Expression in liver was imaged at 85 days post injection.

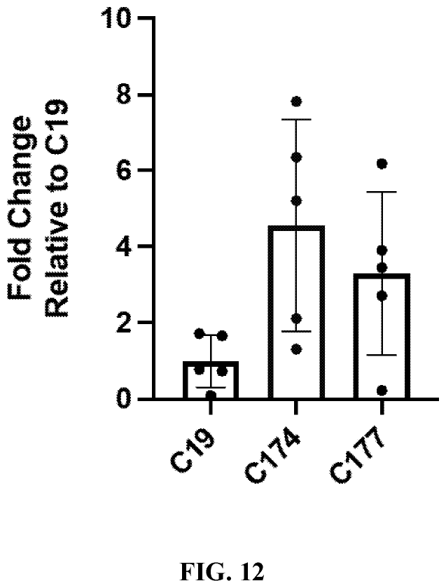

Nucleic acid regulatory elements C174 and C177 showed 3-5-fold higher expression as compared to the CAG promoter in the liver (FIG. 12).

An overview of sequences is provided in Tables 7-9.

Lengthy table referenced here
US20260109978A1-20260423-T00001
Please refer to the end of the specification for access instructions.

Lengthy table referenced here
US20260109978A1-20260423-T00002
Please refer to the end of the specification for access instructions.

Lengthy table referenced here
US20260109978A1-20260423-T00003
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260109978A1). An electronic copy of the table will also be available from the USPTO upon request and payment of the fee set forth in 37 CFR 1.19(b)(3).

We claim:

1. A nucleic acid regulatory element (NARE) comprising: (i) a sequence that is at least 90% identical to any one of SEQ ID NOs:344-349, 498, (ii) a sequence that is at least 90% identical to any one of SEQ ID NOs:337, 338, 355, 357, 358, 359, 407, 411, 429-431, 434, 501, 503, or 504; (iii) optionally, a sequence that is at least 90% identical to any one of SEQ ID NOs: 323, 420, or 488, and (iv) optionally, a sequence that is at least 90% identical to SEQ ID NO:402 or 444.

2. The NARE of claim 1, comprising: (i) a sequence that is at least 95% identical to any one of SEQ ID NOs:344-349, or 498, (ii) a sequence that is at least 95% identical to any one of SEQ ID NOs:337, 338, 355, 357, 358, 359, 407, 411, 429-431, 434, 501, 503, or 504; (iii) optionally, a sequence that is at least 95% identical to any one of SEQ ID NOs: 323, 420, 488, and (iv) optionally, a sequence that is at least 95% identical to SEQ ID NO:402 or 444.

3. The NARE of claim 1, comprising: (i) any one of SEQ ID NOs:344-349, or 498, (ii) any one of SEQ ID NOs:337, 338, 355, 357, 358, 359, 407, 411, 429-431, 434, 501, 503, or 504; (iii) optionally, any one of SEQ ID NOs: 323, 420, 488, and (iv) optionally, SEQ ID NO:402 or 444.

4. A NARE comprising: (i) a sequence that is at least 90% identical to any one of SEQ ID NOs:381, 384, 392, 393, 394, 395, 396, 397, 400, or 401; (ii) a sequence that is at least 90% identical to SEQ ID NO: 334 or SEQ ID NO: 342; and (iii) a sequence that is at least 90% identical to SEQ ID NO:426.

5. The NARE of claim 4, comprising: (i) a sequence that is at least 95% identical to any one of SEQ ID NOs:381, 384, 392, 393, 394, 395, 396, 397, 400, or 401; (ii) a sequence that is at least 95% identical to SEQ ID NO: 334 or SEQ ID NO: 342; and (iii) a sequence that is at least 95% identical to SEQ ID NO:426.

6. A NARE comprising: (i) any one of SEQ ID NOs:381, 384, 392, 393, 394, 395, 396, 397, 400, or 401; and (ii) SEQ ID NO: 334 or SEQ ID NO: 342; and (iii) SEQ ID NO:426.

7. A NARE comprising: (i) a sequence that is at least 90% identical to any one of SEQ ID NOs:344-349, or 498; (ii) a sequence that is at least 90% identical to SEQ ID NO:334 or SEQ ID NO: 342; (iii) a sequence that is at least 90% identical to any one of SEQ ID NOs:302-318, 320-322, 324-327, 331, 332, or 487, (iv) optionally, SEQ ID NO:333; and (v) optionally, a sequence that is at least 90% identical to SEQ ID NO:319.

8. The NARE of claim 7, comprising: (i) a sequence that is at least 95% identical to any one of SEQ ID NOs:344-349, or 498; (ii) a sequence that is at least 95% identical to SEQ ID NO:334 or SEQ ID NO: 342; (iii) a sequence that is at least 95% identical to any one of SEQ ID NOs:302-318, 320-322, 324-327, 331, 332, or 487, (iv) optionally, SEQ ID NO:333; and (v) optionally, a sequence that is at least 95% identical to SEQ ID NO:319.

9. The NARE of claim 7, comprising: (i) any one of SEQ ID NOs:344-349, or 498; (ii) SEQ ID NO:334 or SEQ ID NO: 342; (iii) any one of SEQ ID NOs:302-318, 320-322, 324-327, 331, 332, or 487, (iv) optionally, SEQ ID NO:333; and (v) optionally, SEQ ID NO:319.

10. The NARE of claim 7, comprising: (i) a sequence that is at least 90% identical to any one of SEQ ID NOs:344-349, or 498; (ii) a sequence that is at least 90% identical to SEQ ID NO: 334 or SEQ ID NO: 342; and (iii) a sequence that is at least 90% identical to any one of SEQ ID NOs:302-317, 320-322, 324-327, 331, 332, or 487.

11. The NARE of claim 7, comprising: (i) a sequence that is at least 95% identical to any one of SEQ ID NOs:344-349, or 498; (ii) a sequence that is at least 95% identical to SEQ ID NO: 334 or SEQ ID NO: 342; and (iii) a sequence that is at least 95% identical to any one of SEQ ID NOs:302-317, 320-322, 324-327, 331, 332, or 487.

12. The NARE of claim 7, comprising: (i) any one of SEQ ID NOs:344-349, or 498; (ii) SEQ ID NO: 334 or SEQ ID NO: 342; and (iii) any one of SEQ ID NOs:302-317, 320-322, 324-327, 331, 332, or 487.

13. The NARE of claim 7, comprising: (i) a sequence that is at least 90% identical to SEQ ID NOs:344-349, 498; (ii) a sequence that is at least 90% identical to SEQ ID NOs:334 or 342; and (iii) a sequence that is at least 90% identical to any one of SEQ ID NOs:302-317, 320-322, 324-327, 331, or 332.

14. The NARE of claim 7, comprising: (i) a sequence that is at least 95% identical to SEQ ID NOs:344-349, 498; (ii) a sequence that is at least 95% identical to SEQ ID NOs:334 or 342; and (iii) a sequence that is at least 95% identical to any one of SEQ ID NOs:302-317, 320-322, 324-327, 331, or 332.

15. The NARE of claim 7, comprising: (i) SEQ ID NOs:344-349, 498; (ii) SEQ ID NOs:334 or 342; and (iii) any one of SEQ ID NOs:302-317, 320-322, 324-327, 331, or 332.

16. A NARE comprising: (i) a sequence that is at least 90% identical to any one of SEQ ID NOs:344-349, or 498; (ii) a sequence that is at least 90% identical to any one of SEQ ID NOs:351-356, 499, or 500; (iii) a sequence that is at least 90% identical to any one of SEQ ID NOs: 200, 370, 417-420, 502, 441, 482-485, 489, or 491-496; and (iv) optionally, a sequence that is at least 90% identical to any one of SEQ ID NOs:444, 452, or 488; (v) optionally, a sequence that is at least 90% identical to SEQ ID NO:402; and (vi) optionally, SEQ ID NO:433.

17. The NARE of claim 16, comprising: (i) a sequence that is at least 95% identical to any one of SEQ ID NOs:344-349, or 498; (ii) a sequence that is at least 95% identical to any one of SEQ ID NOs:351-356, 499, or 500; (iii) a sequence that is at least 95% identical to any one of SEQ ID NOs: 200, 370, 417-420, 502, 441, 482-485, 489, or 491-496; and (iv) optionally, a sequence that is at least 95% identical to any one of SEQ ID NOs:444, 452, or 488; (v) optionally, a sequence that is at least 95% identical to SEQ ID NO:402; and (vi) optionally, SEQ ID NO:433.

18. The NARE of claim 16, comprising: (i) any one of SEQ ID NOs:344-349, or 498; (ii) any one of SEQ ID NOs:351-356, 499, or 500; (iii) any one of SEQ ID NOs: 200, 370, 417-420, 502, 441, 482-485, 489, or 491-496; and (iv) optionally, any one of SEQ ID NOs:444, 452, or 488; (v) optionally, SEQ ID NO:402; and (vi) optionally, SEQ ID NO:433.

19. The NARE of claim 16, comprising: (i) a sequence that is at least 90% identical to any one of SEQ ID NOs:344-349, or 498; (ii) a sequence that is at least 90% identical to any one of SEQ ID NOs:351-356, 499, or 500; (iii) a sequence that is at least 90% identical to any one of SEQ ID NOs: 200, 370, 417-420, 502, 482-485, 489, or 491-496; and (iv) optionally, a sequence that is at least 90% identical to SEQ ID NOs:444 or 452.

20. The NARE of claim 16, comprising: (i) a sequence that is at least 95% identical to any one of SEQ ID NOs:344-349, or 498; (ii) a sequence that is at least 95% identical to any one of SEQ ID NOs:351-356, 499, or 500; (iii) a sequence that is at least 95% identical to any one of SEQ ID NOs: 200, 370, 417-420, 502, 482-485, 489, or 491-496; and (iv) optionally, a sequence that is at least 95% identical to SEQ ID NOs:444 or 452.

21. The NARE of claim 16, comprising: (i) any one of SEQ ID NOs:344-349, 498; (ii) any one of SEQ ID NOs:351-356, 499, or 500; (iii) any one of SEQ ID NOs: 200, 370, 417-420, 502, 482-485, 489, or 491-496; and (iv) optionally, SEQ ID NOs:444 or 452.

22. The NARE of claim 16, comprising: (i) a sequence that is at least 90% identical to any one of SEQ ID NOs:344-349, or 498; (ii) a sequence that is at least 90% identical to any one of SEQ ID NOs:351-356, 499, or 500; (iii) a sequence that is at least 90% identical to any one of SEQ ID NOs:200, 417-420, 502, or 485; and (iv) a sequence that is at least 90% identical to optionally, SEQ ID NOs:444 or 452.

23. The NARE of claim 16, comprising: (i) a sequence that is at least 95% identical to any one of SEQ ID NOs:344-349, or 498; (ii) a sequence that is at least 95% identical to any one of SEQ ID NOs:351-356, 499, or 500; (iii) a sequence that is at least 95% identical to any one of SEQ ID NOs:200, 417-420, 502, or 485; and (iv) a sequence that is at least 95% identical to optionally, SEQ ID NOs:444 or 452.

24. The NARE of claim 16, comprising: (i) any one of SEQ ID NOs:344-349, 498; (ii) any one of SEQ ID NOs:351-356, 499, or 500; (iii) any one of SEQ ID NOs:200, 417-420, 502, or 485; and (iv) optionally, SEQ ID NOs:444 or 452.

25. The NARE of claim 16, comprising: (i) a sequence that is at least 90% identical to any one of SEQ ID NOs:344-349, or 498; (ii) a sequence that is at least 90% identical to any one of SEQ ID NOs:351-356, 499, or 500; and (iii) a sequence that is at least 90% identical to any one of SEQ ID NOs:370, 482-484, 489, or 491-496.

26. The NARE of claim 16, comprising: (i) a sequence that is at least 95% identical to any one of SEQ ID NOs:344-349, or 498; (ii) a sequence that is at least 95% identical to any one of SEQ ID NOs:351-356, 499, or 500; and (iii) a sequence that is at least 95% identical to any one of SEQ ID NOs:370, 482-484, 489, or 491-496.

27. The NARE of claim 16, comprising: (i) any one of SEQ ID NOs:344-349, 498; (ii) any one of SEQ ID NOs:351-356, 499, or 500; and (iii) any one of SEQ ID NOs:370, 482-484, 489, or 491-496.

28. A NARE comprising: (i) a sequence that is at least 90% identical to any one of SEQ ID NOs:344-349, or 498; (ii) a sequence that is at least 90% identical to SEQ ID NO:335; and (iii) a sequence that is at least 90% identical to any one of SEQ ID NOs: 294, 295, 296, 299, 323, 328, 329, 330, 407, 501, 430, 431, 503, 434, or 504; and (iv) optionally, a sequence that is at least 90% identical to SEQ ID NO:444.

29. The NARE of claim 28, comprising: (i) a sequence that is at least 95% identical to any one of SEQ ID NOs:344-349, or 498; (ii) a sequence that is at least 95% identical to SEQ ID NO:335; and (iii) a sequence that is at least 95% identical to any one of SEQ ID NOs: 294, 295, 296, 299, 323, 328, 329, 330, 407, 501, 430, 431, 503, 434, or 504; and (iv) optionally, a sequence that is at least 95% identical to SEQ ID NO:444.

30. The NARE of claim 28, comprising: (i) any one of SEQ ID NOs:344-349, or 498; (ii) SEQ ID NO:335; and (iii) any one of SEQ ID NOs: 294, 295, 296, 299, 323, 328, 329, 330, 407, 501, 430, 431, 503, 434, or 504; and (iv) optionally, SEQ ID NO:444.

31. The NARE of claim 28, comprising: (i) a sequence that is at least 90% identical to any one of SEQ ID NOs:344-349, or 498; (ii) a sequence that is at least 90% identical to SEQ ID NO:335; and (iii) a sequence that is at least 90% identical to any one of SEQ ID NOs: 294, 295, 296, 407, 501, 430, 431, 503, 434, or 504; and (iv) a sequence that is at least 90% identical to SEQ ID NO:444.

32. The NARE of claim 28, comprising: (i) a sequence that is at least 95% identical to any one of SEQ ID NOs:344-349, or 498; (ii) a sequence that is at least 95% identical to SEQ ID NO:335; and (iii) a sequence that is at least 95% identical to any one of SEQ ID NOs: 294, 295, 296, 407, 501, 430, 431, 503, 434, or 504; and (iv) a sequence that is at least 95% identical to SEQ ID NO:444.

33. The NARE of claim 28, comprising: (i) any one of SEQ ID NOs:344-349, or 498; (ii) SEQ ID NO:335; and (iii) any one of SEQ ID NOs: 294, 295, 296, 407, 501, 430, 431, 503, 434, or 504; and (iv) SEQ ID NO:444.

34. A NARE comprising: (i) a sequence that is at least 90% identical to any one of SEQ ID NOs:339, 363-369, 378, 379, 382, 385, 387-389, 391, 398, or 399; and (ii) a sequence that is at least 90% identical to any one of SEQ ID NOs:430, 431, or 503.

35. The NARE of claim 34, comprising: (i) a sequence that is at least 95% identical to any one of SEQ ID NOs:339, 363-369, 378, 379, 382, 385, 387-389, 391, 398, or 399; and (ii) a sequence that is at least 95% identical to any one of SEQ ID NOs:430, 431, or 503.

36. The NARE of claim 34, comprising: (i) any one of SEQ ID NOs:339, 363-369, 378, 379, 382, 385, 387-389, 391, 398, or 399; and (ii) any one of SEQ ID NOs:430, 431, or 503.

37. A NARE comprising a sequence that is at least 90% identical to any one of SEQ ID NOs:138-161.

38. The NARE of claim 37, comprising a sequence that is at least 95% identical to any one of SEQ ID NOs:138-161.

39. The NARE of claim 37, comprising any one of SEQ ID NOs:138-161.

40. A NARE comprising: (i) a sequence that is at least 90% identical to SEQ ID NO:298; (ii) a sequence that is at least 90% identical to SEQ ID NO:444; and (iii) optionally, a sequence that is at least 90% identical to SEQ ID NO:475.

41. The NARE of claim 40, comprising: (i) a sequence that is at least 95% identical to SEQ ID NO:298; (ii) a sequence that is at least 95% identical to SEQ ID NO:444; and (iii) optionally, a sequence that is at least 95% identical to SEQ ID NO:475.

42. The NARE of claim 40, comprising: (i) SEQ ID NO:298; (ii) SEQ ID NO:444, and (iii) optionally, SEQ ID NO:475.

43. A NARE comprising: (i) a sequence that is at least 90% identical to any one of SEQ ID NOs:339, 380, 383, 386, or 390; (ii) a sequence that is at least 90% identical to SEQ ID NOs:439 or 497; and (iii) optionally, a sequence that is at least 90% identical to SEQ ID NO:444.

44. The NARE of claim 43, comprising: (i) a sequence that is at least 95% identical to any one of SEQ ID NOs:339, 380, 383, 386, or 390; (ii) a sequence that is at least 95% identical to SEQ ID NOs:439 or 497; and (iii) optionally, a sequence that is at least 95% identical to SEQ ID NO:444.

45. The NARE of claim 43, comprising: (i) any one of SEQ ID NOs:339, 380, 383, 386, or 390; (ii) SEQ ID NOs:439 or 497; and (iii) optionally, SEQ ID NO:444.

46. The NARE of claim 43, comprising: (i) a sequence that is at least 90% identical to any one of SEQ ID NOs:339, 380, 383, 386, or 390; and (ii) a sequence that is at least 90% identical to SEQ ID NOs:439 or 497.

47. The NARE of claim 43, comprising: (i) a sequence that is at least 95% identical to any one of SEQ ID NOs:339, 380, 383, 386, or 390; and (ii) a sequence that is at least 95% identical to SEQ ID NOs:439 or 497.

48. The NARE of claim 43, comprising: (i) any one of SEQ ID NOs:339, 380, 383, 386, or 390; and (ii) SEQ ID NOs:439 or 497.

49. A NARE comprising: (i) a sequence that is at least 90% identical to any one of SEQ ID NOs:344-349, or 498; (ii) a sequence that is at least 90% identical to SEQ ID NO:336; and (iii) a sequence that is at least 90% identical to any one of SEQ ID NOs:201, 284, 285, 301, 362, 372, 373, 403-406, 408-410, 412, 427, 444, 453-456, 486, or 490.

50. The NARE of claim 49, comprising: (i) a sequence that is at least 95% identical to any one of SEQ ID NOs:344-349, or 498; (ii) a sequence that is at least 95% identical to SEQ ID NO:336; and (iii) a sequence that is at least 95% identical to any one of SEQ ID NOs:201, 284, 285, 301, 362, 372, 373, 403-406, 408-410, 412, 427, 444, 453-456, 486, or 490.

51. The NARE of claim 49, comprising: (i) any one of SEQ ID NOs:344-349, or 498; (ii) SEQ ID NO:336; and (iii) any one of SEQ ID NOs:201, 284, 285, 301, 362, 372, 373, 403-406, 408-410, 412, 427, 444, 453-456, 486, or 490.

52. A NARE comprising: (i) a sequence that is at least 90% identical to any one of SEQ ID NOs:374-376; (ii) optionally, a sequence that is at least 90% identical to any one of SEQ ID NOs:344-349, 498 or 393; (iii) a sequence that is at least 90% identical to any one of SEQ ID NOs:93-98, 103, 109, 128-136, 195-198, 201, 284, 285, 301, 362, 371, 373, 403-406, 408-410, 412, 417-420, 502, 427, 453-456, 460-474, 486, or 490; and (iv) optionally, a sequence that is at least 90% identical to SEQ ID NOs:443 or 448.

53. The NARE of claim 52, comprising: (i) a sequence that is at least 95% identical to any one of SEQ ID NOs:374-376; (ii) optionally, a sequence that is at least 95% identical to any one of SEQ ID NOs:344-349, 498 or 393; (iii) a sequence that is at least 95% identical to any one of SEQ ID NOs:93-98, 103, 109, 128-136, 195-198, 201, 284, 285, 301, 362, 371, 373, 403-406, 408-410, 412, 417-420, 502, 427, 453-456, 460-474, 486, or 490; and (iv) optionally, a sequence that is at least 95% identical to SEQ ID NOs:443 or 448.

54. The NARE of claim 52, comprising: (i) any one of SEQ ID NOs:374-376; (ii) optionally, any one of SEQ ID NOs:344-349, 498 or 393; (iii) any one of SEQ ID NOs:93-98, 103, 109, 128-136, 195-198, 201, 284, 285, 301, 362, 371, 373, 403-406, 408-410, 412, 417-420, 502, 427, 453-456, 460-474, 486, or 490; and (iv) optionally, SEQ ID NOs:443 or 448.

55. The NARE of claim 52, comprising: (i) a sequence that is at least 90% identical to any one of SEQ ID NOs:374-376; and (ii) a sequence that is at least 90% identical to any one of SEQ ID NOs:93-98, 103, 109, 128-136, 195-198, 201, 284, 285, 301, 362, 371, 373, 403-406, 408-410, 412, 427, 453-456, 460-474, 486, or 490.

56. The NARE of claim 52, comprising: (i) a sequence that is at least 95% identical to any one of SEQ ID NOs:374-376; and (ii) a sequence that is at least 95% identical to any one of SEQ ID NOs:93-98, 103, 109, 128-136, 195-198, 201, 284, 285, 301, 362, 371, 373, 403-406, 408-410, 412, 427, 453-456, 460-474, 486, or 490.

57. The NARE of claim 52, comprising: (i) any one of SEQ ID NOs:374-376; and (ii) any one of SEQ ID NOs:93-98, 103, 109, 128-136, 195-198, 201, 284, 285, 301, 362, 371, 373, 403-406, 408-410, 412, 427, 453-456, 460-474, 486, or 490.

58. The NARE of claim 52, comprising: (i) a sequence that is at least 90% identical to any one of SEQ ID NOs:374-376; and (ii) a sequence that is at least 90% identical to any one of SEQ ID NOs:201, 284, 285, 301, 362, 371, 373, 403-406, 408-410, 412, 427, 453-456, 486, or 490.

59. The NARE of claim 52, comprising: (i) a sequence that is at least 95% identical to any one of SEQ ID NOs:374-376; and (ii) a sequence that is at least 95% identical to any one of SEQ ID NOs:201, 284, 285, 301, 362, 371, 373, 403-406, 408-410, 412, 427, 453-456, 486, or 490.

60. The NARE of claim 52, comprising: (i) any one of SEQ ID NOs:374-376; and (ii) any one of SEQ ID NOs:201, 284, 285, 301, 362, 371, 373, 403-406, 408-410, 412, 427, 453-456, 486, or 490.

61. The NARE of claim 52, comprising: (i) a sequence that is at least 90% identical to any one of SEQ ID NOs:374-376; and (ii) a sequence that is at least 90% identical to any one of SEQ ID NOs:93-98, 103, 109, 128-136, 195-198, or 460-474.

62. The NARE of claim 52, comprising: (i) a sequence that is at least 95% identical to any one of SEQ ID NOs:374-376; and (ii) a sequence that is at least 95% identical to any one of SEQ ID NOs:93-98, 103, 109, 128-136, 195-198, or 460-474.

63. The NARE of claim 52, comprising: (i) any one of SEQ ID NOs:374-376; and (ii) any one of SEQ ID NOs:93-98, 103, 109, 128-136, 195-198, or 460-474.

64. The NARE of claim 52, comprising: (i) a sequence that is at least 90% identical to any one of SEQ ID NOs:344-349, 498, or 393; (ii) a sequence that is at least 90% identical to any one of SEQ ID NOs:374-376; (iii) a sequence that is at least 90% identical to any one of SEQ ID NOs:417-420, or 502; and (iv) optionally, a sequence that is at least 90% identical to SEQ ID NOs:443 or 448.

65. The NARE of claim 52, comprising: (i) a sequence that is at least 95% identical to any one of SEQ ID NOs:344-349, 498 or 393; (ii) a sequence that is at least 95% identical to any one of SEQ ID NOs:374-376; (iii) a sequence that is at least 95% identical to any one of SEQ ID NOs:417-420, or 502; and (iv) optionally, a sequence that is at least 95% identical to SEQ ID NOs:443 or 448.

66. The NARE of claim 52, comprising: (i) any one of SEQ ID NOs:344-349, 498 or 393; (ii) any one of SEQ ID NOs:374-376; (iii) any one of SEQ ID NOs:417-420, or 502; and (iv) optionally, SEQ ID NOs:443 or 448.

67. A NARE comprising: (i) a sequence that is at least 90% identical to SEQ ID NOs:478 or 479; (ii) a sequence that is at least 90% identical to SEQ ID NO:350; (iii) a sequence that is at least 90% identical to any one of SEQ ID NOs:351-356, 499, or 500; and (iv) a sequence that is at least 90% identical to any one of SEQ ID NOs:361, 421, 422, 423, 428, 444, or 480.

68. The NARE of claim 67, comprising: (i) a sequence that is at least 95% identical to SEQ ID NOs:478 or 479; (ii) a sequence that is at least 95% identical to SEQ ID NO:350; (iii) a sequence that is at least 95% identical to any one of SEQ ID NOs:351-356, 499, or 500; and (iv) a sequence that is at least 95% identical to any one of SEQ ID NOs:361, 421, 422, 423, 428, 444, or 480.

69. The NARE of claim 67, comprising: (i) SEQ ID NOs:478 or 479; (ii) SEQ ID NO:350; (iii) any one of SEQ ID NOs:351-356, 499, or 500; and (iv) any one of SEQ ID NOs:361, 421, 422, 423, 428, 444, or 480.

70. A NARE comprising: (i) a sequence that is at least 90% identical to SEQ ID NO:377; and (ii) a sequence that is at least 90% identical to any one of SEQ ID NOs:444, 457, 458, 469, or 470.

71. The NARE of claim 70, comprising: (i) a sequence that is at least 95% identical to SEQ ID NO:377; and (ii) a sequence that is at least 95% identical to any one of SEQ ID NOs:444, 457, 458, 469, or 470.

72. The NARE of claim 70, comprising: (i) SEQ ID NO:377; and (ii) any one of SEQ ID NOs:444, 457, 458, 469, or 470.

73. A NARE comprising: (i) a sequence that is at least 90% identical to any one of SEQ ID NOs:199, 476, or 477; and (ii) a sequence that is at least 90% identical to SEQ ID NO:300.

74. The NARE of claim 73, comprising: (i) a sequence that is at least 95% identical to any one of SEQ ID NOs:199, 476, or 477; and (ii) a sequence that is at least 95% identical to SEQ ID NO:300.

75. The NARE of claim 73, comprising: (i) any one of SEQ ID NOs:199, 476, or 477; and (ii) SEQ ID NO:300.

76. A NARE comprising: (i) a sequence that is at least 90% identical to SEQ ID NO:474; (ii) a sequence that is at least 90% identical to SEQ ID NO:340 or 341; and (iii) optionally, a sequence that is at least 90% identical to SEQ ID NO:393.

77. The NARE of claim 76, comprising: (i) a sequence that is at least 95% identical to SEQ ID NO:474; (ii) a sequence that is at least 95% identical to SEQ ID NO:340 or 341; and (iii) optionally, a sequence that is at least 95% identical to SEQ ID NO:393.

78. The NARE of claim 76, comprising: (i) NO:474; (ii) SEQ ID NO:340 or 341; and (iii) optionally, SEQ ID NO:393.

79. A NARE comprising a sequence that is at least 90% identical to any one of SEQ ID NOs: 6, 7, 9-12, 14, 16-18, 21-32, 34, 37-91, 99-102, 104-108, 110-127, 138-194, 202-283, or 286-293.

80. The NARE of claim 79, comprising a sequence that is at least 95% identical to any one of SEQ ID NOs: 6, 7, 9-12, 14, 16-18, 21-32, 34, 37-91, 99-102, 104-108, 110-127, 138-194, 202-283, or 286-293.

81. The NARE of claim 79, comprising any one of SEQ ID NOs: 6, 7, 9-12, 14, 16-18, 21-32, 34, 37-91, 99-102, 104-108, 110-127, 138-194, 202-283, or 286-293.

82. The NARE of claim 79, comprising a sequence that is at least 90% identical to any one of SEQ ID NOs: 6, 14, 16, 18, 34, 48, 104-107, 162-172, 179, 180, 183, 184, or 293.

83. The NARE of claim 79, comprising a sequence that is at least 95% identical to any one of SEQ ID NOs: 6, 14, 16, 18, 34, 48, 104-107, 162-172, 179, 180, 183, 184, or 293.

84. The NARE of claim 79, comprising any one of SEQ ID NOs: 6, 14, 16, 18, 34, 48, 104-107, 162-172, 179, 180, 183, 184, or 293.

85. An expression construct comprising the NARE of any one of claims 1-84 and an operatively linked transgene.

86. The expression construct of claim 85, further comprising a polyadenylation sequence.

87. A vector comprising the expression construct of claim 85.

88. The vector of claim 87, wherein the vector is a non-viral vector.

89. The vector of claim 87, wherein the vector is a viral vector.

90. The vector of claim 89, wherein the vector is an adeno-associated virus (AAV) vector.

91. A vector comprising a nucleic acid sequence comprising (i) the expression construct of 85, and (ii) one or more inverted terminal repeats (ITR).

92. The vector of claim 91, wherein the nucleic acid sequence comprises a 5′ ITR and a 3′ ITR.

93. The vector of claim 92, wherein the 5′ ITR and a 3′ ITR are derived from AAV serotype AAV2.

94. A cell comprising the expression construct of claim 84 or the vector of any one of claims 87-93.

95. The cell of claim 94, wherein the cell is a neuronal, muscle, or liver cell.

96. A pharmaceutical composition comprising (i) the expression construct of claim 85 or the vector of any one of claims 87-93 and (ii) a pharmaceutically acceptable excipient.

97. A method for expressing a transgene in a cell comprising the expression construct of claim 85 or the vector of any one of claims 87-93.

98. A method for regulating transgene expression in a cell comprising the expression construct of claim 84 or the vector of any one of claims 86-93.

99. The method of claim 97 or 98, wherein the cell is a neuronal, muscle, or liver cell.


SEQUENCE LISTING (505 sequences)

SEQ ID NO: 1 - DNA, 700 bp, synthetic construct
  source 1..700: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaataatacgactcactatagggaagcttggcattccggtactgttggatccgccacc

SEQ ID NO: 2 - DNA, 763 bp, synthetic construct
  source 1..763: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaataatacgactaagaggtaagggtttaagggatggttggttggtggggtattaatgtttaattacctggagcacctgcctgaaatcactttttttcaggttggccgcggatccgccacc

SEQ ID NO: 3 - DNA, 700 bp, synthetic construct
  source 1..700: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaataatacgactcactatagggaagcttggcattccggtactgttggatccgccacc

SEQ ID NO: 4 - DNA, 763 bp, synthetic construct
  source 1..763: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaataatacgactaagaggtaagggtttaagggatggttggttggtggggtattaatgtttaattacctggagcacctgcctgaaatcactttttttcaggttggccgcggatccgccacc

SEQ ID NO: 5 - DNA, 207 bp, synthetic construct
  source 1..207: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacaggatccgccacc

SEQ ID NO: 6 - DNA, 209 bp, synthetic construct
  source 1..209: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 7 - DNA, 693 bp, synthetic construct
  source 1..693: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacataatacgactaagaggtaagggtttaagggatggttggttggtggggtattaatgtttaattacctggagcacctgcctgaaatcactttttttcaggttggccgcggatccgccacc

SEQ ID NO: 8 - DNA, 106 bp, synthetic construct
  source 1..106: mol_type = other DNA
ggtgttcctgaagggggctataaaagggggtgggggcgcgttcgtcctcactctcttccgcatcgctgtctgcgagggccagctgttggggtgaggatccgccacc

SEQ ID NO: 9 - DNA, 657 bp, synthetic construct
  source 1..657: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggggggctataaaagggggtgggggcgcgttcgtcctcactctcttccgcatcgctgtctgcgagggccagctgttggggtgaggatccgccacc

SEQ ID NO: 10 - DNA, 700 bp, synthetic construct
  source 1..700: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggggtgttcctgaagggggctataaaagggggtgggggcgcgttcgtcctcactctcttccgcatcctcgagccgagtggtcgtctgttggggtgataatacgactcactatagggaagcttggcattccggtactgttggatccgccacc

SEQ ID NO: 11 - DNA, 763 bp, synthetic construct
  source 1..763: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggggtgttcctgaagggggctataaaagggggtgggggcgcgttcgtcctcactctcttccgcatcctcgagccgagtggtcgtctgttggggtgataatacgactaagaggtaagggtttaagggatggttggttggtggggtattaatgtttaattacctggagcacctgcctgaaatcactttttttcaggttggccgcggatccgccacc

SEQ ID NO: 12 - DNA, 857 bp, synthetic construct
  source 1..857: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaataatacgactaagaggtaagggtttaagggatggttggttggtggggtattattgtttaattacctggagcggtgttcctgaagggggctataaaagggggtgggggcgcgttcgtcctcactctcttccgcatcgctgtctgcgagggccagctgttggggtgaacctgcctgaaatcactttttttcaggttggccgcggatccgccacc

SEQ ID NO: 13 - DNA, 1347 bp, synthetic construct
  source 1..1347: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 14 - DNA, 1366 bp, synthetic construct
  source 1..1366: mol_type = other DNA
ttcgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 15 - DNA, 603 bp, synthetic construct
  source 1..603: mol_type = other DNA
gtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacaaacagtagttgacaattaatcatcggcatagtatatcggcatagtataatacgactcactataggagggcggatccgccacc

SEQ ID NO: 16 - DNA, 695 bp, synthetic construct
  source 1..695: mol_type = other DNA
gtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcaagaggtaagggtttaagggatggttggttggtggggtattaatgtttaattacctggagcacctgcctgaaatcactttttttcaggttggcaagctgtgaccggcgcctacaaacagtagttgacaattaatcatcggcatagtatatcggcatagtataatacgactcactataggagggcggatccgccacc

SEQ ID NO: 17 - DNA, 1042 bp, synthetic construct
  source 1..1042: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacaaacagtagttgacaattaatcatcggcatagtatatcggcatagtataatacgactcactataggagggcggatccgccacc

SEQ ID NO: 18 - DNA, 1134 bp, synthetic construct
  source 1..1134: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcaagaggtaagggtttaagggatggttggttggtggggtattaatgtttaattacctggagcacctgcctgaaatcactttttttcaggttggcaagctgtgaccggcgcctacaaacagtagttgacaattaatcatcggcatagtatatcggcatagtataatacgactcactataggagggcggatccgccacc

SEQ ID NO: 19 - DNA, 1761 bp, synthetic construct
  source 1..1761: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgaggtggaaattaatacgacgtggcggatccgccacc

SEQ ID NO: 20 - DNA, 818 bp, synthetic construct
  source 1..818: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccgccacc

SEQ ID NO: 21 - DNA, 883 bp, synthetic construct
  source 1..883: mol_type = other DNA
gtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgccgggtcggcgcgcgcgcacgcagcggccacaccctcgggcggccagcggctcgggcaggaagtgcgcgcaagcgcccgggagccgcggcgaccccaccccttccggccgagcccgccttcgccccagcccaggccgcggcaccccgggccccagaacgcacgcggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccgccacc

SEQ ID NO: 22 - DNA, 882 bp, synthetic construct
  source 1..882: mol_type = other DNA
agcaggaatgaggggtggagggaagggcctcatttctggggaaggcccagggaagggcctcatttctggggaaggcccagggaagggccagggtgcagagaagccaatgacagctggagagttgggactgctgccaggacagactagacggagactgctcttgggtgccacctagtgggaaccttctttactgtcatccctggagagatggacctgtgacgtccccaccggatgccccgcccaccatgggcatccccatcccaatcatccctcaccacccccaggctcgggtcgcgtccccccaggctttgatggacaagtggccatggaaagcatcctcagctgccccagtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccgccacc

SEQ ID NO: 23 - DNA, 878 bp, synthetic construct
  source 1..878: mol_type = other DNA
ttacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccgccacc

SEQ ID NO: 24 - DNA, 883 bp, synthetic construct
  source 1..883: mol_type = other DNA
gcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgcatgctcttctccacctcagtgatgacgagagcgggcgggtgagggggcgggaacgcagcgatctctgggttctacgttagtgggagtttaacgacggtccctgggattccccaaggcaggggcgagtccttttgtatgaattactctcagctccggtcggggcggggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccgccacc

SEQ ID NO: 25 - DNA, 883 bp, synthetic construct
  source 1..883: mol_type = other DNA
acgtggtgtggaaagggttaaaactggcgctcggcgggatacagggctctaggtgccttaaagaacacgcggacacacttgtttggcttctgtcgcgctccgtagaatgagttccctgggcctcggtgggccacgcctccgcaaagaaggacaacgcgaagctgcccgcgtagtgcctgttgcactcgccaaggaccggcctggcagagatagaaaactcgcagtacgtttgcctcagaaccgcctcaatccggaaacagaaactccccaggcccggcactgcgggagccacctctttcgagggtgggagtgcacatgcgcacaggggacctgaaaaacccttaaataccggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccgccacc

SEQ ID NO: 26 - DNA, 882 bp, synthetic construct
  source 1..882: mol_type = other DNA
cgagtcttcaaaccttaagcctcagtgcaaaagggagcagcgaggagaggcggtgggattaaagaaaaaaattcttctctccctgtcacttcaaaaggtcgcccaaccagcgagcggggggtttacccacgactctggctcgcaccccttgcgggcctgcgcaggcggccccgtagcgcaagggagggcgggaaaggaaggggcggggacgcgagggcgaatctataagaagcgtcgttcagcgagttcgctgctcagaagcgccgagagcgcggccgggacggttggagaagaaggcggctcccggaagggggagagacaaactgccgtaacctctgccgttcaggaacgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccgccacc

SEQ ID NO: 27 - DNA, 889 bp, synthetic construct
  source 1..889: mol_type = other DNA
actttgatctcgttggggtcgaacttcggcggcatggtggaggcggctggtgtcggatgaacccggattcgggacgaccgaaggaagttgcaccttggcctcctccgagccgaaagccgagaggccggaaatcgcgcggacaagccagatataggcaggaaaacgcggctatcgcgagaactgtcgtcactgcccggccttccagagacgcccttgtcgccatgtttgttgtgggcaggcgcctgaggctgacggctggcaagcagggcaccgcggtgcgctgaagctagagattccgaaagggggttcgggtagttcgctccggagaagtctgagaagggtggctccggtgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcgcggccggatccgccacc

SEQ ID NO: 28 - DNA, 696 bp, synthetic construct
  source 1..696: mol_type = other DNA
agccacagccctctcagcgtctttccggggttggacacgccaccctctgcctcttctcgaaacagcacagagcgcgaggcgggccacccgctcctccctcgggtgtgggcagctccgaaactcacaccggggaggcgtggagagagccccgtcgcccgctaaccgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccgccacc

SEQ ID NO: 29 - DNA, 716 bp, synthetic construct
  source 1..716: mol_type = other DNA
taactgtagcctttctctctttccctgtagaaacctctgcgccatgagagccaaggtgagcggttcctggtagtaagcttgggaggtaggagttggcgagtagtagcgaggagacgaaggcaagtccgccatacctcctgaactactgggtttcaagggtgcccaagagctggtgggagagagagtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccgccacc

SEQ ID NO: 30 - DNA, 705 bp, synthetic construct
  source 1..705: mol_type = other DNA
ggagccttggatcgcgtccctggaaagggacaccaaagatttccaattccggagagcgggcccgaggaagggtcactgctcgggcgcacgaaagctgtctaaggcttgggcgtatatggggaactctggcttttgccacgcacttttgggaatgggcaggagacctgcttcctgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccgccacc

SEQ ID NO: 31 - DNA, 1053 bp, synthetic construct
  source 1..1053: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgcagcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgatttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccacc

SEQ ID NO: 32 - DNA, 1037 bp, synthetic construct
  source 1..1037: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagagcgtggagacagttcaggagggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcaagaggtaagggtttaagggatggttggttggtggggtattaatgtttaattacctggagcacctgcctgaaatcactttttttcaggttggggatccgccacc

SEQ ID NO: 33 - DNA, 636 bp, synthetic construct
  source 1..636: mol_type = other DNA
gtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactcgcccagttccgcccattctccgccccatggctgactaattttttttatttatgcagaggccgaggccgcctcggcctctgagctattccagaagtagtgaggaggcttttttggaggcctaggcttttgcaaaaagctcctcgatcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctcctcgggatccgccacc

SEQ ID NO: 34 - DNA, 728 bp, synthetic construct
  source 1..728: mol_type = other DNA
gtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactcgcccagttccgcccattctccgccccatggctgactaattttttttatttatgcagaggccgaggccgcctcggcctctgagctattccagaagtagtgaggaggcttttttggaggcctaggcttttgcaaaaagctcctcgatcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctcctcgaagaggtaagggtttaagggatggttggttggtggggtattaatgtttaattacctggagcacctgcctgaaatcactttttttcaggttggggatccgccacc

SEQ ID NO: 35 - DNA, 1248 bp, synthetic construct
  source 1..1248: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagagcgtggagacagttcaggagggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcaagaggtaagggtttaagggatggttggttggtggggtattaatgtttaattacctggagcacctgcctgaaatcactttttttcaggttggggatccgccacc

SEQ ID NO: 36 - DNA, 1245 bp, synthetic construct
  source 1..1245: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccacc

SEQ ID NO: 37 - DNA, 1005 bp, synthetic construct
  source 1..1005: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccacc

SEQ ID NO: 38 - DNA, 980 bp, synthetic construct
  source 1..980: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgaagaggtaagggtttaagggatggttggttggtggggtattaatgtttaattacctggagcacctgcctgaaatcactttttttcaggttggttgaaaagaagaagacgaagaagacgaagaagacaaaccgtcggatccgccacc

SEQ ID NO: 39 - DNA, 1030 bp, synthetic construct
  source 1..1030: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgggtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggttgaaaagaagaagacgaagaagacgaagaagacaaaccgtcggatccgccacc

SEQ ID NO: 40 - DNA, 987 bp, synthetic construct
  source 1..987: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccggtaagtttagtctttttgtcttttatttcaggtcccggatccggtggtggtgcaaatcaaagaactgctcctcagtggatgttgcctttacttctaggcttgaaaagaagaagacgaagaagacgaagaagacaaaccgtcggatccgccacc

SEQ ID NO: 41 - DNA, 1115 bp, synthetic construct
  source 1..1115: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtttgaaaagaagaagacgaagaagacgaagaagacaaaccgtcggatccgccacc

SEQ ID NO: 42 - DNA, 1116 bp, synthetic construct
  source 1..1116: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccacc

SEQ ID NO: 43 - DNA, 1023 bp, synthetic construct
  source 1..1023: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgggtaagtatcaaggttacaagacaggtttaaggagaccaatagaaactgggcttgtcgagacagagaagactcttgcgtttctgataggcacctattggtcttactgacatccactttgcctttctctccacaggttgaaaagaagaagacgaagaagacgaagaagacaaaccgtcggatccgccacc

SEQ ID NO: 44 - DNA, 1000 bp, synthetic construct
  source 1..1000: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacaaacagtagttgacggatccgccacc

SEQ ID NO: 45 - DNA, 1176 bp, synthetic construct
  source 1..1176: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgaattggtttgatctgattataactagtggagaagagcatgcttgagggctgagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcaagaggtaagggtttaagggatggttggttggtggggtattaatgtttaattacctggagcaggtctatataagcagagctcgtttagtgaaccgtcagatcgcctagatacgccatccacgctgttttgacctccatagaagacacctgcctgaaatcactttttttcaggttggcaagctgtgaccggcgcctacaaacagtagttgacggatccgccacc

SEQ ID NO: 46 - DNA, 966 bp, synthetic construct
  source 1..966: mol_type = other DNA
ttacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacaattggtttgatctgattataactagtggagaagagcatgcttgagggctgagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacaaacagtagttgacggatccgccacc

SEQ ID NO: 47 - DNA, 1142 bp, synthetic construct
  source 1..1142: mol_type = other DNA
ttacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacaattggtttgatctgattataactagtggagaagagcatgcttgagggctgagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcaagaggtaagggtttaagggatggttggttggtggggtattaatgtttaattacctggagcaggtctatataagcagagctcgtttagtgaaccgtcagatcgcctagatacgccatccacgctgttttgacctccatagaagacacctgcctgaaatcactttttttcaggttggcaagctgtgaccggcgcctacaaacagtagttgacggatccgccacc

SEQ ID NO: 48 - DNA, 700 bp, synthetic construct
  source 1..700: mol_type = other DNA
gacattgattattgactagttattaatagtaatcaattaaggggtcattagttcatagcccatatatggagttccgcgttacataacttaaggtaaatggcccgcctggctgaccgcccaacgacccccgcccattgacgtcaataatgacgtatgttcccatagtaacgccaatagggactttccattgacgtcaatgggtggactatttatggtaaactgcccacttggcagtacatcaagtgtatcatatgccaagtacgccccctattgacgtcaatgactgtaaatggcccgcctggcattatgcccagtacatgaccttatgggactttcctacttggcagtacatctacatattagtcatccctattaccatggtgatgcggttttggcagtacatcaatgggcgtggatagcggtttgactcatggggatttccaagtctccaccccattgacgtcaatgggagtttgttttggcaccaaaatcaacgggactttccaaaatgtggtaacaactccgccccattgacgcaaatgggcggtaggggtgtatggtgggaggtctatataagcagagctggtttagtgaactgtcagatcgcctggagatgccatccacgctgttttgacctccatagaataatacgactcactatagggaagcttggcattccggtactgttggatccgccacc

SEQ ID NO: 49 - DNA, 1707 bp, synthetic construct
  source 1..1707: mol_type = other DNA
cggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcaggaattcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccacc

SEQ ID NO: 50 - DNA, 1775 bp, synthetic construct
  source 1..1775: mol_type = other DNA
agcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaagggggcgcggcaggagggggcaccagggagcggagccctggccctcctgacgtcctgcccgcccacgcgtccgcaggtgaggtgcatcctgacaggtcacgagctgccctgccgcctgccggagctccaggtctacacccgcggcaaaaagtaccagcggctggtccgcgcctccccggccttcgactatgcagagttcgagccgcacatcgtgcccagcaccaagaacccgtaggtggtcgaattcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccacc

SEQ ID NO: 51 - DNA, 2012 bp, synthetic construct
  source 1..2012: mol_type = other DNA
gcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaagaattcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccacc

SEQ ID NO: 52 - DNA, 1267 bp, synthetic construct
  source 1..1267: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagtccgcctggagacctcgagccgagtggtcgtggccatccacgctgttttgacctccatagtggacaccgggaccgatccagcctccgcgtctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtggatccaactcctaaaaaaccgccacc

SEQ ID NO: 53 - DNA, 1672 bp, synthetic construct
  source 1..1672: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggggtttatatcttcccttctctgttcctccgcagccagccgccacc

SEQ ID NO: 54 - DNA, 2055 bp, synthetic construct
  source 1..2055: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcctcggcttctggcgtgtgaccggcggggtttatatcttcccttctctgttcctccgcagccagcgccacc

SEQ ID NO: 55 - DNA, 1862 bp, synthetic construct
  source 1..1862: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacatcggcttctggcgtgtgaccggcggggtttatatcttcccttctctgttcctccgcagccagcgccacc

SEQ ID NO: 56 - DNA, 1746 bp, synthetic construct
  source 1..1746: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 57 - DNA, 1930 bp, synthetic construct
  source 1..1930: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 58 - DNA, 1929 bp, synthetic construct
  source 1..1929: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagatcggcttctggcgtgtgaccggcggggtttatatcttcccttctctgttcctccgcagccagcgccacc

SEQ ID NO: 59 - DNA, 2142 bp, synthetic construct
  source 1..2142: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagatcggcttctggcgtgtgaccggcggggtttatatcttcccttctctgttcctccgcagccagccgccacc

SEQ ID NO: 60 - DNA, 2143 bp, synthetic construct
  source 1..2143: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagccgccacc

SEQ ID NO: 61 - DNA, 2075 bp, synthetic construct
  source 1..2075: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacatcggcttctggcgtgtgaccggcggggtttatatcttcccttctctgttcctccgcagccagccgccacc

SEQ ID NO: 62 - DNA, 2268 bp, synthetic construct
  source 1..2268: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcctcggcttctggcgtgtgaccggcggggtttatatcttcccttctctgttcctccgcagccagccgccacc

SEQ ID NO: 63 - DNA, 1959 bp, synthetic construct
  source 1..1959: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagccgccacc

SEQ ID NO: 64 - DNA, 1715 bp, synthetic construct
  source 1..1715: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacgccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagccgccacca

SEQ ID NO: 65 - DNA, 1509 bp, synthetic construct
  source 1..1509: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggaccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagccgccacc

SEQ ID NO: 66 - DNA, 1139 bp, synthetic construct
  source 1..1139: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacgggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagccgccacc

SEQ ID NO: 67 - DNA, 1680 bp, synthetic construct
  source 1..1680: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 68 - DNA, 1439 bp, synthetic construct
  source 1..1439: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 69 - DNA, 1180 bp, synthetic construct
  source 1..1180: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 70 - DNA, 1070 bp, synthetic construct
  source 1..1070: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctgggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 71 - DNA, 721 bp, synthetic construct
  source 1..721: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctgcattccaaccttccagcctgcgacctgcggagaaaaaaaattacttattttcttgccccatacataccttgaggcgagcaaaaaaattaaattttaaccggatccgccacc

SEQ ID NO: 72 - DNA, 721 bp, synthetic construct
  source 1..721: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctgccggtggcggcaggatacagcggcttctgcgcgacttataagagctccttgtgcggcgccattttaagcctctcggtctgtggcagcagcgttggcccgggatccgccacc

SEQ ID NO: 73 - DNA, 721 bp, synthetic construct
  source 1..721: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctcattctgtggtctgatcatcctgtggtttcgtcgccgccatcctcgtcgcgacacgctgttttcggttctcggcccgacgagccatcgccatcctacagcggatccgccacc

SEQ ID NO: 74 - DNA, 721 bp, synthetic construct
  source 1..721: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctcactcgcgctgccatcactcttccgccgtcttcgccgccatcctcggcgcgactcgcttctttcggttctaccaggtagagtccgccgccatcctccaccggatccgccacc

SEQ ID NO: 75 - DNA, 721 bp, synthetic construct
  source 1..721: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctcttgtctcgctccggggaacgctcggaaactcccggccgccgccacccgcgtctgttctgttacacaagggaagaaaagccgctgccgcactccgagtgtggatccgccacc

SEQ ID NO: 76 - DNA, 827 bp, synthetic construct
  source 1..827: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctcattctgtggtctgatcatcctgtggtttcgtcgccgccatcctcgtcgcgacacgctgttttcggttctcggcccgacgagccatcgccatcctacagccaacaacttgtctcgctccggggaacgctcggaaactcccggccgccgccacccgcgtctgttctgttacacaagggaagaaaagccgctgccgcactccgagtgtggatccgccacc

SEQ ID NO: 77 - DNA, 827 bp, synthetic construct
  source 1..827: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctcactcgcgctgccatcactcttccgccgtcttcgccgccatcctcggcgcgactcgcttctttcggttctaccaggtagagtccgccgccatcctccacccaacaacttgtctcgctccggggaacgctcggaaactcccggccgccgccacccgcgtctgttctgttacacaagggaagaaaagccgctgccgcactccgagtgtggatccgccacc

SEQ ID NO: 78 - DNA, 827 bp, synthetic construct
  source 1..827: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctcttgtctcgctccggggaacgctcggaaactcccggccgccgccacccgcgtctgttctgttacacaagggaagaaaagccgctgccgcactccgagtgtcaacaacactcgcgctgccatcactcttccgccgtcttcgccgccatcctcggcgcgactcgcttctttcggttctaccaggtagagtccgccgccatcctccaccggatccgccacc

SEQ ID NO: 79 - DNA, 815 bp, synthetic construct
  source 1..815: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaacccactcgcgctgccatcactcttccgccgtcttcgccgccatcctcggcgcgactcgcttctttcggttctaccaggtagagtccgccgccatcctccacccaacaacttgtctcgctccggggaacgctcggaaactcccggccgccgccacccgcgtctgttctgttacacaagggaagaaaagccgctgccgcactccgagtgtggatccgccacc

SEQ ID NO: 80 - DNA, 851 bp, synthetic construct
  source 1..851: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtcactcgcgctgccatcactcttccgccgtcttcgccgccatcctcggcgcgactcgcttctttcggttctaccaggtagagtccgccgccatcctccacccaacaacttgtctcgctccggggaacgctcggaaactcccggccgccgccacccgcgtctgttctgttacacaagggaagaaaagccgctgccgcactccgagtgtggatccgccacc

SEQ ID NO: 81 - DNA, 903 bp, synthetic construct
  source 1..903: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctgctagctcgggccgggaggagccgcagccggaggagggggaggaggaagaagagaaggaagaggagagggggccgcagtggcgactcggcgctcggaagccgggctcgtggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaacc

SEQ ID NO: 82 - DNA, 616 bp, synthetic construct
  source 1..616: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcaagatggcggc

SEQ ID NO: 83 - DNA, 1930 bp, synthetic construct
  source 1..1930: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 84 - DNA, 1990 bp, synthetic construct
  source 1..1990: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaatcggcttctggcgtgtgaccggcggggtttatatcttcccttctctgttcctccgcagccagcgccacc

SEQ ID NO: 85 - DNA, 1979 bp, synthetic construct
  source 1..1979: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaaggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 86 - DNA, 1990 bp, synthetic construct
  source 1..1990: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaatcggcttctggcgtgtgaccggcggggtttatatcttcccttctctgttcctccgcagccagcgccacc

SEQ ID NO: 87 - DNA, 1959 bp, synthetic construct
  source 1..1959: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtcccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaatcggcttctggcgtgtgaccggcggggtttatatcttcccttctctgttcctccgcagccagcgccacc

SEQ ID NO: 88 - DNA, 1948 bp, synthetic construct
  source 1..1948: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggcccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaaggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 89 - DNA, 1980 bp, synthetic construct
  source 1..1980: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttctgggcctacggagccagcacccatcgccaaaactcttcatcctcttcctcaatctcgctttctctctcgctttttttttttttcttcttctttttttttttttttttcaaaaggaggggagagggggtaaaaaaatgctgcactgtgcggcgaggccggtgagtgagcgacgcggagccaatcagcgcccgccgttccgaaagttgccttttatggctcgagtggccgctgtggcgtcctataaaacccggcggcgcaacgcgcagtcagtccgcctggagacctcgagccgagtggtcgtgcctccatagaatcggcttctggcgtgtgaccggcggggtttatatcttcccttctctgttcctccgcagccagcgccacc

SEQ ID NO: 90 - DNA, 1969 bp, synthetic construct
  source 1..1969: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtctgggcctacggagccagcacccatcgccaaaactcttcatcctcttcctcaatctcgctttctctctcgctttttttttttttcttcttctttttttttttttttttcaaaaggaggggagagggggtaaaaaaatgctgcactgtgcggcgaggccggtgagtgagcgacgcggagccaatcagcgcccgccgttccgaaagttgccttttatggctcgagtggccgctgtggcgtcctataaaacccggcggcgcaacgcgcagtcagtccgcctggagacctcgagccgagtggtcgtgcctccatagaaggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 91 - DNA, 2223 bp, synthetic construct
  source 1..2223: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcggggctccgggagggccgcgtggggagcttgtgcgctccgcgtgtgcgcgaggggagcgcggccgggggcggcccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaacccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatgtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaattcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaatcggcttctggcgtgtgaccggcggggtttatatcttcccttctctgttcctccgcagccagcgccacc

SEQ ID NO: 92 - DNA, 325 bp, synthetic construct
  source 1..325: mol_type = other DNA
tcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaa

SEQ ID NO: 93 - DNA, 382 bp, synthetic construct
  source 1..382: mol_type = other DNA
cccgcgtcccggaaggtgagtgggggagggtcccttcccctccacacgtcaacgattctatttgaagttgggcaggggggtggcgctgctcaccacacacaagtgttataggaggagtctggcccttgagtaccgggtacgcaggggtgcctcaaccacactccgtccacggactctccgttattttaggaggtgagtgtagtgccagtatctactctccttcttaaaaaaaaccagggctccagagaatcagaacagccaccatcaccgcagggagtcaagggaggagggagattagagaaggagccagggagggtggcagggaggccacgtgatccgagtcccctcacccctttccttcccacaggtccctggccaaaga

SEQ ID NO: 94 - DNA, 412 bp, synthetic construct
  source 1..412: mol_type = other DNA
cccgcgtcccggaaggtgagtgggggagggggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctgagtgcctgggagggacacccgcctacactctgcaagaaactcaaaaagggagttgaggggatcgtgggagggaggtagggagggaggagggtgccactgatcccctgaacccctgcctctgcctccagagtgcccctccggcc

SEQ ID NO: 95 - DNA, 266 bp, synthetic construct
  source 1..266: mol_type = other DNA
cccgcgtcccggaaggtgagtgggggaggggagagggaggagggtcccggaccctaggagtgggaaggaaaggctcggatcccctgatccccaggaggaggggacccggctgcctcccggttggggccgcgcgagggcggggcgcggaaggatccgggagggccgtgctccgccacccagtatatatctgtccccagtccccggggccgcctcattccctgtcctcggatcacagtctcttctcactacagtgtcgccgcctctgc

SEQ ID NO: 96 - DNA, 201 bp, synthetic construct
  source 1..201: mol_type = other DNA
cccgcgtcccggaaggtgagtgggggaggggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagt

SEQ ID NO: 97 - DNA, 530 bp, synthetic construct
  source 1..530: mol_type = other DNA
cccgcgtcccggaaggtgagtgggggagggccatcactgctctcttccaaggactcacaaagagttaatgtccctggggctcagcctaggaagattccagtccctgcccaggcccaagatagttgctggcctgattcccctggcattcaggactggaaaggaggaggaggggcacactacgccggctcccatcctccccccaccccgcgtgcctgcttgggattcctgactctgtaccagcttcagagaacaggggtgggggtgggtgccattgggtgtggacagaaagctagtgaaacaagaccttgacaagtcactggccggctcagacgtgtttgtgtctctcttttcttagctcagtgagtactgggtttgtgtcacattgccaaatcccggatcacaagtctccttgaactgctggtgagctaggataataaaacccctgacatcaccattccagaagcttcacaagactgcatatataaggggctggctgtagctgcagctgaaggagctgaccagccagctga

SEQ ID NO: 98 - DNA, 335 bp, synthetic construct
  source 1..335: mol_type = other DNA
cccgcgtcccggaaggtgagtgggggagggaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaacta

SEQ ID NO: 99 - DNA, 2223 bp, synthetic construct
  source 1..2223: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcggggctccgggagggccgcgtggggagcttgtgcgctccgcgtgtgcgcgaggggagcgcggccgggggcggcccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaacccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttgtggtagtgtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaattcctttgtgccaaatctgtgcggagccgaaatctgggaggcgccgccgcaccccctctagcgggcgcggggcgaagcggtgcggcgccggcaggaaggaattgggcggggagggccttcgtgcgtcgccgcgccgccgtccccttctccctctccagcctcggggctgtccgcggggggacggctgccttcgggggggacggggcagggcggggttcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaatcggcttctggcgtgtgaccggcggggtttatatcttcccttctctgttcctccgcagccagcgccacc

SEQ ID NO: 100 - DNA, 1994 bp, synthetic construct
  source 1..1994: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaaggagtcgctgcgttgccttcgccccgtgccccgctccgcgccgcctcgcgccgcccgccccggctctgactgaccgcgttactcccacaggtgagcgggcgggaccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaacccctccggggcgtggcgcggggctcgccgtgccgggcggggggtggcggcaggtgggggtgccgggcggggcggggccgcctcgggccggggagggctcgggggaggggtcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaaaaggaattgggcggggagggccttcgtgcgtcgccgcgccgccgtccccttctccctctccagcctcggggctgtccgcggggggacggctgccttcgggggggacggggcagggcgggggtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 101 - DNA, 1964 bp, synthetic construct
  source 1..1964: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaaggagtcgctgcgttgccttcgccccgtgccccgctccgcgccgcctcgcgccgcccgccccggctctgactgaccgcgttactcccacaggtgagcgggcgggaccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaagccggtggcggcaggatacagcggcttctgcgcgacttataagagctccttgtgcggcgccattttaagcctctcggtctgtggcagcagcgttggcccgtcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaagcattccaaccttccagcctgcgacctgcggagaaaaaaaattacttattttcttgccccatacataccttgaggcgagcaaaaaaattaaattttaaccgtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 102 - DNA, 1954 bp, synthetic construct
  source 1..1954: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaaggagtcgctgcgttgccttcgccccgtgccccgctccgcgccgcctcgcgccgcccgccccggctctgactgaccgcgttactcccacaggtgagcgggcgggaccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaagccggtggcggcaggatacagcggcttctgcgcgacttataagagctccttgtgcggcgccattttaagcctctcggtctgtggcagcagcgttggcccgtctgggcctacggagccagcacccatcgccaaaactcttcatcctcttcctcaatctcgctttctctctcgctttttttttttttcttcttctttttttttttttttttcaaaaggaggggagagggggtaaaaaattgctgcactgtgcggcgaggccggtgagtgagcgacgcggagccaatcagcgcccgccgttccgaaagttgccttttgtggctcgagtggccgctgtggcgtcctataaaacccggcggcgcaacgcgcagtcagtccgcctggagacctcgagccgagtggtcgtgcctccatagaagcattccaaccttccagcctgcgacctgcggagaaaaaaaattacttattttcttgccccatacataccttgaggcgagcaaaaaaattaaattttaaccgtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 103 - DNA, 310 bp, synthetic construct
  source 1..310: mol_type = other DNA
cccgcgtcccggaaggtgagtgggggagggatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacacc

SEQ ID NO: 104 - DNA, 695 bp, synthetic construct
  source 1..695: mol_type = other DNA
aaatgccctatttttttccctgtgtctcccaagagaaaaagtaaatttcagattgtgttccccctcctagcgccagagaaaggaactacaacaaagcctagctgctgagtccagagcgcacagcacagtcgccctcccagctcttccgtcggccgggaggagtctcctttgctcttgtctactgctggagctgctaataaagtttcattcgaatacttgagctgccgcgccctgcgtttccctcatgagcttcttccgcccgcaacacgagactcagcaccacacaatcagcgtggcaaagcctgaagggcgggacaggaagtgaggtcactcctcaccggggtgaaaggttagcggaagtgtccttctttcctttttgctggtagggccgggtggttgctgccggtaagtagaagcttgggttgaatctttcaatccgctgccatccgcggcttgggggtcgaaggtccgggcggcgccgggtgcattccttccctaggtctctggcggctacgtatccgatcctgtcctgcgagcagggccctcgggcggccttgaccaaggcgacagtgaacacagtctgaagttccggcccaagacctgggcttgctggcaggagtcagctctgggcatctttggctttacggatttttaagtggccctttctccttgctctctgcagaggatccgccacc

SEQ ID NO: 105 - DNA, 417 bp, synthetic construct
  source 1..417: mol_type = other DNA
aaaaataaataaataaataaattatatttgtttacgttgttacagaataaatttgcgtcataatttgcatgattgatttaatgtagttttggcgttctcatcacacaaggaccaccagctcgtgacactgggaggagagaacgtcctcaaggtccggaaactcaaggacgtgctgattcgggagctttcaaaaactatttttggaaaacccagatcccggtcttcagccagcgtcgcctgcagtggctagcactacccacaagggagaagccgacgctgcagggctttgccaaatgcgcctctcttcctcgtcttcatctctgtggttagcctgggaaaagagcccgcctcctggcccataaggccctcggccggaagctccgctttctcttcctgctctccatcggatccgccacc

SEQ ID NO: 106 - DNA, 1344 bp, synthetic construct
  source 1..1344: mol_type = other DNA
atggctaatgggtacaaaaataaaatatcatcataaataagacctactatttgatagcaaaacagggtgactataatcaataataacttgtgtattttatttttttgagacagagttttactccatcacccaggctggagtgagtgcattggtgccatctcggctcactggaacctccacctcccggttcaagtgattctccttcttcagcctccagacggagttgctggaattacaggcgcccgccaccacgcccggctaattttgttttgtttgtttgttttgttttgagacggagtttcgctctgtcgccagactggagtgcagtggtgcgatctcggctcactgcaacctccagctccctggttcaagcgattctcctgcctcagcctccctgagtagctgggattacaggtgtgcgtcaccatgctcaactaaattttgtatttttttagtagacacgcggtttcaccatgttggccaggctggtctcgaactcctgacctcgtgatccgcccgcctccgcctcccaaaagtgctgagattacaggcgtgagccaccgcgccaggcctaaaaattttttttaattaaaaaatagatatccttctagctcagaaaagttagctgatccctcctttatactctcttcctaagttccgttcattctggtggctttaattaccatttataagccaacgattcccaaaataacatcttcggtgcagatcccttataaggatgaccagtcattaatattcagcttcctatctgttagtcccactcaacatgacttagcaagatctcaaaatgaacgtgtccagaacagctcctaccttagcctaccccatctcagtaaatggccccaatatttatccgattgatcaagtgacaatcttaggaatccttgacaccgccctctccctcaatccccatgtccaatctaccaccaaacccttcaaaacaaaacccaagtccacactcgtttccaattccattgccttccactctttttcaacgcagccgcacccacatctcatcctaaacgaccgcagcagcctctgaaactagtctaaacacgcccagatctgccgacgccgcagacccccttcccccagcccagggtcggaggcgagtcgccgggggtcggcagaggtcagcagcctgcgagaccctcctaggctcgtggccctaggcgcggcagctgacacgtaagtctcgtcgcccgacgttgtttcggcgctcagaaacaacgtaaagtaaaggggcggggcagcgttttacaaaccgaaccgtgaatctttgcggtttctctttccagccagcgccgagcgggatccgccacc

SEQ ID NO: 107 - DNA, 694 bp, synthetic construct
  source 1..694: mol_type = other DNA
tcccttctcccagctctaacggtgagcagctgataccctgctaggtgctggagagtcagaggtggacatcagggcccacctcgaaaagttgacagtctgtggtgggctgaagccagtagaaagctgctgtaggaaggcagaggagtggcccctatcccaggcttggggctctcagggaaggcttcctgggggtgagatgggagggtgggtccatatgctcttggaccagaatgagtagcccagagaaggcaagagggctcccaagcaaacttgtcctatttaattttttttttttttcctgcttagaaaagcaaaatgcgttatgaacaaaacaaaacaaaacaaaacaaaacaaaacaaaacaaaacaaaacaaaacgctgtagcaaagaaagcgaaaggctccagtaatcctccctgctcctcctcaccgcacggatcctgtgagataccccgagcattggaacctggagagcttgggcaggcattcaccctccttccagcagggggcggaacgtaaaacgccttgtggccgacgccctctctctttcacacgtcactggcgtgaccgtccgcgctacatactgcgcctgcgcaagggctgtggcccttttcccaccccctagcgccgctgggcctgcaggtctctgtcgagcagcggacgccggtctctgttccgcaggggatccgccacc

SEQ ID NO: 108 - DNA, 1689 bp, synthetic construct
  source 1..1689: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaaggagtcgctgcgttgccttcgccccgtgccccgctccgcgccgcctcgcgccgcccgccccggctctgactgaccgcgttactcccacaggtgagcgggcgggaccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaagccggtggcggcaggatacagcggcttctgcgcgacttataagagctccttgtgcggcgccattttaagcctctcggtctgtggcagcagcgttggcccgtctgggcctacggagccagcacccatcgccaaaactcttcatcctcttcctcaatctcgctttctctctcgctttttttttttttcttcttctttttttttttttttttcaaaaggaggggagagggggtaaaaaattgctgcactgtgcggcgaggccggtgagtgagcgacgcggagccaatcagcgcccgccgttccgaaagttgccttttgtggctcgagtggccgctgtggcgtcctataaaacccggcggcgcaacgcgcagtcagtccgcctggagacctcgagccgagtggtcgtgcctccatagaagcattccaaccttccagcctgcgacctgcggagaaaaaaaattacttattttcttgccccatacataccttgaggcgagcaaaaaaattaaattttaacctcggcttctggcgtgtgaccggcggggtttatatcttcccttctctgttcctccgcagccagcgccacc

SEQ ID NO: 109 - DNA, 203 bp, synthetic construct
  source 1..203: mol_type = other DNA
cccgcgtcccggaaggtgagtgggggagggtggcagttttcctttaactctaagggttgtaaatacaaagtagaatgtgttaccatgagccatttcataggaagttgactttatttttttagaattcagaactttgcttctaaaaaggtaactttgcctcttgattctcatttttttccctaacttaggcaccttggaagttg

SEQ ID NO: 110 - DNA, 1110 bp, synthetic construct
  source 1..1110: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggccgggctggtagcccagcggggggcgcggcagggcgaggagcagcccgcgctgctcccgcccctcgggtgccagcaccgcccctgctgcggcgggtgaggggcggggcggggcgcggcgtatataaggctaggggcgggcgccgctcttcagtccgcctggagacctcgagccgagtggtcgtcaggatctcgggctcggaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaaggatccgccacc

SEQ ID NO: 111 - DNA, 594 bp, synthetic construct
  source 1..594: mol_type = other DNA
acgaattaattcaagtcactacgcccgtgggggtggcaacccaaaatagatatcccgacactgcaaataccaatcatacgctaattgcgcacgaacgaaagcgccatgatacgaccttcggcctctccaaaaattacgcagaatcaacatacattatggtgactttccggaaaagcatgtcaaagctatcggcctgccacgttttcctccgacgttcaactttcgggcggaattgccgttctcgattctttatattacgccatccgaacgtcacggccccggaaggcgtacgctagagcttgatgtcgggaggaattacgccttttccattcggcgacgtccggaagtttcgccgtattcgtttccggaagtccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 112 - DNA, 594 bp, synthetic construct
  source 1..594: mol_type = other DNA
tttccggaactgaatatcgggcgcgccccggagtcaggctttaaatgcatgggaagctctcttcctacggtgatcccatggcatagactcaagaccggaatttcctcgccggtgtttccggccatttagatattacgacagggttcaaaaacgatcgaagctatttccggaagaccaggtagtttccggttctcgccgacgtcttctcaaacacaagtggtagcggccgaaagcgacatttcgcctactacatcctataatacctagagtcagattgtacggaaggcgtacgagccgttacgcgccgttacgaaaggcagttttcgggattcgtcgacgtccggaagtttcgccgttttcgtttccggaattccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 113 - DNA, 594 bp, synthetic construct
  source 1..594: mol_type = other DNA
acgaattcacccctaaatttgagaccgttcgtatggcacaccagatttaaccccacaacagatcaaacacaaatcatagcccaatccggcattaacagaagcgccccagcacgtaattcggcccatgcacaacttacggcaaaccaaccccgtttccgccgcctttacgaaaaggagggaccaatttatcggcctgcaaaaatcgcgcggacgtaagtcgtttcggccgaaagctacgttttagattaacttttttgcgacaatgcgtagtttcgcgcagacgttgccgatgaaccgaaagccagtgttccggaattattctttcgccattcggccggccgaaatttcacgaccgttgtcgtttccggaagtccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 114 - DNA, 594 bp, synthetic construct
  source 1..594: mol_type = other DNA
aatagggctctcacttctttccgaccgtccttcggtttcgcttaccactaaccaacaaaatcgcctacaaaaatcatacatcttggcctcattaatgaaagaggcaatacgcggaaatcggtgttttccactcttacggaattgtcacgccatttcccccgcctttccgaaaaggggccggacgattctccgctggtaaaatagccgttccgaaatatagtggcggccgaaagcgacgtttcgcttaaaactcggcaaagttcggtagtatctattagtcggccattaacgaaaaaaccgggcgaggttccggaagctcgttttcggccttcgtcggtggccggaagtttcgcccttttcggttccggaattccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 115 - DNA, 594 bp, synthetic construct
  source 1..594: mol_type = other DNA
acgaagtcccccggcactttgcgaccgtaaatgtactacacaaaaagttgacctacatttagataaagctgcatcataccgtaaaacggcattcctttacgttaaccattacggaattcggcctattctaagtttacgtcgtgccgacacaatatcagttattgttccgcctattcgcggaatttcggcttattcgtcaagtcatcggtgacgaaagtcgttgcggccgaaattttcgacaagtctgccgctcctgttcggggttcttcaaacacgaaagtttccggcctgagacgataacgcgggttttcgaaagtccggtttcgccgttgatcggcgagcggaagtttcgccgttctggtttccggaagtccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 116 - DNA, 594 bp, synthetic construct
  source 1..594: mol_type = other DNA
acgaaatagcggtgcaccccacagctttttcttggacagcacacatttaaaccaaaaaggattcccacacaattcacaggcaaatccgcctttaatgaaaaccccgttagacgaattccggcctactgtaaaatgacgttatacccatcacttttcccccgccattccggaaaacgaagtcgaaagcataatgtttccgcctacgcggccacgaaattgtttccggccgaaattgtcgatggatccttaccgcttcgatatccagtcgaattcataattccgccctaagcgaaagtcacgggcgggtttccggaagtacggtttcggccttcgtcggcggacggaagtctctccgttttcgtttacgaaagtccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 117 - DNA, 594 bp, synthetic construct
  source 1..594: mol_type = other DNA
acgtattccctccgctctttacgaccggaaatgtaccacaccgaaaattgaggcacaattcgtcaaacataagtcatgccgtaatacgccgtctgcgaaagcgacctattacgtaattcgggttattcttaccttacgtcaccgcaaccctatttccccgcccgttcccttaatcacggaagtcctttacggcagtccgccttggcggtgacgaaagacattccggccggaatattcgaaaagtctttcgcttctgctcggggttggtgtaagacgaaattttcgtgcccggaaaggtctcgaagtcgtccggaagtcggttttcggcgtccgtcgacgaccggaagtagtgacttttacggttccggaagtccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 118 - DNA, 972 bp, synthetic construct
  source 1..972: mol_type = other DNA
tccatctttccagaaacttctctgagaaactcagcaaaaagcatccaatttcgccactgcttccccctccgcgcatgcgcgagacgccaagcggaaagcctttggcgcgccggagtgtgagagtggtgcgtgagcgcgcgcgcgctcgccgtgcgggggcggttctctctgacaatgaagggaaagaattccccctcccctcaacattccgtcctcgcgcaagcgcgtcggagccacttccttggcctttgagggaagggggaggggtcgtcccgaagcgcgctagtcgtgtttccgcgcgcctgcgcacggggccgttttcgtctcgctgtgttgtccgcgagtctttgacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 119 - DNA, 928 bp, synthetic construct
  source 1..928: mol_type = other DNA
taaacacgaccactttacgcagcttaacagaatgactttaatgcttcttcaagttttccattttcttccacagggctttgtttcgaaaaataacaaaatgaggtaataagttaatgtatgtacacgttataaacactgtgtcagtttacgatggcagcaacggaagttgttacaaagaaagtgactgcggggtggctggagctcagccgcccccccattgagaagattcgtcgtcctgagaagtcgcgtgctggaggtggagtgtgtctagaagcatttgcggtggacgatggaagggccggcctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 120 - DNA, 916 bp, synthetic construct
  source 1..916: mol_type = other DNA
aaatatggatcaacaaaatacagtatatccataccctaaatatggatcaacaaaatacagtatatccatacactaaatatgaaaatacggtatatccatataataaatatgggtcaacaaaatacggtatatccataccctaaatatggatcaacaaaatacggtatatccatacactaaatatgggtcaacaaaatacagtatatccatacaataaatatgggtcaacaaaatacggtatatccatacactaaatatgggtcaacaaaatacagtatatccataaaataaataacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 121 - DNA, 870 bp, synthetic construct
  source 1..870: mol_type = other DNA
caccaacacctttgccacaaacacattcatgtctaacaacacatcaattaccaacaccagcgccacgaacacattcacgtccatcaacacatcaaccaccaacaccatcagcacgagcacattcacatccaataacacatcaactaccaacaccagcaccatgaacacattcatgcccaacaacacgtaaacccctaacactgtcaccacaaacaccttacagccagcagaacgccagtcactaacaccatcgccatcagcacttcgtggttagcaacacctcagctgacgccaatgtcaccacaaacacctcatggccagaagcagctcaaccaccaacaccgttatacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 122 - DNA, 571 bp, synthetic construct
  source 1..571: mol_type = other DNA
aactaggacctcgtccccttctctatcccttcccctgatccatctcgtctctgttctccttcccttttccgttcccgaagccggaaggagccgaggtacccacggaaaaagtcgaagctgtccccggagagtgaggccttcacgaagcggtggcggaaggagccgaagttctctgatcggaggcgtgagcaagtggcatctccggaaagagccgaaacacggactcgagcttaatcccaggaggggcccgagggcgagatccggaaatctccggaaagagccgaaggccgggacggttaggattgtcggaagtggccgattgcttggacagggccggcggagaagatcgccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 123 - DNA, 972 bp, synthetic construct
  source 1..972: mol_type = other DNA
gatgcccaccgggatggagaaggaggaggaatcggtagggactcgagtgtttattacccccccttccctcctcccccctcccctttgtcagtcgggcctcgccattcacagagcgctttacgctcgctggagggcgccgctgccgctctaacgcacgggactctacgccggcggagtagcgtaaaccctctcggccggcgtagcgcccgcccgcggccgccatgttgttgcgtcccagtgttgtgattagctcgcagcgctgcttacgctgccgtaagggggccttgccgtaagcggcggccgggaatggcgcaggggatgcgctggccgctcttggcgtacggtctctgacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 124 - DNA, 961 bp, synthetic construct
  source 1..961: mol_type = other DNA
gcccgttagtcccggatcccagcggccgcttcaccactgagcccgagccgccgagggaattttccaaatgcaaatcaaacacccttggcgccccgcttcttcattaattaagaggaacgtttcgacaaaaccggcgtaatctttattaattacgcagcccttagcgcgccgttcgcatcaatcacgcccaaagcgaaagtgcagggggcgcctaatcccttcccagagcgcgtttggagaaggagccgcgtggccggcgtttccaccgtgacttccacctttctgggctcgcctggcactggctgctcagccggcgcgcagggaggcctttccggaaacacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 125 - DNA, 571 bp, synthetic construct
  source 1..571: mol_type = other DNA
ttacactagcttaagaggtggaggcggagacgttacgctaccgctaagaggtggaggtggagacgttacgctggcgtaagaggtggcggtggagacgctacgctggcgtaagacgcggcggtggagacgctacgctggcgtaagaggcggcggtggagacgctacgctggcgtaagaggcggcggtggaggcgctacgctggcgtaagaggcggcggtggaggcgctacgctggcgtaagaggtggcggtggagacgtttcgctggcgtaagaggtggaggtggagacgttacgctggcgtaagaggtggaggtggagcgtccttactgcacagggtgtggagggaatgccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 126 - DNA, 972 bp, synthetic construct
  source 1..972: mol_type = other DNA
gcgcgcttcggtgcccgcgcgcccccacactttctcgcgcgtcttcgccccgcccacccctcgccgctccctcacgttctccagtttcttcgcctcgcctaccgcgcgcctctcccgccctttgtcgccttccttcgctccgcccgccccttccttccgtgcgcaccttcgccccgcccaccccactgtcctcccgccgtttctctcgcgcttccattcggcaaacccgtaagctcccgcgttcctctgcgcgcctttcgtccctcccacctgtgcgctcacgtcctccctcgcgtgcggcgctcagtttccggccctacccgtgggcgtcggcgcctagtggcgctcttacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 127 - DNA, 972 bp, synthetic construct
  source 1..972: mol_type = other DNA
aaagccctgtggcccacgcttccggcctcgaccgactacgtcatcaccaaaccccgtgatacctcactccaccttcaggtcctccgtcccaacggttccgtgtcctggaactttccacgttcctgcgcacgcgctctgcggcccttaacgtcccgacttcgccctcctctggccacgcccctgcccgttaagggccctgccacccccgtgacgtacctatcttccctcgggcaacctcgcaacagatctctgattggctgacccaggaagcggactttgcgaggcggcggcgacgataggctggcagccggccgacttccgcagaaggtgattggtcggcgctgctgtccacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacac

SEQ ID NO: 128 - DNA, 338 bp, synthetic construct
  source 1..338: mol_type = other DNA
cccgcgtcccggaaggtgagtgggggaggggggggcctgctgcgggaggtggaggcccaagggaggccccccggggactgtgtgtctcacccccgtccctgctacgttgtgttgttgtgtgatcccatcgtggaggttgttttggtgacactgtgtccccacgaagctggggatacccgtttctctagcttggagccaccaagatagaggacaaacacttctgtgattcagtccccagactgtctctgacttaatcccttgggttcaagccctttgtgggagagcaagggcacacactgcctaatccgtggtgtcccccccaggacaattgcgtctct

SEQ ID NO: 129 - DNA, 193 bp, synthetic construct
  source 1..193: mol_type = other DNA
cccgcgtcccggaaggtgagtgggggaggggcaggacggggacgggcggagcagggacggccaccccaggagatgccaggggagaagggccagggacgaggacctgggtcgagggcggggcctgaacgttacccccgggctagaggcggggcctggggccgggggccgggcctcgaaacctccccaagtcggg

SEQ ID NO: 130 - DNA, 160 bp, synthetic construct
  source 1..160: mol_type = other DNA
cccgcgtcccggaaggtgagtgggggagggacaagacaggtttaaggagaccaatagaaactgggcatgtggagacagagaagactcttgggtttctgataggcactgactctctctgcctattggtctattttcccacccttaggctgctggtggtcta

SEQ ID NO: 131 - DNA, 880 bp, synthetic construct
  source 1..880: mol_type = other DNA
cccgcgtcccggaaggtgagtgggggaggggcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtg

SEQ ID NO: 132 - DNA, 506 bp, synthetic construct
  source 1..506: mol_type = other DNA
cccgcgtcccggaaggtgagtgggggagggccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtg

SEQ ID NO: 133 - DNA, 237 bp, synthetic construct
  source 1..237: mol_type = other DNA
cccgcgtcccggaaggtgagtgggggagggcgggacctgcttggggttaaatactccggcccagcactccccaggcctctagtcccagtaggaggtttgactaagatcaatcttggtaagtgccttggccatctccccttgggtgggacatcttcagtaacgcttccagaagcaccttgtgttggaacctccaagtctcactgtccttctttttctccttagacgacttacagtgac

SEQ ID NO: 134 - DNA, 287 bp, synthetic construct
  source 1..287: mol_type = other DNA
cccgcgtcccggaaggtgagtgggggagggtggcgggctgggctgggggcaggcttccccaaccccagcccaggaagggacagggaatagcttcattccctgacttcatcaaccagctgacctactgactccctctccctcccttcctccctccccttcccttcctccctccgctccttgcttccctccccctccctccctttctgcccttctctctcctctttctcccctttcccatctccctctgccttcccctggcactctctctctagaacgggcagctgctg

SEQ ID NO: 135 - DNA, 345 bp, synthetic construct
  source 1..345: mol_type = other DNA
cccgcgtcccggaaggtgagtgggggagggttatgcaagtgcccccggcgcctccccttcggtctttcaccccgcgcggttacgaaagcgcgaccccctccccccggcgctataaagcagcggggcggccgcggcgcgctcgcctccctcgctccacgcgcgcccggactcggcggccaggcttgcgcgcggttcccctcccggtggtgagtcgcggccggggcctcggggccggcggcgggttgggccggggtggggtggggtggggtgaggtgggatacggcggcgggggtcggctgccgggcactgacggaggcgcgtctcccgcagggcggattcctgggc

SEQ ID NO: 136 - DNA, 576 bp, synthetic construct
  source 1..576: mol_type = other DNA
cccgcgtcccggaaggtgagtgggggagggaatttccagaccacacttgcccactgggaggcttttaggacccgagacgtgtgcaggcttttccaggttcgggttggtgggccttattcattttggcggggctatacgtattcactcttaagcctgtagaagatgaacgttgattgtaccttctaaaacgtgaggttatagttaagagaattgcgttgtcggtctgctcagcagagatgtaagtccagtcttgatttcctcaggcactacttgaggagttgggagagtgtaactaaaaggtcttataactaggacaagtgtttttgatgcaaaagtctttagtccgtaaacatatacagaaaacacccgaataaaacaaaaaataaaaagtttcgagcttcggttgataggcccttccgatcaaacagtttttgttgaagttgcacaacatgacactgcctatgatcattctagactatcaagataaaatcattgcggttatttgtttggaagctggatgagattttggtcatttaagagtctttttaactttttatttagccaaattgaagttta

SEQ ID NO: 137 - DNA, 1062 bp, synthetic construct
  source 1..1062: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacaggctagcgccgccacc

SEQ ID NO: 138 - DNA, 215 bp, synthetic construct
  source 1..215: mol_type = other DNA
gaattcgaaatcggacgacattacggccgcgccaatcggcgttcgccgttccggaagtcgccttttacggtttggccgaaaaatggccgctaaagccgttaattatatacggaagtgtcggttgtggcgccgttagttccggcgtggccgattcgagccgagtggtcgtcttgtttgtggattggttttttcgtcacgtgacaggatccgccacc

SEQ ID NO: 139 - DNA, 209 bp, synthetic construct
  source 1..209: mol_type = other DNA
gaattcgggcggagttagggcggagccaatcagcgtgcgccgttccggaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 140 - DNA, 209 bp, synthetic construct
  source 1..209: mol_type = other DNA
gaattcgggcggagttagggcggagccaatcggcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 141 - DNA, 209 bp, synthetic construct
  source 1..209: mol_type = other DNA
gaattcgggcggagttagggcggagccaatcagcgtgcgccgttccgaaagttgccttttatggctgggcggagaatgggcggtgaacgccgatgattatatacggacgcgccgggtgtggcacagctagttccgtcgcagccggctcgagccgagtggtcgtcttgtttgtggatccctgtgatcgtcacttgacaggatccgccacc

SEQ ID NO: 142 - DNA, 209 bp, synthetic construct
  source 1..209: mol_type = other DNA
gaattcgggcggaattagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 143 - DNA, 209 bp, synthetic construct
  source 1..209: mol_type = other DNA
gaattcgggcggagttagggcggagccaatcagcgtgcgccgttccgaaagttgccttttatggctgggcggaaaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 144 - DNA, 209 bp, synthetic construct
  source 1..209: mol_type = other DNA
gaattcgggcggcgttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 145 - DNA, 209 bp, synthetic construct
  source 1..209: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcaccgctagttccgtcgcagccggctcgagccgagtggtcgtcttgtttgtggatccctgtgatcgtcacttgacaggatccgccacc

SEQ ID NO: 146 - DNA, 209 bp, synthetic construct
  source 1..209: mol_type = other DNA
gaattcgggcggagttagggcggagccaatcagcgtgcgccgttccgaaagttgccttttacggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 147 - DNA, 209 bp, synthetic construct
  source 1..209: mol_type = other DNA
gaattcgggcggagttagggcggcgccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 148 - DNA, 209 bp, synthetic construct
  source 1..209: mol_type = other DNA
gaattcgggcggagttacggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 149 - DNA, 209 bp, synthetic construct
  source 1..209: mol_type = other DNA
gaattcgggcggagttagggcggagccaatccgcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 150 - DNA, 209 bp, synthetic construct; identical to SEQ ID NO: 149
  source 1..209: mol_type = other DNA

SEQ ID NO: 151 - DNA, 209 bp, synthetic construct
  source 1..209: mol_type = other DNA
gaaatcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 152 - DNA, 209 bp, synthetic construct
  source 1..209: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccgtgtgatcgtcacttgacaggatccgccacc

SEQ ID NO: 153 - DNA, 209 bp, synthetic construct
  source 1..209: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcggccggctcgagccgagtggtcgtcttgtttgtggatccctgtgatcgtcacttgacaggatccgccacc

SEQ ID NO: 154 - DNA, 209 bp, synthetic construct
  source 1..209: mol_type = other DNA
gaattcgggcggagttagggcggagccaatcagcgtgcgccgttccgaaagttgccttttatggctgggcggagaatgggcggttaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 155 - DNA, 209 bp, synthetic construct
  source 1..209: mol_type = other DNA
gaattcgggcggagttagggcggagccaatcagcgtgcgccgttccgaaagttgccttttatggctgggcggagaatgggcggtaaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 156 - DNA, 209 bp, synthetic construct
  source 1..209: mol_type = other DNA
gaattcgggcggagttagggcggagccaatcagcgtgcgccgttccgaaagttgccttttatggctgggcggagaatgggcggtgaacgccgatgattatataaggacgcgtcgggtgtggcacagctagttccgtcgcagccggctcgagccgagtggtcgtcttgtttgtggatccctgtgatcgtcacttgacaggatccgccacc

SEQ ID NO: 157 - DNA, 209 bp, synthetic construct
  source 1..209: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatcgctgtgatcgtcacttgacaggatccgccacc

SEQ ID NO: 158 - DNA, 209 bp, synthetic construct
  source 1..209: mol_type = other DNA
gaattcgggcgaagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 159 - DNA, 209 bp, synthetic construct
  source 1..209: mol_type = other DNA
gaaatcgggcggaattagggcggcgccaatcggcgtgcggcgttccggaagttgacttttacggctgggcggaaaatgggcggttaacgccgataattatatacggacgcgccggttgtggcaccgctagttacgtcgcggccggttcgagccgattggtcggcttgtttgcggatccgtgtgaccgtcacgtgacaggatccgccacc

SEQ ID NO: 160 - DNA, 209 bp, synthetic construct
  source 1..209: mol_type = other DNA
gaattcgggcggaattagggcggagccaatcggcgtgcgccgttccggaagttgccttttctggctgggcggaaaatgggcggtgaaagccgatgattatatacggacgcgccgggtgtggcaccgctagttccgtcgcggccggctcgagccgattggtcgtcttgtttgtggatccgtgtgatcgtcacgtgacaggatccgccacc

SEQ ID NO: 161 - DNA, 209 bp, synthetic construct
  source 1..209: mol_type = other DNA
gaattcgggcggaattagggcggagccaatcagcgtgcgccgttccggaagttgccttttatggctgggcggaaaatgggcggtgaacgccgatgattatatacggacgcgccgggtgtggcacagctagttccgtcgcggccggctcgagccgagtggtcgtcttgtttgtggatccgtgtgatcgtcacttgacaggatccgccacc

SEQ ID NO: 162 - DNA, 763 bp, synthetic construct
  source 1..763: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggccggcctggcattatgcccagtacatgacgttatgggactttcctacttggcagtacatctacgtattagtcatcgctattaccatggtctctccgtttccgcggtacgttaatcggcgccgatctccgttcgacgtgcgccaatttcggagtctccgcgggattgacgataatcggcgttcgtttccgcgcggaaatcgtcccaacgttcggaaatgacggaatgattccgccccattgacgggatacggcggaaggggtgttccggacgccggcgaaaaaaggcggccccgtcggtttcggcctcactcttttccggtttgtcgagccgaatggtcgtcggtccgggtgatattacgactaagccgtaagggtttacgggaccgtcggttggcggcgtattaatgtttaattacggggagcacctgccggaaatcactttttttcaggttggccgcggatccgccacc

SEQ ID NO: 163 - DNA, 1761 bp, synthetic construct
  source 1..1761: mol_type = other DNA
acattgattattgactagttattaatagtaatcaattacggggtcattagttcatagcccatatatggagttccgcgttacataacttacggtaaatggccggcctggctgaccgcccaacgacccccgcccattgacgtcaataatgacgtaagttcccatagtaacgccaaaagggactttccattcacgtcaatgggcggactatttacggtaaactgcccactcggcagtacatcaagtgtatcatacgcggcttacgccccctattgacgtcaatgacgataaatggcattcctggcattatgcccagtacatgaccttatgggactttcctacttggcagtacatttacgtattcgtcatcgctattacgatggtacgccgtgagccccacgttaggcttcacgctcgccgtttcccccccctccccaccgcggttttcgtatttccttcctctttaattatttcgccacgcgaacgcgtcggggggggggggggcgcgcgcaattccgggcggggcggggcgagggtcggctcgccgcgaagccacatagttcgtcctacgccaatcgtaacggcctgttccggaagtttcggttaaccacgaatccgccttcttctccgccgtataaaaagcgaaatgggcgtccgaccgaaatcgtgacgtttctgtcggcccgtttcgcgtttcgggcggttttttgccggcggtgccggctcggacgtgtggcgttacgccggttctctatcggtccgttcggccttttcctttcgggccgtaattcaggtttcgtttagttgcgggccgtttcgtttcggtcgtgtcgcaaaagccgtaaagggttccggaagggccgtttgtgcgggggggagcggttcggggggttcgtacgtgtgtgtgtgcgtggggagcgccgcgttcggcccgcgctgcccggcggtcgtgagcgtttcggacgcggcgcggggctttgtgcgttccgcgttttcgcgaggggagcgcggccgggggcggtgccgcgcgttacgggggggccgcgagcggaacaaaggctgcgtgcggggtgtgtgcgtgggggggtgagcagggggtgtgggcgcggcggtcgggccgtaacccccccctgtgcccccctccccgagttgctgagcacggcccggtttcgggtgcggggctccgtacgggacgtgacgcggggctcgtcgtttcgggcggggggtggcggacgtttggggtttcggtcggggcggggccgcttcgggccggggagggttcggggaaagggcgcggcggcccccgtaacgccggcggctgtcgaggcgcggcgagccgcggccattgcggtttacggtattcgtgcgaaaggccggcgggacgtccgttgggcggaggtgtgtcggagccgaaattgggtaggcgccgccggggggcctttagcgggcgcggggcgaaacgtttcggcgccggacggaaggaaatgggcggggagggccttcgtgcgtcgccgcgccgccgtcccgttctccctctccggcttcggggcgttcggcgggcggacggcgtctttcggggcggacggggaagggcggggttccgcttctgacgtgttaccggcggctctagagcctctgctaaccatgttcatgccttcttctttttcctacagctcctgggcaacgtgctggttattgtgctgtctcatcattttggcaaagaattcgaggtggaaattaatacgacgtgacggatccgccacc

SEQ ID NO: 164 - DNA, 1079 bp, synthetic construct
  source 1..1079: mol_type = other DNA
acttacggtaaatggccattgttgatgaccgccgaacgacccccgcccattgacgtcaataatgacgttagttcccatagtaacgccgttacggcctttccatttacgtaaatgggtgaagtatttccggcaaagcgcccacgtcgcggtacgtcaaatgtatcgaacgcgacttccggcccttatttacgtcaattacggaaaacgggcggtgccgcgttatgcccgttacacgacgttacggtaatttcggcagccgcgataagtttacgtaatccccaacgccattaccatgctcagcccgtatccgcggtacgccaatcgccgacaaacgcgcaacaacgtgccccaatttcggagtcgcggcggggttgccgaaaatccgaattcgtttccgcgcggaaatgaacggaacgttcgggaatggcggaatagtgggggcccgttaacgacatacggcggacggctctttcggccggcagacgaaatacgacgagttcgtttcgggatccggagttccggggcgcgcagcggttggcgctgtttcggcggcggtatcggatttcgtagccgtattggttttcgcgttttgccgtcatttgcgtttcgcgagccgtacgtgcggtccttctttccgattcgtcgttggcgaaggcgggtcttccgtttggccgaagccacagaaagtttcatcgcctttccgttagtatttgtccggaatgtggtcggctgccgtaagttattccgccgtcgaggggaggtaacggtgcggagtcggccgggccggaatgtggttcgcgaaaggcggctaacgggtcacggtcggaaggtcggcggagtaccgtgtcgggcggcagcgggtgtcggtcggggttgtttcgggcggaagtgctgctgatgacgtaattaaagtaggcggtcttgagacggcggaaggtcgaggtgacgtgtggcaggcttgagatccagctgttggggtgagtactccctctcaaaagcgggcattacttctgcgctaagattgtcagtttccaaaaacgaggaggatttgatattcacctggcacgggatccgccacc

SEQ ID NO: 165 - DNA, 308 bp, synthetic construct
  source 1..308: mol_type = other DNA
gaaatcgccaaaacgccttacggaagccgttccgtaatgctaggcttccattgggaattacgtttccgctttcggttcgttttggcgaaatggcggaagcggtttactccgggatttccgtctccccccattacgtaatgggagtttgttttggcccaaaatacgggactttccaaaagtcgtaacaatccgccccattacgaaatgggcggtaggcgttacggtgggaggtctaataagcggccgtttagtgaaccgtcagtcgccggaagccatccgctgttttgactccgaatggatccgccacc

SEQ ID NO: 166 - DNA, 174 bp, synthetic construct
  source 1..174: mol_type = other DNA
gattcggcattggcgggccaatccgtcgccgttccgaaagttgccttttatggcgggcggaaagggcggtgacgccgatattatataggcggccgggtgtggccgctagttccgtcgagccggccgagccgagtgtcgtcttgttttggtcccgtacgttacggatccgccacc

SEQ ID NO: 167 - DNA, 773 bp, synthetic construct
  source 1..773: mol_type = other DNA
aaatcggcctttcgcacgaaaagccgccattgccgtaagacttcggcttcctttggcgtaattacgatatccgctattaccatgctgtgcggttttggcgtaaccaatcggaagcggttttccggggatttccaagtctccaccccattacgtcaatgggagtttgttttggaccaaaatccgggactttccaaaatgtcgtaataaccccgccccgttgacgaaatgggcggtaggcgttacggtgggaggtctatataagcagagccgtttagtgaaccgtcagatcgccggaaggccatccacgctgttttgacctccatagtggaaccgggccgatccagcctccgcgttcggggagatctcgtttagtgaaccgtcgatcctcactctcttccgcatcgctgtctgcgagggccgctgttgggccgcggttgaggaaaactttcgcggtctttccgtactcttggacggaaacccgtcggcctccgaacggtactccgccaccgagggccggcgagtccgctcgaccggtcggaaaactctcgagaaaggcgtctaaccgtacgtcgcaaggtaggcgagacgtggcgggcggccggtggcggtcgggttgtttcggcggaggtgctgctgatgatgtaattaaagtaggcggtcttgaacggcggatggtcgggtgaggtgtggcggtagtccgcgttggggtagtactcccttaaagcggattacttccgtaagtcgtttaaaacgtttcggccgggatcgccacc

SEQ ID NO: 168 - DNA, 655 bp, synthetic construct
  source 1..655: mol_type = other DNA
gttacgacatcgcgaaatccacgccgaaattacttccggccccgttacgacatcgccaataaacgtaagtccgccttacgacgttaacgccaaatttccggaataagtcatagccaaacgaccaacgtccatttacgtccacaattccggaagttacgacatgcacgacattccggaagtcccgtttacgtcaatcggcggaatctttccggtaatcggccgacgtgtgtctacatcaaatccatgatgcacaaaagacgtccattattgacatcaatgcacacaaaaggtacgaccagaagtatgcacaacacatgacgacatgcacaaatccaacacgcgaaaaaatcaacgttttcggcctcgccatcacgaacgtaacgccgttccggccttacgtcaaaaggcgccgaaagccgttatcgtttcgcggacgtcggcggttccggcctaacgccgttaaacgaaagtcgtttcggcgccgaaattggccggccttttcgaagtgacggaagtattcggcccgttttacgacgtcggccggaaggcctttcgggaaatcggccggaaggcgacgttacggtttcgggggccgacgtttcggcggaagtgtcggtttccggtgtttcggccgaaatcgtgaggatccgccacc

SEQ ID NO: 169 - DNA, 209 bp, synthetic construct
  source 1..209: mol_type = other DNA
ggaagccgacgacgttacgcggccgcctttcggcttacgtcattccggaagttgccgttttcggccttccggaagttagtcgttaaaagccgacgtttcgttccggaagtgtcggcctattcggcggaagtttcgtggcggccgagtcgggccggaaagcggaagtgtgttacgtttcgggtcgccgttacgtgtcgggatccgccacc

SEQ ID NO: 170 - DNA, 1079 bp, synthetic construct
  source 1..1079: mol_type = other DNA
acaaacgtaaaatggaccggaattctatccgccaaaagccttccggccttttacgacaaaaatgacgtaattccccttttttacgccattccggaagaaaaattcacgtaaatcggctcattgtttacggcaaaatgcaaacaatccggaatatcaatcggatcaaacgacaattccggccatcctttacgtcattgacacaaaatttccggccttccgttacgaaattaacacgaaattacgtcaaaatcccaatacacaaaacgtggacgaattcgtcatcgtttttaccattcgaccggcctaacgccgttacacgaaaggccgacgaaagcctttcggccttccggaattacgaaaaatgcacggcctttacgtcatagccaatccgtttcggggccggaagtgacgtcccgttacgaaatcgcggaattacggcggcccgtttacgaaattcggcggaagtcgtttccggtcggcggcctattacggaagtggccgtttccggtagtgtcgtttcggcggaagttgccgttaacgccgtttcggccttattcgtggcggccgaaatcgttccggccgtcgcgttttacgttacgtcgcgtttccggaagcggccggtcgttacgttattccgtttcggggccggcggaagtgggccgttattttcgcggaagcggactaatttttcgccgcctttccggaagtgttgggccgaaattacgtcggcctccggccgttattcgggcggcgtccggccgtcgcgaatcggtttcggccgaagcggacgtcgtttcgcgaaagtcgtatttcccgtttcggccggaagtgtggcggacttacgtttcggccggaagtcggtggcggaatactatgcacacatcggaaatcccattgtttacgtaatgaattagggccgacgtgaattggcggaagttttccgttacgtcgggccgacgaaatttccggccgttaccggaagttggtactgtgattggccggaagtgtttcggcctttcgcttgtcggcttccggaatcggggaagttacgtgtttcggtcggcccgggatccgccacc

SEQ ID NO: 171 - DNA, 763 bp, synthetic construct
  source 1..763: mol_type = other DNA
ggcctttgacgttttcgcgaaagcgaaagcctttacgcaattcgcgaatcctttccggccggaagtgccccatcggcgtaacacgacgtacgaaagatgaacggaatcgcttccggccggaatgccgccattcggttacgacataaaagtcagaagttacaatgtttacgtccttccggaagttccgtttacgtcaaaggccggcctttttacgacgaaagaaacaattcgccgaaaatcaatcggacttatcgacacgaaagaacatcacacacgtcctttacggtcaacaggaggccatcaaaaatccacacgacatgacgacacaaaacgtcataaaaatgcacaacgttaatccattcggccacgacattacgaacgttacgcctttccggaagtccgacaaacgccgaaagttccggccttcctcacgccgttttcgccgtttccggcctattgacgaaagtgggaagccgtttcggcgccgaaattggccggcctagccgacgttacggaagtattcggcctttcttacgaaaatcggcggaagcggccttccggaagtggcggaaccgactcgttttcggccgggccgacgttaatcgtttccggaatgtcgggccgaaatgtcgacgtttccggtgataatacgactaagaggtaagggtttaagggatggttggttggtggggtattaatgtttaattacctggagcacctgcctgaaatcactttttttcaggttggccgcggatccgccacc

SEQ ID NO: 172 - DNA, 1761 bp, synthetic construct
  source 1..1761: mol_type = other DNA
gcggccttttacgacacgaaaggtcacgaaatccgtttcgcgtccttccggttctagccgaaatttcgtttcggccgttacgttaaaaacgccgaaaggccggccttccattccggcctacgaaattcgcccattgacgttataaatgacggaagttccattgtttacgccattccggaagatcaatacacgaaaatcggcggcctatttccggaagaatgcacacgacgtcattcgggacgtcaagcggaagccgtttacgcgctttgtttacgtcgtttccggaagttggccttccatgcaaaatgcacaaatcatgacgtaatacgccaaatcaacatgcaaacacacgcacaaaaacagaaaatccattacgtcaaaacgccgaaagtcccacgttacggtttcctcacgacgtttccggcctaacgaaatcgcgaattccgccattcggtttccgttacgaaagcctttacggaattgcggcgtacttcggcctccgttacgaaaggccggaagttcggcggcctaggccgggccgcggcctttacgaaattttcggcggaagtcgactaattcggccttttccggaagtgtcggttttcggcgtggccgacgtaccggccggaagttacgtcgcgaaagtcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgaggtggaaattaatacgacgtggcggatccgccacc

SEQ ID NO: 173 - DNA, 1089 bp, synthetic construct
  source 1..1089: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaagtgttgcggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaaatgggcggtaggggtgttcctgaagggggctataaaagggggtgggggcgcgttcgtcctcactctcttccgcatcctcgagccgagtggtcgtctgttggggtgataatacgactaagaggtaagggtttaagggatggttggttggtggggtattaatgtttaattacctggagcacctgcctgaaatcactttttttcaggttggccgcggatccgccacc

SEQ ID NO: 174 - DNA, 1021 bp, synthetic construct
  source 1..1021: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaagaattcgggcggagttagggcggagccaatcagcgtgcgccgttccgaaagttgcctttattggctgggcggagattgggcggtgaacgccgttgattatataaggacgcgccgggtgtggcacagctagttccgtcgcagccggctcgagccgagtggtcgtcttgtttgtggatccctgtgatcgtcacttgacataatacgactaagaggtaagggtttaagggatggttggttggtggggtattaatgtttaattacctggagcacctgcctgaaatcactttttttcaggttggccgcggatccgccacc

SEQ ID NO: 175 - DNA, 1089 bp, synthetic construct
  source 1..1089: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaagtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaataatacgactaagaggtaagggtttaagggatggttggttggtggggtattaatgtttaattacctggagcacctgcctgaaatcactttttttcaggttggccgcggatccgccacc

SEQ ID NO: 176 - DNA, 1118 bp, synthetic construct
  source 1..1118: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaacccgggcccagcaccccaaggcggccaacgccaaaactctccctcctcctcttcctcaatctcgctctcgctctttttttttttcgcaaaaggaggggagagggggtaaaaaattgctgcactgtgcggcgaagccggtgagtgagcggcgcggggccaatcagcgtgcgccgttccgaaagttgccttttaaggctcgagcggccgcggcggcgccctataaaacccagcggcgcgacgcgccacctcagtccgcctggagacctcgagccgagtggtcgtgcctccatagaataatacgactaagaggtaagggtttaagggatggttggttggtggggtattaatgtttaattacctggagcacctgcctgaaatcactttttttcaggttggccgcggatccgccacc

SEQ ID NO: 177 - DNA, 1139 bp, synthetic construct
  source 1..1139: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaatctgggcctacggagccagcacccatcgccaaaactcttcatcctcttcctcaatctcgctttctctctcgctttttttttttttcttcttctttttttttttttttttcaaaaggaggggagagggggtaaaaaattgctgcactgtgcggcgaggccggtgagtgagcgacgcggagccaatcagcgcccgccgttccgaaagttgccttttgtggctcgagtggccgctgtggcgtcctataaaacccggcggcgcaacgcgcagtcagtccgcctggagacctcgagccgagtggtcgtgcctccatagaataatacgactaagaggtaagggtttaagggatggttggttggtggggtattaatgtttaattacctggagcacctgcctgaaatcactttttttcaggttggccgcggatccgccacc

SEQ ID NO: 178 - DNA, 1149 bp, synthetic construct
  source 1..1149: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaatcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaataatacgactaagaggtaagggtttaagggatggttggttggtggggtattaatgtttaattacctggagcacctgcctgaaatcactttttttcaggttggccgcggatccgccacc

SEQ ID NO: 179 - DNA, 80 bp, synthetic construct
  source 1..80: mol_type = other DNA
tatataaggagagcccgggtgtggcacagctagttccgtcgcagccggcgagccgaacggtcgtccgcggatccgccacc

SEQ ID NO: 180 - DNA, 87 bp, synthetic construct
  source 1..87: mol_type = other DNA
cggcgcctatataaggtgtgggcgggtgtggcacagctagttccgtcgcagccggcgagccgaacggtcgtccgcggatccgccacc

SEQ ID NO: 181 - DNA, 185 bp, synthetic construct
  source 1..185: mol_type = other DNA
cggcgcctatataaggtgtgggcgggtgtggcacagctagttccgtcgcagccggcgagccgaacggtcgttgagccaagaggtaagggtttaagggatggttggttggtggggtattaatgtttaattacctggagcacctgcctgaaatcactttttttcaggttggccgcggatccgccacc

SEQ ID NO: 182 - DNA, 268 bp, synthetic construct
  source 1..268: mol_type = other DNA
ggcgaagccggtccccgccccgcggggccccgccccgcgtgcgccgttccgaaagttgctttctagatctcgccccgccccggcggcgcctatataaggtgtgggcgggtgtggcacagctagttccgtcgcagccggcgagccgaacggtcgttgagccaagaggtaagggtttaagggatggttggttggtggggtattaatgtttaattacctggagcacctgcctgaaatcactttttttcaggttggccgcggatccgccacc

SEQ ID NO: 183 - DNA, 209 bp, synthetic construct
  source 1..209: mol_type = other DNA
gaaatcgggcggagttagggcggagccaatcagcgtgcgccgttccgaaagttgccttttatggctgggcggaaaatgggcggtgaacgccgatgattatataaggacgcgccggttgtggcacagctagttccgtcgcagccggctcgagccgagtggtcgtcttgtttgtggatcggtgtgatcgtcacttgacaggatccgccacc

SEQ ID NO: 184 - DNA, 209 bp, synthetic construct
  source 1..209: mol_type = other DNA
gaaatcgggcggagttagggcggagccaatcagcgtgcgccgttccgaaagttgccttttatggctgggcggagaatgggcggtgaacgccgatgattatataaggacgcgccggttgtggcacagctagttccgtcgcagccggctcgagccgagtggtcgtcttgtttgtggatccctgtgatcgtcacttgacaggatccgccacc

SEQ ID NO: 185 - DNA, 583 bp, synthetic construct
  source 1..583: mol_type = other DNA
gcccgttagtcccggatcccagcggccgcttcaccactgagcccgagccgccgagggaattttccaaatgcaaatcaaacacccttgggtggttagcaacacctcagctgacgccaatgtcaccacaaacacctcatggccagaagcagctcaaccactgggtcaacaaaatacagtatatccatacaataaatatgggtcaacaaaatacggtatatccatacactaaatatgggtcaacaaaatacagtatatccataaaataaataggctggagctcagccgcccccccattgagaagattcgtcgtcctgagaagtcgcgtgctggaggtggagtgtgtctagaagcatttgcggtggacgatggaaggggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 186 - DNA, 583 bp, synthetic construct
  source 1..583: mol_type = other DNA
gcccgttagtcccggatcccagcggccgcttcaccactgagcccgagccgccgagggaattttccaaatgcaaatcaaacacccttgggtggttagcaacacctcagctgacgccaatgtcaccacaaacacctcatggccagaagcagctcaaccactgggtcaacaaaatacagtatatccatacaataaatatgggtcaacaaaatacggtatatccatacactaaatatgggtcaacaaaatacagtatatccataaaataaataggctggagctcagccgcccccccattgagaagattcgtcgtcctgagaagtcgcgtgctggaggtggagtgtgtctagaagcatttgcggtggacgatggaaggggaaatcgggcggagttagggcggagccaatcagcgtgcgccgttccgaaagttgccttttatggctgggcggaaaatgggcggtgaacgccgatgattatataaggacgcgccggttgtggcacagctagttccgtcgcagccggctcgagccgagtggtcgtcttgtttgtggatcggtgtgatcgtcacttgacaggatccgccacc

SEQ ID NO: 187 - DNA, 539 bp, synthetic construct
  source 1..539: mol_type = other DNA
taaacacgaccactttacgcagcttaacagaatgactttaatgcttcttcaagttttccattttcttccacagggctttgtttcgaaaaataacaaaatgaggtaataagttaatgtatgtacacgttataaacactgtgtcagtttacgatggcagcaacggaagttgttacaaagaaagtgactgcggggtggctggagctcagccgcccccccattgagaagattcgtcgtcctgagaagtcgcgtgctggaggtggagtgtgtctagaagcatttgcggtggacgatggaagggccggcctcccaatgatggcgcctgctgaattcgaaatcgggcggagttagggcggagccaatcagcgtgcgccgttccgaaagttgccttttatggctgggcggaaaatgggcggtgaacgccgatgattatataaggacgcgccggttgtggcacagctagttccgtcgcagccggctcgagccgagtggtcgtcttgtttgtggatcggtgtgatcgtcacttgacaggatccgccacc

SEQ ID NO: 188 - DNA, 527 bp, synthetic construct
  source 1..527: mol_type = other DNA
aaatatggatcaacaaaatacagtatatccataccctaaatatggatcaacaaaatacagtatatccatacactaaatatgaaaatacggtatatccatataataaatatgggtcaacaaaatacggtatatccataccctaaatatggatcaacaaaatacggtatatccatacactaaatatgggtcaacaaaatacagtatatccatacaataaatatgggtcaacaaaatacggtatatccatacactaaatatgggtcaacaaaatacagtatatccataaaataaataccaatgatggcgcctgctgaattcgaaatcgggcggagttagggcggagccaatcagcgtgcgccgttccgaaagttgccttttatggctgggcggaaaatgggcggtgaacgccgatgattatataaggacgcgccggttgtggcacagctagttccgtcgcagccggctcgagccgagtggtcgtcttgtttgtggatcggtgtgatcgtcacttgacaggatccgccacc

SEQ ID NO: 189 - DNA, 581 bp, synthetic construct
  source 1..581: mol_type = other DNA
caccaacacctttgccacaaacacattcatgtctaacaacacatcaattaccaacaccagcgccacgaacacattcacgtccatcaacacatcaaccaccaacaccatcagcacgagcacattcacatccaataacacatcaactaccaacaccagcaccatgaacacattcatgcccaacaacacgtaaacccctaacactgtcaccacaaacaccttacagccagcagaacgccagtcactaacaccatcgccatcagcacttcgtggttagcaacacctcagctgacgccaatgtcaccacaaacacctcatggccagaagcagctcaaccaccaacaccgttatccaatgatggcgcctgctgaattcgaaatcgggcggagttagggcggagccaatcagcgtgcgccgttccgaaagttgccttttatggctgggcggaaaatgggcggtgaacgccgatgattatataaggacgcgccggttgtggcacagctagttccgtcgcagccggctcgagccgagtggtcgtcttgtttgtggatcggtgtgatcgtcacttgacaggatccgccacc

SEQ ID NO: 190 - DNA, 571 bp, synthetic construct
  source 1..571: mol_type = other DNA
gcccgttagtcccggatcccagcggccgcttcaccactgagcccgagccgccgagggaattttccaaatgcaaatcaaacacccttggcgccccgcttcttcattaattaagaggaacgtttcgacaaaaccggcgtaatctttattaattacgcagcccttagcgcgccgttcgcatcaatcacgcccaaagcgaaagtgcagggggcgcctaatcccttcccagagcgcgtttggagaaggagccgcgtggccggcgtttccaccgtgacttccacctttctgggctcgcctggcactggctgctcagccggcgcgcagggaggcctttccggaaaccaatgatggcgcctgctgaattcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgccacc

SEQ ID NO: 191 - DNA, 571 bp, synthetic construct
  source 1..571: mol_type = other DNA
gcccgttagtcccggatcccagcggccgcttcaccactgagcccgagccgccgagggaattttccaaatgcaaatcaaacacccttggcgccccgcttcttcattaattaagaggaacgtttcgacaaaaccggcgtaatctttattaattacgcagcccttagcgcgccgttcgcatcaatcacgcccaaagcgaaagtgcagggggcgcctaatcccttcccagagcgcgtttggagaaggagccgcgtggccggcgtttccaccgtgacttccacctttctgggctcgcctggcactggctgctcagccggcgcgcagggaggcctttccggaaaccaatgatggcgcctgctgaattcgaaatcgggcggagttagggcggagccaatcagcgtgcgccgttccgaaagttgccttttatggctgggcggaaaatgggcggtgaacgccgatgattatataaggacgcgccggttgtggcacagctagttccgtcgcagccggctcgagccgagtggtcgtcttgtttgtggatcggtgtgatcgtcacttgacaggatccgccacc

SEQ ID NO: 192 - DNA, 677 bp, synthetic construct
  source 1..677: mol_type = other DNA
gcccgttagtcccggatcccagcggccgcttcaccactgagcccgagccgccgagggaattttccaaatgcaaatcaaacacccttggcgccccgcttcttcattaattaagaggaacgtttcgacaaaaccggcgtaatctttattaattacgcagcccttagcgcgccgttcgcatcaatcacgcccaaagcgaaagtgcagggggcgcctaatcccttcccagagcgcgtttggagaaggagccgcgtggccggcgtttccaccgtgacttccacctttctgggctcgcctggcactggctgctcagccggcgcgcagggaggcctttccggaaaccaatgatggcgcctgctgaattcgaaatcgggcggagttagggcggagccaatcagcgtgcgccgttccgaaagttgccttttatggctgggcggaaaatgggcggtgaacgccgatgattatataaggacgcgccggttgtggcacagctagttccgtcgcagccggctcgagccgagtggtcgtcttgtttgtggatcggtgtgatcgtcacttgacataatacgactaagaggtaagggtttaagggatggttggttggtggggtattaatgtttaattacctggagcacctgcctgaaatcactttttttcaggttggccgcggatccgccacc

SEQ ID NO: 193 - DNA, 806 bp, synthetic construct
  source 1..806: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagccaagaggtaagggtttaagggatggttggttggtggggtattaatgtttaattacctggagcacctgcctgaaatcactttttttcaggttggccgcggatccgcc

SEQ ID NO: 194 - DNA, 716 bp, synthetic construct
  source 1..716: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagccaagaggtaagggtttaagggatggttggttggtggggtattaatgtttaattacctggagcacctgcctgaaatcactttttttcaggttggccgcggatccgccacc

SEQ ID NO: 195 - DNA, 397 bp, synthetic construct
  source 1..397: mol_type = other DNA
cccgcgtcccggaaggtgagtgggggagggctcctcaccggggtgaaaggttagcggaagtgtccttctttcctttttgctggtagggccgggtggttgctgccggtaagtagaagcttgggttgaatctttcaatccgctgccatccgcggcttgggggtcgaaggtccgggcggcgccgggtgcattccttccctaggtctctggcggctacgtatccgatcctgtcctgcgagcagggccctcgggcggccttgaccaaggcgacagtgaacacagtctgaagttccggcccaagacctgggcttgctggcaggagtcagctctgggcatctttggctttacggatttttaagtggccctttctccttgctctctgcagaaaggggcaagttca

SEQ ID NO: 196 - DNA, 204 bp, synthetic construct
  source 1..204: mol_type = other DNA
cccgcgtcccggaaggtgagtgggggaggggggtggggcgcgggcgggacttgggcgggcggggtgggcttggccgagctggcctccggggcaccgaccgctataaggccagtcggactgcgacacagcccatcccctcgaccgctcgcgtcgcatttggccgcctccctaccggtgagttctctccaggagccctgggtactt

SEQ ID NO: 197 - DNA, 315 bp, synthetic construct
  source 1..315: mol_type = other DNA
cccgcgtcccggaaggtgagtgggggaggggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaa

SEQ ID NO: 198 - DNA, 176 bp, synthetic construct
  source 1..176: mol_type = other DNA
cccgcgtcccggaaggtgagtgggggaggggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccggg

SEQ ID NO: 199 - DNA, 456 bp, synthetic construct
  source 1..456: mol_type = other DNA
cggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcccggcggggggaggggccgcgcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcag

SEQ ID NO: 200 - DNA, 94 bp, synthetic construct
  source 1..94: mol_type = other DNA
ggtgttcctgaagggggctataaaagggggtgggggcgcgttcgtcctcactctcttccgcatcgctgtctgcgagggccagctgttggggtga

SEQ ID NO: 201 - DNA, 382 bp, synthetic construct
  source 1..382: mol_type = other DNA
ggggctagtgctcgggcaggtgccctggcttcccttcccctccacacgtcaacgattctatttgaagttgggcaggggggtggcgctgctcaccacacacaagtgttataggaggagtctggcccttgagtaccgggtacgcaggggtgcctcaaccacactccgtccacggactctccgttattttaggaggtgagtgtagtgccagtatctactctccttcttaaaaaaaaccagggctccagagaatcagaacagccaccatcaccgcagggagtcaagggaggagggagattagagaaggagccagggagggtggcagggaggccacgtgatccgagtcccctcacccctttccttcccacaggtccctggccaaaga

SEQ ID NO: 202 - DNA, 1051 bp, synthetic construct
  source 1..1051: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactaccgcggatccgccacc

SEQ ID NO: 203 - DNA, 1026 bp, synthetic construct
  source 1..1026: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcctcgggagccgtggaggtacgaacttaagacatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacaccccgcggatccgccacc

SEQ ID NO: 204 - DNA, 1031 bp, synthetic construct
  source 1..1031: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagccgaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaaccgcggatccgccacc

SEQ ID NO: 205 - DNA, 920 bp, synthetic construct
  source 1..920: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagccgcgattgtgcgttgtgtaagccacggcgcagggtggggcgcgggcgggacttgggcgggcggggtgggcttggccgagctggcctccggggcaccgaccgctataaggccagtcggactgcgacacagcccatcccctcgaccgctcgcgtcgcatttggccgcctccctaccggtgagttctctccaggagccctgggtacttccgcggatccgccacc

SEQ ID NO: 206 - DNA, 1112 bp, synthetic construct
  source 1..1112: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagccgcctgaagggcgggacaggaagtgaggtcactcctcaccggggtgaaaggttagcggaagtgtccttctttcctttttgctggtagggccgggtggttgctgccggtaagtagaagcttgggttgaatctttcaatccgctgccatccgcggcttgggggtcgaaggtccgggcggcgccgggtgcattccttccctaggtctctggcggctacgtatccgatcctgtcctgcgagcagggccctcgggcggccttgaccaaggcgacagtgaacacagtctgaagttccggcccaagacctgggcttgctggcaggagtcagctctgggcatctttggctttacggatttttaagtggccctttctccttgctctctgcagaaaggggcaagttcaccgcggatccgccac

SEQ ID NO: 207 - DNA, 909 bp, synthetic construct
  source 1..909: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcctctctgcgcggcacggtgagcgcggccagggcaggacggggacgggcggagcagggacggccaccccaggagatgccaggggagaagggccagggacgaggacctgggtcgagggcggggcctgaacgttacccccgggctagaggcggggcctggggccgggggccgggcctcgaaacctccccaagtcgggccgcggatccgccacc

SEQ ID NO: 208 - DNA, 891 bp, synthetic construct
  source 1..891: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccgggccgcggatccgccac

SEQ ID NO: 209 - DNA, 1098 bp, synthetic construct
  source 1..1098: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagccggggctagtgctcgggcaggtgccctggcttcccttcccctccacacgtcaacgattctatttgaagttgggcaggggggtggcgctgctcaccacacacaagtgttataggaggagtctggcccttgagtaccgggtacgcaggggtgcctcaaccacactccgtccacggactctccgttattttaggaggtgagtgtagtgccagtatctactctccttcttaaaaaaaaccagggctccagagaatcagaacagccaccatcaccgcagggagtcaagggaggagggagattagagaaggagccagggagggtggcagggaggccacgtgatccgagtcccctcacccctttccttcccacaggtccctggccaaagaccgcggatccgccacc

SEQ ID NO: 210 - DNA, 917 bp, synthetic construct
  source 1..917: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagtccgcggatccgccacc

SEQ ID NO: 211 - DNA, 917 bp, synthetic construct
  source 1..917: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccagacatttggagaagtatgcagaacatcttggcagttttcctttaactctaagggttgtaaatacaaagtagaatgtgttaccatgagccatttcataggaagttgactttatttttttagaattcagaactttgcttctaaaaaggtaactttgcctcttgattctcatttttttccctaacttaggcaccttggaagttgccgcggatccgcca

SEQ ID NO: 212 - DNA, 1246 bp, synthetic construct
  source 1..1246: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccgaggcagttggctggtgctgacatgttgaccatcactgctctcttccaaggactcacaaagagttaatgtccctggggctcagcctaggaagattccagtccctgcccaggcccaagatagttgctggcctgattcccctggcattcaggactggaaaggaggaggaggggcacactacgccggctcccatcctccccccaccccgcgtgcctgcttgggattcctgactctgtaccagcttcagagaacaggggtgggggtgggtgccattgggtgtggacagaaagctagtgaaacaagaccttgacaagtcactggccggctcagacgtgtttgtgtctctcttttcttagctcagtgagtactgggtttgtgtcacattgccaaatcccggatcacaagtctccttgaactgctggtgagctaggataataaaacccctgacatcaccattccagaagcttcacaagactgcatatataaggggctggctgtagctgcagctgaaggagctgaccagccagctgaccgcggatccgccacc

SEQ ID NO: 213 - DNA, 1054 bp, synthetic construct
  source 1..1054: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagccctctgccctgccagggtaggtctgggaatcgggggcctgctgcgggaggtggaggcccaagggaggccccccggggactgtgtgtctcacccccgtccctgctacgttgtgttgttgtgtgatcccatcgtggaggttgttttggtgacactgtgtccccacgaagctggggatacccgtttctctagcttggagccaccaagatagaggacaaacacttctgtgattcagtccccagactgtctctgacttaatcccttgggttcaagccctttgtgggagagcaagggcacacactgcctaatccgtggtgtcccccccaggacaattgcgtctctccgcggatccgccacc

SEQ ID NO: 214 - DNA, 1061 bp, synthetic construct
  source 1..1061: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccgtgcccgggctccggtgagtcagggcgcgttatgcaagtgcccccggcgcctccccttcggtctttcaccccgcgcggttacgaaagcgcgaccccctccccccggcgctataaagcagcggggcggccgcggcgcgctcgcctccctcgctccacgcgcgcccggactcggcggccaggcttgcgcgcggttcccctcccggtggtgagtcgcggccggggcctcggggccggcggcgggttgggccggggtggggtggggtggggtgaggtgggatacggcggcgggggtcggctgccgggcactgacggaggcgcgtctcccgcagggcggattcctgggcccgcggatccgccacc

SEQ ID NO: 215 - DNA, 1128 bp, synthetic construct
  source 1..1128: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccaggaagattgagaggtgagagtgctgaacggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctgagtgcctgggagggacacccgcctacactctgcaagaaactcaaaaagggagttgaggggatcgtgggagggaggtagggagggaggagggtgccactgatcccctgaacccctgcctctgcctccagagtgcccctccggccccgcggatccgccacc

SEQ ID NO: 216 - DNA, 982 bp, synthetic construct
  source 1..982: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagccgctcagagggaagaggtcggatccccagctgagagggaggagggtcccggaccctaggagtgggaaggaaaggctcggatcccctgatccccaggaggaggggacccggctgcctcccggttggggccgcgcgagggcggggcgcggaaggatccgggagggccgtgctccgccacccagtatatatctgtccccagtccccggggccgcctcattccctgtcctcggatcacagtctcttctcactacagtgtcgccgcctctgcccgcggatccgccacc

SEQ ID NO: 217 - DNA, 953 bp, synthetic construct
  source 1..953: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagccccatcccccaggtaggccaggaggcctgggcgggacctgcttggggttaaatactccggcccagcactccccaggcctctagtcccagtaggaggtttgactaagatcaatcttggtaagtgccttggccatctccccttgggtgggacatcttcagtaacgcttccagaagcaccttgtgttggaacctccaagtctcactgtccttctttttctccttagacgacttacagtgacccgcggatccgccacc

SEQ ID NO: 218 - DNA, 1068 bp, synthetic construct
  source 1..1068: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccccgcgtcccggaaggtgagtgggggaggggagatggagcctggggctgccgagaagcagggagagaggggaaaggtctgcaggcctctgggcgcgggggacctttatcaaagggagcggcgaggggaagggagggaccatacgcaggcggagaagaggtagctggggatcttaaggattttgtagggaaaagccttagcggacccatgggtgtagaagggacgggccgtggcggatgcagactgggagggcctgacgggaaagggaaaggatgggcccctgtggacgccacagcgcgccttccccatccgcaaaggctttccctccctctcctccagctccagcctgaaccccgcggatccgccacc

SEQ ID NO: 219 - DNA, 876 bp, synthetic construct
  source 1..876: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcctgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctaccgcggatccgccacc

SEQ ID NO: 220 - DNA, 1596 bp, synthetic construct
  source 1..1596: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgccgcggatccgccacc

SEQ ID NO: 221 - DNA, 1222 bp, synthetic construct
  source 1..1222: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgccgcggatccgccacc

SEQ ID NO: 222 - DNA, 1003 bp, synthetic construct
  source 1..1003: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcctgttgagtgagtcttgcttccccagggggctggcgggctgggctgggggcaggcttccccaaccccagcccaggaagggacagggaatagcttcattccctgacttcatcaaccagctgacctactgactccctctccctcccttcctccctccccttcccttcctccctccgctccttgcttccctccccctccctccctttctgcccttctctctcctctttctcccctttcccatctccctctgccttcccctggcactctctctctagaacgggcagctgctgccgcggatccgccacc

SEQ ID NO: 223 - DNA, 1292 bp, synthetic construct
  source 1..1292: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcccgaagtaacttctcggtaaagtcttcacggaatttccagaccacacttgcccactgggaggcttttaggacccgagacgtgtgcaggcttttccaggttcgggttggtgggccttattcattttggcggggctatacgtattcactcttaagcctgtagaagatgaacgttgattgtaccttctaaaacgtgaggttatagttaagagaattgcgttgtcggtctgctcagcagagatgtaagtccagtcttgatttcctcaggcactacttgaggagttgggagagtgtaactaaaaggtcttataactaggacaagtgtttttgatgcaaaagtctttagtccgtaaacatatacagaaaacacccgaataaaacaaaaaataaaaagtttcgagcttcggttgataggcccttccgatcaaacagtttttgttgaagttgcacaacatgacactgcctatgatcattctagactatcaagataaaatcattgcggttatttgtttggaagctggatgagattttggtcatttaagagtctttttaactttttatttagccaaattgaagtttaccgcggatccgccacc

SEQ ID NO: 224 - DNA, 737 bp, synthetic construct
  source 1..737: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactaggatccgccacc

SEQ ID NO: 225 - DNA, 712 bp, synthetic construct
  source 1..712: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcctcgggagccgtggaggtacgaacttaagacatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacaccggatccgccacc

SEQ ID NO: 226 - DNA, 717 bp, synthetic construct
  source 1..717: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccgaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaaggatccgccacc

SEQ ID NO: 227 - DNA, 606 bp, synthetic construct
  source 1..606: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccgcgattgtgcgttgtgtaagccacggcgcagggtggggcgcgggcgggacttgggcgggcggggtgggcttggccgagctggcctccggggcaccgaccgctataaggccagtcggactgcgacacagcccatcccctcgaccgctcgcgtcgcatttggccgcctccctaccggtgagttctctccaggagccctgggtacttggatccgccacc

SEQ ID NO: 228 - DNA, 799 bp, synthetic construct
  source 1..799: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccgcctgaagggcgggacaggaagtgaggtcactcctcaccggggtgaaaggttagcggaagtgtccttctttcctttttgctggtagggccgggtggttgctgccggtaagtagaagcttgggttgaatctttcaatccgctgccatccgcggcttgggggtcgaaggtccgggcggcgccgggtgcattccttccctaggtctctggcggctacgtatccgatcctgtcctgcgagcagggccctcgggcggccttgaccaaggcgacagtgaacacagtctgaagttccggcccaagacctgggcttgctggcaggagtcagctctgggcatctttggctttacggatttttaagtggccctttctccttgctctctgcagaaaggggcaagttcaggatccgccacc

SEQ ID NO: 229 - DNA, 595 bp, synthetic construct
  source 1..595: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcctctctgcgcggcacggtgagcgcggccagggcaggacggggacgggcggagcagggacggccaccccaggagatgccaggggagaagggccagggacgaggacctgggtcgagggcggggcctgaacgttacccccgggctagaggcggggcctggggccgggggccgggcctcgaaacctccccaagtcgggggatccgccacc

SEQ ID NO: 230 - DNA, 578 bp, synthetic construct
  source 1..578: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccgggggatccgccacc

SEQ ID NO: 231 - DNA, 784 bp, synthetic construct
  source 1..784: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccggggctagtgctcgggcaggtgccctggcttcccttcccctccacacgtcaacgattctatttgaagttgggcaggggggtggcgctgctcaccacacacaagtgttataggaggagtctggcccttgagtaccgggtacgcaggggtgcctcaaccacactccgtccacggactctccgttattttaggaggtgagtgtagtgccagtatctactctccttcttaaaaaaaaccagggctccagagaatcagaacagccaccatcaccgcagggagtcaagggaggagggagattagagaaggagccagggagggtggcagggaggccacgtgatccgagtcccctcacccctttccttcccacaggtccctggccaaagaggatccgccacc

SEQ ID NO: 232 - DNA, 603 bp, synthetic construct
  source 1..603: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagtggatccgccacc

SEQ ID NO: 233 - DNA, 605 bp, synthetic construct
  source 1..605: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccagacatttggagaagtatgcagaacatcttggcagttttcctttaactctaagggttgtaaatacaaagtagaatgtgttaccatgagccatttcataggaagttgactttatttttttagaattcagaactttgcttctaaaaaggtaactttgcctcttgattctcatttttttccctaacttaggcaccttggaagttgggatccgccacc

SEQ ID NO: 234 - DNA, 932 bp, synthetic construct
  source 1..932: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccgaggcagttggctggtgctgacatgttgaccatcactgctctcttccaaggactcacaaagagttaatgtccctggggctcagcctaggaagattccagtccctgcccaggcccaagatagttgctggcctgattcccctggcattcaggactggaaaggaggaggaggggcacactacgccggctcccatcctccccccaccccgcgtgcctgcttgggattcctgactctgtaccagcttcagagaacaggggtgggggtgggtgccattgggtgtggacagaaagctagtgaaacaagaccttgacaagtcactggccggctcagacgtgtttgtgtctctcttttcttagctcagtgagtactgggtttgtgtcacattgccaaatcccggatcacaagtctccttgaactgctggtgagctaggataataaaacccctgacatcaccattccagaagcttcacaagactgcatatataaggggctggctgtagctgcagctgaaggagctgaccagccagctgaggatccgccacc

SEQ ID NO: 235 - DNA, 740 bp, synthetic construct
  source 1..740: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccctctgccctgccagggtaggtctgggaatcgggggcctgctgcgggaggtggaggcccaagggaggccccccggggactgtgtgtctcacccccgtccctgctacgttgtgttgttgtgtgatcccatcgtggaggttgttttggtgacactgtgtccccacgaagctggggatacccgtttctctagcttggagccaccaagatagaggacaaacacttctgtgattcagtccccagactgtctctgacttaatcccttgggttcaagccctttgtgggagagcaagggcacacactgcctaatccgtggtgtcccccccaggacaattgcgtctctggatccgccacc

SEQ ID NO: 236 - DNA, 747 bp, synthetic construct
  source 1..747: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccgtgcccgggctccggtgagtcagggcgcgttatgcaagtgcccccggcgcctccccttcggtctttcaccccgcgcggttacgaaagcgcgaccccctccccccggcgctataaagcagcggggcggccgcggcgcgctcgcctccctcgctccacgcgcgcccggactcggcggccaggcttgcgcgcggttcccctcccggtggtgagtcgcggccggggcctcggggccggcggcgggttgggccggggtggggtggggtggggtgaggtgggatacggcggcgggggtcggctgccgggcactgacggaggcgcgtctcccgcagggcggattcctgggcggatccgccacc

SEQ ID NO: 237 - DNA, 814 bp, synthetic construct
  source 1..814: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccaggaagattgagaggtgagagtgctgaacggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctgagtgcctgggagggacacccgcctacactctgcaagaaactcaaaaagggagttgaggggatcgtgggagggaggtagggagggaggagggtgccactgatcccctgaacccctgcctctgcctccagagtgcccctccggccggatccgccacc

SEQ ID NO: 238 - DNA, 668 bp, synthetic construct
  source 1..668: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccgctcagagggaagaggtcggatccccagctgagagggaggagggtcccggaccctaggagtgggaaggaaaggctcggatcccctgatccccaggaggaggggacccggctgcctcccggttggggccgcgcgagggcggggcgcggaaggatccgggagggccgtgctccgccacccagtatatatctgtccccagtccccggggccgcctcattccctgtcctcggatcacagtctcttctcactacagtgtcgccgcctctgcggatccgccacc

SEQ ID NO: 239 - DNA, 639 bp, synthetic construct
  source 1..639: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccccatcccccaggtaggccaggaggcctgggcgggacctgcttggggttaaatactccggcccagcactccccaggcctctagtcccagtaggaggtttgactaagatcaatcttggtaagtgccttggccatctccccttgggtgggacatcttcagtaacgcttccagaagcaccttgtgttggaacctccaagtctcactgtccttctttttctccttagacgacttacagtgacggatccgccacc

SEQ ID NO: 240 - DNA, 754 bp, synthetic construct
  source 1..754: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggaggggagatggagcctggggctgccgagaagcagggagagaggggaaaggtctgcaggcctctgggcgcgggggacctttatcaaagggagcggcgaggggaagggagggaccatacgcaggcggagaagaggtagctggggatcttaaggattttgtagggaaaagccttagcggacccttgggtgtagaagggacgggccgtggcggttgcagactgggagggcctgacgggaaagggaaaggttgggcccctgtggacgccacagcgcgccttccccatccgcaaaggctttccctccctctcctccagctccagcctgaaccggatccgccacc

SEQ ID NO: 241 - DNA, 562 bp, synthetic construct
  source 1..562: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcctgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctaggatccgccacc

SEQ ID NO: 242 - DNA, 1282 bp, synthetic construct
  source 1..1282: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgggatccgccacc

SEQ ID NO: 243 - DNA, 908 bp, synthetic construct
  source 1..908: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgggatccgccacc

SEQ ID NO: 244 - DNA, 689 bp, synthetic construct
  source 1..689: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcctgttgagtgagtcttgcttccccagggggctggcgggctgggctgggggcaggcttccccaaccccagcccaggaagggacagggaatagcttcattccctgacttcatcaaccagctgacctactgactccctctccctcccttcctccctccccttcccttcctccctccgctccttgcttccctccccctccctccctttctgcccttctctctcctctttctcccctttcccatctccctctgccttcccctggcactctctctctagaacgggcagctgctgggatccgccacc

SEQ ID NO: 245 - DNA, 978 bp, synthetic construct
  source 1..978: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccgaagtaacttctcggtaaagtcttcacggaatttccagaccacacttgcccactgggaggcttttaggacccgagacgtgtgcaggcttttccaggttcgggttggtgggccttattcattttggcggggctatacgtattcactcttaagcctgtagaagatgaacgttgattgtaccttctaaaacgtgaggttatagttaagagaattgcgttgtcggtctgctcagcagagatgtaagtccagtcttgatttcctcaggcactacttgaggagttgggagagtgtaactaaaaggtcttataactaggacaagtgtttttgatgcaaaagtctttagtccgtaaacatatacagaaaacacccgaataaaacaaaaaataaaaagtttcgagcttcggttgataggcccttccgatcaaacagtttttgttgaagttgcacaacatgacactgcctatgatcattctagactatcaagataaaatcattgcggttatttgtttggaagctggatgagattttggtcatttaagagtctttttaactttttatttagccaaattgaagtttaggatccgccacc

SEQ ID NO: 246 - DNA, 589 bp, synthetic construct
  source 1..589: mol_type = other DNA
cgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgggcgaagccggtccccgccccgcggggccccgccccgcgtgcgccgttccgaaagttgctttctagatctcgccccgccccggcggcgcctatataaggtgtgggcgggtgtggcacagctagttccgtcgcagccggcgagccgaacggtcgttgagccaagaggtaagggtttaagggatggttggttggtggggtattaatgtttaattacctggagcacctgcctgaaatcactttttttcaggttggccgcggatccgccacc

SEQ ID NO: 247 - DNA, 809 bp, synthetic construct
  source 1..809: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgggcgaagccggtccccgccccgcggggccccgccccgcgtgcgccgttccgaaagttgctttctagatctcgccccgccccggcggcgcctatataaggtgtgggcgggtgtggcacagctagttccgtcgcagccggcgagccgaacggtcgttgagcccgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 248 - DNA, 731 bp, synthetic construct
  source 1..731: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactaggatccgccacc

SEQ ID NO: 249 - DNA, 1195 bp, synthetic construct
  source 1..1195: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 250 - DNA, 1240 bp, synthetic construct
  source 1..1240: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgagaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 251 - DNA, 1298 bp, synthetic construct
  source 1..1298: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgggcgaagccggtccccgccccgcggggccccgccccgcgtgcgccgttccgaaagttgctttctagatctcgccccgccccggcggcgcctatataaggtgtgggcgggtgtggcacagctagttccgtcgcagccggcgagccgaacggtcgttgagcccgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 252 - DNA, 1339 bp, synthetic construct
  source 1..1339: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 253 - DNA, 1384 bp, synthetic construct
  source 1..1384: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 254 - DNA, 1430 bp, synthetic construct
  source 1..1430: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgggcgaagccggtccccgccccgcggggccccgccccgcgtgcgccgttccgaaagttgctttctagatctcgccccgccccggcggcgcctatataaggtgtgggcgggtgtggcacagctagttccgtcgcagccggcgagccgaacggtcgttgagcccgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 255 - DNA, 1120 bp, synthetic construct
  source 1..1120: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 256 - DNA, 1165 bp, synthetic construct
  source 1..1165: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 257 - DNA, 1211 bp, synthetic construct
  source 1..1211: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaataatacgactaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 258 - DNA, 1271 bp, synthetic construct
  source 1..1271: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagatcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaataatacgactaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 259 - DNA, 1719 bp, synthetic construct
  source 1..1719: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtaatacgactaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaaagtaattctccttggaatttgccctttttgagtttggatcttggttcattctcaagcctcagacagtggttcaaagtttttttcttccatttcaggtgtcgtgaggatccgccacc

SEQ ID NO: 260 - DNA, 1659 bp, synthetic construct
  source 1..1659: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtaatacgactaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgagtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaaagtaattctccttggaatttgccctttttgagtttggatcttggttcattctcaagcctcagacagtggttcaaagtttttttcttccatttcaggtgtcgtgaggatccgccacc

SEQ ID NO: 261 - DNA, 1304 bp, synthetic construct
  source 1..1304: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagtcagatcgcctggagaggccatccacgctgttttgacctccatagtggacaccgggaccgatccagcctccgcgtctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccacc

SEQ ID NO: 262 - DNA, 820 bp, synthetic construct
  source 1..820: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagtcagatcgcctggagaggccatccacgctgttttgacctccaagaggtaagggtttaagggatggttggttggtggggtattaatgtttaattacctggagcacctgcctgaaatcactttttttcaggttggcccaggtccaagtttaaacgccgccacc

SEQ ID NO: 263 - DNA, 737 bp, synthetic construct
  source 1..737: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactaggatccgccacc

SEQ ID NO: 264 - DNA, 712 bp, synthetic construct
  source 1..712: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacaccggatccgccacc

SEQ ID NO: 265 - DNA, 717 bp, synthetic construct
  source 1..717: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggaggggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaaggatccgccacc

SEQ ID NO: 266 - DNA, 606 bp, synthetic construct
  source 1..606: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggaggggggtggggcgcgggcgggacttgggcgggcggggtgggcttggccgagctggcctccggggcaccgaccgctataaggccagtcggactgcgacacagcccatcccctcgaccgctcgcgtcgcatttggccgcctccctaccggtgagttctctccaggagccctgggtacttggatccgccacc

SEQ ID NO: 267 - DNA, 799 bp, synthetic construct
  source 1..799: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggctcctcaccggggtgaaaggttagcggaagtgtccttctttcctttttgctggtagggccgggtggttgctgccggtaagtagaagcttgggttgaatctttcaatccgctgccatccgcggcttgggggtcgaaggtccgggcggcgccgggtgcattccttccctaggtctctggcggctacgtatccgatcctgtcctgcgagcagggccctcgggcggccttgaccaaggcgacagtgaacacagtctgaagttccggcccaagacctgggcttgctggcaggagtcagctctgggcatctttggctttacggatttttaagtggccctttctccttgctctctgcagaaaggggcaagttcaggatccgccacc

SEQ ID NO: 268 - DNA, 595 bp, synthetic construct
  source 1..595: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggaggggcaggacggggacgggcggagcagggacggccaccccaggagatgccaggggagaagggccagggacgaggacctgggtcgagggcggggcctgaacgttacccccgggctagaggcggggcctggggccgggggccgggcctcgaaacctccccaagtcgggggatccgccacc

SEQ ID NO: 269 - DNA, 578 bp, synthetic construct
  source 1..578: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggaggggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccgggggatccgccacc

SEQ ID NO: 270 - DNA, 784 bp, synthetic construct
  source 1..784: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggtcccttcccctccacacgtcaacgattctatttgaagttgggcaggggggtggcgctgctcaccacacacaagtgttataggaggagtctggcccttgagtaccgggtacgcaggggtgcctcaaccacactccgtccacggactctccgttattttaggaggtgagtgtagtgccagtatctactctccttcttaaaaaaaaccagggctccagagaatcagaacagccaccatcaccgcagggagtcaagggaggagggagattagagaaggagccagggagggtggcagggaggccacgtgatccgagtcccctcacccctttccttcccacaggtccctggccaaagaggatccgccacc

SEQ ID NO: 271 - DNA, 603 bp, synthetic construct
  source 1..603: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggaggggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagtggatccgccacc

SEQ ID NO: 272 - DNA, 605 bp, synthetic construct
  source 1..605: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggtggcagttttcctttaactctaagggttgtaaatacaaagtagaatgtgttaccatgagccatttcataggaagttgactttatttttttagaattcagaactttgcttctaaaaaggtaactttgcctcttgattctcatttttttccctaacttaggcaccttggaagttgggatccgccacc

SEQ ID NO: 273 - DNA, 932 bp, synthetic construct
  source 1..932: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggccatcactgctctcttccaaggactcacaaagagttaatgtccctggggctcagcctaggaagattccagtccctgcccaggcccaagatagttgctggcctgattcccctggcattcaggactggaaaggaggaggaggggcacactacgccggctcccatcctccccccaccccgcgtgcctgcttgggattcctgactctgtaccagcttcagagaacaggggtgggggtgggtgccattgggtgtggacagaaagctagtgaaacaagaccttgacaagtcactggccggctcagacgtgtttgtgtctctcttttcttagctcagtgagtactgggtttgtgtcacattgccaaatcccggatcacaagtctccttgaactgctggtgagctaggataataaaacccctgacatcaccattccagaagcttcacaagactgcatatataaggggctggctgtagctgcagctgaaggagctgaccagccagctgaggatccgccacc

SEQ ID NO: 274 - DNA, 740 bp, synthetic construct
  source 1..740: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggaggggggggcctgctgcgggaggtggaggcccaagggaggccccccggggactgtgtgtctcacccccgtccctgctacgttgtgttgttgtgtgatcccatcgtggaggttgttttggtgacactgtgtccccacgaagctggggatacccgtttctctagcttggagccaccaagatagaggacaaacacttctgtgattcagtccccagactgtctctgacttaatcccttgggttcaagccctttgtgggagagcaagggcacacactgcctaatccgtggtgtcccccccaggacaattgcgtctctggatccgccacc

SEQ ID NO: 275 - DNA, 747 bp, synthetic construct
  source 1..747: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggttatgcaagtgcccccggcgcctccccttcggtctttcaccccgcgcggttacgaaagcgcgaccccctccccccggcgctataaagcagcggggcggccgcggcgcgctcgcctccctcgctccacgcgcgcccggactcggcggccaggcttgcgcgcggttcccctcccggtggtgagtcgcggccggggcctcggggccggcggcgggttgggccggggtggggtggggtggggtgaggtgggatacggcggcgggggtcggctgccgggcactgacggaggcgcgtctcccgcagggcggattcctgggcggatccgccacc

SEQ ID NO: 276 - DNA, 814 bp, synthetic construct
  source 1..814: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctgagtgcctgggagggacacccgcctacactctgcaagaaactcaaaaagggagttgaggggatcgtgggagggaggtagggagggaggagggtgccactgatcccctgaacccctgcctctgcctccagagtgcccctccggccggatccgccacc

SEQ ID NO: 277 - DNA, 668 bp, synthetic construct
  source 1..668: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggaggggagagggaggagggtcccggaccctaggagtgggaaggaaaggctcggatcccctgatccccaggaggaggggacccggctgcctcccggttggggccgcgcgagggcggggcgcggaaggatccgggagggccgtgctccgccacccagtatatatctgtccccagtccccggggccgcctcattccctgtcctcggatcacagtctcttctcactacagtgtcgccgcctctgcggatccgccacc

SEQ ID NO: 278 - DNA, 639 bp, synthetic construct
  source 1..639: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggcgggacctgcttggggttaaatactccggcccagcactccccaggcctctagtcccagtaggaggtttgactaagatcaatcttggtaagtgccttggccatctccccttgggtgggacatcttcagtaacgcttccagaagcaccttgtgttggaacctccaagtctcactgtccttctttttctccttagacgacttacagtgacggatccgccacc

SEQ ID NO: 279 - DNA, 562 bp, synthetic construct
  source 1..562: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggacaagacaggtttaaggagaccaatagaaactgggcatgtggagacagagaagactcttgggtttctgataggcactgactctctctgcctattggtctattttcccacccttaggctgctggtggtctaggatccgccacc

SEQ ID NO: 280 - DNA, 1282 bp, synthetic construct
  source 1..1282: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggaggggcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgggatccgccacc

SEQ ID NO: 281 - DNA, 908 bp, synthetic construct
  source 1..908: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgggatccgccacc

SEQ ID NO: 282 - DNA, 689 bp, synthetic construct
  source 1..689: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggtggcgggctgggctgggggcaggcttccccaaccccagcccaggaagggacagggaatagcttcattccctgacttcatcaaccagctgacctactgactccctctccctcccttcctccctccccttcccttcctccctccgctccttgcttccctccccctccctccctttctgcccttctctctcctctttctcccctttcccatctccctctgccttcccctggcactctctctctagaacgggcagctgctgggatccgccacc

SEQ ID NO: 283 - DNA, 978 bp, synthetic construct
  source 1..978: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcccccgcgtcccggaaggtgagtgggggagggaatttccagaccacacttgcccactgggaggcttttaggacccgagacgtgtgcaggcttttccaggttcgggttggtgggccttattcattttggcggggctatacgtattcactcttaagcctgtagaagatgaacgttgattgtaccttctaaaacgtgaggttatagttaagagaattgcgttgtcggtctgctcagcagagatgtaagtccagtcttgatttcctcaggcactacttgaggagttgggagagtgtaactaaaaggtcttataactaggacaagtgtttttgatgcaaaagtctttagtccgtaaacatatacagaaaacacccgaataaaacaaaaaataaaaagtttcgagcttcggttgataggcccttccgatcaaacagtttttgttgaagttgcacaacatgacactgcctatgatcattctagactatcaagataaaatcattgcggttatttgtttggaagctggatgagattttggtcatttaagagtctttttaactttttatttagccaaattgaagtttaggatccgccacc

SEQ ID NO: 284 - DNA, 412 bp, synthetic construct
  source 1..412: mol_type = other DNA
caggaagattgagaggtgagagtgctgaacggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctgagtgcctgggagggacacccgcctacactctgcaagaaactcaaaaagggagttgaggggatcgtgggagggaggtagggagggaggagggtgccactgatcccctgaacccctgcctctgcctccagagtgcccctccggcc

SEQ ID NO: 285 - DNA, 266 bp, synthetic construct
  source 1..266: mol_type = other DNA
gctcagagggaagaggtcggatccccagctgagagggaggagggtcccggaccctaggagtgggaaggaaaggctcggatcccctgatccccaggaggaggggacccggctgcctcccggttggggccgcgcgagggcggggcgcggaaggatccgggagggccgtgctccgccacccagtatatatctgtccccagtccccggggccgcctcattccctgtcctcggatcacagtctcttctcactacagtgtcgccgcctctgc

SEQ ID NO: 286 - DNA, 1421 bp, synthetic construct
  source 1..1421: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatttcagcaccgcggacagtgccagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccgccacc

SEQ ID NO: 287 - DNA, 916 bp, synthetic construct
  source 1..916: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgccgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatggaggacgcggcgctcgggagagcgggcggggcagttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactaggatccgccacc

SEQ ID NO: 288 - DNA, 838 bp, synthetic construct
  source 1..838: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgccgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatggaggacgcggcgctcgggagagcgggcggggcagtggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactaggatccgccacc

SEQ ID NO: 289 - DNA, 667 bp, synthetic construct
  source 1..667: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgccgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatggaggacgcggcgctcgggagagcgggcggggcagtggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactaggatccgccacc

SEQ ID NO: 290 - DNA, 1077 bp, synthetic construct
  source 1..1077: mol_type = other DNA
ttacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacaccccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgccgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatggaggacgcggcgctcgggagagcgggcggggcagtggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactaggatccgccacc

SEQ ID NO: 291 - DNA, 862 bp, synthetic construct
  source 1..862: mol_type = other DNA
gggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatttcagcaccgcggacagtgccgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacaggtaagtgccgtgtgtggttcccgccgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatggaggacgcggcgctcgggagagcgggcggggcagtggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactaggatccgccacc

SEQ ID NO: 292 - DNA, 695 bp, synthetic construct
  source 1..695: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacataatacgactaagaggtaagggtttaagggatggttggttggtggggtattaatgtttaattacctggagcacctgcctgaaatcactttttttcaggttggccgcggatccgccacc

SEQ ID NO: 293 - DNA, 1377 bp, synthetic construct
  source 1..1377: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccacc

SEQ ID NO: 294 - DNA, 265 bp, synthetic construct
  source 1..265: mol_type = other DNA
gtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaa

SEQ ID NO: 295 - DNA, 263 bp, synthetic construct
  source 1..263: mol_type = other DNA
gatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaa

SEQ ID NO: 296 - DNA, 325 bp, synthetic construct; identical to SEQ ID NO: 92
  source 1..325: mol_type = other DNA

SEQ ID NO: 297 - DNA, 315 bp, synthetic construct
  source 1..315: mol_type = other DNA
tctgggcctacggagccagcacccatcgccaaaactcttcatcctcttcctcaatctcgctttctctctcgctttttttttttttcttcttctttttttttttttttttcaaaaggaggggagagggggtaaaaaattgctgcactgtgcggcgaggccggtgagtgagcgacgcggagccaatcagcgcccgccgttccgaaagttgccttttgtggctcgagtggccgctgtggcgtcctataaaacccggcggcgcaacgcgcagtcagtccgcctggagacctcgagccgagtggtcgtgcctccatagaa

SEQ ID NO: 298 - DNA, 71 bp, synthetic construct
  source 1..71: mol_type = other DNA
cggcgcctatataaggtgtgggcgggtgtggcacagctagttccgtcgcagccggcgagccgaacggtcgt

SEQ ID NO: 299 - DNA, 383 bp, synthetic construct
  source 1..383: mol_type = other DNA
gtgttgcggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaaatgggcggtaggggtgttcctgaagggggctataaaagggggtgggggcgcgttcgtcctcactctcttccgcatcctcgagccgagtggtcgtctgttggggtgataatacgactaagaggtaagggtttaagggatggttggttggtggggtattaatgtttaattacctggagcacctgcctgaaatcactttttttcaggttggccgcggatccgccacc

SEQ ID NO: 300 - DNA, 1245 bp, synthetic construct; identical to SEQ ID NO: 36
  source 1..1245: mol_type = other DNA

SEQ ID NO: 301 - DNA, 201 bp, synthetic construct
  source 1..201: mol_type = other DNA
cgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagt

SEQ ID NO: 302 - DNA, 1272 bp, synthetic construct
  source 1..1272: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagatcggcttctggcgtgtgaccggcggggtttatatcttcccttctctgttcctccgcagccagcgccacc

SEQ ID NO: 303 - DNA, 1273 bp, synthetic construct
  source 1..1273: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 304 - DNA, 1398 bp, synthetic construct
  source 1..1398: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcctcggcttctggcgtgtgaccggcggggtttatatcttcccttctctgttcctccgcagccagcgccacc

SEQ ID NO: 305 - DNA, 1205 bp, synthetic construct
  source 1..1205: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacatcggcttctggcgtgtgaccggcggggtttatatcttcccttctctgttcctccgcagccagcgccacc

SEQ ID NO: 306 - DNA, 1485 bp, synthetic construct
  source 1..1485: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagatcggcttctggcgtgtgaccggcggggtttatatcttcccttctctgttcctccgcagccagccgccacc

SEQ ID NO: 307 - DNA, 1486 bp, synthetic construct
  source 1..1486: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagccgccacc

SEQ ID NO: 308 - DNA, 1611 bp, synthetic construct
  source 1..1611: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcctcggcttctggcgtgtgaccggcggggtttatatcttcccttctctgttcctccgcagccagccgccacc

SEQ ID NO: 309 - DNA, 1418 bp, synthetic construct
  source 1..1418: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacatcggcttctggcgtgtgaccggcggggtttatatcttcccttctctgttcctccgcagccagccgccacc

SEQ ID NO: 310 - DNA, 1302 bp, synthetic construct
  source 1..1302: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagccgccacc

SEQ ID NO: 311 - DNA, 413 bp, synthetic construct
  source 1..413: mol_type = other DNA
ggagtcgctgcgttgccttcgccccgtgccccgctccgcgccgcctcgcgccgcccgccccggctgggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 312 - DNA, 482 bp, synthetic construct
  source 1..482: mol_type = other DNA
ggagtcgctgcgttgccttcgccccgtgccccgctccgcgccgcctcgcgccgcccgccccggctctgactgaccgcgttactcccacaggtgagcgggcgggacgggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagccgccacc

SEQ ID NO: 313 - DNA, 523 bp, synthetic construct
  source 1..523: mol_type = other DNA
ggagtcgctgcgttgccttcgccccgtgccccgctccgcgccgcctcgcgccgcccgccccggctaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 314 - DNA, 782 bp, synthetic construct
  source 1..782: mol_type = other DNA
ggagtcgctgcgttgccttcgccccgtgccccgctccgcgccgcctcgcgccgcccgccccggctgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 315 - DNA, 852 bp, synthetic construct
  source 1..852: mol_type = other DNA
ggagtcgctgcgttgccttcgccccgtgccccgctccgcgccgcctcgcgccgcccgccccggctctgactgaccgcgttactcccacaggtgagcgggcgggaccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagccgccacc

SEQ ID NO: 316 - DNA, 1023 bp, synthetic construct
  source 1..1023: mol_type = other DNA
ggagtcgctgcgttgccttcgccccgtgccccgctccgcgccgcctcgcgccgcccgccccggctctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 317 - DNA, 1057 bp, synthetic construct
  source 1..1057: mol_type = other DNA
ggagtcgctgcgttgccttcgccccgtgccccgctccgcgccgcctcgcgccgcccgccccggctctgactgaccgcgttactcccacaggtgagcgggcgggacgccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagccgccacc

SEQ ID NO: 318 - DNA, 90 bp, synthetic construct
  source 1..90: mol_type = other DNA
gtcgctgcgcgctgccttcgccccgtgccccgctccgccgccgcctcgcgccgcccgccccggctctgactgaccgcgttactcccacag

SEQ ID NO: 319 - skipped

SEQ ID NO: 320 - DNA, 1333 bp, synthetic construct
  source 1..1333: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaatcggcttctggcgtgtgaccggcggggtttatatcttcccttctctgttcctccgcagccagcgccacc

SEQ ID NO: 321 - DNA, 1273 bp, synthetic construct
  source 1..1273: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtgtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 322 - DNA, 1333 bp, synthetic construct
  source 1..1333: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaatcggcttctggcgtgtgaccggcggggtttatatcttcccttctctgttcctccgcagccagcgccacc

SEQ ID NO: 323 - DNA, 1273 bp, synthetic construct; identical to SEQ ID NO: 303
  source 1..1273: mol_type = other DNA

SEQ ID NO: 324 - DNA, 1302 bp, synthetic construct
  source 1..1302: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtcccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaatcggcttctggcgtgtgaccggcggggtttatatcttcccttctctgttcctccgcagccagcgccacc

SEQ ID NO: 325 - DNA, 1566 bp, synthetic construct
  source 1..1566: mol_type = other DNA
ggagtcgctgcgttgccttcgccccgtgccccgctccgcgccgcctcgcgccgcccgccccggctctgactgaccgcgttactcccacaggtgagcgggcggggctccgggagggccgcgtggggagcttgtgcgctccgcgtgtgcgcgaggggagcgcggccgggggcggcccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaacccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttgtggtagtgtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaattcctttgtgccaaatctgtgcggagccgaaatctgggaggcgccgccgcaccccctctagcgggcgcggggcgaagcggtgcggcgccggcaggaaggaattgggcggggagggccttcgtgcgtcgccgcgccgccgtccccttctccctctccagcctcggggctgtccgcggggggacggctgccttcgggggggacggggcagggcggggttcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaatcggcttctggcgtgtgaccggcggggtttatatcttcccttctctgttcctccgcagccagcgccacc

SEQ ID NO: 326 - DNA, 1288 bp, synthetic construct
  source 1..1288: mol_type = other DNA
ggagtcgctgcgttgccttcgccccgtgccccgctccgcgccgcctcgcgccgcccgccccggctctgactgaccgcgttactcccacaggtgagcgggcgggaccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaacccctccggggcgtggcgcggggctcgccgtgccgggcggggggtggcggcaggtgggggtgccgggcggggcggggccgcctcgggccggggagggctcgggggaggggtcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaaaaggaattgggcggggagggccttcgtgcgtcgccgcgccgccgtccccttctccctctccagcctcggggctgtccgcggggggacggctgccttcgggggggacggggcagggcgggggtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 327 - DNA, 1566 bp, synthetic construct
  source 1..1566: mol_type = other DNA
ggagtcgctgcgttgccttcgccccgtgccccgctccgcgccgcctcgcgccgcccgccccggctctgactgaccgcgttactcccacaggtgagcgggcggggctccgggagggccgcgtggggagcttgtgcgctccgcgtgtgcgcgaggggagcgcggccgggggcggcccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaacccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatgtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaattcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaatcggcttctggcgtgtgaccggcggggtttatatcttcccttctctgttcctccgcagccagcgccacc

SEQ ID NO: 328 - DNA, 1258 bp, synthetic construct
  source 1..1258: mol_type = other DNA
ggagtcgctgcgttgccttcgccccgtgccccgctccgcgccgcctcgcgccgcccgccccggctctgactgaccgcgttactcccacaggtgagcgggcgggaccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaagccggtggcggcaggatacagcggcttctgcgcgacttataagagctccttgtgcggcgccattttaagcctctcggtctgtggcagcagcgttggcccgtcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaagcattccaaccttccagcctgcgacctgcggagaaaaaaaattacttattttcttgccccatacataccttgaggcgagcaaaaaaattaaattttaaccgtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 329 - DNA, 983 bp, synthetic construct
  source 1..983: mol_type = other DNA
ggagtcgctgcgttgccttcgccccgtgccccgctccgcgccgcctcgcgccgcccgccccggctctgactgaccgcgttactcccacaggtgagcgggcgggaccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaagccggtggcggcaggatacagcggcttctgcgcgacttataagagctccttgtgcggcgccattttaagcctctcggtctgtggcagcagcgttggcccgtctgggcctacggagccagcacccatcgccaaaactcttcatcctcttcctcaatctcgctttctctctcgctttttttttttttcttcttctttttttttttttttttcaaaaggaggggagagggggtaaaaaattgctgcactgtgcggcgaggccggtgagtgagcgacgcggagccaatcagcgcccgccgttccgaaagttgccttttgtggctcgagtggccgctgtggcgtcctataaaacccggcggcgcaacgcgcagtcagtccgcctggagacctcgagccgagtggtcgtgcctccatagaagcattccaaccttccagcctgcgacctgcggagaaaaaaaattacttattttcttgccccatacataccttgaggcgagcaaaaaaattaaattttaacctcggcttctggcgtgtgaccggcggggtttatatcttcccttctctgttcctccgcagccagcgccacc

SEQ ID NO: 330 - DNA, 1248 bp, synthetic construct
  source 1..1248: mol_type = other DNA
ggagtcgctgcgttgccttcgccccgtgccccgctccgcgccgcctcgcgccgcccgccccggctctgactgaccgcgttactcccacaggtgagcgggcgggaccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaagccggtggcggcaggatacagcggcttctgcgcgacttataagagctccttgtgcggcgccattttaagcctctcggtctgtggcagcagcgttggcccgtctgggcctacggagccagcacccatcgccaaaactcttcatcctcttcctcaatctcgctttctctctcgctttttttttttttcttcttctttttttttttttttttcaaaaggaggggagagggggtaaaaaattgctgcactgtgcggcgaggccggtgagtgagcgacgcggagccaatcagcgcccgccgttccgaaagttgccttttgtggctcgagtggccgctgtggcgtcctataaaacccggcggcgcaacgcgcagtcagtccgcctggagacctcgagccgagtggtcgtgcctccatagaagcattccaaccttccagcctgcgacctgcggagaaaaaaaattacttattttcttgccccatacataccttgaggcgagcaaaaaaattaaattttaaccgtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 331 - DNA, 1323 bp, synthetic construct
  source 1..1323: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttctgggcctacggagccagcacccatcgccaaaactcttcatcctcttcctcaatctcgctttctctctcgctttttttttttttcttcttctttttttttttttttttcaaaaggaggggagagggggtaaaaaaatgctgcactgtgcggcgaggccggtgagtgagcgacgcggagccaatcagcgcccgccgttccgaaagttgccttttatggctcgagtggccgctgtggcgtcctataaaacccggcggcgcaacgcgcagtcagtccgcctggagacctcgagccgagtggtcgtgcctccatagaatcggcttctggcgtgtgaccggcggggtttatatcttcccttctctgttcctccgcagccagcgccacc

SEQ ID NO: 332 - DNA, 1089 bp, synthetic construct
  source 1..1089: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggtaggtctatataagcagagctcgtttagtgaaccgtcagtccgcctggagacctcgagccgagtggtcgtgcctccatagaatcggcttctggcgtgtgaccggcggggtttatatcttcccttctctgttcctccgcagccagcgccacc

SEQ ID NO: 333 - DNA, 910 bp, synthetic construct
  source 1..910: mol_type = other DNA
gtgagcgggcgggacggcccttctcctccgggctgtaattagcgcttggtttaatgacggctcgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgagaggagcgcggccgggggcggtgccacgcggtgcggggggggctgcgaggggaacaaaggctgcgtgcggggtgtgtgcgtggggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcgggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggggtttatatcttcccttctctgttcctccgcag

SEQ ID NO: 334 - DNA, 276 bp, synthetic construct
  source 1..276: mol_type = other DNA
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 335 - DNA, 325 bp, synthetic construct
  source 1..325: mol_type = other DNA
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggtcagtccgcctggagacctcgagccgagtggtcgtgcctccatagaa

SEQ ID NO: 336 - DNA, 319 bp, synthetic construct
  source 1..319: mol_type = other DNA
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcc

SEQ ID NO: 337 - DNA, 321 bp, synthetic construct
  source 1..321: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagctagttccgtcgcagccggcgagccgaacggtcgttgagcc

SEQ ID NO: 338 - DNA, 319 bp, synthetic construct
  source 1..319: mol_type = other DNA
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcgcggcgcctatataaggtgtgggcgggtgtggcacagtcagatcgcctggagaggccatccacgctgttttgacctcc

SEQ ID NO: 339 - DNA, 374 bp, synthetic construct
  source 1..374: mol_type = other DNA
gcccgttagtcccggatcccagcggccgcttcaccactgagcccgagccgccgagggaattttccaaatgcaaatcaaacacccttgggtggttagcaacacctcagctgacgccaatgtcaccacaaacacctcatggccagaagcagctcaaccactgggtcaacaaaatacagtatatccatacaataaatatgggtcaacaaaatacggtatatccatacactaaatatgggtcaacaaaatacagtatatccataaaataaataggctggagctcagccgcccccccattgagaagattcgtcgtcctgagaagtcgcgtgctggaggtggagtgtgtctagaagcatttgcggtggacgatggaaggg

SEQ ID NO: 340 - DNA, 212 bp, synthetic construct
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgt

SEQ ID NO: 341 - DNA, 407 bp, synthetic construct
  source 1..407: mol_type = other DNA
gggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatttcagcaccgcggacagtgccgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgt

SEQ ID NO: 342 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 343 - DNA, 1013 bp, synthetic construct
  source 1..1013: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtgccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 344 - DNA, 286 bp, synthetic construct
  source 1..286: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatg

SEQ ID NO: 345 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 346 - DNA, 364 bp, synthetic construct
  source 1..364: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 347 - DNA, 377 bp, synthetic construct
  source 1..377: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattacca

SEQ ID NO: 348 - DNA, 379 bp, synthetic construct
  source 1..379: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 349 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 350 - DNA, 344 bp, synthetic construct
  source 1..344: mol_type = other DNA
ttacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 351 - DNA, 220 bp, synthetic construct
  source 1..220: mol_type = other DNA
tgctgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 352 - DNA, 224 bp, synthetic construct
  source 1..224: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagat

SEQ ID NO: 353 - DNA, 229 bp, synthetic construct
  source 1..229: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcct

SEQ ID NO: 354 - DNA, 264 bp, synthetic construct
  source 1..264: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccataga

SEQ ID NO: 355 - DNA, 265 bp, synthetic construct
  source 1..265: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaa

SEQ ID NO: 356 - DNA, 267 bp, synthetic construct
  source 1..267: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaaga

SEQ ID NO: 357 - DNA, 265 bp, synthetic construct
  source 1..265: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggggggctataaaagggggtgggggcgcgttcgtcctcactctcttccgcatcgctgtctgcgagggccagctgttggggtga

SEQ ID NO: 358 - DNA, 308 bp, synthetic construct
  source 1..308: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggggtgttcctgaagggggctataaaagggggtgggggcgcgttcgtcctcactctcttccgcatcctcgagccgagtggtcgtctgttggggtgataatacgactcactatagggaagcttggcattccggtactgtt

SEQ ID NO: 359 - DNA, 367 bp, synthetic construct
  source 1..367: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggggtgttcctgaagggggctataaaagggggtgggggcgcgttcgtcctcactctcttccgcatcctcgagccgagtggtcgtctgttggggtgataatacgactaagaggtaagggtttaagggatggttggttggtggggtattaatgtttaattacctggagcacctgcctgaaatcactttttttcaggttgg

SEQ ID NO: 360 - DNA, 265 bp, synthetic construct
  source 1..265: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagtccgcctggagacctcgagccgagtggtcgtgcctccatagaa

SEQ ID NO: 361 - DNA, 117 bp, synthetic construct
  source 1..117: mol_type = other DNA
agttctctcgttaacttaatgagacagatagaaactggtcttgtagaaacagagtagtcgcctgcttttctgccaggtgctgacttctctcccctgggcttttttctttttctcagg

SEQ ID NO: 362 - DNA, 530 bp, synthetic construct
  source 1..530: mol_type = other DNA
cgaggcagttggctggtgctgacatgttgaccatcactgctctcttccaaggactcacaaagagttaatgtccctggggctcagcctaggaagattccagtccctgcccaggcccaagatagttgctggcctgattcccctggcattcaggactggaaaggaggaggaggggcacactacgccggctcccatcctccccccaccccgcgtgcctgcttgggattcctgactctgtaccagcttcagagaacaggggtgggggtgggtgccattgggtgtggacagaaagctagtgaaacaagaccttgacaagtcactggccggctcagacgtgtttgtgtctctcttttcttagctcagtgagtactgggtttgtgtcacattgccaaatcccggatcacaagtctccttgaactgctggtgagctaggataataaaacccctgacatcaccattccagaagcttcacaagactgcatatataaggggctggctgtagctgcagctgaaggagctgaccagccagctga

SEQ ID NO: 363 - DNA, 372 bp, synthetic construct
  source 1..372: mol_type = other DNA
acgtattccctccgctctttacgaccggaaatgtaccacaccgaaaattgaggcacaattcgtcaaacataagtcatgccgtaatacgccgtctgcgaaagcgacctattacgtaattcgggttattcttaccttacgtcaccgcaaccctatttccccgcccgttcccttaatcacggaagtcctttacggcagtccgccttggcggtgacgaaagacattccggccggaatattcgaaaagtctttcgcttctgctcggggttggtgtaagacgaaattttcgtgcccggaaaggtctcgaagtcgtccggaagtcggttttcggcgtccgtcgacgaccggaagtagtgacttttacggttccggaagt

SEQ ID NO: 364 - DNA, 372 bp, synthetic construct
  source 1..372: mol_type = other DNA
tttccggaactgaatatcgggcgcgccccggagtcaggctttaaatgcatgggaagctctcttcctacggtgatcccatggcatagactcaagaccggaatttcctcgccggtgtttccggccatttagatattacgacagggttcaaaaacgatcgaagctatttccggaagaccaggtagtttccggttctcgccgacgtcttctcaaacacaagtggtagcggccgaaagcgacatttcgcctactacatcctataatacctagagtcagattgtacggaaggcgtacgagccgttacgcgccgttacgaaaggcagttttcgggattcgtcgacgtccggaagtttcgccgttttcgtttccggaatt

SEQ ID NO: 365 - DNA, 372 bp, synthetic construct
  source 1..372: mol_type = other DNA
acgaattcacccctaaatttgagaccgttcgtatggcacaccagatttaaccccacaacagatcaaacacaaatcatagcccaatccggcattaacagaagcgccccagcacgtaattcggcccatgcacaacttacggcaaaccaaccccgtttccgccgcctttacgaaaaggagggaccaatttatcggcctgcaaaaatcgcgcggacgtaagtcgtttcggccgaaagctacgttttagattaacttttttgcgacaatgcgtagtttcgcgcagacgttgccgatgaaccgaaagccagtgttccggaattattctttcgccattcggccggccgaaatttcacgaccgttgtcgtttccggaagt

SEQ ID NO: 366 - DNA, 372 bp, synthetic construct
  source 1..372: mol_type = other DNA
aatagggctctcacttctttccgaccgtccttcggtttcgcttaccactaaccaacaaaatcgcctacaaaaatcatacatcttggcctcattaatgaaagaggcaatacgcggaaatcggtgttttccactcttacggaattgtcacgccatttcccccgcctttccgaaaaggggccggacgattctccgctggtaaaatagccgttccgaaatatagtggcggccgaaagcgacgtttcgcttaaaactcggcaaagttcggtagtatctattagtcggccattaacgaaaaaaccgggcgaggttccggaagctcgttttcggccttcgtcggtggccggaagtttcgcccttttcggttccggaatt

SEQ ID NO: 367 - DNA, 372 bp, synthetic construct
  source 1..372: mol_type = other DNA
acgaagtcccccggcactttgcgaccgtaaatgtactacacaaaaagttgacctacatttagataaagctgcatcataccgtaaaacggcattcctttacgttaaccattacggaattcggcctattctaagtttacgtcgtgccgacacaatatcagttattgttccgcctattcgcggaatttcggcttattcgtcaagtcatcggtgacgaaagtcgttgcggccgaaattttcgacaagtctgccgctcctgttcggggttcttcaaacacgaaagtttccggcctgagacgataacgcgggttttcgaaagtccggtttcgccgttgatcggcgagcggaagtttcgccgttctggtttccggaagt

SEQ ID NO: 368 - DNA, 372 bp, synthetic construct
  source 1..372: mol_type = other DNA
acgaaatagcggtgcaccccacagctttttcttggacagcacacatttaaaccaaaaaggattcccacacaattcacaggcaaatccgcctttaatgaaaaccccgttagacgaattccggcctactgtaaaatgacgttatacccatcacttttcccccgccattccggaaaacgaagtcgaaagcataatgtttccgcctacgcggccacgaaattgtttccggccgaaattgtcgatggatccttaccgcttcgatatccagtcgaattcataattccgccctaagcgaaagtcacgggcgggtttccggaagtacggtttcggccttcgtcggcggacggaagtctctccgttttcgtttacgaaagt

SEQ ID NO: 369 - DNA, 372 bp, synthetic construct
  source 1..372: mol_type = other DNA
acgaattaattcaagtcactacgcccgtgggggtggcaacccaaaatagatatcccgacactgcaaataccaatcatacgctaattgcgcacgaacgaaagcgccatgatacgaccttcggcctctccaaaaattacgcagaatcaacatacattatggtgactttccggaaaagcatgtcaaagctatcggcctgccacgttttcctccgacgttcaactttcgggcggaattgccgttctcgattctttatattacgccatccgaacgtcacggccccggaaggcgtacgctagagcttgatgtcgggaggaattacgccttttccattcggcgacgtccggaagtttcgccgtattcgtttccggaagt

SEQ ID NO: 370 - DNA, 100 bp, synthetic construct
  source 1..100: mol_type = other DNA
gccggtggcggcaggatacagcggcttctgcgcgacttataagagctccttgtgcggcgccattttaagcctctcggtctgtggcagcagcgttggcccg

SEQ ID NO: 371 - DNA, 335 bp, synthetic construct
  source 1..335: mol_type = other DNA
cgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaacta

SEQ ID NO: 372 - DNA, 351 bp, synthetic construct
  source 1..351: mol_type = other DNA
cgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactaccgcggatccgccacc

SEQ ID NO: 373 - DNA, 310 bp, synthetic construct
  source 1..310: mol_type = other DNA
tcgggagccgtggaggtacgaacttaagacatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacacc

SEQ ID NO: 374 - DNA, 265 bp, synthetic construct
  source 1..265: mol_type = other DNA
ggagaagagcatgcttgagggctgagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacag

SEQ ID NO: 375 - DNA, 383 bp, synthetic construct
  source 1..383: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgt

SEQ ID NO: 376 - DNA, 390 bp, synthetic construct
  source 1..390: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgcc

SEQ ID NO: 377 - DNA, 474 bp, synthetic construct
  source 1..474: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgggcgaagccggtccccgccccgcggggccccgccccgcgtgcgccgttccgaaagttgctttctagatctcgccccgccccggcggcgcctatataaggtgtgggcgggtgtggcacagctagttccgtcgcagccggcgagccgaacggtcgt

SEQ ID NO: 378 - DNA, 750 bp, synthetic construct
  source 1..750: mol_type = other DNA
tccatctttccagaaacttctctgagaaactcagcaaaaagcatccaatttcgccactgcttccccctccgcgcatgcgcgagacgccaagcggaaagcctttggcgcgccggagtgtgagagtggtgcgtgagcgcgcgcgcgctcgccgtgcgggggcggttctctctgacaatgaagggaaagaattccccctcccctcaacattccgtcctcgcgcaagcgcgtcggagccacttccttggcctttgagggaagggggaggggtcgtcccgaagcgcgctagtcgtgtttccgcgcgcctgcgcacggggccgttttcgtctcgctgtgttgtccgcgagtctttgacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactag

SEQ ID NO: 379 - DNA, 706 bp, synthetic construct
  source 1..706: mol_type = other DNA
taaacacgaccactttacgcagcttaacagaatgactttaatgcttcttcaagttttccattttcttccacagggctttgtttcgaaaaataacaaaatgaggtaataagttaatgtatgtacacgttataaacactgtgtcagtttacgatggcagcaacggaagttgttacaaagaaagtgactgcggggtggctggagctcagccgcccccccattgagaagattcgtcgtcctgagaagtcgcgtgctggaggtggagtgtgtctagaagcatttgcggtggacgatggaagggccggcctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactag

SEQ ID NO: 380 - DNA, 330 bp, synthetic construct
  source 1..330: mol_type = other DNA
taaacacgaccactttacgcagcttaacagaatgactttaatgcttcttcaagttttccattttcttccacagggctttgtttcgaaaaataacaaaatgaggtaataagttaatgtatgtacacgttataaacactgtgtcagtttacgatggcagcaacggaagttgttacaaagaaagtgactgcggggtggctggagctcagccgcccccccattgagaagattcgtcgtcctgagaagtcgcgtgctggaggtggagtgtgtctagaagcatttgcggtggacgatggaagggccggcctcccaatgatggcgcctgctgaattc

SEQ ID NO: 381 - DNA, 184 bp, synthetic construct
  source 1..184: mol_type = other DNA
taactgtagcctttctctctttccctgtagaaacctctgcgccatgagagccaaggtgagcggttcctggtagtaagcttgggaggtaggagttggcgagtagtagcgaggagacgaaggcaagtccgccatacctcctgaactactgggtttcaagggtgcccaagagctggtgggagagaga

SEQ ID NO: 382 - DNA, 694 bp, synthetic construct
  source 1..694: mol_type = other DNA
aaatatggatcaacaaaatacagtatatccataccctaaatatggatcaacaaaatacagtatatccatacactaaatatgaaaatacggtatatccatataataaatatgggtcaacaaaatacggtatatccataccctaaatatggatcaacaaaatacggtatatccatacactaaatatgggtcaacaaaatacagtatatccatacaataaatatgggtcaacaaaatacggtatatccatacactaaatatgggtcaacaaaatacagtatatccataaaataaataacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactag

SEQ ID NO: 383 - DNA, 318 bp, synthetic construct
  source 1..318: mol_type = other DNA
aaatatggatcaacaaaatacagtatatccataccctaaatatggatcaacaaaatacagtatatccatacactaaatatgaaaatacggtatatccatataataaatatgggtcaacaaaatacggtatatccataccctaaatatggatcaacaaaatacggtatatccatacactaaatatgggtcaacaaaatacagtatatccatacaataaatatgggtcaacaaaatacggtatatccatacactaaatatgggtcaacaaaatacagtatatccataaaataaataccaatgatggcgcctgctgaattc

SEQ ID NO: 384 - DNA, 350 bp, synthetic construct
  source 1..350: mol_type = other DNA
cgagtcttcaaaccttaagcctcagtgcaaaagggagcagcgaggagaggcggtgggattaaagaaaaaaattcttctctccctgtcacttcaaaaggtcgcccaaccagcgagcggggggtttacccacgactctggctcgcaccccttgcgggcctgcgcaggcggccccgtagcgcaagggagggcgggaaaggaaggggcggggacgcgagggcgaatctataagaagcgtcgttcagcgagttcgctgctcagaagcgccgagagcgcggccgggacggttggagaagaaggcggctcccggaagggggagagacaaactgccgtaacctctgccgttcaggaac

SEQ ID NO: 385 - DNA, 648 bp, synthetic construct
  source 1..648: mol_type = other DNA
caccaacacctttgccacaaacacattcatgtctaacaacacatcaattaccaacaccagcgccacgaacacattcacgtccatcaacacatcaaccaccaacaccatcagcacgagcacattcacatccaataacacatcaactaccaacaccagcaccatgaacacattcatgcccaacaacacgtaaacccctaacactgtcaccacaaacaccttacagccagcagaacgccagtcactaacaccatcgccatcagcacttcgtggttagcaacacctcagctgacgccaatgtcaccacaaacacctcatggccagaagcagctcaaccaccaacaccgttatacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaa

SEQ ID NO: 386 - DNA, 372 bp, synthetic construct
  source 1..372: mol_type = other DNA
caccaacacctttgccacaaacacattcatgtctaacaacacatcaattaccaacaccagcgccacgaacacattcacgtccatcaacacatcaaccaccaacaccatcagcacgagcacattcacatccaataacacatcaactaccaacaccagcaccatgaacacattcatgcccaacaacacgtaaacccctaacactgtcaccacaaacaccttacagccagcagaacgccagtcactaacaccatcgccatcagcacttcgtggttagcaacacctcagctgacgccaatgtcaccacaaacacctcatggccagaagcagctcaaccaccaacaccgttatccaatgatggcgcctgctgaattc

SEQ ID NO: 387 - DNA, 349 bp, synthetic construct
  source 1..349: mol_type = other DNA
aactaggacctcgtccccttctctatcccttcccctgatccatctcgtctctgttctccttcccttttccgttcccgaagccggaaggagccgaggtacccacggaaaaagtcgaagctgtccccggagagtgaggccttcacgaagcggtggcggaaggagccgaagttctctgatcggaggcgtgagcaagtggcatctccggaaagagccgaaacacggactcgagcttaatcccaggaggggcccgagggcgagatccggaaatctccggaaagagccgaaggccgggacggttaggattgtcggaagtggccgattgcttggacagggccggcggagaagatcg

SEQ ID NO: 388 - DNA, 750 bp, synthetic construct
  source 1..750: mol_type = other DNA
gatgcccaccgggatggagaaggaggaggaatcggtagggactcgagtgtttattacccccccttccctcctcccccctcccctttgtcagtcgggcctcgccattcacagagcgctttacgctcgctggagggcgccgctgccgctctaacgcacgggactctacgccggcggagtagcgtaaaccctctcggccggcgtagcgcccgcccgcggccgccatgttgttgcgtcccagtgttgtgattagctcgcagcgctgcttacgctgccgtaagggggccttgccgtaagcggcggccgggaatggcgcaggggatgcgctggccgctcttggcgtacggtctctgacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactag

SEQ ID NO: 389 - DNA, 739 bp, synthetic construct
  source 1..739: mol_type = other DNA
gcccgttagtcccggatcccagcggccgcttcaccactgagcccgagccgccgagggaattttccaaatgcaaatcaaacacccttggcgccccgcttcttcattaattaagaggaacgtttcgacaaaaccggcgtaatctttattaattacgcagcccttagcgcgccgttcgcatcaatcacgcccaaagcgaaagtgcagggggcgcctaatcccttcccagagcgcgtttggagaaggagccgcgtggccggcgtttccaccgtgacttccacctttctgggctcgcctggcactggctgctcagccggcgcgcagggaggcctttccggaaacacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactag

SEQ ID NO: 390 - DNA, 362 bp, synthetic construct
  source 1..362: mol_type = other DNA
gcccgttagtcccggatcccagcggccgcttcaccactgagcccgagccgccgagggaattttccaaatgcaaatcaaacacccttggcgccccgcttcttcattaattaagaggaacgtttcgacaaaaccggcgtaatctttattaattacgcagcccttagcgcgccgttcgcatcaatcacgcccaaagcgaaagtgcagggggcgcctaatcccttcccagagcgcgtttggagaaggagccgcgtggccggcgtttccaccgtgacttccacctttctgggctcgcctggcactggctgctcagccggcgcgcagggaggcctttccggaaaccaatgatggcgcctgctgaattc

SEQ ID NO: 391 - DNA, 349 bp, synthetic construct
  source 1..349: mol_type = other DNA
ttacactagcttaagaggtggaggcggagacgttacgctaccgctaagaggtggaggtggagacgttacgctggcgtaagaggtggcggtggagacgctacgctggcgtaagacgcggcggtggagacgctacgctggcgtaagaggcggcggtggagacgctacgctggcgtaagaggcggcggtggaggcgctacgctggcgtaagaggcggcggtggaggcgctacgctggcgtaagaggtggcggtggagacgtttcgctggcgtaagaggtggaggtggagacgttacgctggcgtaagaggtggaggtggagcgtccttactgcacagggtgtggagggaatg

SEQ ID NO: 392 - DNA, 173 bp, synthetic construct
  source 1..173: mol_type = other DNA
ggagccttggatcgcgtccctggaaagggacaccaaagatttccaattccggagagcgggcccgaggaagggtcactgctcgggcgcacgaaagctgtctaaggcttgggcgtatatggggaactctggcttttgccacgcacttttgggaatgggcaggagacctgcttcct

SEQ ID NO: 393 - DNA, 346 bp, synthetic construct
  source 1..346: mol_type = other DNA
ttacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacac

SEQ ID NO: 394 - DNA, 351 bp, synthetic construct
  source 1..351: mol_type = other DNA
gcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagcctcacgcatgctcttctccacctcagtgatgacgagagcgggcgggtgagggggcgggaacgcagcgatctctgggttctacgttagtgggagtttaacgacggtccctgggattccccaaggcaggggcgagtccttttgtatgaattactctcagctccggtcggggcggg

SEQ ID NO: 395 - DNA, 351 bp, synthetic construct
  source 1..351: mol_type = other DNA
acgtggtgtggaaagggttaaaactggcgctcggcgggatacagggctctaggtgccttaaagaacacgcggacacacttgtttggcttctgtcgcgctccgtagaatgagttccctgggcctcggtgggccacgcctccgcaaagaaggacaacgcgaagctgcccgcgtagtgcctgttgcactcgccaaggaccggcctggcagagatagaaaactcgcagtacgtttgcctcagaaccgcctcaatccggaaacagaaactccccaggcccggcactgcgggagccacctctttcgagggtgggagtgcacatgcgcacaggggacctgaaaaacccttaaataccg

SEQ ID NO: 396 - DNA, 351 bp, synthetic construct
  source 1..351: mol_type = other DNA
gtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgccgggtcggcgcgcgcgcacgcagcggccacaccctcgggcggccagcggctcgggcaggaagtgcgcgcaagcgcccgggagccgcggcgaccccaccccttccggccgagcccgccttcgccccagcccaggccgcggcaccccgggccccagaacgcacgcg

SEQ ID NO: 397 - DNA, 350 bp, synthetic construct
  source 1..350: mol_type = other DNA
agcaggaatgaggggtggagggaagggcctcatttctggggaaggcccagggaagggcctcatttctggggaaggcccagggaagggccagggtgcagagaagccaatgacagctggagagttgggactgctgccaggacagactagacggagactgctcttgggtgccacctagtgggaaccttctttactgtcatccctggagagatggacctgtgacgtccccaccggatgccccgcccaccatgggcatccccatcccaatcatccctcaccacccccaggctcgggtcgcgtccccccaggctttgatggacaagtggccatggaaagcatcctcagctgcccca

SEQ ID NO: 398 - DNA, 750 bp, synthetic construct
  source 1..750: mol_type = other DNA
gcgcgcttcggtgcccgcgcgcccccacactttctcgcgcgtcttcgccccgcccacccctcgccgctccctcacgttctccagtttcttcgcctcgcctaccgcgcgcctctcccgccctttgtcgccttccttcgctccgcccgccccttccttccgtgcgcaccttcgccccgcccaccccactgtcctcccgccgtttctctcgcgcttccattcggcaaacccgtaagctcccgcgttcctctgcgcgcctttcgtccctcccacctgtgcgctcacgtcctccctcgcgtgcggcgctcagtttccggccctacccgtgggcgtcggcgcctagtggcgctcttacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactag

SEQ ID NO: 399 - DNA, 750 bp, synthetic construct
  source 1..750: mol_type = other DNA
aaagccctgtggcccacgcttccggcctcgaccgactacgtcatcaccaaaccccgtgatacctcactccaccttcaggtcctccgtcccaacggttccgtgtcctggaactttccacgttcctgcgcacgcgctctgcggcccttaacgtcccgacttcgccctcctctggccacgcccctgcccgttaagggccctgccacccccgtgacgtacctatcttccctcgggcaacctcgcaacagatctctgattggctgacccaggaagcggactttgcgaggcggcggcgacgataggctggcagccggccgacttccgcagaaggtgattggtcggcgctgctgtccacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactag

SEQ ID NO: 400 - DNA, 164 bp, synthetic construct
  source 1..164: mol_type = other DNA
agccacagccctctcagcgtctttccggggttggacacgccaccctctgcctcttctcgaaacagcacagagcgcgaggcgggccacccgctcctccctcgggtgtgggcagctccgaaactcacaccggggaggcgtggagagagccccgtcgcccgctaacc

SEQ ID NO: 401 - DNA, 351 bp, synthetic construct
  source 1..351: mol_type = other DNA
actttgatctcgttggggtcgaacttcggcggcatggtggaggcggctggtgtcggatgaacccggattcgggacgaccgaaggaagttgcaccttggcctcctccgagccgaaagccgagaggccggaaatcgcgcggacaagccagatataggcaggaaaacgcggctatcgcgagaactgtcgtcactgcccggccttccagagacgcccttgtcgccatgtttgttgtgggcaggcgcctgaggctgacggctggcaagcagggcaccgcggtgcgctgaagctagagattccgaaagggggttcgggtagttcgctccggagaagtctgagaagggtggctccggt

SEQ ID NO: 402 - DNA, 102 bp, synthetic construct
  source 1..102: mol_type = other DNA
ccagctgttggggtgagtactccctctcaaaagcgggcattacttctgcgctaagattgtcagtttccaaaaacgaggaggatttgatattcacctggcccg

SEQ ID NO: 403 - DNA, 203 bp, synthetic construct
  source 1..203: mol_type = other DNA
cagacatttggagaagtatgcagaacatcttggcagttttcctttaactctaagggttgtaaatacaaagtagaatgtgttaccatgagccatttcataggaagttgactttatttttttagaattcagaactttgcttctaaaaaggtaactttgcctcttgattctcatttttttccctaacttaggcaccttggaagttg

SEQ ID NO: 404 - DNA, 352 bp, synthetic construct
  source 1..352: mol_type = other DNA
cccgcgtcccggaaggtgagtgggggaggggagatggagcctggggctgccgagaagcagggagagaggggaaaggtctgcaggcctctgggcgcgggggacctttatcaaagggagcggcgaggggaagggagggaccatacgcaggcggagaagaggtagctggggatcttaaggattttgtagggaaaagccttagcggacccttgggtgtagaagggacgggccgtggcggttgcagactgggagggcctgacgggaaagggaaaggttgggcccctgtggacgccacagcgcgccttccccatccgcaaaggctttccctccctctcctccagctccagcctgaacc

SEQ ID NO: 405 - DNA, 338 bp, synthetic construct
  source 1..338: mol_type = other DNA
ctctgccctgccagggtaggtctgggaatcgggggcctgctgcgggaggtggaggcccaagggaggccccccggggactgtgtgtctcacccccgtccctgctacgttgtgttgttgtgtgatcccatcgtggaggttgttttggtgacactgtgtccccacgaagctggggatacccgtttctctagcttggagccaccaagatagaggacaaacacttctgtgattcagtccccagactgtctctgacttaatcccttgggttcaagccctttgtgggagagcaagggcacacactgcctaatccgtggtgtcccccccaggacaattgcgtctct

SEQ ID NO: 406 - DNA, 193 bp, synthetic construct
  source 1..193: mol_type = other DNA
tctctgcgcggcacggtgagcgcggccagggcaggacggggacgggcggagcagggacggccaccccaggagatgccaggggagaagggccagggacgaggacctgggtcgagggcggggcctgaacgttacccccgggctagaggcggggcctggggccgggggccgggcctcgaaacctccccaagtcggg

SEQ ID NO: 407 - DNA, 294 bp, synthetic construct
  source 1..294: mol_type = other DNA
cccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccacctcagtccgcctggagacctcgagccgagtggtcgtgcctccatagaa

SEQ ID NO: 408 - DNA, 160 bp, synthetic construct
  source 1..160: mol_type = other DNA
tgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtcta

SEQ ID NO: 409 - DNA, 880 bp, synthetic construct
  source 1..880: mol_type = other DNA
cctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtg

SEQ ID NO: 410 - DNA, 506 bp, synthetic construct
  source 1..506: mol_type = other DNA
cctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtg

SEQ ID NO: 411 - DNA, 250 bp, synthetic construct
  source 1..250: mol_type = other DNA
ggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattctttttcgcaacgggtttgccgccagaacacag

SEQ ID NO: 412 - DNA, 237 bp, synthetic construct
  source 1..237: mol_type = other DNA
ccatcccccaggtaggccaggaggcctgggcgggacctgcttggggttaaatactccggcccagcactccccaggcctctagtcccagtaggaggtttgactaagatcaatcttggtaagtgccttggccatctccccttgggtgggacatcttcagtaacgcttccagaagcaccttgtgttggaacctccaagtctcactgtccttctttttctccttagacgacttacagtgac

SEQ ID NO: 413 - DNA, 447 bp, synthetic construct
  source 1..447: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgca

SEQ ID NO: 414 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 415 - DNA, 447 bp, synthetic construct; identical to SEQ ID NO: 413
  source 1..447: mol_type = other DNA

SEQ ID NO: 416 - DNA, 836 bp, synthetic construct
  source 1..836: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 417 - DNA, 228 bp, synthetic construct
  source 1..228: mol_type = other DNA
ggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgatttgcctgaccctgcttgctcaactcta

SEQ ID NO: 418 - DNA, 240 bp, synthetic construct
  source 1..240: mol_type = other DNA
atccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactct

SEQ ID NO: 419 - DNA, 269 bp, synthetic construct
  source 1..269: mol_type = other DNA
ggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatc

SEQ ID NO: 420 - DNA, 281 bp, synthetic construct
  source 1..281: mol_type = other DNA
ctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatc

SEQ ID NO: 421 - DNA, 112 bp, synthetic construct
  source 1..112: mol_type = other DNA
aacggtggagggcagtgtagtctgagcagtactcgttgctgccgcgcgcgccaccagacataatagctgacagactaacagactgttcctttccatgggtcttttctgcagt

SEQ ID NO: 422 - DNA, 228 bp, synthetic construct
  source 1..228: mol_type = other DNA
aggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcagg

SEQ ID NO: 423 - DNA, 135 bp, synthetic construct
  source 1..135: mol_type = other DNA
ggtaagtatcaaggttacaagacaggtttaaggagaccaatagaaactgggcttgtcgagacagagaagactcttgcgtttctgataggcacctattggtcttactgacatccactttgcctttctctccacagg

SEQ ID NO: 424 - DNA, 943 bp, synthetic construct
  source 1..943: mol_type = other DNA
gtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 425 - DNA, 383 bp, synthetic construct
  source 1..383: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 426 - DNA, 228 bp, synthetic construct
  source 1..228: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 427 - DNA, 287 bp, synthetic construct
  source 1..287: mol_type = other DNA
tgttgagtgagtcttgcttccccagggggctggcgggctgggctgggggcaggcttccccaaccccagcccaggaagggacagggaatagcttcattccctgacttcatcaaccagctgacctactgactccctctccctcccttcctccctccccttcccttcctccctccgctccttgcttccctccccctccctccctttctgcccttctctctcctctttctcccctttcccatctccctctgccttcccctggcactctctctctagaacgggcagctgctg

SEQ ID NO: 428 - DNA, 142 bp, synthetic construct
  source 1..142: mol_type = other DNA
ggtaagtcactgactgtctatgcctgggaaagggtgggcaggagatggggcagtgcaggaaaagtggcactatgaaccctgcagccctaggaatgcatctagacaattgtactaaccttcttctctttcctctcctgacagg

SEQ ID NO: 429 - DNA, 195 bp, synthetic construct
  source 1..195: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 430 - DNA, 197 bp, synthetic construct
  source 1..197: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgaca

SEQ ID NO: 431 - DNA, 207 bp, synthetic construct
  source 1..207: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccggctcgagccgagtggtcgtcttgtttgtggatccctgtgatcgtcacttgacaggatccgcca

SEQ ID NO: 432 - DNA, 15 bp, synthetic construct
  source 1..15: mol_type = other DNA
caactcctaaaaaac

SEQ ID NO: 433 - DNA, 21 bp, synthetic construct
  source 1..21: mol_type = other DNA
aactcctaaaaaaccgccacc

SEQ ID NO: 434 - DNA, 315 bp, synthetic construct
  source 1..315: mol_type = other DNA
tctgggcctacggagccagcacccatcgccaaaactcttcatcctcttcctcaatctcgctttctctctcgctttttttttttttcttcttctttttttttttttttttcaaaaggaggggagagggggtaaaaaaatgctgcactgtgcggcgaggccggtgagtgagcgacgcggagccaatcagcgcccgccgttccgaaagttgccttttatggctcgagtggccgctgtggcgtcctataaaacccggcggcgcaacgcgcagtcagtccgcctggagacctcgagccgagtggtcgtgcctccatagaa

SEQ ID NO: 435 - DNA, 229 bp, synthetic construct
  source 1..229: mol_type = other DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 436 - DNA, 617 bp, synthetic construct
  source 1..617: mol_type = other DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 437 - DNA, 218 bp, synthetic construct
  source 1..218: mol_type = other DNA
gtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 438 - DNA, 81 bp, synthetic construct
  source 1..81: mol_type = other DNA
aggtctatataagcagagctcgtttagtgaaccgtcagtccgcctggagacctcgagccgagtggtcgtgcctccatagaa

SEQ ID NO: 439 - DNA, 197 bp, synthetic construct
  source 1..197: mol_type = other DNA
gaaatcgggcggagttagggcggagccaatcagcgtgcgccgttccgaaagttgccttttatggctgggcggaaaatgggcggtgaacgccgatgattatataaggacgcgccggttgtggcacagctagttccgtcgcagccggctcgagccgagtggtcgtcttgtttgtggatcggtgtgatcgtcacttgaca

SEQ ID NO: 440 - DNA, 265 bp, synthetic construct; identical to SEQ ID NO: 360
  source 1..265: mol_type = other DNA

SEQ ID NO: 441 - DNA, 16 bp, synthetic construct
  source 1..16: mol_type = other DNA
cgagccgagtggtcgt

SEQ ID NO: 442 - DNA, 18 bp, synthetic construct
  source 1..18: mol_type = other DNA
ctcgagccgagtggtcgt

SEQ ID NO: 443 - DNA, 176 bp, synthetic construct
  source 1..176: mol_type = other DNA
aagaggtaagggtttaagggatggttggttggtggggtattaatgtttaattacctggagcaggtctatataagcagagctcgtttagtgaaccgtcagatcgcctagatacgccatccacgctgttttgacctccatagaagacacctgcctgaaatcactttttttcaggttgg

SEQ ID NO: 444 - DNA, 92 bp, synthetic construct
  source 1..92: mol_type = other DNA
aagaggtaagggtttaagggatggttggttggtggggtattaatgtttaattacctggagcacctgcctgaaatcactttttttcaggttgg

SEQ ID NO: 445 - DNA, 388 bp, synthetic construct
  source 1..388: mol_type = other DNA
aagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 446 - DNA, 713 bp, synthetic construct
  source 1..713: mol_type = other DNA
aagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaaagtaattctccttggaatttgccctttttgagtttggatcttggttcattctcaagcctcagacagtggttcaaagtttttttcttccatttcaggtgtcgtga

SEQ ID NO: 447 - DNA, 579 bp, synthetic construct
  source 1..579: mol_type = other DNA
gtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgagtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaaagtaattctccttggaatttgccctttttgagtttggatcttggttcattctcaagcctcagacagtggttcaaagtttttttcttccatttcaggtgtcgtga

SEQ ID NO: 448 - DNA, 176 bp, synthetic construct; identical to SEQ ID NO: 443
  source 1..176: mol_type = other DNA

SEQ ID NO: 449 - DNA, 304 bp, synthetic construct
  source 1..304: mol_type = other DNA
gttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgag

SEQ ID NO: 450 - DNA, 303 bp, synthetic construct
  source 1..303: mol_type = other DNA
gttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcga

SEQ ID NO: 451 - DNA, 692 bp, synthetic construct
  source 1..692: mol_type = other DNA
gttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgagaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 452 - DNA, 116 bp, synthetic construct
  source 1..116: mol_type = other DNA
gttctctcgttaacttaatgagacagatagaaactggtcttgtagaaacagagtagtcgcctgcttttctgccaggtgctgacttctctcccctgggcttttttctttttctcagg

SEQ ID NO: 453 - DNA, 345 bp, synthetic construct
  source 1..345: mol_type = other DNA
cgtgcccgggctccggtgagtcagggcgcgttatgcaagtgcccccggcgcctccccttcggtctttcaccccgcgcggttacgaaagcgcgaccccctccccccggcgctataaagcagcggggcggccgcggcgcgctcgcctccctcgctccacgcgcgcccggactcggcggccaggcttgcgcgcggttcccctcccggtggtgagtcgcggccggggcctcggggccggcggcgggttgggccggggtggggtggggtggggtgaggtgggatacggcggcgggggtcggctgccgggcactgacggaggcgcgtctcccgcagggcggattcctgggc

SEQ ID NO: 454 - DNA, 576 bp, synthetic construct
  source 1..576: mol_type = other DNA
cgaagtaacttctcggtaaagtcttcacggaatttccagaccacacttgcccactgggaggcttttaggacccgagacgtgtgcaggcttttccaggttcgggttggtgggccttattcattttggcggggctatacgtattcactcttaagcctgtagaagatgaacgttgattgtaccttctaaaacgtgaggttatagttaagagaattgcgttgtcggtctgctcagcagagatgtaagtccagtcttgatttcctcaggcactacttgaggagttgggagagtgtaactaaaaggtcttataactaggacaagtgtttttgatgcaaaagtctttagtccgtaaacatatacagaaaacacccgaataaaacaaaaaataaaaagtttcgagcttcggttgataggcccttccgatcaaacagtttttgttgaagttgcacaacatgacactgcctatgatcattctagactatcaagataaaatcattgcggttatttgtttggaagctggatgagattttggtcatttaagagtctttttaactttttatttagccaaattgaagttta

SEQ ID NO: 455 - DNA, 397 bp, synthetic construct
  source 1..397: mol_type = other DNA
gcctgaagggcgggacaggaagtgaggtcactcctcaccggggtgaaaggttagcggaagtgtccttctttcctttttgctggtagggccgggtggttgctgccggtaagtagaagcttgggttgaatctttcaatccgctgccatccgcggcttgggggtcgaaggtccgggcggcgccgggtgcattccttccctaggtctctggcggctacgtatccgatcctgtcctgcgagcagggccctcgggcggccttgaccaaggcgacagtgaacacagtctgaagttccggcccaagacctgggcttgctggcaggagtcagctctgggcatctttggctttacggatttttaagtggccctttctccttgctctctgcagaaaggggcaagttca

SEQ ID NO: 456 - DNA, 204 bp, synthetic construct
  source 1..204: mol_type = other DNA
gcgattgtgcgttgtgtaagccacggcgcagggtggggcgcgggcgggacttgggcgggcggggtgggcttggccgagctggcctccggggcaccgaccgctataaggccagtcggactgcgacacagcccatcccctcgaccgctcgcgtcgcatttggccgcctccctaccggtgagttctctccaggagccctgggtactt

SEQ ID NO: 457 - DNA, 154 bp, synthetic construct
  source 1..154: mol_type = other DNA
ggcgaagccggtccccgccccgcggggccccgccccgcgtgcgccgttccgaaagttgctttctagatctcgccccgccccggcggcgcctatataaggtgtgggcgggtgtggcacagctagttccgtcgcagccggcgagccgaacggtcgt

SEQ ID NO: 458 - DNA, 950 bp, synthetic construct
  source 1..950: mol_type = other DNA
cgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 459 - DNA, 329 bp, synthetic construct
  source 1..329: mol_type = other DNA
cgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 460 - DNA, 876 bp, synthetic construct
  source 1..876: mol_type = other DNA
tgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagatcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaataatacgactaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 461 - DNA, 816 bp, synthetic construct
  source 1..816: mol_type = other DNA
tgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaataatacgactaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 462 - DNA, 944 bp, synthetic construct
  source 1..944: mol_type = other DNA
tgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 463 - DNA, 1026 bp, synthetic construct
  source 1..1026: mol_type = other DNA
tgccgccagaacacaggtaagtgccgtgtgtggttcccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatttcagcaccgcggacagtgccagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 464 - DNA, 989 bp, synthetic construct
  source 1..989: mol_type = other DNA
tgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 465 - DNA, 1324 bp, synthetic construct
  source 1..1324: mol_type = other DNA
tgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtaatacgactaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatcgaggtgagccccacgttctgcttcactctccccatctcccccccctccccacccccaattttgtatttatttattttttaattattttgtgcagcgttgggggcggggggggggggggcgcgcgccaggcggggcggggcggggcgaggggcggggcggggcgaggcggagaggtgcggcggcagccaatcagagcggcgcgctccgaaagtttccttttatagcgaggcggcggcggcggcggccctataaaaagcgaagcgcgcggcgggcgggtcagtccgcctggagacctcgagccgagtggtcgtgcctccatagaaagtaattctccttggaatttgccctttttgagtttggatcttggttcattctcaagcctcagacagtggttcaaagtttttttcttccatttcaggtgtcgtga

SEQ ID NO: 466 - DNA, 1264 bp, synthetic construct
  source 1..1264: mol_type = other DNA
tgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtaatacgactaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgagtgatccggttttggcagtacatcattgggcgtggatagcggtttgactcacggggatttccaagtctccaccccattgacgtcattgggagtttgttttggcaccaaaatcaacgggactttccaaattgtcgtaacaactccgccccattgacgcaattgggcggtaggcgtgtacggtgggaggtctatataagcagagctcgtttagtgaaccgtcagtccgcctggagacctcgagccgagtggtcgtgcctccatagaaagtaattctccttggaatttgccctttttgagtttggatcttggttcattctcaagcctcagacagtggttcaaagtttttttcttccatttcaggtgtcgtga

SEQ ID NO: 467 - DNA, 725 bp, synthetic construct
  source 1..725: mol_type = other DNA
tgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 468 - DNA, 770 bp, synthetic construct
  source 1..770: mol_type = other DNA
tgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagaagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 469 - DNA, 800 bp, synthetic construct
  source 1..800: mol_type = other DNA
tgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 470 - DNA, 806 bp, synthetic construct
  source 1..806: mol_type = other DNA
cgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgaggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 471 - DNA, 845 bp, synthetic construct
  source 1..845: mol_type = other DNA
tgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctcgagaagaggtaagggtttaagggatggttggttggtggggtattaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 472 - DNA, 336 bp, synthetic construct
  source 1..336: mol_type = other DNA
tgccgccagaacacaggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaacta

SEQ ID NO: 473 - DNA, 521 bp, synthetic construct
  source 1..521: mol_type = other DNA
tgccgccagaacacaggtaagtgccgtgtgtggttcccgccgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatggaggacgcggcgctcgggagagcgggcggggcagttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaacta

SEQ ID NO: 474 - DNA, 443 bp, synthetic construct
  source 1..443: mol_type = other DNA
tgccgccagaacacaggtaagtgccgtgtgtggttcccgccgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggagctcaaaatggaggacgcggcgctcgggagagcgggcggggcagtggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaacta

SEQ ID NO: 475 - DNA, 83 bp, synthetic construct
  source 1..83: mol_type = other DNA
ggcgaagccggtccccgccccgcggggccccgccccgcgtgcgccgttccgaaagttgctttctagatctcgccccgccccgg

SEQ ID NO: 476 - DNA, 524 bp, synthetic construct
  source 1..524: mol_type = other DNA
agcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaagggggcgcggcaggagggggcaccagggagcggagccctggccctcctgacgtcctgcccgcccacgcgtccgcaggtgaggtgcatcctgacaggtcacgagctgccctgccgcctgccggagctccaggtctacacccgcggcaaaaagtaccagcggctggtccgcgcctccccggccttcgactatgcagagttcgagccgcacatcgtgcccagcaccaagaacccgtaggtggtc

SEQ ID NO: 477 - DNA, 761 bp, synthetic construct
  source 1..761: mol_type = other DNA
gcacacgaccacaattccactgaaagcattttaatacggaacttgtcactcccagggagcctccgctcagccggcagttggttcatttcaatccccacgacaacccttcaaagtgcagggcagacagcaggtggctctgcccaggcgcctggatcacagcccggcctgcagccctcacctgggcgcggggagaccctgaggacgctcctccaggcggcgctggccggggcctgcggacacggacgggcgggctgagctccgggacccctccccgcgccccgcaccccgcaccccgcaccccgcaccccgcacccggcgctcacccgtcccagccccgccgcccgcagccccagctgcaacgcagccaccgccgccatcgcacccggccccgcgggcgcttccgggacgcaggaggcatctgcatccggggcgccgctgagtcccgcccagagccccgcccccggctccaggttctgcgagcggcttccgccgggctgctccgcgggcgcgtcggccatgagcgagttgccgggcgacgtgcgggcgtttctgcgggagcacccgagcctgcggctccagacggacgcccgcaaggttcgcagcgcgggaggggaacggagtggcggagaagggcgcagttgggatgaggggctgaggggagggcaggggagaggagagggcaggggagaggggagaggggagagcaggagagaggggaaggcaggggagagggcgcggcgggatcaggggaggagagggaa

SEQ ID NO: 478 - DNA, 211 bp, synthetic construct
  source 1..211: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgccca

SEQ ID NO: 479 - DNA, 212 bp, synthetic construct
  source 1..212: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccag

SEQ ID NO: 480 - DNA, 99 bp, synthetic construct
  source 1..99: mol_type = other DNA
gtaagtttagtctttttgtcttttatttcaggtcccggatccggtggtggtgcaaatcaaagaactgctcctcagtggatgttgcctttacttctaggc

SEQ ID NO: 481 - DNA, 329 bp, synthetic construct
  source 1..329: mol_type = other DNA
gtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactcgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 482 - DNA, 100 bp, synthetic construct
  source 1..100: mol_type = other DNA
cttgtctcgctccggggaacgctcggaaactcccggccgccgccacccgcgtctgttctgttacacaagggaagaaaagccgctgccgcactccgagtgt

SEQ ID NO: 483 - DNA, 100 bp, synthetic construct
  source 1..100: mol_type = other DNA
cattctgtggtctgatcatcctgtggtttcgtcgccgccatcctcgtcgcgacacgctgttttcggttctcggcccgacgagccatcgccatcctacagc

SEQ ID NO: 484 - DNA, 100 bp, synthetic construct
  source 1..100: mol_type = other DNA
cactcgcgctgccatcactcttccgccgtcttcgccgccatcctcggcgcgactcgcttctttcggttctaccaggtagagtccgccgccatcctccacc

SEQ ID NO: 485 - DNA, 12 bp, synthetic construct
  source 1..12: mol_type = other DNA
caagatggcggc

SEQ ID NO: 486 - DNA, 315 bp, synthetic construct
  source 1..315: mol_type = other DNA
gaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaa

SEQ ID NO: 487 - DNA, 515 bp, synthetic construct
  source 1..515: mol_type = other DNA
gccgggctggtagcccagcggggggcgcggcagggcgaggagcagcccgcgctgctcccgcccctcgggtgccagcaccgcccctgctgcggcgggtgaggggcggggcggggcgcggcgtatataaggctaggggcgggcgccgctcttcagtccgcctggagacctcgagccgagtggtcgtcaggatctcgggctcggaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaa

SEQ ID NO: 488 - DNA, 318 bp, synthetic construct
  source 1..318: mol_type = other DNA
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcga

SEQ ID NO: 489 - DNA, 100 bp, synthetic construct
  source 1..100: mol_type = other DNA
gcattccaaccttccagcctgcgacctgcggagaaaaaaaattacttattttcttgccccatacataccttgaggcgagcaaaaaaattaaattttaacc

SEQ ID NO: 490 - DNA, 176 bp, synthetic construct
  source 1..176: mol_type = other DNA
cgctgcgcacggggggttcgtacccatctggccgcgaccgtccgtttccccctcgcttggttctgcccctgctccccctgcacaggcctcacagtgcgtctggccggcgctttatagctgcagcctgggcggctccgctagctgtttttcgtcttccctaggctatttctgccggg

SEQ ID NO: 491 - DNA, 206 bp, synthetic construct
  source 1..206: mol_type = other DNA
cattctgtggtctgatcatcctgtggtttcgtcgccgccatcctcgtcgcgacacgctgttttcggttctcggcccgacgagccatcgccatcctacagccaacaacttgtctcgctccggggaacgctcggaaactcccggccgccgccacccgcgtctgttctgttacacaagggaagaaaagccgctgccgcactccgagtgt

SEQ ID NO: 492 - DNA, 206 bp, synthetic construct
  source 1..206: mol_type = other DNA
cactcgcgctgccatcactcttccgccgtcttcgccgccatcctcggcgcgactcgcttctttcggttctaccaggtagagtccgccgccatcctccacccaacaacttgtctcgctccggggaacgctcggaaactcccggccgccgccacccgcgtctgttctgttacacaagggaagaaaagccgctgccgcactccgagtgt

SEQ ID NO: 493 - DNA, 206 bp, synthetic construct; identical to SEQ ID NO: 492
  source 1..206: mol_type = other DNA

SEQ ID NO: 494 - DNA, 231 bp, synthetic construct
  source 1..231: mol_type = other DNA
ggagacctcgagccgagtggtcgtcactcgcgctgccatcactcttccgccgtcttcgccgccatcctcggcgcgactcgcttctttcggttctaccaggtagagtccgccgccatcctccacccaacaacttgtctcgctccggggaacgctcggaaactcccggccgccgccacccgcgtctgttctgttacacaagggaagaaaagccgctgccgcactccgagtgtg

SEQ ID NO: 495 - DNA, 206 bp, synthetic construct
  source 1..206: mol_type = other DNA
cttgtctcgctccggggaacgctcggaaactcccggccgccgccacccgcgtctgttctgttacacaagggaagaaaagccgctgccgcactccgagtgtcaacaacactcgcgctgccatcactcttccgccgtcttcgccgccatcctcggcgcgactcgcttctttcggttctaccaggtagagtccgccgccatcctccacc

SEQ ID NO: 496 - DNA, 294 bp, synthetic construct
  source 1..294: mol_type = other DNA
gctagctcgggccgggaggagccgcagccggaggagggggaggaggaagaagagaaggaagaggagagggggccgcagtggcgactcggcgctcggaagccgggctcgtggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaacc

SEQ ID NO: 497 - DNA, 197 bp, synthetic construct; identical to SEQ ID NO: 430
  source 1..197: mol_type = other DNA

SEQ ID NO: 498 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 499 - DNA, 217 bp, synthetic construct
  source 1..217: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaacc

SEQ ID NO: 500 - DNA, 264 bp, synthetic construct
  source 1..264: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgcagcaaatgggcggtaggcgtgtacggtgggaggtctatataagcagagctcgtttagtgaaccgtcagatcgcctggagacgccatccacgctgttttgacctccataga

SEQ ID NO: 501 - DNA, 294 bp, synthetic construct
  source 1..294: mol_type = other DNA
cccgggcccagcaccccaaggcggccaacgccaaaactctccctcctcctcttcctcaatctcgctctcgctctttttttttttcgcaaaaggaggggagagggggtaaaaaattgctgcactgtgcggcgaagccggtgagtgagcggcgcggggccaatcagcgtgcgccgttccgaaagttgccttttaaggctcgagcggccgcggcggcgccctataaaacccagcggcgcgacgcgccacctcagtccgcctggagacctcgagccgagtggtcgtgcctccatagaa

SEQ ID NO: 502 - DNA, 228 bp, synthetic construct
  source 1..228: mol_type = other DNA
ggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactcta

SEQ ID NO: 503 - DNA, 197 bp, synthetic construct
  source 1..197: mol_type = other DNA
gaattcgggcggagttagggcggagccaatcagcgtgcgccgttccgaaagttgcctttattggctgggcggagattgggcggtgaacgccgttgattatataaggacgcgccgggtgtggcacagctagttccgtcgcagccggctcgagccgagtggtcgtcttgtttgtggatccctgtgatcgtcacttgaca

SEQ ID NO: 504 - DNA, 315 bp, synthetic construct; identical to SEQ ID NO: 297
  source 1..315: mol_type = other DNA

SEQ ID NO: 505 - DNA, 81 bp, synthetic construct; identical to SEQ ID NO: 438
  source 1..81: mol_type = other DNA